SEQUENCE LISTING (123 sequences)

SEQ ID NO: 1 - protein, 15 aa, synthetic construct
RASKSVSFLGINLIY

SEQ ID NO: 2 - protein, 7 aa, synthetic construct
QASNRDT

SEQ ID NO: 3 - protein, 9 aa, synthetic construct
LQTPFFPRT

SEQ ID NO: 4 - protein, 5 aa, synthetic construct
DYSIN

SEQ ID NO: 5 - protein, 17 aa, synthetic construct
WINTETGNPTYAQGFTG

SEQ ID NO: 6 - protein, 8 aa, synthetic construct
DKHLAMDY

SEQ ID NO: 7 - protein, 111 aa, synthetic construct
DIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIK

SEQ ID NO: 8 - protein, 117 aa, synthetic construct
QVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSS

SEQ ID NO: 9 - protein, 248 aa, synthetic construct
DIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKGGGGSGGGGSGGGGSGGGGSQVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSS

SEQ ID NO: 10 - protein, 15 aa, synthetic construct
RASQSVDYDGDSYLN

SEQ ID NO: 11 - protein, 7 aa, synthetic construct
DASNLVS

SEQ ID NO: 12 - protein, 9 aa, synthetic construct
QQSTEDPWT

SEQ ID NO: 13 - protein, 5 aa, synthetic construct
SYWMN

SEQ ID NO: 14 - protein, 17 aa, synthetic construct
QIWPGDGDTNYNGKFKG

SEQ ID NO: 15 - protein, 15 aa, synthetic construct
RETTTVGRYYYAMDY

SEQ ID NO: 16 - protein, 112 aa, synthetic construct
EIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLG

SEQ ID NO: 17 - protein, 124 aa, synthetic construct
EVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 18 - protein, 256 aa, synthetic construct
EIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLGGGGGSGGGGSGGGGSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 19 - protein, 14 aa, synthetic construct
GSSTGAVTTSNYAN

SEQ ID NO: 20 - protein, 7 aa, synthetic construct
GTNKRAP

SEQ ID NO: 21 - protein, 9 aa, synthetic construct
ALWYSNHWV

SEQ ID NO: 22 - protein, 5 aa, synthetic construct
TYAMN

SEQ ID NO: 23 - protein, 19 aa, synthetic construct
RIRSKYNNYATYYADSVKG

SEQ ID NO: 24 - protein, 14 aa, synthetic construct
HGNFGDSYVSWFAY

SEQ ID NO: 25 - protein, 110 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLG

SEQ ID NO: 26 - protein, 125 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 27 - protein, 215 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 228 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 29 - protein, 481 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKGGGGSGGGGSGGGGSGGGGSQVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSS

SEQ ID NO: 30 - protein, 476 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLGGGGGSGGGGSGGGGSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 31 - protein, 5 aa, synthetic construct
GGGGS

SEQ ID NO: 32 - protein, 10 aa, synthetic construct
GGGGSGGGGS

SEQ ID NO: 33 - protein, 15 aa, synthetic construct
GGGGSGGGGSGGGGS

SEQ ID NO: 34 - protein, 20 aa, synthetic construct
GGGGSGGGGSGGGGSGGGGS

SEQ ID NO: 35 - protein, 21 aa, synthetic construct
GGGGGSGGGGSGGGGSGGGGS

SEQ ID NO: 36 - protein, 21 aa, synthetic construct
GGGGSGGGGSGGGGSGGGGAS

SEQ ID NO: 37 - protein, 5 aa, synthetic construct
EPKSC

SEQ ID NO: 38 - protein, 25 aa, synthetic construct
  VARIANT 1..25: note = This sequence may encompass 1-5 GGGGS repeating units
GGGGSGGGGSGGGGSGGGGSGGGGS

SEQ ID NO: 39 - protein, 5 aa, synthetic construct
DGGGS

SEQ ID NO: 40 - protein, 5 aa, synthetic construct
TGEKP

SEQ ID NO: 41 - protein, 4 aa, synthetic construct
GGRR

SEQ ID NO: 42 - protein, 14 aa, synthetic construct
EGKSSGSGSESKVD

SEQ ID NO: 43 - protein, 18 aa, synthetic construct
KESGSVSSEQLAQFRSLD

SEQ ID NO: 44 - protein, 8 aa, synthetic construct
GGRRGGGS

SEQ ID NO: 45 - protein, 9 aa, synthetic construct
LRQRDGERP

SEQ ID NO: 46 - protein, 12 aa, synthetic construct
LRQKDGGGSERP

SEQ ID NO: 47 - protein, 18 aa, synthetic construct
GSTSGSGKPGSGEGSTKG

SEQ ID NO: 48 - protein, 489 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLGGGGGSGGGGSGGGGSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 49 - protein, 215 aa, synthetic construct; identical to SEQ ID NO: 27

SEQ ID NO: 50 - protein, 481 aa, synthetic construct; identical to SEQ ID NO: 29

SEQ ID NO: 51 - protein, 504 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 52 - protein, 448 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 214 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

SEQ ID NO: 54 - protein, 452 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 55 - protein, 215 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 57 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 58 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 59 - protein, 98 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 60 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 61 - protein, 15 aa, Homo sapiens
EPKSCDKTHTCPPCP

SEQ ID NO: 62 - protein, 12 aa, Homo sapiens
ERKCCVECPPCP

SEQ ID NO: 63 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 64 - protein, 12 aa, Homo sapiens
ESKYGPPCPSCP

SEQ ID NO: 65 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 66 - protein, 109 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 67 - protein, 110 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 68 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 69 - protein, 106 aa, Homo sapiens [sequence omitted]

SEQ ID NO: 70 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 106 aa, synthetic construct
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPG

SEQ ID NO: 72 - protein, 106 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 73 - protein, 106 aa, Homo sapiens
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 74 - protein, 106 aa, Homo sapiens
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 75 - protein, 106 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 107 aa, Homo sapiens [sequence omitted]

SEQ ID NO: 77 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 78 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 80 - protein, 106 aa, Homo sapiens
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 81 - protein, 106 aa, Homo sapiens
GQPKAAPSVTLFPPSSEELQANKATLVCLVSDFNPGAVTVAWKADGSPVKVGVETTKPSKQSNNKYAASSYLSLTPEQWKSHRSYSCRVTHEGSTVEKTVAPAECS

SEQ ID NO: 82 - protein, 112 aa, synthetic construct
DIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKG

SEQ ID NO: 83 - protein, 249 aa, synthetic construct
DIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKGGGGGSGGGGSGGGGSGGGGSQVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSS

SEQ ID NO: 84 - protein, 248 aa, synthetic construct
QVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSSGGGGSGGGGSGGGGSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIK

SEQ ID NO: 85 - protein, 249 aa, synthetic construct
QVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSSGGGGGSGGGGSGGGGSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIK

SEQ ID NO: 86 - protein, 250 aa, synthetic construct
QVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSSGGGGGSGGGGSGGGGSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKG

SEQ ID NO: 87 - protein, 249 aa, synthetic construct
QVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSSGGGGSGGGGSGGGGSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKG

SEQ ID NO: 88 - protein, 111 aa, synthetic construct
EIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 89 - protein, 255 aa, synthetic construct
EIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLGGGGSGGGGSGGGGSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 90 - protein, 256 aa, synthetic construct
EVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLG

SEQ ID NO: 91 - protein, 256 aa, synthetic construct
EVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 92 - protein, 257 aa, synthetic construct
EVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLG

SEQ ID NO: 93 - protein, 255 aa, synthetic construct
EVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 94 - protein, 109 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 95 - protein, 255 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGGSGGGGSGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 96 - protein, 254 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGSGGGGSGGGGSGGGGSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSS

SEQ ID NO: 97 - protein, 255 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLG

SEQ ID NO: 98 - protein, 255 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 99 - protein, 255 aa, synthetic construct; identical to SEQ ID NO: 97

SEQ ID NO: 100 - protein, 254 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGGGGSGGGGSGGGGSGGGGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 101 - protein, 481 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSQVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSSGGGGSGGGGSGGGGSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIK

SEQ ID NO: 102 - protein, 476 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLG

SEQ ID NO: 103 - protein, 476 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 104 - protein, 489 aa, synthetic construct; identical to SEQ ID NO: 48

SEQ ID NO: 105 - protein, 468 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIKGGGGSGGGGSGGGGSGGGGSQVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSS

SEQ ID NO: 106 - protein, 489 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLG

SEQ ID NO: 107 - protein, 489 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 108 - protein, 468 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSQVQLVQSGGGSVKPGASVKVSCKASGYTFTDYSINWVRQAPGKCLEWMGWINTETGNPTYAQGFTGRFTISLDTSVSTAYLQISSLKAEDTASYYCALDKHLAMDYWGQGTSVTVSSGGGGSGGGGSGGGGSGGGGSDIVLTQSPASLAVSPGERATITCRASKSVSFLGINLIYWYQQKPGQPPKLLIYQASNRDTGVPARFSGSGSGTDFTLTINPVEAEDTANYYCLQTPFFPRTFGCGTKLEIK

SEQ ID NO: 109 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLGGGGSGGGGSGGGGSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 110 - protein, 475 aa, synthetic construct
QAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 111 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVLGGGGSGGGGSGGGGSGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSS

SEQ ID NO: 112 - protein, 488 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSEVQLVESGGGSVQPGGSLRLSCKASGYAFSSYWMNWVRQAPGKCLEWIGQIWPGDGDTNYNGKFKGRFTISADESKNTAYLQMNSLRAEDTATYYCARRETTTVGRYYYAMDYWGQGTTVTVSSGGGGSGGGGSGGGGSGGGGSEIVMTQSPSTLSASVGDRVTITCRASQSVDYDGDSYLNWYQQKPGKAPKLLIYDASNLVSGVPSRFSGSGSGTDFTLTISSLQPEDFATYYCQQSTEDPWTFGCGTKLTVL

SEQ ID NO: 113 - protein, 111 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 114 - protein, 124 aa, synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 115 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 116 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 117 - protein, 108 aa, synthetic construct
SYVLTQPPSVSVAPGQTARITCGGNNIGSKSVHWYQQPPGQAPVVVVYDDSDRPSGIPERFSGSNSGNTATLTISRVEAGDEAVYYCQVWDSSSDHVVFGGGTKLTVL

SEQ ID NO: 118 - protein, 121 aa, synthetic construct
QLQLQESGPGLVKPSETLSLTCTVSGGSISSGSYFWGWIRQPPGKGLEWIGSIYYSGITYYNPSLKSRVTISVDTSKNQFSLKLSSVTAADTAVYYCARHDGAVAGLFDYWGQGTLVTVSS

SEQ ID NO: 119 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 120 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 121 - protein, 6 aa, synthetic construct
TDYSIN

SEQ ID NO: 122 - protein, 19 aa, synthetic construct
RIRSKFNNYATYYADSVKG

SEQ ID NO: 123 - protein, 72 aa, synthetic construct
  VARIANT 1..8: note = This region may encompass 1-8 residues
  VARIANT 10..17: note = This region may encompass 1-8 residues
  VARIANT 19..26: note = This region may encompass 1-8 residues
  VARIANT 28..35: note = This region may encompass 1-8 residues
  VARIANT 37..44: note = This region may encompass 1-8 residues
  VARIANT 46..53: note = This region may encompass 1-8 residues
  VARIANT 55..62: note = This region may encompass 1-8 residues
  VARIANT 64..71: note = This region may encompass 1-8 residues
  VARIANT 1..72: note = This sequence may encompass 1-8 GnS repeating units, wherein n is 1-8
GGGGGGGGSGGGGGGGGSGGGGGGGGSGGGGGGGGSGGGGGGGGSGGGGGGGGSGGGGGGGGSGGGGGGGGS

(19) United States
(12) Patent Application Publication
(10) Pub. No.: US 2026/0337289 A1
(43) Pub. Date: Oct. 29, 2026

(54) MULTISPECIFIC ANTIGEN BINDING PROTEINS AND USE THEREOF

(71) Applicant: CDR-LIFE AG, Horgen (CH)

(72) Inventors: Leonardo BORRAS, Birmensdorf (CH); Stephanie JUNGMICHEL, Zurich (CH); Iuliano Maria LOREDANA, Zurich (CH); Philipp Robert RICHLE, Zurich (CH); Fabian Bert SCHEIFELE, Maegenwil (CH); Alessio VANTELLINI, Zurich (CH)

(21) Appl. No.: 19/653,294

(22) Filed: Apr. 21, 2026

Related U.S. Application Data
(60) Provisional application No. 63/791,965, filed on Apr. 21, 2025.
(60) Provisional application No. 63/866,471, filed on Aug. 19, 2025.
(60) Provisional application No. 63/896,960, filed on Oct. 10, 2025.

(51) Int. Cl. C07K 16/28; A61K 38/00; A61K 47/68; A61P 35/00; A61P 37/06; C07K 14/705; C12N 5/0783

(52) U.S. Cl. CPC C07K 16/2878; A61K 47/6849; A61P 35/00; A61P 37/06; C07K 14/7051; C07K 16/2803; C07K 16/2809; C12N 5/0636; A61K 38/00; C07K 2317/31; C07K 2317/622; C07K 2317/92; C12N 2510/00

(57) ABSTRACT

Provided are multispecific antigen binding proteins and their individual binding domains that specifically bind to BCMA, CD19 and an immune cell target antigen (e.g., CD3). Also provided are conjugates thereof, pharmaceutical compositions comprising the antigen binding proteins, as well as nucleic acids, recombinant expression vectors and host cells engineered to express such antigen binding proteins. Also disclosed are methods of using the antigen binding proteins in the prevention, and/or treatment of diseases or disorders, in particular B cell related disease such as an autoimmune disease or cancer.

### CROSS-REFERENCE TO RELATED APPLICATIONS

This application claims priority to: U.S. Provisional Application No. 63/791,965, filed on Apr. 21, 2025, entitled “MULTISPECIFIC ANTIGEN BINDING PROTEINS AND USE THEREOF”; U.S. Provisional Application No. 63/866,471, filed on Aug. 19, 2025, entitled “MULTISPECIFIC ANTIGEN BINDING PROTEINS AND USE THEREOF”; and U.S. Provisional Application No. 63/896,960, filed on Oct. 10, 2025, entitled “MULTISPECIFIC ANTIGEN BINDING PROTEINS AND USE THEREOF.” The contents of said applications are incorporated by reference in their entirety herein.

### REFERENCE TO AN ELECTRONIC SEQUENCE LISTING

The contents of the electronic sequence listing (1157443_008613_SL.xml; Size: 129,779 bytes; and Date of Creation: Apr. 1, 2026) is herein incorporated by reference in its entirety.

### FIELD OF THE INVENTION

This disclosure relates to compositions comprising and methods of using multispecific antigen binding proteins with specificity to B cells, plasma cells, and a T cell-recruiting antigen. This disclosure relates to compositions comprising and methods of using antigen binding proteins (e.g., antibodies and antibody fragments) with specificity to B cells and/or plasma cells.

### BACKGROUND

In difficult-to-treat B cell-mediated autoimmune diseases, current standards of care do not induce cure or treatment-free remission. The majority of patients do not respond optimally, if at all, to current therapies and require lifelong immune suppression, which is associated with infections and secondary malignancies.

B cells exhibit widespread expression of CD19 during most stages of differentiation. However, as these cells undergo their final transformation into plasma cells, CD19 expression is downregulated. B cell depletion has shown efficacy in several autoimmune diseases. However, CD19 CAR-Ts for example, require lymphodepleting chemotherapy and come with the challenges of CAR-Ts that may limit broad uptake in autoimmune diseases. On the other hand, CD19 targeted B cell and plasma cell depletion does not reach into the terminally differentiated long-lived plasma cells, which are a source of autoantibodies that can survive for years or even decades in the lymphoid tissues.

B-Cell Maturation Antigen (BCMA) is mainly expressed on plasma blasts (PB) and plasma cells (PC) with the highest expression level on fully differentiated long-lived plasma cells. Both PBs and PCs are the producers of autoantibodies, i.e., antibodies that attack one's own tissue instead of pathogens. While BCMA targeting therapies have shown some success in reducing autoantibody-producing cells, the reduction is often insufficient and the treatment does not address autoreactive B cells.

Hence, there is an unmet need for off-the-shelf therapy able to deliver full immune reset for cure or durable treatment-free remissions.

### SUMMARY

The present invention provides antigen binding proteins with specificity to human BCMA, antigen binding proteins with specificity to human CD3, and antigen binding proteins with specificity to human CD19. These antigen binding proteins can be used as building blocks for multispecific binding proteins, such as bispecific binding proteins or trispecific binding proteins.

In certain embodiments, a multispecific antigen binding protein, such as a trispecific antigen binding protein, is provided that specifically binds to BCMA, CD3 and CD19. Bispecific antibodies which specifically bind to CD3 and CD19 have been shown to provide some clinical improvements in certain autoimmune diseases such as systemic sclerosis or systemic lupus erythematosus (SLE). However, such bispecific antibodies are not able to deplete autoantibody-producing cells and thus do not sufficiently or completely treat autoimmune diseases mediated by autoantibodies. The present disclosure contemplates that by additionally depleting long-lived plasma cells via targeting BCMA, a complete reset of autoantibody production can be achieved. Depleting the entire B cell and plasma cell compartment with a CD3xBCMAxCD19 trispecific T cell engager (TCE) resets the production of autoantibodies and eradicates all autoreactive information from the B cells and plasma cells. Therefore, this is a unique opportunity to treat or even cure autoantibody mediated autoimmune diseases.

The exemplary trispecific antigen binding protein achieves broad coverage of the B cell compartment by targeting CD19 and BCMA positive B cells, i.e., both B cells and plasma cells.

The BCMA-targeting arm removes plasma cells, i.e., the source of autoantibodies, whereas the CD19-targeting arm prevents accumulation of new autoreactive plasma cells. Thereby, a broader depletion of B cells compared to corresponding bispecific BCMA or CD19 T-cell engagers is achieved, translating into broader efficacy compared to these bispecifics. Thus, the exemplary trispecific antigen binding protein may deliver stronger and more durable responses by depleting a broader range of pathological B-cell populations and therefore offers the chance of treatment-free remission across various autoimmune diseases.

Moreover, the exemplary trispecific antigen binding protein is an off-the-shelf drug product with excellent drug properties.

In one aspect, the disclosure provides a multispecific antigen binding protein, such as a trispecific antigen binding protein, comprising a first binding domain that specifically binds to human BCMA, a second binding domain that specifically binds to human CD3, and a third binding domain that specifically binds to human CD19.

In some embodiments, each binding domain may comprise: a variable light chain (VL) comprising a CDR-L1, a CDR-L2, and a CDR-L3; and a variable heavy chain (VH) comprising a CDR-H1, a CDR-H2, and a CDR-H3.

In certain embodiments, in the VL of the first binding domain, the CDR-L1, the CDR-L2, and the CDR-L3 may comprise the CDR-L1, CDR-L2, and CDR-L3 sequences, respectively, contained in SEQ ID NO: 7 or 82, and in the VH of the first binding domain, the CDR-H1, the CDR-H2, and the CDR-H3 may comprise the CDR-H1, CDR-H2, and CDR-H3 sequences, respectively, contained in SEQ ID NO: 8.

In certain embodiments, in the VL of the second binding domain, the CDR-L1, the CDR-L2, and the CDR-L3 may comprise the CDR-L1, CDR-L2, and CDR-L3 sequences, respectively, contained in SEQ ID NO: 25 or 94, and in the VH of the second binding domain, the CDR-H1, the CDR-H2, and the CDR-H3 may comprise the CDR-H1, CDR-H2, and CDR-H3 sequences, respectively, contained in SEQ ID NO: 26.

In certain embodiments, in the VL of the third binding domain, the CDR-L1, the CDR-L2, and the CDR-L3 may comprise the CDR-L1, CDR-L2, and CDR-L3 sequences, respectively, contained in SEQ ID NO: 16 or 88, and in the VH of the third binding domain, the CDR-H1, the CDR-H2, and the CDR-H3 may comprise the CDR-H1, CDR-H2, and CDR-H3 sequences, respectively, contained in SEQ ID NO: 17.

Such CDR-L1, CDR-L2, and CDR-L3 sequences and the CDR-H1, CDR-H2, and CDR-H3 sequences in may be the sequences defined by any appropriate CDR-defining scheme including but not limited to Kabat, Chothia, AHo, Contact, or IMGT.

In certain embodiments, each binding domain comprises a variable light chain (VL) and variable heavy chain (VH), wherein: (a) the first binding domain specifically binds to human BCMA and comprises: (a1) a VL comprising a CDR-L1 sequence of RASKSVSFLGINLIY (SEQ ID NO: 1), a CDR-L2 sequence of QASNRDT (SEQ ID NO: 2) and a CDR-L3 sequence of LQTPFFPRT (SEQ ID NO: 3), and (a2) a VH comprising a CDR-H1 sequence of DYSIN (SEQ ID NO: 4), a CDR-H2 sequence of WINTETGNPTYAQGFTG (SEQ ID NO: 5), and a CDR-H3 sequence of DKHLAMDY (SEQ ID NO: 6); (b) the second binding domain specifically binds to human CD3 and comprises: (b1) a VL comprising a CDR-L1 sequence of GSSTGAVTTSNYAN (SEQ ID NO: 19), a CDR-L2 sequence of GTNKRAP (SEQ ID NO: 20) and a CDR-L3 sequence of ALWYSNHWV (SEQ ID NO: 21), and (b2) a VH comprising a CDR-H1 sequence of TYAMN (SEQ ID NO: 22), a CDR-H2 sequence of RIRSKYNNYATYYADSVKG (SEQ ID NO: 23), and a CDR-H3 sequence of HGNFGDSYVSWFAY (SEQ ID NO: 24); and (c) the third binding domain specifically binds to human CD19 and comprises: (c1) a VL comprising a CDR-L1 sequence of RASQSVDYDGDSYLN (SEQ ID NO: 10), a CDR-L2 sequence of DASNLVS (SEQ ID NO: 11) and a CDR-L3 sequence of QQSTEDPWT (SEQ ID NO: 12), and (c2) a VH comprising a CDR-H1 sequence of SYWMN (SEQ ID NO: 13), a CDR-H2 sequence of QIWPGDGDTNYNGKFKG (SEQ ID NO: 14), and a CDR-H3 sequence of RETTTVGRYYYAMDY (SEQ ID NO: 15); or variants thereof comprising up to one, two, or three amino acid substitutions, insertions or deletions per CDR sequence.

In certain embodiments, the first binding domain comprises: (i) a VL comprising an amino acid sequence that is at least about 90%, 95%, 96%, 97%, 98%, 99% or 100% identical to the amino acid sequence of SEQ ID NO: 7 or 82; and/or (ii) a VH comprising an amino acid sequence that is at least about 90%, 95%, 96%, 97%, 98%, 99% or 100% identical to the amino acid sequence of SEQ ID NO: 8.

In certain embodiments, the second binding domain comprises: (i) a VL comprising an amino acid sequence that is at least about 90%, 95%, 96%, 97%, 98%, 99% or 100% identical to the amino acid sequence of SEQ ID NO: 25 or 94; and/or (ii) a VH comprising an amino acid sequence that is at least about 90%, 95%, 96%, 97%, 98%, 99% or 100% identical to the amino acid sequence of SEQ ID NO: 26.

In certain embodiments, the third binding domain comprises: (i) a VL comprising an amino acid sequence that is at least about 90%, 95%, 96%, 97%, 98%, 99% or 100% identical to the amino acid sequence of SEQ ID NO: 16 or 88; and/or (ii) a VH comprising an amino acid sequence that is at least about 90%, 95%, 96%, 97%, 98%, 99% or 100% identical to the amino acid sequence of SEQ ID NO: 17.

For example, any of such VL and/or VH sequence identity may be met while comprising the CDR-L1, CDR-L2, and CDR-L3 sequences and/or CDR-H1, CDR-H2, and CDR-H3 sequences as described above.

In certain embodiments, said trispecific antigen binding protein is a Fab-(scFv)₂, a Tri-MAB, a CODV-Ig, an OrthoTsAb, a 2+1 common LC, a Triplebody, a Triflex, a MATCH3, or a triabody, in particular a Fab-(scFv)₂.

In certain embodiments, the trispecific antigen binding protein is a Fab-(scFv)₂ (a format in which two scFv's are connected to a Fab), wherein (i) the Fab molecule comprises the CD3-binding domain comprising a heavy chain with a CH1 domain and the VH, and a light chain with a CL domain and the VL, (ii) one scFv comprises the BCMA-binding domain, and (iii) one scFv comprises the CD19-binding domain.

In some cases, the heavy chain of the CD3 binding Fab comprises the CD3-binding VH and the CH1 in the direction from the N-terminus to the C-terminus, and the light chain of the CD3 binding Fab comprises the CD3-binding VL and the CL in the direction from the N-terminus to the C-terminus.

In some cases, the BCMA-binding scFv may comprise the BCMA-binding VL, a linker, and the BCMA-binding VH in the direction from the N-terminus to the C-terminus. In some cases, the BCMA-binding scFv may comprise the BCMA-binding VH, a linker, and the BCMA-binding VL in the direction from the N-terminus to the C-terminus.

In some cases, the CD19-binding scFv may comprise the CD19-binding VL, a linker, and the CD19-binding VH in the direction from the N-terminus to the C-terminus. In some cases, the CD19-binding scFv may comprise the CD19-binding VH, a linker, and the CD19-binding VL in the direction from the N-terminus to the C-terminus.

In some cases, the linker in the BCMA-binding scFv and/or the CD19-binding scFv may comprise glycine (G) and/or serine(S), such as GGGGS (SEQ ID NO: 31), or comprise one or more repeats of GGGGS (SEQ ID NO: 31), e.g., SEQ ID NO: 34, 35, 33, 32, or 36.

In certain embodiments, (a) said CD3 binding Fab heavy chain is at least about 80%, 85%, 90%, 95%, 96%, 97%, 98%, 99% or 100% identical to the amino acid sequence of SEQ ID NO: 28, and said CD3 binding Fab light chain is at least about 80%, 85%, 90%, 95%, 96%, 97%, 98%, 99% or 100% identical to the amino acid sequence of SEQ ID NO: 27; (b) said BCMA-binding scFv is at least about 80%, 85%, 90%, 95%, 96%, 97%, 98%, 99% or 100% identical to the amino acid sequence of SEQ ID NO: 9 or 83; and/or (c) said CD19-binding scFv is at least about 80%, 85%, 90%, 95%, 96%, 97%, 98%, 99% or 100% identical to the amino acid sequence of SEQ ID NO: 18 or 89.

In some cases, a BCMA-binding scFv which comprises the BCMA-binding VH, a linker, and the BCMA-binding VL in the direction from the N-terminus to the C-terminus and is at least about 80%, 85%, 90%, 95%, 96%, 97%, 98%, 99% or 100% identical to the amino acid sequence of SEQ ID NO: 84, 85, 86, or 87 may be used. In some cases, a CD19-binding scFv which comprises the CD19-binding VH, a linker, and the CD19-binding VL in the direction from the N-terminus to the C-terminus and is at least about 80%, 85%, 90%, 95%, 96%, 97%, 98%, 99% or 100% identical to the amino acid sequence of SEQ ID NO: 90, 91, 92, or 93 may be used.

In certain embodiments, said BCMA binding domain and said CD19 binding domain are fused to the CD3-binding domain via a peptide linker.

In particular embodiments, said BCMA binding domain and said CD19 binding domain are fused to the CD3-binding domain via a peptide linker at the C-terminus of the Fab heavy and light chains, respectively.

In certain embodiments, the BCMA binding domain and the CD19 binding domain are fused to the CD3-binding domain via a peptide linker at the C-terminus of the Fab light and heavy chains, respectively. In certain embodiments, said linker is or comprises GGGGS (SEQ ID NO: 31).

In certain embodiments, the heavy chain of the CD3 binding Fab comprises, at the C-terminus of the CH1, a hinge or a partial hinge, e.g., an amino acid sequence EPKSC (SEQ ID NO: 37).

In some embodiments, the trispecific antigen binding protein comprises a first polypeptide (in some cases referred to as chain 1 or a light chain of the trispecific antigen binding protein) and a second polypeptide (in some cases referred to as chain 2 or a heavy chain of the trispecific antigen binding protein).

In certain embodiments, the first polypeptide may comprise the CD3-binding VL, a/the CL, and a linker in the direction from the N-terminus to the C-terminus, and the second polypeptide may comprise the CD3-binding VH, a/the CH1, and an optional hinge or a partial hinge in the direction from the N-terminus to the C-terminus.

In some particular embodiments, the first polypeptide may further comprise the CD19-binding scFv, and the second polypeptide may further comprise the BCMA-binding scFv. In particular cases, the CD19-binding scFv may comprise the CD19-binding VL, a linker, and the CD19 binding VH in the direction from the N-terminus to the C-terminus. In particular cases, the BCMA-binding scFv may comprise the BCMA-binding VL, a linker, and the BCMA-binding VH in the direction from the N-terminus to the C-terminus.

In certain embodiments, the trispecific antigen binding protein comprises (a) an amino acid sequence that is at least about 80%, 85%, 90%, 95%, 96%, 97%, 98%, 99% or 100% identical to the amino acid sequence of SEQ ID NO: 29 (e.g., the second polypeptide); and (b)

an amino acid sequence that is at least about 80%, 85%, 90%, 95%, 96%, 97%, 98%, 99% or 100% identical to the amino acid sequence of SEQ ID NO: 30 (e.g., the first polypeptide).

In certain embodiments, a glutamine (Q) or glutamate (E) at position 1 of the light chain and/or heavy chain is replaced by pyroglutamate (pE). In certain embodiments, the trispecific antigen binding protein comprises a pyroglutamate (pE) at position 1 of the light chain instead of glutamine (Q).

In certain embodiments, the trispecific antigen binding protein consists of (a) the amino acid sequence of SEQ ID NO: 29 (e.g., the second polypeptide); and/or (b) the amino acid sequence of SEQ ID NO: 30 (e.g., the first polypeptide).

In certain embodiments, the affinity of the CD3 binding domain is at least about 20 times, 50 times, 100 times, or at least about 150 times lower than the affinity of the BCMA binding domain (i.e., the equilibrium dissociation constant of the CD3 binding domain is higher than the one of the BCMA binding domain).

In certain embodiments, the affinity of the CD19 binding domain is at least about 20 times, about 50 times, or at least about 100 times lower than the affinity of the BCMA binding domain (i.e., the equilibrium dissociation constant of the CD19 binding domain is higher than the one of the BCMA binding domain).

In certain embodiments, the trispecific antigen binding protein comprises (i) an affinity (K_{D}) between about 0.5 μM to about 0.5 nM to human BCMA; (ii) an affinity (K_{D}) between about 0.01 nM to about 5 nM to human CD19; and/or (iii) an affinity (K_{D}) between about 1 nM to about 10 nM to human CD3.

In certain embodiments, the trispecific antigen binding protein is chemically or biologically modified.

In certain embodiments, the trispecific antigen binding protein is glycosylated, PEGylated, PASylated, XTENylated or HESylated.

In certain embodiments, the antigen binding protein (e.g., multispecific antigen binding protein, such as a bispecific or trispecific antigen binding protein) comprises a light chain and/or a heavy chain comprising an N-terminal and/or C-terminal truncation of 1, 2, 3, 4 or 5 amino acids.

In certain embodiments, the trispecific antigen binding protein is linked to or combined with a functional entity such as a detectable label, a therapeutic agent or a PK modifying moiety.

In certain embodiments, the trispecific antigen binding protein further comprises one or more additional binding domains.

In one aspect, the disclosure provides an isolated polynucleotide encoding the trispecific antigen binding protein described herein.

In one aspect, the disclosure provides a vector comprising the polynucleotide described herein, in particular an expression vector or a viral vector.

In one aspect, the disclosure provides a host cell comprising the isolated polynucleotide described herein or the vector described herein.

In one aspect, the disclosure provides a method of producing the trispecific antigen binding protein described herein, comprising the steps of (a) culturing the host cell described herein under conditions suitable for the expression of the antigen binding protein; and optionally (b) recovering the antigen binding protein.

In one aspect, the disclosure provides a pharmaceutical composition comprising the trispecific antigen binding protein described herein, the polynucleotide described herein, the vector described herein or the host cell described herein and a pharmaceutically acceptable carrier.

In one aspect, the disclosure provides the trispecific antigen binding protein described herein, the polynucleotide described herein, the vector described herein or the host cell described herein, or the pharmaceutical composition described herein for use as a medicament.

In one aspect, the disclosure provides the trispecific antigen binding protein described herein, the polynucleotide described herein, the vector described herein or the host cell described herein, or the pharmaceutical composition described herein for use in the treatment of a disease, in particular a B cell related disease, more particularly an autoimmune disorder.

In one aspect, the disclosure provides the use of trispecific antigen binding protein described herein, the polynucleotide described herein, the vector described herein or the host cell described herein, or the pharmaceutical composition described herein in the manufacture of a medicament.

In one aspect, the disclosure provides a method of treating a disease in a subject, comprising administering to said subject an effective amount of the trispecific antigen binding protein described herein, the polynucleotide described herein, the vector described herein or the host cell described herein, or the pharmaceutical composition described herein. In certain embodiments, the disease is a B cell related disease, in particular an autoimmune disease, or cancer, such as B cell malignancies.

In one aspect, the disclosure provides an antigen binding protein that specifically binds to human BCMA.

In some embodiments, the BCMA antigen binding protein may comprise: a variable light chain (VL) comprising a CDR-L1, a CDR-L2, and a CDR-L3; and a variable heavy chain (VH) comprising a CDR-H1, a CDR-H2, and a CDR-H3.

In some embodiments, in the VL of the BCMA antigen binding domain, the CDR-L1, the CDR-L2, and the CDR-L3 may comprise the CDR-L1, CDR-L2, and CDR-L3 sequences, respectively, contained in SEQ ID NO: 7 or 82, and in the VH of the BCMA binding protein, the CDR-H1, the CDR-H2, and the CDR-H3 may comprise the CDR-H1, CDR-H2, and CDR-H3 sequences, respectively, contained in SEQ ID NO: 8. Such CDR-L1, CDR-L2, and CDR-L3 sequences and CDR-H1, CDR-H2, and CDR-H3 sequences may be the sequences defined by any appropriate CDR-defining scheme including but not limited to Kabat, Chothia, AHo, Contact, or IMGT.

In some embodiments, the antigen binding protein may comprise (a) a VL comprising a CDR-L1 sequence of RASKSVSFLGINLIY (SEQ ID NO: 1), a CDR-L2 sequence of QASNRDT (SEQ ID NO: 2) and a CDR-L3 sequence of LQTPFFPRT (SEQ ID NO: 3), and
(a) a VH comprising a CDR-H1 sequence of DYSIN (SEQ ID NO: 4), a CDR-H2 sequence of WINTETGNPTYAQGFTG (SEQ ID NO: 5), and a CDR-H3 sequence of DKHLAMDY (SEQ ID NO: 6).

In certain embodiments, the antigen binding protein may comprise:
(a) a variable light chain (VL) comprising an amino acid sequence that is at least about 90%, 95%, 96%, 97%, 98%, 99% or 100% identical to the amino acid sequence of SEQ ID NO: 7 or 82; and
(b) a variable heavy chain (VH) comprising an amino acid sequence that is at least about 90%, 95%, 96%, 97%, 98%, 99% or 100% identical to the amino acid sequence of SEQ ID NO: 8.

For example, such VL and/or VH sequence identity may be met while comprising the CDR-L1, CDR-L2, and CDR-L3 sequences and/or CDR-H1, CDR-H2, and CDR-H3 sequences as described above.

In certain embodiments, the antigen binding protein has an affinity (K_{D}) between about 0.5 μM to about 0.5 nM to human BCMA.

In certain embodiments, the antigen binding protein is an antibody, such as a full-length immunoglobulin or an antibody fragment.

In certain embodiments, the antibody fragment is a Fab, a Fab′, a F(ab′)₂, a scFv, a Fv fragment, or a scFab.

In particular embodiments, the antigen binding protein is or comprises a scFv comprising the BCMA-binding VL, a linker, and the BCMA-binding VH in the direction from the N-terminus to the C-terminus.

In certain embodiments, the antigen binding protein is or comprises a scFv with an amino acid sequence that is at least about 80%, 85%, 90%, 95%, 96%, 97%, 98% or 100% identical to the amino acid sequence of SEQ ID NO: 9 or 83.

In certain embodiments, the antigen binding protein is or comprises a scFv comprising the BCMA-binding VH, a linker, and the BCMA-binding VL in the direction from the N-terminus to the C-terminus.

In certain embodiments, the antigen binding protein is or comprises a scFv with an amino acid sequence that is at least about 80%, 85%, 90%, 95%, 96%, 97%, 98% or 100% identical to the amino acid sequence of SEQ ID NO: 84, 85, 86, or 87.

In certain embodiments, the antigen binding protein is chemically or biologically modified.

In certain embodiments, the antigen binding protein is glycosylated, PEGylated, PASylated, XTENylated or HESylated.

In certain embodiments, the antigen binding protein comprises a light chain and/or a heavy chain comprising an N-terminal and/or C-terminal truncation of 1, 2, 3, 4 or 5 amino acids.

In certain embodiments, the antigen binding protein is linked to or combined with a functional entity such as a detectable label, a therapeutic agent or a PK modifying moiety.

In one aspect, the disclosure provides a chimeric antigen receptor (CAR) comprising the antigen binding protein that specifically binds to human BCMA described herein.

In one aspect, the disclosure provides an immune cell expressing the CAR described herein, in particular wherein the immune cell is a T cell.

In one aspect, the disclosure provides an antibody drug conjugate (ADC) comprising the antigen binding protein that specifically binds to human BCMA described herein.

In one aspect, the disclosure provides a multispecific antigen binding protein comprising: (i) a first binding domain, being the antigen binding protein that specifically binds to human BCMA described herein; and (ii) further comprising at least one second antigen binding domain against a second antigen.

In certain embodiments, the multispecific antigen binding protein is bispecific or trispecific.

In certain embodiments, the multispecific antigen binding protein comprises an immune cell engager, such as an immune cell engager which binds to CD3, TCRa, TCRB, CD16, NKG2D, CD89, CD64, or CD32, in particular CD3.

In certain embodiments, the immune cell engager is or comprises a CD3 binding domain derived from huSP34, huOKT, or huUCHT1.

In some embodiments, the CD3 binding domain may comprise: a variable light chain (VL) comprising a CDR-L1, a CDR-L2, and a CDR-L3; and a variable heavy chain (VH) comprising a CDR-H1, a CDR-H2, and a CDR-H3.

In certain embodiments, in the VL of the CD3 binding domain, the CDR-L1, the CDR-L2, and the CDR-L3 may comprise the CDR-L1, CDR-L2, and CDR-L3 sequences, respectively, contained in SEQ ID NO: 25 or 94, and in the VH of the CD3 binding domain, the CDR-H1, the CDR-H2, and the CDR-H3 may comprise the CDR-H1, CDR-H2, and CDR-H3 sequences, respectively, contained in SEQ ID NO: 26. Such CDR-L1, CDR-L2, and CDR-L3 sequences and CDR-H1, CDR-H2, and CDR-H3 sequences may be the sequences defined by any appropriate CDR-defining scheme including but not limited to Kabat, Chothia, AHo, Contact, or IMGT.

In certain embodiments, the CD3 binding domain may comprise: (a) a VL comprising a CDR-L1, a CDR-L2, and a CDR-L3, which comprise the sequences of GSSTGAVTTSNYAN (SEQ ID NO: 19), GTNKRAP (SEQ ID NO: 20), and ALWYSNHWV (SEQ ID NO: 21), respectively; and (b) a VH comprising a CDR-H1, a CDR-H2, and a CDR-H3, which comprise: the sequences of TYAMN (SEQ ID NO: 22), RIRSKYNNYATYYADSVKG (SEQ ID NO: 23), and HGNFGDSYVSWFAY (SEQ ID NO: 24), respectively.

In certain embodiments, the CD3 binding domain is or comprises (a) a variable light chain (VL) comprising an amino acid sequence that is at least about 90%, 95%, 96%, 97%, 98%, 99% or 100% identical to the amino acid sequence of SEQ ID NO: 25 or 94; and (b) a variable heavy chain (VH) comprising an amino acid sequence that is at least about 90%, 95%, 96%, 97%, 98%, 99% or 100% identical to the amino acid sequence of SEQ ID NO: 26. For example, any of such VL and/or VH sequence identity may be met while comprising the CDR-L1, CDR-L2, and CDR-L3 sequences and/or CDR-H1, CDR-H2, and CDR-H3 sequences as described above.

In certain embodiments, the CD3 binding domain is or comprises a Fab comprising a heavy chain comprising the CD3-binding VH and a CH and a light chain comprising the CD3-binding VL and a CL.

In certain embodiments, the CD3 binding domain is or comprises a Fab comprising a Fab heavy chain that is at least about 80%, 85%, 90%, 95%, 96%, 97%, 98%, 99% or 100% identical to the amino acid sequence of SEQ ID NO: 28, and a Fab light chain that is at least about 80%, 85%, 90%, 95%, 96%, 97%, 98%, 99% or 100% identical to the amino acid sequence of SEQ ID NO: 27.

In certain embodiments, the CD3 binding domain has an affinity (K_{D}) to human CD3 of about 1 nM to about 10 nM.

In certain embodiments, the multispecific antigen binding protein comprises an antigen binding domain targeting CD19.

In some embodiments, the CD19 binding domain may comprise: a variable light chain (VL) comprising a CDR-L1, a CDR-L2, and a CDR-L3; and a variable heavy chain (VH) comprising a CDR-H1, a CDR-H2, and a CDR-H3.

In certain embodiments, in the VL of the CD19 binding domain, the CDR-L1, the CDR-L2, and the CDR-L3 may comprise the CDR-L1, CDR-L2, and CDR-L3 sequences, respectively, contained in SEQ ID NO: 16 or 88, and in the VH of the CD19 binding domain, the CDR-H1, the CDR-H2, and the CDR-H3 may comprise the CDR-H1, CDR-H2, and CDR-H3 sequences, respectively, contained in SEQ ID NO: 17. Such CDR-L1, CDR-L2, and CDR-L3 sequences and CDR-H1, CDR-H2, and CDR-H3 sequences may be the sequences defined by any appropriate CDR-defining scheme including but not limited to Kabat, Chothia, AHo, Contact, or IMGT.

In certain embodiments, the antigen binding domain targeting CD19 comprises (a) a variable light chain (VL) comprising an amino acid sequence that is at least about 90%, 95%, 96%, 97%, 98%, 99% or 100% identical to the amino acid sequence of SEQ ID NO: 16 or 88; and/or (b) a variable heavy chain (VH) comprising an amino acid sequence that is at least about 90%, 95%, 96%, 97%, 98%, 99% or 100% identical to the amino acid sequence of SEQ ID NO: 17. For example, any of such VL and/or VH sequence identity may be met while comprising the CDR-L1, CDR-L2, and CDR-L3 sequences and/or CDR-H1, CDR-H2, and CDR-H3 sequences as described above.

In certain embodiments, the CD19-binding scFv may comprise the CD19-binding VL, a/the linker, and the CD19-binding VH in the direction from the N-terminus to the C-terminus. In certain embodiments, the CD19 binding domain is or comprises a scFv being at least about 80%, 85%, 90%, 95%, 96%, 97%, 98%, 99% or 100% identical to the amino acid sequence of SEQ ID NO: 18 or 89.

In certain embodiments, the CD19-binding scFv may comprise the CD19-binding VH, a/the linker, and the CD19-binding VL in the direction from the N-terminus to the C-terminus.

In certain embodiments, the CD19 binding domain is or comprises a scFv being at least about 80%, 85%, 90%, 95%, 96%, 97%, 98%, 99% or 100% identical to the amino acid sequence of SEQ ID NO: 90, 91, 92, or 93.

In certain embodiments, the CD19 binding domain has an affinity (K_{D}) to human CD19 of about 0.01 to about 5 nM.

In one aspect, the disclosure provides an antigen binding protein that specifically binds to human CD19.

In some embodiments, a CD19 antigen binding protein may comprise: a variable light chain (VL) comprising a CDR-L1, a CDR-L2, and a CDR-L3; and a variable heavy chain (VH) comprising a CDR-H1, a CDR-H2, and a CDR-H3.

In certain embodiments, in the VL of the CD19 antigen binding protein, the CDR-L1, the CDR-L2, and the CDR-L3 may comprise the CDR-L1, CDR-L2, and CDR-L3 sequences, respectively, contained in SEQ ID NO: 16 or 88, and in the VH of the CD19 antigen binding protein, the CDR-H1, the CDR-H2, and the CDR-H3 may comprise the CDR-H1, CDR-H2, and CDR-H3 sequences, respectively, contained in SEQ ID NO: 17. Such CDR-L1, CDR-L2, and CDR-L3 sequences and CDR-H1, CDR-H2, and CDR-H3 sequences in may be the sequences defined by any appropriate CDR-defining scheme including but not limited to Kabat, Chothia, Contact, AHo, or IMGT.

In certain embodiments, the CD19 antigen binding protein may comprise: (a) a variable light chain (VL), wherein said VL comprises a CDR-L1 sequence of RASQSVDYDGDSYLN (SEQ ID NO: 10), a CDR-L2 sequence of DASNLVS (SEQ ID NO: 11) and a CDR-L3 sequence of QQSTEDPWT (SEQ ID NO: 12), and (b) a variable heavy chain (VH), wherein said VH comprises a CDR-H1 sequence of SYWMN (SEQ ID NO: 13), a CDR-H2 sequence of QIWPGDGDTNYNGKFKG (SEQ ID NO: 14), and a CDR-H3 sequence of RETTTVGRYYYAMDY (SEQ ID NO: 15).

In certain embodiments, said antigen binding domain comprises (a) a variable light chain (VL) comprising an amino acid sequence that is at least about 90%, 95%, 96%, 97%, 98%, 99% or 100% identical to the amino acid sequence of SEQ ID NO: 16 or 88; and/or (b) a variable heavy chain (VH) comprising an amino acid sequence that is at least about 90%, 95%, 96%, 97%, 98%, 99% or 100% identical to the amino acid sequence of SEQ ID NO: 17. For example, such VL and/or VH sequence identity may be met while comprising the CDR-L1, CDR-L2, and CDR-L3 sequences and/or CDR-H1, CDR-H2, and CDR-H3 as described above.

In certain embodiments, the antigen binding protein is or comprises a scFv comprising the CD19-binding VL, a linker, and the CD19-binding VH in the direction from the N-terminus to the C-terminus.

In certain embodiments, the antigen binding protein is or comprises a scFv having at least about 80%, 85%, 90%, 95%, 96%, 97%, 98%, 99% or 100% identical to the amino acid sequence of SEQ ID NO: 18 or 89.

In certain embodiments, the antigen binding protein is or comprises a scFv comprising the CD19-binding VH, a linker, and the CD19-binding VL in the direction from the N-terminus to the C-terminus.

In certain embodiments, the antigen binding protein is or comprises a scFv having at least about 80%, 85%, 90%, 95%, 96%, 97%, 98%, 99% or 100% identical to the amino acid sequence of SEQ ID NO: 90, 91, 92, or 93.

In certain embodiments, the antigen binding protein has an affinity (K_{D}) to human CD19 of about 0.01 to about 5 nM.

In certain embodiments, the antigen binding protein is an antibody, such as a full-length immunoglobulin or an antibody fragment.

In certain embodiments, the antibody fragment is a Fab, a Fab′, a F(ab′)₂, a scFv, a Fv fragment or a scFab.

In certain embodiments, the antigen binding protein is chemically or biologically modified.

In certain embodiments, the antigen binding protein is glycosylated, PEGylated, PASylated, XTENylated or HESylated.

In certain embodiments, the antigen binding protein comprises a light chain and/or a heavy chain comprising an N-terminal and/or C-terminal truncation of 1, 2, 3, 4 or 5 amino acids.

In certain embodiments, the antigen binding protein is linked to or combined with a functional entity such as a detectable label, a therapeutic agent or a PK modifying moiety.

In one aspect, the disclosure provides a chimeric antigen receptor (CAR) comprising the antigen binding protein that specifically binds to human CD19 described herein.

In one aspect, the disclosure provides an immune cell expressing the CAR as described herein, in particular wherein the immune cell is a T cell.

In one aspect, the disclosure provides an antibody drug conjugate (ADC) comprising the antigen binding protein that specifically binds to human CD19 described herein.

In one aspect, the disclosure provides a multispecific antigen binding protein comprising: (i) a first binding domain, being the antigen binding protein that specifically binds to human CD19 described herein; and (ii) further comprising at least one second antigen binding domain against a second antigen.

In certain embodiments, the antigen binding protein is bispecific or trispecific.

In certain embodiments, the antigen binding protein comprises an immune cell engager, such as an immune cell engager which binds to CD3, TCRα, TCRβ, CD16, NKG2D, CD89, CD64, or a CD32 binding domain, in particular a CD3 binding domain.

In certain embodiments, said immune cell engager is a CD3 binding domain derived from huSP34, huOKT, or huUCHT1.

In some embodiments, the CD3 binding domain may comprise: a variable light chain (VL) comprising a CDR-L1, a CDR-L2, and a CDR-L3; and a variable heavy chain (VH) comprising a CDR-H1, a CDR-H2, and a CDR-H3.

In certain embodiments, in the VL of the CD3 binding domain, the CDR-L1, the CDR-L2, and the CDR-L3 may comprise the CDR-L1, CDR-L2, and CDR-L3 sequences, respectively, contained in SEQ ID NO: 25 or 94, and in the VH of the CD3 binding domain, the CDR-H1, the CDR-H2, and the CDR-H3 may comprise the CDR-H1, CDR-H2, and CDR-H3 sequences, respectively, contained in SEQ ID NO: 26. Such CDR-L1, CDR-L2, and CDR-L3 sequences and CDR-H1, CDR-H2, and CDR-H3 sequences in may be the sequences defined by any appropriate CDR-defining scheme including but not limited to Kabat, Chothia, Contact, AHo, or IMGT.

In certain embodiments, said CD3 binding domain is or comprises (a) a variable light chain (VL) comprising an amino acid sequence that is at least about 90%, 95%, 96%, 97%, 98%, 99% or 100% identical to the amino acid sequence of SEQ ID NO: 25 or 94; and (b) a variable heavy chain (VH) comprising an amino acid sequence that is at least about 90%, 95%, 96%, 97%, 98%, 99% or 100% identical to the amino acid sequence of SEQ ID NO: 26. For example, any of such VL and/or VH sequence identity may be met while comprising the CDR-L1, CDR-L2, and CDR-L3 sequences and/or CDR-H1, CDR-H2, and CDR-H3 sequences as described above.

In certain embodiments, the CD3 binding domain is or comprises a Fab comprising a heavy chain comprising the CD3-binding VH and a CH and a light chain comprising the CD3-binding VL and a CL.

In certain embodiments, said CD3 binding domain is or comprises a Fab comprising a Fab heavy chain that is at least about 80%, 85%, 90%, 95%, 96%, 97%, 98%, 99% or 100% identical to the amino acid sequence of SEQ ID NO: 28, and a Fab light chain that is at least about 80%, 85%, 90%, 95%, 96%, 97%, 98%, 99% or 100% identical to the amino acid sequence of SEQ ID NO: 27.

In certain embodiments, said CD3 binding domain has an affinity (K_{D}) to human CD3 of about 1 nM to about 10 nM.

In certain embodiments, the multispecific antigen binding protein further comprises a BCMA binding domain, in particular the BCMA binding protein described herein.

In one aspect, the disclosure provides an isolated polynucleotide encoding (i) the antigen binding protein described herein; (ii) the CAR described herein; or (iii) the ADC described herein.

In one aspect, the disclosure provides a vector comprising the polynucleotide described herein, in particular an expression vector or a viral vector.

In one aspect, the disclosure provides a host cell comprising the isolated polynucleotide described herein or the vector described herein.

In one aspect, the disclosure provides a method of producing the antigen binding protein described herein, comprising the steps of (a) culturing the host cell described herein under conditions suitable for the expression of the protein; and optionally (b) recovering the protein.

In one aspect, the disclosure provides a pharmaceutical composition comprising the antigen binding protein described herein; the CAR described herein; the ADC described herein; the polynucleotide described herein; the vector described herein; or the cell described herein and a pharmaceutically acceptable carrier.

In one aspect, the disclosure provides the antigen binding protein described herein; the CAR described herein; the ADC described herein; the polynucleotide described herein; the vector described herein; or the cell described herein or the pharmaceutical composition described herein for use as a medicament.

In one aspect, the disclosure provides the antigen binding protein described herein; the CAR described herein; the ADC described herein; the polynucleotide described herein; the vector described herein; or the cell described herein or the pharmaceutical composition described herein for use in the treatment of a disease, in particular a B cell related disease such as autoimmunity or cancer.

In one aspect, the disclosure provides the use of the antigen binding protein described herein; the CAR described herein; the ADC described herein; the polynucleotide described herein; the vector described herein; or the cell described herein or the pharmaceutical composition described herein in the manufacture of a medicament.

In one aspect, the disclosure provides the use of the antigen binding protein described herein; the CAR described herein; the ADC described herein; the polynucleotide described herein; the vector described herein; or the cell described herein or the pharmaceutical composition described herein in the manufacture of a medicament for the treatment of a disease. In certain embodiments, the disease is a B cell-related disease. In certain embodiments, the B cell related disease is (i) an autoimmune disease; and/or (ii) cancer, in particular B cell malignancies.

In one aspect, the disclosure provides a method of treating a disease in a subject, comprising administering to said subject an effective amount of the antigen binding protein described herein; the CAR described herein; the ADC described herein; the polynucleotide described herein; the vector described herein; or the cell described herein or the pharmaceutical composition described herein. In certain embodiments, the disease is a B cell-related disease. In certain embodiments, the B cell related disease is (i) an autoimmune disease; and/or (ii) cancer, in particular B cell malignancies.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1 shows expression of CD19 and BCMA during the B cell development pathway.

FIGS. 2A-2F show a comparison of trispecific M4148 with bispecific controls M3984 and M4141 in in vitro Granzyme B release assay. BCMA-positive/CD19-negative cell line RPMI-8226 (FIG. 2A), BCMA-negative/CD19-positive cell line NALM-6 (FIG. 2B, BCMA-negative/CD19-negative cell lines NCI-H1703 (FIG. 2C), and BCMA-positive/CD19-positive cell line Daudi and Raji (FIG. 2D, 2E) were incubated for 48 h with isolated T cells from healthy donors (E:T ratio of 5:1) and compounds at various concentrations. T cell cytotoxic activity was assessed by Granzyme B release ELISA assay, which is a surrogate marker of target cell killing. FIG. 2F shows trispecific M4148 on various double negative cell lines (NCI-H1703, PC3, HL-60, A375 and SKMEL-30) with NALM-6 as a positive control.

FIGS. 3A-3D show a comparison of trispecific M4148 with a CD19xCD3 bispecific comparator and a BCMAxCD3 bispecific comparator in in vitro Granzyme B release assay on BCMA-positive/CD19-positive cell lines Daudi (FIG. 3A, 3C) and Raji (FIG. 3B, 3D). Cells were incubated for 48 h with isolated T cells from healthy donors (E:T ratio of 5:1) and compounds at various concentrations. T cell cytotoxic activity was assessed by Granzyme B release ELISA assay, which is a surrogate marker of target cell killing.

FIGS. 4A-4B show a comparison of trispecific M4148 with the CD19xCD3 bispecific comparator in in vitro Granzyme B release assay on BCMA-negative/CD19-positive cell lines with high or low levels of CD19. NALM-6 (CD19 high) (FIG. 4A) and Reh (CD19 low) (FIG. 4B) cell lines were incubated for 48 h with isolated T cells from healthy donors (E:T ratio of 5:1) and compounds at various concentrations. T cell cytotoxic activity was assessed by Granzyme B release ELISA assay, which is a surrogate marker of target cell killing.

FIGS. 5A-5D show in vitro potency of M4148 on B cells from healthy donors. Isolated B cells were incubated for 48 h with isolated T cells from healthy donors (E:T ratio of 2:1) and compound M4148 (FIG. 5A) or compounds M4148 and the CD19xCD3 bispecific comparator (FIG. 5B) at various concentrations. T cell cytotoxic activity was assessed by Granzyme B release ELISA assay. Relative B cell depletion was assessed via flow cytometry after cultivation of 100,000 PBMCs from a healthy donor with various concentrations of compounds M4148 or the CD19xCD3 bispecific comparator for 48 h (FIG. 5C). Potency of trispecific M4148 was compared to bispecific control M3984 (BCMAxCD3) on expanded and stimulated B cells. PC-enriched B cells were incubated for 48 h with isolated T cells from healthy donors (E:T ratio of 2:1) at various concentrations of compounds (FIG. 5D). T cell cytotoxic activity was assessed by Granzyme B release ELISA assay, which is a surrogate marker of target cell killing.

FIGS. 6A-6B show a comparison of M4148 with a BCMAxCD3 bispecific comparator in in vitro Granzyme B and Interferon γ release assay on bone marrow samples from a donor without known autoimmune diseases. 100,000 bone marrow cells were co-cultured with isolated T cells (E:T ratio of 1:2) for 48 h at various compound concentrations. Granzyme B (FIG. 6A) and Interferon γ (FIG. 6B) release were analyzed in the supernatants with a multiplex immune assay via MSD U-plex plates (Meso Scale Discovery).

FIGS. 7A-7C show in vitro safety assessment of M4148 on normal human primary cells in comparison to BCMA-negative/CD19-positive cell line NALM-6. Human lung fibroblasts (NHLF) and human pulmonary artery smooth muscle cells (PASMC) (FIG. 7A), human pulmonary fibroblasts (HPF) and human cardiac fibroblasts (HCF) (FIG. 7B) or human aortic smooth muscle cells (HAoSMC) (FIG. 7C) were incubated for 24 h with isolated T cells from healthy donors (E:T ratio of 5:1) and M4148 at various concentrations. T cell cytotoxic activity was assessed by Granzyme B release ELISA assay, which is a surrogate marker of target cell killing.

FIGS. 8A-8C show a comparison of the potency of M4148 and the CD19xCD3 bispecific comparator on PBMCs from a donor with systemic lupus erythematosus (SLE). 100,000 PBMCs were incubated for 48 h at various compound concentrations. Total live B cells (FIG. 8A), total live PC (FIG. 8B) and the total live population which was negative for T cell, B cell and PC marker (FIG. 8C) were analyzed via flow cytometry.

FIGS. 9A-9B show a comparison of M4148 and the CD19xCD3 bispecific comparator in in vitro Granzyme B and Interferon γ release assay on PBMCs from a donor with SLE. 100,000 PBMCs were incubated for 48 h at various compound concentrations. Granzyme B (FIG. 9A) and Interferon γ (FIG. 9B) release were analyzed in the supernatants with a multiplex immune assay via MSD U-plex plates (Meso Scale Discovery).

FIGS. 10A-10D show the potency of M4148 on PBMCs from two donors with SLE and two donors with rheumatoid arthritis (RA). 100,000 PBMCs were incubated for 48 h at various compound concentrations. The percentage of B cell viability was analyzed via flow cytometry (FIG. 10A, 10B). Interferon γ release was analyzed in the supernatants with a multiplex immune assay via MSD U-plex plates (Meso Scale Discovery) (FIG. 10C, 10D).

FIG. 11 shows a schematic overview of the in vivo efficacy study design of M4148 performed in a SLE-induced humanized mouse model. During the efficacy study, mice were treated biweekly on days 0, 4, 7, 11, 14, 18 and 21 with buffer control (PBS) or M4148. Peripheral blood sampling occurred pre-treatment on day −4 and during the treatment on days 3, 11, 17 and at study endpoint day 24. Spleen and bone marrow were collected at study endpoint.

FIG. 12 shows the comparison of human IgG depletion between M4148-treated and vehicle control-treated groups in peripheral blood samples of SLE-induced humanized mice.

FIGS. 13A-13B show the comparison of B cell depletion between M4148-treated and vehicle control-treated groups in peripheral blood samples of SLE-induced humanized mice determined with flow cytometry. The total counts of CD19⁺ (FIG. 13A) or CD19⁺/BCMA⁺ (FIG. 13B) B cells per ml of peripheral blood (PB) are shown.

FIGS. 14A-14D show the comparison of B cell depletion between M4148-treated and vehicle control-treated groups in excised spleen and bone marrow samples at study endpoint of SLE-induced humanized mice determined with flow cytometry. In spleen, the total counts of CD19⁺ (FIG. 14A) or CD19⁺/BCMA⁺ (FIG. 14B) B cells per 0.1 g of tissue are shown. In bone marrow, the total counts of CD19⁺ (FIG. 14C) or CD19⁺/BCMA⁺ (FIG. 14D) B cells per femur are shown.

FIGS. 15A-15E show stability and solubility measurements of M4148. Stability was assessed via SEC analysis of 1 mg/ml of M4148 in PBS incubated for up to seven days at 37 °C (FIG. 15A). Shelf-life stability under accelerated conditions was performed at 50 mg/ml of M4148 in 10 mM Histidine buffer pH 5.5 for up to seven days at 40 °C (FIG. 15B). Stability at simulated physiological conditions was performed at 1 mg/ml in artificial subcutaneous fluid buffer for up to 14 days at 34 °C (FIG. 15C). Solubility was assessed via SEC analysis of concentrated M4148 at 50 mg/ml in PBS and compared to 1 mg/ml reference sample (FIG. 15D or concentrated M4148 at 120 mg/ml in 10 mM Histidine buffer pH 5.5 and compared to 10 mg/ml reference sample (FIG. 15E).

FIGS. 16A-16B show exemplary results from the flow cytometric assessment of B cell depletion in cynomolgus monkeys as described in Example 10-2. FIG. 16A provides quantification of CD19+, CD20+ and CD19+/CD20+ B cell populations in peripheral blood over time (left) and comparison between experimental groups at Day 15 (right). Absolute counts (cells/μL) are presented as mean±/−standard deviation (SD). Dotted vertical lines: dosing days. FIG. 16B provides quantification of CD19+, CD20+ and CD19+/CD20+ B cell populations in lymphoid tissues by flow cytometry. Absolute cell counts (cells/mg for lymph nodes and spleen samples, and cells/μL for bone marrow samples) presented as mean±/−standard deviation (SD).

FIGS. 17A-17B show exemplary results from the flow cytometric assessment of plasma cell depletion in cynomolgus monkeys as described in Example 10-2. FIG. 17A provides total BCMA+ plasma cells in the peripheral blood over time (top left) or on Day 15 (top right) and in lymphoid organs (spleen, lymph nodes, and bone marrow) on Day 15 (bottom). FIG. 17B provides quantification of CD19−/BCMA+ long-lived plasma cells (LLPCs) in the peripheral blood over time (left) or on Day 15 (center) and in the bone marrow on Day 15 (right). Absolute cell counts (cells/μL) are presented as mean±/−standard deviation (SD). Dotted line: dosing days.

FIGS. 18A-18B show exemplary results from the flow cytometric assessment of the kinetics of T cells and activated T cells in cynomolgus monkeys as described in Example 10-2. FIG. 18A provides quantification of CD3+ (top), CD3+/CD4+ helper T cells (middle), CD3+/CD4− (bottom) cytotoxic T cells in the peripheral blood over time. Data shown as absolute cell counts (cells/μL). Dotted vertical lines: dosing days. FIG. 18B provides quantification of CD25+ (top) and CD69+ (bottom) cells in the peripheral blood over time. Data shown as Mean Fluorescence Intensity (netMFI). Dotted vertical lines: dosing days.

FIG. 19 shows exemplary results from the quantification of cytokines in the plasma of cynomolgus monkeys by Luminex as described in Example 10-5. *Samples with values exceeding the upper limit of quantification (ULOQ). Dotted line: ULOQ.

FIG. 20 shows exemplary results from the quantification of cynomolgus IgGs in the serum of cynomolgus monkeys by ELISA as described in Example 10-6. The left provides absolute cynomolgus IgG concentrations in mg/mL. The right provides normalization (%) to pre-dose values. Vertical dotted lines: dosing days. Horizontal dotted line: threshold.

FIG. 21 shows exemplary results from the individual pharmacokinetic profiles of M4437 in cynomolgus monkeys as described in Example 10-7. Horizontal dotted line: PK assay LLOQ (0.4 ng/ml). Vertical dotted: dosing days. BLQ: Below the limit of quantification.

FIGS. 22A-22B, 23A-23C, and 24A24B provide exemplary and non-limiting structures and formats of various antigen binding molecules, e.g., antibodies, antigen binding antibody fragments, multispecific binding molecules, and multispecific antibodies disclosed herein, and antigen binding units (ABUs) contained in any of the foregoing.

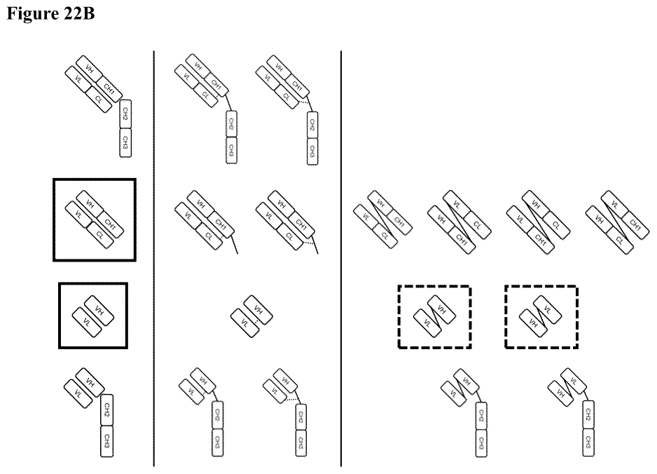

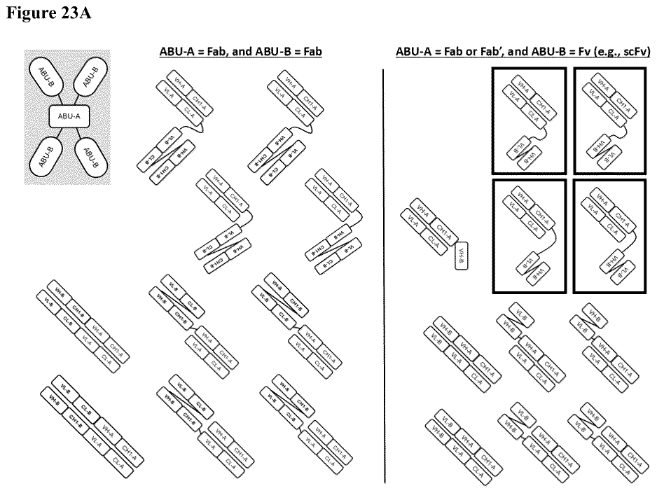

In FIGS. 22-24, the following rules apply unless otherwise indicated: (1) Each domain is presented as a rectangle with a text therein showing the domain name (e.g., CH1, VH, etc); (2) a set of multiple domains connected with each other represents a polypeptide (e.g., a heavy chain polypeptide, a light chain polypeptide, etc); (3) the direction of domains within a polypeptide is according to the direction of the text showing domain names, from the N-terminus to the C-terminus; (4) a linker, spacer, or a hinge may be used between domains as necessary and a disulfide bond(s) may exist between polypeptides (and/or within a domain or within a polypeptide), perhaps to allow correct formation of the antigen binding site(s), even when the Figures do not explicitly show a linker, a spacer, a hinge, or a disulfide bond; (5) any of the constant domains (e.g., CH1, CH2, and/or CH3 domain(s)) shown in Figures may be omitted or replaced with another constant domain when possible and/or desired and, when appropriate, may be replaced with a hinge or a linker; (6) rectangles with no pattern (i.e., open) are domains which individually may comprise a corresponding wild-type sequence or may comprise one or more amino acid substitutions relative to a wild-type sequence; (7) hinge and CH1, CH2, and CH3 domains may individually be of any (heavy chain) isotype; (8) when more than one hinges are present in a structure, the hinges may or may not be of the same isotype, when more than one CH1 domains are present in a structure, the CH1 domains may or may not be of the same isotype, when more than one CH2 domains are present in a structure, the CH2 domains may or may not be of the same isotype, and when more than one CH3 domains are present in a structure, the CH3 domains may or may not be of the same isotype; (9) light chain constant (CL) domain may be or may be derived from a kappa CL domain or a lambda CL domain; (10) when more than one CL domains are present in a structure, all CL domains may be kappa CLs or all CL domains may be lambda CLs, or alternatively at least one CL may be a kappa CL and at least one CL may be a lambda CL domain; (11) when both kappa and lambda CL domains are present, the CH1 domains paired to the CL domains may, in some instances, be variant CH1 domains, one of which may be a variant CH1 that preferentially binds to a kappa CL and another CH1 domain may be a variant CH1 that preferentially binds to a lambda CL (e.g., having kappa and lambda CLs and kappa-preferring CH1 and lambda-preferring CH1 in a molecule potentially allows for efficient manufacturing); (12) VH and VL domains paired with each other in Figures form an antigen binding site for an epitope; (13) in a given VH-VL pair, the VL may be omitted (or replaced with another VH, e.g., to provide bivalent binding with the other VH) even if VL is shown in Figures, if the VH alone gives sufficient specificity to a cognate antigen (e.g., nanobody); and (14) when there are more than one antigen binding sites, the antigen binding sites may all bind to the same epitope or at least one or more antigen binding sites may bind to a different epitope.

FIGS. 22A-22B provide some exemplary and non-limiting embodiments of antigen binding units which may be comprised by or contained in (i) various anti-CD3 binding molecules, antibodies, and antigen binding antibody fragments, (ii) various anti-CD19 binding molecules, antibodies and antigen binding antibody fragments, (iii) various anti-BCMA binding molecules, antibodies and antigen binding antibody fragments, and/or (iv) various ABUs which may be contained in multispecific binding molecules and multispecific antibodies which specifically bind to CD3, CD19, and BCMA according to the present disclosure. FIG. 22A provides some basic, exemplary antibody structures.

Top panel: The structure in the top left (boxed, double line) is of an exemplary, standard full-size antibody, in which hinges or disulfide bonds are not explicitly shown. The boxed antibody may, for example, comprise a hinge between CH1 and CH2 (solid line between CH1 and CH2) and one or more disulfide bond(s) (dotted line(s)) may be present between the hinges and/or between CH1 and CL (center). Alternatively, the boxed antibody may, for example, comprise a hinge between CH1 and CH2 (solid line between CH1 and CH2) and one or more disulfide bonds (dotted line(s)) may be present between hinges and/or between CL and hinge (right). Hinges and disulfide bonds, such as but not limited to those shown in middle and right antibody structures may be present, even if not explicitly shown, in any of the structures shown in Figures and described herein.

Bottom left panel: Additional variants of the top left structure (boxed, double line) in which in which certain domain positions are swapped are provided. In some variants, VH and VL positions are swapped and/or CH1 and CL positions are swapped, relative to the boxed structure. Variations equivalent thereto (swapping VH-VL and/or swapping CH1-CL positions) may be further applied to any of the structures shown in FIGS. 22-24 and the description thereof or variations thereof as appropriate, even if not explicitly shown.

Bottom center and right panels: Further variants of the top left structure (boxed, double line) in which one or more of the constant domains are omitted are provided. For example, in some variant structures, a CH3 domain(s) is/are omitted (center panel, top), a CH2 domain(s) is/are omitted (center panel, middle), a CH1 and a CL domain(s) is/are omitted (center panel, bottom), CH1 and CH2 domains are omitted (right panel, top), CH1 and CH3 domains are omitted (right panel, second from top), CH2 and CH3 domains are omitted (right panel, second from the bottom), and/or CH1, CH2, and CH3 domains are omitted (right panel, bottom). Even when one or more of such constant domains may be omitted, a hinge(s) (or a partial hinge(s)) and/or a disulfide bond(s) may be present or a linker or a spacer may be present to connect domains (in case of the bottom two structure in the right panel, the potential presence of the hinge, partial hinge, and/or disulfide bonds are shown with a dashed curved line). The structure of the second from the bottom in the right panel may be referred to as a F(ab′)₂. The bottom structure in the center panel may be referred to as a Fv-Fc. Variations equivalent thereto (omitting one or more CH3, CH2, CH1, and/or CL domains) may be further applied to any of the structures shown in FIGS. 22-24 and the description thereof or variations thereof as appropriate, even if not explicitly shown.

Additional variations: Although not explicitly shown, further encompassed are variants in which two or more certain domains (e.g., domains not explicitly depicted to be in the same polypeptide in Figures) are connected, optionally via a linker, to form a single chain. In some variants, VH and VL domains which together form an antigen binding site are connected to form a VL-VH or VH-VL chain (VL-linker-VH or VH-linker-VL, in the presence of a linker), i.e., a single-chain variable fragment (scFv). When this scFv variation is applied to the bottom structures in the center panel, the structure may be referred to as scFv-Fc. In some variants, VH-CH1 and VL-CL which together form an antigen binding site are connected to form a (VL-CL)-(VH-CH1) or (VH-CH1)-(VL-CL) chain ((VL-CL)-linker-(VH-CH1) or (VH-CH1)-linker-(VL-CL), in the presence of a linker), i.e., a single-chain Fab (scFab). When this scFab variation is applied to the top panel structures, the structure may be referred to as scFab-Fc. Variations equivalent thereto (single-chain variants) may be further applied to any of the structures shown in FIGS. 22-24 and the description thereof or variations thereof as appropriate, even if not explicitly shown.

FIG. 22B provides exemplary “half-antibody” variants of any of the structures described herein.

Left panel: The structures (top, 2nd, 3rd, and bottom rows) are “half-antibody” variants of the antibody structures provided (in the top panel, bottom right panel second from the bottom, bottom right panel bottom, and center panel bottom, respectively) in FIG. 22A.

Center panel: “Half-antibody” variants may also contain a hinge and/or disulfide bond(s) as appropriate, perhaps to allow correct formation of the antigen binding site(s). When the parent antibody is an Ig (e.g., IgG), the top structure may be referred to as a half-Ig (e.g., half IgG). The structure in the 2nd row may be referred to as a Fab. The structure in the 3rd row may be referred to as a Fv. The structure in the bottom row may be referred to as a Fv-CH2-CH3.

Right panel: Exemplary single-chain variants of the “half-antibody” variants are provided. The left two structures in the top may be referred to as scFab, in which VL may be placed upstream (at the N-terminal side) of VH, or VH may be placed upstream (at the N-terminal side) of VL. The right two structures in the top may be referred to as scFab-like, in which VH-VL positions and/or CH1-CL positions may be swapped relative to scFab. The structures in the middle row may be referred to as scFv (dashed box), in which VL may be placed upstream (at the N-terminal side) of VH, or VH may be placed upstream (at the N-terminal side) of VL. Further encompassed are variants containing multiple scFvs, such as: tandem scFv, in which multiple (two, three, four, etc) scFv fragments are placed in tandem within a polypeptide; and a diabody, a triabody, and a tetrabody. The structures in the bottom row may be referred to as scFv-CH2-CH3 or a half antibody variant of scFv-Fc.

Variations equivalent thereto (half-antibody variants) may be further applied to any of the structures shown in FIGS. 22-24 and the description thereof or variations thereof as appropriate, even if not explicitly shown. Furthermore, any two or more of the structures provided in FIGS. 22-24 and descriptions thereof may be combined to form an antibody structure.

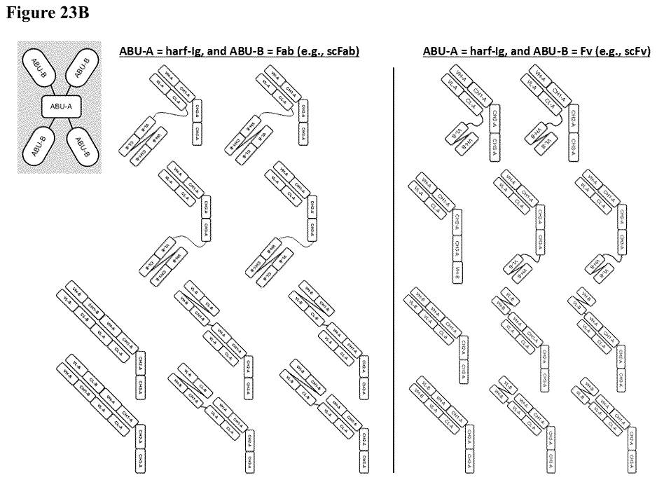

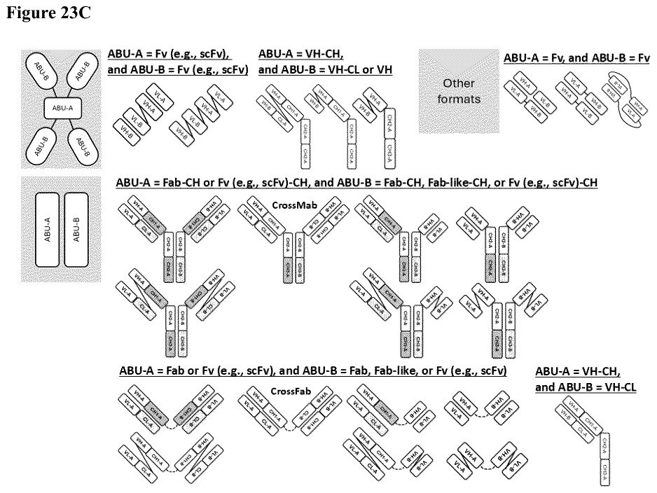

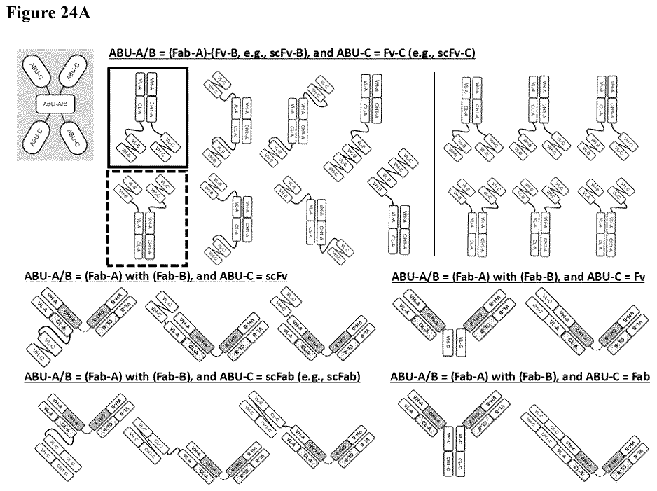

FIGS. 23A-23C provide some exemplary and non-limiting structures which may be comprised by or contained in antibodies comprising more than one antigen binding units (ABUs) such as multispecific antibodies according to the present disclosure. In addition to the above-noted rules (1)-(14), the following rules apply unless otherwise indicated: (15) VH-A and VL-A form an antigen binding site specific for antigen A or epitope A, and VH-B and VL-B form an antigen binding site for antigen B or epitope B; (16) antigens A and B or epitopes A and B may be different from or same as each other; (17) an ABU which binds to antigen A or epitope A may be referred to as ABU-A, an ABU which binds to antigen B or epitope B may be referred to as ABU-B, and an ABU which binds to antigen A or epitope A and antigen B or epitope B may be referred to as ABU-A/B and may comprise at least one ABU-A and at least one ABU-B; and (18) stripe and dotted constant domains paired with each other (e.g., a set of CH1 and CL domains or a set of two CH3 domains) in Figures may or may not be a set of constant domains one or both of which domains is/are engineered to promote preferential pairing between the constant domains of the set (e.g., over pairing with another constant domain). In some embodiments, antigens A and B may be CD3 and CD19, respectively, CD3 and BCMA, respectively, CD19 and CD3, respectively, CD19 and BCMA, respectively, BCMA and CD3, respectively, or BCMA and CD19, respectively.

FIG. 23A provides some exemplary ABU-A/B structures comprising at least one ABU-A connected to at least one ABU-B (a generic schematic of ABU-A/B depicted in the shaded box). Although one ABU-A and four ABU-Bs are shown, an ABU-A/B structure may comprise any number of, such as one, two, three, four, or more, ABU-As and ABU-Bs. In some cases, an ABU-A may comprise any of the structures provided in FIGS. 22A-22B or description thereof, and an ABU-B may comprise any of the structures provided in FIGS. 22A-22B or description thereof.

Left panel: In some ABU-A/B structures, an ABU-A/B may comprise a Fab (or Fab-like) which binds to antigen A or epitope A (Fab-A (or Fab-like-A)) and a Fab (or Fab-like) which binds to antigen B or epitope B (Fab-B (or Fab-like-B)). In some cases, when the Fab-B (or Fab-like-B) is a scFab (or scFab-like), the N-terminus of scFab-B (or scFab-like-B) may be connected to the C-terminus of the light chain (top row) or the heavy chain (second row) of Fab-A, or the C-terminus of scFab-B (or scFab-like-B) may be connected to the N-terminus of the heavy chain (third row) or the light chain (bottom row) of Fab-A. In some cases, the C-termini of one polypeptide chain (e.g., heavy chain) and the other polypeptide chain (e.g., light chain) of Fab-B (Fab-like-B) may be connected to the N-termini of the heavy and light chains, respectively (third row, left), or the light and heavy chains, respectively (bottom row, left), of Fab-A. While one Fab-B is shown, more than one Fab-B's may be connected (e.g., in tandem or at different positions of ABU-A).

Right panel: In some ABU-A/B structures, an ABU-A/B may comprise a Fab-A and a Fv which binds to antigen B or epitope B (Fv-B). In some cases, when the Fv-B is a scFv, the N-terminus of scFv-B may be connected to the C-terminus of the light chain (top row) or the heavy chain (second row, right two structures) of Fab-A, or the C-terminus of scFv-B may be connected to the N-terminus of the heavy chain (third row, right two structures) or the light chain (bottom row, right two structures) of Fab-A. In some cases, the C-termini of VH-B and VL-B of Fv-B may be connected to the N-termini of the heavy and light chains, respectively (third row, left), or the light and heavy chains, respectively (bottom row, left), of Fab-A. In certain cases, VH-B may be capable of binding to antigen B or epitope B without VL-B (i.e., VH-B as nanobody), and such VH-B may be connected to the C-terminus of the heavy chain (2nd row, left) or light chain of Fab-A or the N-terminus of the heavy or light chain of Fab-A. While one Fv-B is shown per structure, more than one Fv-B's may be connected (e.g., in tandem or at different positions of ABU-A).

FIG. 23B provides some additional exemplary ABU-A/B structures comprising at least one ABU-A connected to at least one ABU-B (a generic schematic of ABU-A/B depicted in shaded box).

Left panel: In some ABU-A/B structures, an ABU-A/B may comprise a half-Ig which binds to antigen A or epitope A (half Ig-A) and a Fab (or Fab-like) which binds to antigen B or epitope B (Fab-B (or Fab-like-B)). In some cases, when the Fab-B (or Fab-like-B) is a scFab (or scFab-like), the N-terminus of scFab-B (or scFab-like-B) may be connected to the C-terminus of the light chain (top row) or the heavy chain (second row) of half Ig-A, or the C-terminus of scFab-B (or scFab-like-B) may be connected to the N-terminus of the heavy chain (third row) or the light chain (bottom row) of half Ig-A. In some cases, the C-termini of one polypeptide chain (e.g., heavy chain) and the other polypeptide chain (e.g., light chain) of Fab-B (Fab-like-B) may be connected to the N-termini of the heavy and light chains, respectively (third row, left), or the light and heavy chains, respectively (bottom row, left), of half Ig-A.

Right panel: In some ABU-A/B structures, an ABU-A/B may comprise a half Ig-A and a Fv which binds to antigen B or epitope B (Fv-B). In some cases, when the Fv-B is a scFv, the N-terminus of scFv-B may be connected to the C-terminus of the light chain (top row) or the heavy chain (second row, right two structures) of half Ig-A, or the C-terminus of scFv-B may be connected to the N-terminus of the heavy chain (third row, right two structures) or the light chain (bottom row, right two structures) of half Ig-A. In some cases, the C-termini of VH-B and VL-B of Fv-B may be connected to the N-termini of the heavy and light chains, respectively (third row, left), or the light and heavy chains, respectively (bottom row, left), of half Ig-A. In certain cases, VH-B may be capable of binding to antigen B or epitope B without VL-B (i.e., VH-B as nanobody), and such VH-B may be connected to the C-terminus of the heavy chain of half-Ig-A (2nd row, left) or the N-terminus of the heavy or light chain of half-Ig-A.

FIG. 23C provides some additional exemplary ABU-A/B structures.

Top left section: Additional ABU-A/B structures comprising at least one ABU-A connected to at least one ABU-B (a generic schematic of ABU-A/B depicted in shaded box) may comprise a Fv which binds to antigen A or epitope A (Fv-A) and a Fv which binds to antigen B or epitope B (Fv-B) (let two structures). In some cases, Fv-A and Fv-B may be connected in tandem. In certain cases, Fv-A may be scFv-A, and/or Fv-B may be scFv-B. When both VH-A and VH-B are capable of binding to antigen or epitope A and antigen or epitope B, respectively, additional ABU-A/B structures may comprise, VH-A-CH (e.g., VH-A-CH1-(hinge)-CH2-CH3 or VH-A-CH2-CH3) and/or a VH-B-CL or VH-B (right three structures). In some cases, VH-A (or VH-A-CH) and VH-B may be connected in tandem.

Bottom section: Some exemplary ABU-A/B structures comprising at least one ABU-A paired with (not necessarily connected to for example via a covalent bond) at least one ABU-B are provided (a generic schematic of ABU-A/B depicted in the shaded box). Although one ABU-A and one ABU-B are shown, an ABU-A/B structure may comprise any number of, such as one, two, three, four, or more, ABU-As and ABU-Bs. In some cases, an ABU-A may comprise any of the structures provided in FIGS. 22A-22B or description thereof, and an ABU-B may comprise any of the structures provided in FIGS. 22A-22B or description thereof.

Bottom section top panel: In some ABU-A/B structures, an ABU-A may comprise a Fab-CH or Fab-like-CH (e.g., Fab-CH2-CH3 such as a half Ig, or Fab-like-CH2-CH3 such as half Ig-like) which binds to antigen A or epitope A (e.g., Fab-A-CH2-CH3 such as half Ig-A, or Fab-like-A-CH2-CH3 such as half Ig-like-A)) or a Fv-CH (e.g., Fv-CH2-CH3) which binds to antigen A or epitope A (e.g., Fv-A-CH2-CH3). In some ABU-A/B structures, an ABU-B may comprise a Fab-CH or Fab-like-CH (e.g., Fab-CH2-CH3 such as a half Ig, or Fab-like-CH2-CH3 such as half Ig-like) which binds to antigen B or epitope B (e.g., Fab-B-CH2-CH3 such as half Ig-B, or Fab-like-B-CH2-CH3 such as half Ig-like-B)) or a Fv-CH (e.g., Fv-CH2-CH3) which binds to antigen B or epitope B (e.g., Fv-B-CH2-CH3). Part of the CH of ABU-A and part of the CH of ABU-B may pair with each other to form ABU-A/B. In certain cases, the Fab-A, Fab-like-A, or Fv-A portion may be a scFab, scFab-like, or scFv, respectively. In certain cases, the Fab-B, Fab-like-B, or Fv-B portion may be a scFab, scFab-like, or scFv, respectively. When an ABU-A comprises a Fab-CH2-CH2 and an ABU-B comprises a Fab-like-CH2-CH3 (in Fab-like the CH1-CL positions are swapped relative to Fab), or vice versa, the ABU-A/B structure may be referred to as a “CrossMab”.

Bottom section bottom left panel: In some ABU-A/B structures, an ABU-A may comprise a Fab or Fab-like which binds to antigen A or epitope A (e.g., Fab-A or Fab-like-A) or a Fv which binds to antigen A or epitope A (e.g., Fv-A). In some ABU-A/B structures, an ABU-B may comprise a Fab or Fab-like which binds to antigen B or epitope B (e.g., Fab-B or Fab-like-B) or a Fv which binds to antigen B or epitope B (e.g., Fv-B). Part of the ABU-A and part of the ABU-B may pair with each other (e.g., via hinge) to form ABU-A/B. In certain cases, the Fab-A, Fab-like-A, or Fv-A portion may be a scFab, scFab-like, or scFv, respectively. In certain cases, the Fab-B, Fab-like-B, or Fv-B portion may be a scFab, scFab-like, or scFv, respectively. When an ABU-A comprises a Fab and an ABU-B comprises a Fab-like (in Fab-like the CH1-CL positions are swapped relative to Fab), or vice versa, the ABU-A/B structure may be referred to as a “CrossFab”.

Bottom section bottom right panel: In some ABU-A/B structures, when VH-A is capable of binding to antigen or epitope A without VL-A (e.g., nanobody A) and VH-B is capable of binding to antigen or epitope B without VL-B (e.g., nanobody B), an ABU-A may comprise VH-A-CH (e.g., VH-A-CH1-CH2-CH3, CH1 (or any other CH) may be omitted as desired) and an ABU-B may comprise VH-B-CL (CL may be omitted as desired). Part of the ABU-A and part of the ABU-B may pair with each other (e.g., via disulfide bond(s)) to form ABU-A/B.

Top right section: Further exemplary ABU-A/B structures comprising at least one ABU-A and at least one ABU-B are provided. In some ABU-A/B structures, an ABU-A may comprise a Fv-A (or a Fab-A), and an ABU-B may comprise a Fv-B (or a Fab-B), wherein at least one chain of the Fv-A (or Fab-A) and one chain of the Fv-B (or Fab-B) are connected. The other chain of the Fv-A (or Fab-A) and the other chain of the Fv-B (Fab-B) may not be connected (left) or may be connected (center and right).

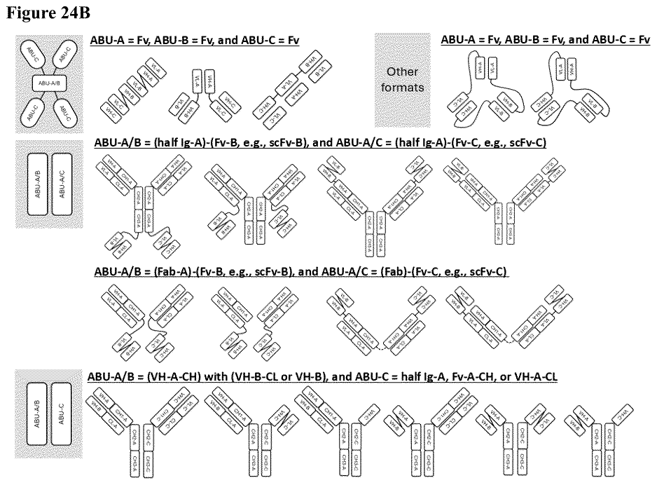

FIGS. 24A-24B provide some exemplary and non-limiting structures which may be comprised by or contained in antibodies comprising more than one antigen binding units (ABUs) such as multispecific antibodies according to the present disclosure. In addition to the above-noted rules (1)-(19), the following rules apply unless otherwise indicated: (20) VH-C and VL-C form an antigen binding site specific for antigen C or epitope C; (21) antigen C or epitope C may be different from or same as antigen A or B or epitope A or B; and (22) an ABU which binds to antigen C or epitope C may be referred to as ABU-C; an ABU which binds to antigen or epitope A and antigen or epitope C may be referred to as ABU-A/C and may comprise at least one ABU-A and at least one ABU-C; an ABU which binds to antigen or epitope B and antigen or epitope C may be referred to as ABU-B/C and may comprise at least one ABU-B and at least one ABU-C; and an ABU which binds to antigen or epitope A, antigen or epitope B, and antigen or epitope C may be referred to as ABU-A/B/C and may comprise at least one ABU-A, at least one ABU-B, and at least one ABU-C. In some embodiments, antigens A, B, and C may be CD3, CD19, and BCA respectively, CD3, BCMA, and CD19, respectively, CD19, CD3, and BCMA, respectively, CD19, BCMA, and CD3, respectively, BCMA, CD3, and CD19, respectively, or BCMA, CD19, and CD3, respectively.

FIG. 24A provides some exemplary ABU-A/B/C structures comprising at least one ABU-C connected to at least one ABU-A/B (a generic schematic of ABU-A/B depicted in the shaded box). Although one ABU-A/B and four ABU-Cs are shown, an ABU-A/B/C structure may comprise any number of, such as one, two, three, four, or more, ABU-Cs and ABU-A/Bs. In some cases, an ABU-C may comprise any of the structures provided in FIGS. 22A-22B or description thereof, and an ABU-A/B may comprise any of the structures provided in FIGS. 23A-23C or description thereof.

Top panel: In some ABU-A/B/C structures, an ABU-A/B may comprise a Fab-A connected to a Fv-B (e.g., scFv-B) (e.g., a structure in FIG. 23A right panel) and an ABU-C may comprise a Fv-C (e.g., scFv-C). Such structures may be referred to as Fab-(scFv)₂. For example, in the boxed (solid) structure, the N-terminus of Fv-C (e.g., scFv-C) (e.g., a structure in the third row in FIG. 22B) is connected to the C-terminus of the heavy chain of an ABU-A/B comprising the top left boxed structure in the right panel of FIG. 23A. The top row of the right panel provides some variations of the boxed ABU-A/B/C structure, in which different VH-VL domain orders are used in Fv-B and/or Fv-C, which may be according to the dash boxed structures in FIG. 22B. Some other variations of the boxed ABU-A/B/C structure include one in which the N-terminus of Fv-C is connected to the C-terminus of the light chain of ABU-A/B (i.e., the C-terminus of Fv-B) (top right in the left panel), one in which the C-terminus of Fv-C (e.g., scFv-C) is connected to the N-terminus of the light chain (top, second from left in the left panel) or the heavy chain (top, third from the left in the left panel) of ABU-A/B. In the bottom left structure (boxed, dashed), the C-terminus of Fv-C (e.g., scFv-C) (e.g., structures in the third row in FIG. 22B) is connected to the N-terminus of the heavy chain of an ABU-A/B comprising the structure second from the right on the bottom row of FIG. 23A. Some variations thereof are shown in the bottom right panel, including ones in which different VH-VL domain orders are used in Fv-B and/or Fv-C, which may be according to the dash boxed structures in FIG. 22B. Some other variations include one in which the C-terminus of Fv-C is connected to the N-terminus of the light chain of ABU-A/B (i.e., the N-terminus of Fv-B) (bottom right structure in the left panel), one in which the N-terminus of Fv-C (e.g., scFv-C) is connected to the C-terminus of the light chain (bottom, second from left in the left panel) or the heavy chain (top, third from the left in the left panel) of ABU-A/B.

Middle panels: In some ABU-A/B/C structures, an ABU-A/B may comprise a Fab-A or Fab-like-A paired with a Fab-B or Fab-like-B (e.g., FIG. 23C bottom panel left three structures) and an ABU-C may comprise a Fv-C (e.g., scFv-C). For example, the N-terminus of the Fv-C may be connected to the C-terminus of the light chain of Fab-A or Fab-like-A (left structure) or of Fab-B or Fab-like-B; the C-terminus of the Fv-C may be connected to the N-terminus of the light chain of Fab-A or Fab-like-A (second from left) or of the heavy chain of Fab-B or Fab-like-B, or to the N-terminus of the heavy chain of Fab-A or Fab-like-A (third from left) or of the heavy chain of Fab-B or Fab-like-B. In some other examples, the N-termini of the VH-C and the VL-C of Fv-C may be connected to the C-termini of the heavy chain of Fab-A or Fab-like-A and the heavy chain of Fab-B or Fab-like-B, respectively (second from right), or to the C-termini of the heavy chain of Fab-B or Fab-like-B and the heavy chain of Fab-A or Fab-like-A, respectively. In further examples, the C-termini of the VH-C and the VL-C of Fv-C may be connected to: the N-termini of the light chain and heavy chain, respectively, of Fab-A or Fab-like-A (right structure); or to the N-termini of the heavy chain and light chain, respectively of Fab-A or Fab-like-A; the N-termini of the light chain and heavy chain, respectively, of Fab-B or Fab-like-B (right structure); or to the N-termini of the heavy chain and light chain, respectively of Fab-B or Fab-like-B.

Bottom panels: In some ABU-A/B/C structures, an ABU-A/B may comprise a Fab-A or Fab-like-A paired with a Fab-B or Fab-like-B (e.g., FIG. 23C bottom panel left three structures) and an ABU-C may comprise a Fab-C or Fab-like-C (e.g., scFab-C or scFab-like-C). For example, the N-terminus of the Fab-C may be connected to the C-terminus of the light chain of Fab-A or Fab-like-A (left structure) or of Fab-B or Fab-like-B; or the C-terminus of the Fab-C may be connected to the N-terminus of the light chain of Fab-A or Fab-like-A (second from left) or of the heavy chain of Fab-B or Fab-like-B, or to the N-terminus of the heavy chain of Fab-A or Fab-like-A (third from left) or of the heavy chain of Fab-B or Fab-like-B. In some other examples, the N-termini of the VH-C and the VL-C of Fab-C may be connected to the C-termini of the heavy chain of Fab-A or Fab-like-A and the heavy chain of Fab-B or Fab-like-B, respectively (second from right), or to the C-termini of the heavy chain of Fab-B or Fab-like-B and the heavy chain of Fab-A or Fab-like-A, respectively. In further examples, the C-termini of the heavy chain and light chain of Fab-C may be connected to: the N-termini of the light chain and heavy chain, respectively, of Fab-A or Fab-like-A (right structure); to the N-termini of the heavy chain and light chain, respectively of Fab-A or Fab-like-A; the N-termini of the light chain and heavy chain, respectively, of Fab-B or Fab-like-B (right structure); or to the N-termini of the heavy chain and light chain, respectively of Fab-B or Fab-like-B.

FIG. 24B provides some additional exemplary ABU-A/B/C structures.

Top left section: Additional ABU-A/B/C structures comprising at least one ABU-C connected to at least one ABU-A/B (a generic schematic of ABU-A/B depicted in shaded box) may comprise a Fv which binds to antigen C or epitope C (Fv-C) connected to a ABU-A/B comprising a Fv-A and a Fv-B. In some cases, Fv-A, Fv-B, and Fv-C may be connected in tandem (left). In some cases, Fv-C may be connected to a VH or VL of ABU-A/B (center and right). In certain cases, Fv-A may be scFv-A, Fv-B may be scFv-B, and/or Fv-C may be scFv-C.

Middle section: Some exemplary ABU-A/B/C structures comprise at least one ABU-A/B paired with (not necessarily connected to for example via a covalent bond) at least one ABU-A/C are provided (a generic schematic of ABU-A/B/C depicted in the shaded box). Although one ABU-A/B and one ABU-A/C are shown, an ABU-A/B/C structure may comprise any number of, such as one, two, three, four, or more, ABU-A/Bs and ABU-A/Cs. In some cases, an ABU-A/B may comprise any of the structures provided in FIGS. 23A-23C or description thereof, and an ABU-A/C may comprise any of the structures provided in FIGS. 23A-23C or description thereof (except that the “B” (for antigen or epitope B) in FIGS. 23A-23C should be replaced with “C”).

Middle section top panel: Some exemplary ABU-A/B/C structures may comprise: an ABU-A/B comprising a structure provided in FIG. 23B or description thereof; paired with an ABU-A/C comprising a structure provided in FIG. 23B or description thereof (except that the “B” (for antigen or epitope B) in FIG. 23B should be replaced with “C”). While in the four structures depicted the ABU-A/B and the ABU-A/C comprise the same format (domain orientation) selected from FIG. 23B, the format (domain orientation) of ABU-A/B and the format domain orientation) of ABU-A/C may differ from each other.

Middle section bottom panel: Some exemplary ABU-A/B/C structures may comprise: an ABU-A/B comprising a structure provided in FIG. 23A or description thereof; paired with an ABU-A/C comprising a structure provided in FIG. 23A or description thereof (except that the “B” (for antigen or epitope B) in FIG. 23A should be replaced with “C”). For example, the left structure comprises: an ABU-A/B comprising a structure of the bottom left among the boxed structures in FIG. 23A or description thereof; paired with an ABU-A/C comprising a structure of the bottom left among the boxed structures in FIG. 23A (except that the “B” should be replaced with “C”). The second from the left structure comprises: an ABU-A/B comprising a structure of the top left among the boxed structures in FIG. 23A or description thereof; paired with an ABU-A/C comprising a structure of the top left among the boxed structures in FIG. 23A (except that the “B” should be replaced with “C”). The second from the right structure comprises: an ABU-A/B comprising a structure of the second from the right of the third row in FIG. 23A or description thereof; paired with an ABU-A/C comprising a structure of the second from the right of the third row in FIG. 23A (except that the “B” should be replaced with “C”). The right structure comprises: an ABU-A/B comprising a structure of the second from the right of the bottom row in FIG. 23A or description thereof; paired with an ABU-A/C comprising a structure of the second from the right of the bottom row in FIG. 23A (except that the “B” should be replaced with “C”). While in the four structures depicted the ABU-A/B and the ABU-A/C comprise the same format (domain orientation) selected from FIG. 23A, the format (domain orientation) of ABU-A/B and the format domain orientation) of ABU-A/C may differ from each other.

Bottom section: Some exemplary ABU-A/B/C structures comprise at least one ABU-A/B paired with (not necessarily connected to for example via a covalent bond) at least one ABU-C are provided (a generic schematic of ABU-A/B/C depicted in the shaded box). Although one ABU-A/B and one ABU-A/C are shown, an ABU-A/B/C structure may comprise any number of, such as one, two, three, four, or more, ABU-A/Bs and ABU-Cs. In some cases, an ABU-A/B may comprise any of the structures provided in FIGS. 23A-23C or description thereof, and an ABU-C may comprise any of the structures provided in FIGS. 22A-22B or description thereof. In some cases, when VH-A is capable of binding to antigen or epitope A without VL-A and VH-B is capable of binding to antigen or epitope B without VL-B, some exemplary ABU-A/B/C structures may comprise an ABU-A/B comprising a structure provided in the right panel of the top left section in FIG. 23C or description thereof or a structure provided in the bottom right panel of the bottom section in FIG. 23C or description thereof. Such an ABU-A/B may for example be paired with an ABU-C comprising a structure of FIG. 22B, such as one in the top or bottom row. The left three ABU-A/B/C's comprise an ABU-A/B having a structure provided in the bottom right panel of the bottom section in FIG. 23C or description thereof, and the right three ABU-A/B/C's comprise an ABU-A/B having a structure provided in the right panel of the top left section in FIG. 23C or description thereof. The left and the fourth from the left ABU-A/B/C's comprise an ABU-C comprising a structure in the top row of FIG. 22B or description thereof, and the second and fifth from the left ABU-A/B/C's comprise an ABU-C comprising a structure in the bottom row of FIG. 22B or description thereof. In certain cases, when VH-C is capable of binding to antigen or epitope C without VL-C, and the right and the fourth from the right ABU-A/B/C's comprise an ABU-C comprising a structure in the top row of FIG. 22B or description thereof, except that VL-B may be omitted.

Top right section: Further exemplary ABU-A/B/C structures comprise at least one ABU-A, at least one ABU-B, and at least one ABU-C are provided. In some ABU-A/B/C structures, an ABU-A may comprise a Fv-A (or a Fab-A), an ABU-B may comprise a Fv-B (or a Fab-B), and an ABU-C may comprise a Fv-C (or a Fab-C), wherein at least one chain of the Fv-A (or Fab-A) and one chain of the Fv-B (or Fab-B) are connected, at least one chain of the Fv-B (or Fab-B) and one chain of the Fv-C (or Fab-C) are connected, and at least one chain of the Fv-C (or Fab-C) and one chain of the Fv-A (or Fab-A) are connected.

### DETAILED DESCRIPTION

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein is well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of the term “including,” as well as other forms, such as “includes” and “included,” is not limiting.

As used herein, the term “about” or “approximately” when used in reference to a particular recited numerical value, means that the value may vary from the recited value by no more than 10%. For example, as used herein, the expression “about 100” includes 90 and 110 and all values in between (e.g., 91, 92, 93, 99, 99.1, 99.2, 99.3, 99.4, 100, 100.8, 100.9, 101, 106, 107, 108, 109, etc.).

So that the invention may be more readily understood, certain terms are first defined. As used herein, the term “antigen binding protein” refers to a protein that specifically binds to or is immunologically reactive with an antigen or epitope. The term “antigen binding protein” includes “antibody” or more particularly “antibody fragment”, but also non-immunoglobulin-based binding proteins. Non-limiting examples of non-immunoglobulin-based binding proteins include DARPins, affimers, monobodies, anticalins, fynomers, and affibodies. Non-immunoglobulin-based binding proteins are described in further detail in Simeon et al. (Protein Cell. 2018. 9(1): 3-14) and Olaleye et al. (Biomolecules. 2021. 11(12): 1791), each of which is incorporated herein by reference. For example, an antigen binding protein which binds to BCMA may be referred to herein as a BCMA binding protein.

The term “antibody” refers to an immunoglobulin molecule or immunoglobulin derived molecule that specifically binds to, or is immunologically reactive with an antigen or epitope.

The terms “intact antibody,” “full length antibody,” and “whole antibody” or the like are used herein interchangeably and refer to an antibody having a structure substantially similar to a native antibody. In some instances, an antibody comprises heavy (H) and light (L) chains interconnected by disulfide bonds. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called a, 8, &, Y, and u, respectively. For example, an intact IgG (or IgD or IgE) antibody comprises two immunoglobulin heavy chains and two immunoglobulin light chains. Therefore, in some instances, an antibody according to the present disclosure may comprise two pairs of heavy and light chains interconnected by disulfide bonds, or an antigen binding fragment(s) thereof. Some intact antibody comprises multiple units, each comprising two pairs of heavy and light chains interconnected by disulfide bonds. For example, an intact IgA comprises two units and an intact IgM comprises five units. Therefore, in other instances, an antibody according to the present disclosure may instead comprise multiple (e.g., two, three, four, five, and so on) units each comprising two pairs of heavy and light chains interconnected by disulfide bonds, or an antigen binding fragment(s) thereof.

In an intact antibody, each heavy chain comprises a variable region, such as a heavy chain variable region (VH) (also referred to as heavy chain variable region), and a heavy chain constant region (CH). In case of an intact antibody, a CH comprises a heavy chain constant domain 1 (CH1), a hinge, a heavy chain constant domain 2 (CH2), and a heavy chain constant domain 3 (CH3), with some exceptions, e.g., a camel IgG2 or camel IgG3, in which a CH comprises a hinge, a CH2, and a CH3, or a shark antibody (e.g., IgNAR), in which a CH comprises a hinge, a CH1, a CH2, a CH3, a CH4, and a CH5. In an intact antibody, each light chain is comprised of: a light chain variable region (VL); and a light chain constant region (CL). A CL is typically comprised of one light chain constant domain, and thus a light chain constant domain may also be referred to as a CL or a CL domain. Typically (with some exceptions such as nanobodies, camelid heavy chain antibodies, IgNARs, and the like), one VH and one VL may form an antigen binding structure. Therefore, “a set of variable domains” or “a variable domain pair” (or the like) of an antibody means the set or combination of the VH and VL of the antibody.

The phrase “heavy chain” or “HC” is used in its broadest sense and encompasses a polypeptide comprising at least one immunoglobulin heavy chain domain or part (i.e., at least one of VH, CH1, hinge, CH2, and/or CH3). Similarly, the phrase “light chain” or “LC” is used in its broadest sense and encompasses a polypeptide comprising at least one immunoglobulin light chain domain (i.e., at least one of VL and/or CL).

An “antibody” includes both polyclonal and monoclonal antibodies, as well as functional antibody fragments that exhibit the desired antigen binding activity, including but not limited to fragment antigen binding (Fab) fragments, F(ab′)2 fragments, Fv fragments, recombinant IgG (rlgG) fragments, single chain variable fragments (scFv) and single domain antibodies (e.g., sdAb, sdFv, nanobody, VHH fragments). The antibody may thus be a single domain antibody or comprise at least one variable light and at least one variable heavy chain.

In one embodiment, the at least one variable light and at least one variable heavy chain are displayed as a single polypeptide chain. In certain embodiments, the term “antibody” or “antigen binding protein” includes germline derived antibodies. The term “antibody” or “antigen binding protein” includes genetically engineered or otherwise modified forms of immunoglobulins, such as intrabodies, peptibodies, chimeric antibodies, fully human antibodies, humanized antibodies, heteroconjugate antibodies (e.g., bispecific antibodies, diabodies, triabodies, tetrabodies, tandem di-scFv, tandem tri-scFv) and the like. Unless otherwise stated, the term “antibody” should be understood to encompass functional antibody fragments thereof. In certain embodiments, the antibody is trispecific and comprises three different antigen binding domains, e.g., binding to three different target molecules. In certain embodiments, the terms “antigen binding protein” and “antigen targeting protein” are used interchangeably herein.

In certain embodiments, the antigen binding protein is multispecific (i.e., binds to two or more different target molecules or to two or more epitopes on the same target molecule). A “multispecific antigen binding protein” (e.g., “multispecific antibody” or “multispecific antibody fragment”) refers to an antigen binding protein (e.g., an antibody or antigen binding antibody fragment) comprising two or more antigen binding units (ABUs) (which term is used to mean parts of an antigen binding protein (e.g., an antibody or antibody fragment) which individually contain a structural element which provides antigen binding), wherein said two or more ABUs include at least two different ABUs which respectively recognize and specifically bind to different antigens and/or different epitopes. ABUs may also be referred to as antigen binding domain. For example, a CD19-binding ABU may also be referred to as a CD19 binding domain.

A “bispecific antigen binding protein” (e.g., “bispecific antibody” or “bispecific antibody fragment””) is a type of multispecific antigen binding protein (e.g., antibody or antibody fragment) comprising two different ABUs which recognize and specifically bind to two different antigens and/or two different epitopes. The two epitopes may or may not be within the same antigen. A bispecific antigen binding protein (e.g., bispecific antibody or antibody fragment) may generally target, for example, two different surface receptors on the same or different cells (e.g., an immune cell and a diseased cell, such as an activated fibroblast or a cancer cell).

A “trispecific antigen binding protein” (e.g., “trispecific antibody” or “trispecific antibody fragment”) is a type of multispecific antigen binding protein (e.g., antibody or antibody fragment) comprising three different ABUs which recognize and specifically bind to three different antigens and/or three different epitopes. The three epitopes may or may not be within the same antigen. A trispecific antigen binding protein (e.g., trispecific antibody or antibody fragment) may target, for example, three different surface receptors on the same or different cells (e.g., an immune cell and a diseased cell, such as an activated fibroblast or a cancer cell). In some embodiments, the three different antigens may be CD3, CD19, and BCMA.

In certain embodiments, the antigen binding protein is bispecific and e.g., binds to two different target molecules or to two different epitopes on the same target molecule. In certain embodiments, the antigen binding protein is trispecific and e.g., binds to at least three different target molecules. In certain embodiments thereof, a trispecific antigen binding protein comprises three binding sites and binds to antigens on at least two distinct cells.

The term “format” as used herein in relation to antibodies and antigen binding antibody fragments including multispecific antigen binding protein described herein refers to the structure of an antibody or antigen binding antibody fragment having all the domains contained in a referenced antibody structure, wherein the domains are placed in essentially the same orientation as in the referenced antibody structure. For example, by “an IgG format” it is meant that all the domains contained in a native IgG antibody are present in the same orientation as in a native IgG antibody. That is, an antibody of an IgG format comprises two sets of a heavy chain (comprising a VH, a CH1, a hinge, a CH2, and a CH3 from the N-terminus to the C-terminus) and a light chain (comprising a VL and a CL from the N-terminus to the C-terminus), wherein one heavy chain and one light chain are paired with each other and the other heavy chain and the other light chain are paired with each other, further wherein the two heavy chains are associated with each other. Each domain or part of an antibody of an IgG format may have the same sequence as the corresponding domain or part of a wild-type or reference IgG or may contain modifications (e.g., amino acid sequence changes, glycosylation, etc.) relative to the corresponding domain or part of a wild-type or reference IgG. Similarly, by “a Fab format” it is meant that all the domains contained in a native Fab fragment (e.g., produced by enzymatic digestion of native Ig molecule such as IgG) are present in the same orientation as in a native Fab. That is, an antibody fragment of a Fab format comprises a set of a heavy chain (comprising a VH and a CH1 from the N-terminus to the C-terminus) and a light chain (comprising a VL and a CL from the N-terminus to the C-terminus) paired with each other. Each domain or part of an antibody fragment of a Fab format may have the same sequence as the corresponding domain or part of a Fab derived from a wild-type or reference IgG or may contain modifications (e.g., amino acid sequence changes, glycosylation, etc.) relative to the corresponding domain or part of a wild-type or reference IgG.

The antigen binding protein may be monovalent or multivalent, i.e., having one or more antigen binding sites. Non-limiting examples of monovalent antigen binding proteins include scFv, Fab, scFab, dAb, VHH, V(NAR), DARPins, affilins and nanobodies. A multivalent antigen binding protein can have two, three, four or more antigen binding sites, accordingly, the antigen binding protein is “bivalent”, “trivalent”, “tetravalent” and so forth. Non-limiting examples of multivalent antigen binding proteins include full-length immunoglobulins, F(ab′)2 fragments, bis-scFv (or tandem scFv or BiTE), DART, diabodies, scDb, DVD-Ig, IgG-scFab, scFab-Fc-scFab, IgG-scFv, scFv-Fc, scFv-fc-scFv, Fv2-Fc, FynomABs, quadroma, CrossMab, DuoBody, triabodies and tetrabodies. In some embodiments, the multivalent antigen binding protein is bivalent, i.e., two binding sites are present. In some embodiments, the multivalent antigen binding protein is bispecific, i.e., the antigen binding protein is directed against two different targets or two different target sites on one target molecule. In some embodiments, the multivalent antigen binding protein includes more than two, e.g., three or four different binding sites for three or four, respectively, different antigens. Such antigen binding protein is multivalent and multispecific, in particular tri- or tetra-specific, respectively. Examples of trispecific formats include, without being limited to, a Fab-(scFv)₂, a Tri-MAB, a CODV-Ig (Crossover Dual Variable-Immunoglobulin), an OrthoTsAb, a 2+1 common LC, a Triplebody, a Triflex, a MATCH3, or a triabody (see, e.g., Tapia-Galisteo A, et al, Theranostics 2023; 13(3):1028-1041). For example, the trispecific Fab-(scFv)2 format may comprise: a Fab specific for antigen or epitope A (Fab-A) comprising a heavy chain comprising a VH and a CH1 and a light chain comprising a VL and a CL; a scFv specific for antigen or epitope B (scFv-B) comprising a VL linked to a VH; and a scFv specific for antigen or epitope C (scFv-C) comprising a VL linked to a VH. Exemplary structures of Fab(scFv)2 are shown in FIG. 24A top panel. For example, the trispecific Tri-MAB format may comprise: a half-Ig specific for antigen or epitope A (half-Ig-A) comprising a heavy chain and a light chain; a half-Ig specific for antigen or epitope B (half-Ig-B) comprising a heavy chain and a light chain; and another antigen binding unit such as a scFv, Fv, Fab, scFab, etc specific for antigen or epitope C (ABU-C), wherein the half-Ig-A and half-Ig-B pair to form an Ig-like structure (e.g., shown in FIG. 23C top left of middle panel), to which ABU-C is attached.

In some embodiments, the antigen binding protein is multispecific (e.g., bispecific), such as, without being limited to, a diabody, a single-chain diabody, a DART, BiTE, BIKE, tandem scFv or IgG-like asymmetric heterobispecific antibodies. In certain embodiments, one of the binding specificities of the multispecific antigen binding protein is an immune cell engager (i.e., comprising affinity to a cell surface protein of an immune cell, also referred to as “immune cell binding domain”). Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells includes, without being limited to, CD3, TCRα, TCRβ, CD16, NKG2D, CD94/NKG2C, NKp30, NKp46, CD89, CD64, and CD32. In certain embodiments, the immune cell target antigen is CD3. In certain embodiments, the multispecific antigen binding protein is a trispecific targeting BCMA, CD19 and an immune cell target antigen.

Examples of immune cells that may be recruited include, but are not limited to, T cells, B cells, natural killer (NK) cells, natural killer T (NKT) cells, neutrophil cells, monocytes, and macrophages. Examples of surface proteins that may be used to recruit immune cells include, but are not limited to, CD3, TCRα, TCRβ, CD16, NKG2D, CD89, CD64, and CD32. Such immune cell redirecting trispecific antigen binding proteins may in some embodiments comprise a Fc domain.

As used herein, a “single-chain variable fragment” (scFv) is an antigen binding protein comprising a heavy chain variable domain (VH) linked to a light chain variable domain (VL). The VH and VL domains of the scFv may be linked via any appropriate art recognized linker. Such linkers include, but are not limited to, repeated GGGGS (SEQ ID NO: 31) amino acid sequences or variants thereof, e.g., SEQ ID NO: 35, 34, 33, 32, 31, or 36. In some cases, the VH and the VL are additionally or alternatively connected by one or more disulfide bonds. In certain cases, the VH and/or the VL sequences may be modified (e.g., one or more amino acids may be substituted) to comprise a cysteine residue to allow for such a disulfide bond (e.g., see Weatherill et al., Protein Eng Des Sel. 2012 July; 25 (7): 321-9.). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. The scFv is generally free of antibody constant domain regions, although an scFv according to the present disclosure may be linked or attached to antibody constant domain regions (e.g., antibody Fc domain) to alter various properties of the scFv, including, but not limited to, increased serum or tissue half-life. An scFv generally has a molecular weight of about 25 kDa and a hydrodynamic radius of about 2.5 nm.

As used herein, a “Fab fragment”, “Fab” or “Fab domain” is an antibody fragment comprising a light chain fragment comprising a variable light (VL) domain and a constant domain of the light chain (CL), and variable heavy (VH) domain and a first constant domain (CH1) of the heavy chain. A F(ab′)₂ comprises two antigen binding regions joined at the hinge through disulfides. When the CH1 and the CL positions in a Fab are swapped, i.e., the VL is joined to a CH1 and the VH is joined to a CL, the resulting molecule may be referred to a Fab-like.

A “half-antibody” variant of a given antibody or antigen binding antibody fragment as used herein refers to an antigen binding antibody structure which comprises a symmetrical half or an essentially symmetrical half of the given antibody or antigen binding antibody fragment. Therefore, when the given antibody or antigen binding fragment is a homodimer (or an essentially homodimer), the half-antibody variant thereof comprises the corresponding monomer. For example, a half-antibody variant of an intact IgG comprises one (but not two) set of a heavy chain comprising a VH, a CH1, a hinge, a CH2, and a CH3 and a light chain comprising a VL and a CL. Similarly, a half-antibody variant of a scFv-Fc comprises one (but not two) polypeptide comprising a scFv, a hinge (or a linker), a CH2, and a CH3, and a half-antibody variant of a minibody comprises one (but not two) polypeptide comprising a scFv, a hinge (or a linker), and a CH3. Since a half-antibody variant of a scFv-Fc and a half-antibody variant of a minibody are both single-chain molecules, they may also be referred to as a single-chain variant of a scFv-Fc and a single-chain variant of a minibody, respectively. Further similarly, a half-antibody variant of a camel Ig (or camel IgG, such as camel IgG2 or camel IgG3) comprises one (but not two) polypeptide comprising a VH, a hinge (or a linker), a CH2, and a CH3, and a half-antibody variant of an IgNAR comprises one (but not two) polypeptide comprising a VH, a hinge (or a linker), a CH1, a CH2, a CH3, a CH4, and a CH5. Since a half-antibody variant of a camel Ig and a half-antibody variant of an IgNAR are both single-chain molecules, they may also be referred to as a single-chain variant of a camel Ig and a single-chain variant of an IgNAR, respectively.

As used herein, a “VHH”, “nanobody”, “heavy-chain only antibody”, “single domain antibody”, or “sdAb” is an antigen binding protein comprising a single heavy chain variable domain derived from the species of the Camelidae family, which includes camels, llama, alpaca. A VHH generally has a molecular weight of about 15 kDa. The term “VHH” as used herein further encompasses a VH domain which is capable of binding to its cognate antigen or epitope without a VL, regardless of the species origin.

As used herein, a “Fab” or “fragment antigen binding” is a portion of an immunoglobulin that retains the antigen binding regions of the original antibody while lacking the Fc portion (i.e., fragment crystallizable region). In an experimental setting, Fc and Fab fragments can be generated through papain digestion of a full-length antibody, resulting in two monovalent Fab fragments and one Fc portion. The Fab fragments, with a molecular weight of approximately 50 kDa, have better permeability characteristics than full-length antibodies. With the Fc portion being absent, Fab fragments do not interact with Fc receptors on cells or trigger any other in vivo Fc-effector function. Fabs are composed of one heavy and one light chain, each chain having a constant (HC or LC) and a variable domain (VH or VL). In certain embodiments, the antigen binding protein comprises an Fc domain. The presence of an Fc domain may be advantageous to induce cytotoxic immune responses and/or activate complement (e.g., ADCC/ADCP or CDC effector function). Exemplary antibody formats including an Fc domain, without being limited to, are full-length immunoglobulins, DVD-Ig, and scFv-Fc. scFv fusions, IgG-scFab, scFab-dsscFv, Fv2-Fc, IgG-scFv fusions (such as e.g., bsAb, Bs1Ab, Bs2Ab, Bs3Ab, Ts1Ab, Ts2Ab, Knob-into-Holes (KiHs)), DuoBody and/or CrossMabs. An active Fc domain may increase the likelihood of pro-inflammatory cytokine release by T cells and other effector cells in the tumor microenvironment which is believed to be part of the therapeutic mechanism of action. The Fc domain may be fully active or partly silenced to avoid over-stimulation of the immune system. In some embodiments, the Fc domain is inactive and does not stimulate pro-inflammatory cytokine release but does still improve half-life and/or stability of the antigen binding protein. In some embodiments, the antigen binding protein comprises a constant region selected from the group consisting of human IgG1, IgG2, IgG3 or IgG4 isotype. In other embodiments, the antigen binding protein comprises a constant region selected from the group consisting of murine IgG1, IgG2A, IgG2B or IgG3 isotype. In certain embodiments, the antigen binding protein does not comprise a Fc domain.

The antigen binding proteins according to the present disclosure may comprise one or more linkers for linking the domains of the antigen binding protein (e.g., linking a VH and VL to form a scFv, or linking multiple binding domains to form a multispecific antigen binding protein). Illustrative examples of linkers include glycine polymers (Gly)n; glycine-serine polymers (GlynSer)n, where n is an integer of at least one, two, three, four, five, six, seven, or eight (SEQ ID NO: 123); glycine-alanine polymers; alanine-serine polymers; and other flexible linkers known in the art.

Glycine and glycine-serine polymers are relatively unstructured, and therefore may be able to serve as a neutral tether between domains of fusion proteins such as the antigen binding proteins described herein. Glycine accesses significantly more phi-psi space than other small side chain amino acids, and is much less restricted than residues with longer side chains (Scheraga, Rev. Computational Chem. 1:1173-142 (1992)). A person skilled in the art will recognize that design of an antigen binding protein in particular embodiments can include linkers that are all or partially flexible, such that the linker can include flexible linker stretches as well as one or more stretches that confer less flexibility to provide a desired structure.

Linker sequences can however be chosen to resemble natural linker sequences, for example, using the amino acid stretches corresponding to the beginning of human CH1 and CK sequences or amino acid stretches corresponding to the lower portion of the hinge region of human IgG.

The design of the peptide linkers connecting VL and VH domains in the scFv moieties are flexible linkers generally composed of small, non-polar or polar residues such as, e.g., Gly, Ser and Thr. A particularly exemplary linker connecting the variable domains of the scFv moieties is the (Gly₄Ser)₄ linker (SEQ ID NO: 34), where 4 is the exemplary number of repeats of the motif. Linkers connecting the scFv antigen binding proteins to the Fab domain are also envisioned. In certain embodiments, the scFv antigen binding proteins are linked to the CH1 and CL domains of the Fab with a Gly-Ser linker. In certain embodiments, the linker comprises the amino acid sequence Linkers connecting the scFv antigen binding proteins to the Fab domain are also envisioned. In certain embodiments, the scFv antigen binding proteins are linked to the CH1 and CL domains of the Fab with a Gly-Ser linker. In certain embodiments, the linker comprises the amino acid sequence GGGGS (SEQ ID NO: 31). In certain embodiments, the linker comprises 3-5 repetitions of the GGGGS (SEQ ID NO: 31) motif. In certain embodiments, the amino acid linker comprises the amino acid sequence GGGGSGGGGSGGGGS (SEQ ID NO: 33), GGGGSGGGGSGGGGSGGGGS (SEQ ID NO: 34), or GGGGSGGGGSGGGGSGGGGAS (SEQ ID NO: 36). Alternatively, linker sequences connecting the variable domains may include glycine polymers (Gly) n; glycine-alanine polymers; alanine-serine polymers; and other flexible linkers known in the art.

Other exemplary linkers include, but are not limited to the following amino acid sequences: GGG; DGGGS (SEQ ID NO: 39); TGEKP (SEQ ID NO: 40) (Liu et al, Proc. Natl. Acad. Sci. 94:5525-5530 (1997)); GGRR (SEQ ID NO: 41); (GGGGS) n (SEQ ID NO: 38) wherein n=1, 2, 3, 4 or 5 (Kim et al, Proc. Natl. Acad. Sci. 93:1156-1160 (1996)); EGKSSGSGSESKVD (SEQ ID NO: 42) (Chaudhary et al., Proc. Natl. Acad. Sci. 87:1066-1070 (1990)); KESGSVSSEQLAQFRSLD (SEQ ID NO: 43) (Bird et al., Science 242:423-426 (1988)), GGRRGGGS (SEQ ID NO: 44); LRQRDGERP (SEQ ID NO: 45); LRQKDGGGSERP (SEQ ID NO: 46); and GSTSGSGKPGSGEGSTKG (SEQ ID NO: 47) (Cooper et al, Blood, 101 (4): 1637-1644 (2003)). Alternatively, flexible linkers can be rationally designed using a computer program capable of modeling the 3D structure of proteins and peptides or by phage display methods.

The antibodies may comprise a variable light (VL) domain and a variable heavy (VH) domain. Each VL and VH domain further comprises a set of three CDRs.

As used herein, the term “complementarity determining region” or “CDR” refers to non-contiguous sequences of amino acids within antibody variable regions, which confer antigen specificity and binding affinity. In general, there are three CDRs in each heavy chain variable domain (CDR-H1, CDR-H2, CDR-H3) and three CDRs in each light chain variable domain (CDR-L1, CDR-L2, CDR-L3). “Framework regions” or “FRs” are known in the art to refer to the non-CDR portions of the variable domains of the heavy and light chains. In general, there are four FRs in each heavy chain variable domain (HFR1, HFR2, HFR3, and HFR4), and four FRs in each light chain variable domain (LFR1, LFR2, LFR3, and LFR4). Accordingly, an antibody variable region amino acid sequence can be represented by the formula FR1-CDR1-FR2-CDR2-FR3-CDR3-FR4. Each segment of the formula, i.e., FR1, CDR1, FR2, CDR2, FR3, CDR3, and FR4, represents a discrete amino acid sequence (or a polynucleotide sequence encoding the same) that can be mutated, including one or more amino acid substitutions, deletions, and insertions. In certain embodiments, an antibody variable light chain amino acid sequence can be represented by the formula LFR1-(CDR-L1)-LFR2-(CDR-L2)-LFR3-(CDR-L3)-LFR4. In certain embodiments, an antibody variable heavy chain amino acid sequence can be represented by the formula HFR1-(CDR-H1)-HFR2-(CDR-H2)-HFR3-(CDR-H3)-HFR4.

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme), and Honegger A and Plückthun A, “Yet another numbering scheme for immunoglobulin variable domains: an automatic modeling and analysis tool,” J Mol Biol, 2001 Jun. 8; 309(3):657-70, (“AHo” numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

Table 1 below lists exemplary position boundaries of CDR-L1, CDR-L2, CDR-L3 and CDR-H1, CDR-H2, CDR-H3 of an antibody, as identified by Kabat, Chothia, and Contact schemes, respectively. For CDR-H1, residue numbering is listed using both the Kabat and Chothia numbering schemes. CDRs are located between FRs, for example, with CDR-L1 located between LFR1 and LFR2, and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Exemplary Position Boundaries of CDRs

| CDR | Kabat | Chothia | Contact |
|---|---|---|---|
| CDR-L1 | L24--L34 | L24--L34 | L30--L36 |
| CDR-L2 | L50--L56 | L50--L56 | L46--L55 |
| CDR-L3 | L89--L97 | L89--L97 | L89--L96 |
| CDR-H1 | H31--H35B | H26--H32 . . . 34 | H30--H35B |
| (Kabat Numbering¹) | | | |
| CDR-H1 | H31--H35 | H26--H32 | H30--H35 |
| (Chothia Numbering²) | | | |
| CDR-H2 | H50--H65 | H52--H56 | H47--H58 |
| CDR-H3 | H95--H102 | H95--H102 | H93--H101 |

¹Kabat et al. (1991), “Sequences of Proteins of Immunological Interest,” 5th Ed. Public Health Service, National Institutes of Health, Bethesda, MD
²Al-Lazikani et al. (1997), J. Mol. Biol. 273: 927-948

Thus, unless otherwise specified, a “CDR” or “complementary determining region,” or individual specified CDRs (e.g., CDR-H1, CDR-H2), of a given antibody or fragment thereof, such as a variable domain thereof, should be understood to encompass a (or the specific) complementary determining region as defined by Kabat. Likewise, unless otherwise specified, an “FR” or “framework region,” or individual specified FRs (e.g., “HFR1,” “HFR2”) of a given antibody or fragment thereof, such as a variable domain thereof, should be understood to encompass a (or the specific) framework region as defined by Kabat. In some instances, the scheme for identification of a particular CDR or FR is specified, such as the CDR as defined by the Kabat, Chothia, Contact, AHo or IMGT method. Therefore, for example, the amino acid sequences of s set of a CDR-L1, a CDR-L2, and a CDR-L3 may be defined as having the light chain CDR1, 2, and 3 sequences contained in a given VL according to any of such CDR definitions, and the amino acid sequences of s set of a CDR-H1, a CDR-H2, and a CDR-H3 may be defined as having the heavy chain CDR1, 2, and 3 sequences contained in a given VH according to any of such CDR definitions. In other cases, the particular amino acid sequence of a CDR or FR is given.

According to IMGT (the international ImMunoGeneTics information system for immunoglobulins or antibodies, T cell receptors, MH, immunoglobulin superfamily IgSF and MhSF), the CH1 domain is the amino acid positions (or simply referred to as “positions” herein) 118-215 (EU numbering) and the hinge region is the amino acid positions 216-230 (EU numbering). The term “CH1 domain” is used in a broad sense herein to encompass any naturally occurring, corresponding heavy chain constant domain and/or region allotypes and variants thereof, which may comprise fewer or more amino acids and/or amino acid modification(s) and to refer to a heavy chain region comprising at least 80% of the heavy chain positions 118-215 (EU numbering)) and in some instances also comprising a portion of the hinge region (a portion of heavy chain positions 216-230 (EU numbering)) is included (e.g., up to position 218 or 220, in particular cases up to position 220).

A human IgG1 CH1 domain reference sequence, corresponding to the amino acid positions 118-215 according to EU numbering, is provided herein as SEQ ID NO: 56, which corresponds to the CH1 domain sequence of human IgG1 Allotype “IGHG1*01 (J00228)”, “IGHG1*04 (JN582178)”, or “IGHG1*07” and is an exemplary amino acid sequence of a wild-type (WT) CH1 domain.

Human IgG1 CH1 domain reference sequence: ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKKV (positions 118-215 according to EU numbering) (SEQ ID NO: 56).

Alternative CH1 domain reference sequences of human IgG1 include but are not limited to SEQ ID NO: 57, which corresponds to the CH1 domain sequence of human IgG1 Allotype “IGHG1*03 (Y14737)” or “IGHG1*08”.

Human IgG1 alternative CH1 domain reference sequence (214R relative to SEQ ID NO: 56): ASTKGPSVFPLAPSSKSTSGGTAALGCLVKDYFPEPVTVSWNSGALTSGVHTFPAVLQ SSGLYSLSSVVTVPSSSLGTQTYICNVNHKPSNTKVDKRV (positions 118-215 according to EU numbering) (SEQ ID NO: 57).

Human IgG2, IgG3, and IgG4 CH1 domain reference sequences are provided herein as SEQ ID NO: 58, 59, and 60, respectively.

Human IgG2 CH1 domain reference sequence:
(SEQ ID NO: 58)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTV
(SEQ ID NO: 59)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRV
(SEQ ID NO: 60)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRV

These CH1 domain reference sequences are exemplary as “CH1 domain” sequences as used herein include any naturally occurring CH1 domain allotype or allelic variant.

According to IMGT, the hinge is the amino acid positions 216-230 (EU numbering). The term “hinge” is used in a broad sense herein to refer to a heavy chain region comprising at least 60% of the hinge positions 216-230 (EU numbering). Human IgG1, IgG2, IgG3, and IgG4 hinge reference sequences, corresponding to the amino acid positions 231-340 according to EU numbering, are provided herein as SEQ ID NOS: 61, 62, 63, and 64, respectively, which are exemplary amino acid sequence of a wild-type (WT) hinge.

Human IgG1 hinge reference sequence: EPKSCDKTHTCPPCP (positions 216-230 according to EU numbering) (SEQ ID NO: 61).

Human IgG2 hinge reference sequence:
(SEQ ID NO: 62)
ERKCCVECPPCP.
Human IgG3 hinge reference sequence:
(SEQ ID NO: 63)
ELKTPLGDTTHTCPRCPEPKSCDTPPPCPRCPEPKSCDTPPPCPRCPEPK
SCDTPPPCPRCP.

Human IgG4 hinge reference sequence: ESKYGPPCPSCP (SEQ ID NO: 64).

According to IMGT, the CH2 domain is the amino acid positions (or simply referred to as “positions” herein) 231-340 (EU numbering). The term “CH2 domain” is used in a broad sense herein to refer to a heavy chain region comprising at least at least 80% of the heavy chain positions 231-340 (EU numbering)).

A human IgG1 CH2 domain reference sequence, corresponding to the amino acid positions 231-340 according to EU numbering, is provided herein as SEQ ID NO: 65, which is an exemplary amino acid sequence of a wild-type (WT) CH2 domain.

Human IgG1 CH2 domain reference sequence:
(SEQ ID NO: 65)
APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAK.

Human IgG2, IgG3, and IgG4 CH2 domain reference sequences are provided herein as SEQ ID NOS: 66, 67, and 68, respectively.

Human IgG2 CH2 domain reference sequence:
(SEQ ID NO: 66)
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTK
Human IgG3 CH2 domain reference sequence:
(SEQ ID NO: 67)
APELLGGPSVFLFPPKPKDTLMISRTPEVTCVVVDVSHEDPEVQFKWYVD
GVEVHNAKTKPREEQYNSTFRVVSVLTVLHQDWLNGKEYKCKVSNKALPA
PIEKTISKTK
Human IgG4 CH2 domain reference sequence:
(SEQ ID NO: 68)
APEFLGGPSVFLFPPKPKDTLMISRTPEVTCVVVDVSQEDPEVQFNWYVD
GVEVHNAKTKPREEQFNSTYRVVSVLTVLHQDWLNGKEYKCKVSNKGLPS
SIEKTISKAK

The recited CH2 domain reference sequence is exemplary as the “CH2 domain” sequences as used herein include any naturally occurring CH2 domain allotype or allelic variant.

According to IMGT, the CH3 domain is the amino acid positions (or simply referred to as “positions” herein) 341-446 (EU numbering). The term “CH3 domain” is used in a broad sense herein to refer to a heavy chain region comprising at least seven consecutive amino acid positions of the heavy chain positions 341-446 (EU numbering)). A CH3 domain reference sequence, corresponding to the amino acid positions 341-446 according to EU numbering, is provided herein as SEQ ID NO: 69, which corresponds to the CH3 domain sequence of human IgG1 Allotype “IGHG1*01 (J00228)” or “IGHG1*08” and is an exemplary amino acid sequence of a wild-type (WT) CH3 domain.

Human IgG1 CH3 domain reference sequence:
(SEQ ID NO: 69)
GQPREPQVYTLPPSRDELTKNQVSLTCLVKGFYPSDIAVEWESNGQPENN
YKTTPPVLDSDGSFFLYSKLTVDKSRWQQGNVFSCSVMHEALHNHYTQKS
LSLSPG.

Alternative CH3 domain reference sequences of human IgG1 may include but are not limited to SEQ ID NO: 70, which corresponds to the CH3 domain sequence of human IgG1 Allotype “IGHG1*03 (Y14737)”, SEQ ID NO: 71, which corresponds to the CH3 domain sequence of human IgG1 Allotype “IGHG1*04 (JN582178)”, and SEQ ID NO: 72, which corresponds to the CH3 domain sequence of human IgG1 Allotype “IGHG1*07”.

Human IgG1 alternative CH3 domain reference sequence 1 (356E and 358M relative to SEQ ID NO: 69):

(SEQ ID NO: 70)
GQPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQKS
LSLSPG.

Human IgG1 alternative CH3 domain reference sequence 2 (422I relative to SEQ ID NO: 69):

(SEQ ID NO: 71)
GQPREPQVYTLPPSRDELTKNQVSLTCLVKGFYPSDIAVEWESNGQPENN
YKTTPPVLDSDGSFFLYSKLTVDKSRWQQGNIFSCSVMHEALHNHYTQKS
LSLSPG.

Human IgG1 alternative CH3 domain reference sequence 3 (431G relative to SEQ ID NO: 69):

(SEQ ID NO: 72)
GQPREPQVYTLPPSRDELTKNQVSLTCLVKGFYPSDIAVEWESNGQPENN
YKTTPPVLDSDGSFFLYSKLTVDKSRWQQGNVFSCSVMHEGLHNHYTQKS
LSLSPG.

Furthermore, CH3 domain reference sequences of human IgG2, IgG3, and IgG4 include but are not limited to SEQ ID NOS: 73, 74, and 75, respectively.

CH3 domain reference sequence of human IgG2:
(SEQ ID NO: 73)
GQPREPQVYTLPPSREEMTKNQVSLTCLVKGFYPSDISVEWESNGQPENN
YKTTPPMLDSDGSFFLYSKLTVDKSRWQQGNVFSCSVMHEALHNHYTQKS
LSLSPG
CH3 domain reference sequence of human IgG3:
(SEQ ID NO: 74)
GQPREPQVYTLPPSREEMTKNQVSLTCLVKGFYPSDIAVEWESSGQPENN
YNTTPPMLDSDGSFFLYSKLTVDKSRWQQGNIFSCSVMHEALHNRFTQKS
LSLSPG
CH3 domain reference sequence of human IgG4:
(SEQ ID NO: 75)
GQPREPQVYTLPPSQEEMTKNQVSLTCLVKGFYPSDIAVEWESNGQPENN
YKTTPPVLDSDGSFFLYSRLTVDKSRWQEGNVFSCSVMHEALHNHYTQKS
LSLSLG

These CH3 domain reference sequences are exemplary as the “CH3 domain” sequences used herein include any naturally occurring CH3 domain allotype or allelic variant. Accordingly, an amino acid modification(s) in variant CH3 domain polypeptides according to the present disclosure may be relative to and/or incorporated to any parent CH3 domain polypeptides, for example but not limited to a wild-type sequence, or any allelic variants thereof. In some embodiments, a CH3 domain may further comprise lysine at the C terminus (i.e., K447, according to EU numbering).

There are two major CL isotypes, kappa (“κ”) and lambda (“λ”), and such CL domains are referred to herein as kappa CL domain (“CLκ” domain) and lambda CL domain (“CLλ” domain).

According to IMGT, the CLκ domain is the amino acid positions 108-214 (EU numbering). The term “CLκ domain” is used in a broad sense herein to refer to a light chain region comprising at least seven consecutive amino acid positions of the kappa light chain positions 108-214 (EU numbering). A human Ig CLκ domain reference sequence, corresponding to the amino acid positions 108-214 (EU numbering), is provided herein as SEQ ID NO: 76, which is an exemplary amino acid sequence of a wild-type (WT) CLκ domain.

Human Ig CLκ domain reference sequence: RTVAAPSVFIFPPSDEQLKSGTASVVCLLNNFYPREAKVQWKVDNALQSGNSQESVT EQDSKDSTYSLSSTLTLSKADYEKHKVYACEVTHQGLSSPVTKSFNRGEC (positions 108 to 214 according to EU numbering) (SEQ ID NO: 76).

According to IMGT, the CLλ domain is the amino acid positions 107-215 (EU numbering). The term “CLλ domain” is used in a broad sense herein to refer to a light chain region comprising at least seven consecutive amino acid positions of the lambda light chain positions 107-215 (EU numbering). CLλ domain reference sequences known as IGLC1, 2, 3, 6, and 7, corresponding to the amino acid positions 107-215 (EU numbering), are provided herein as SEQ ID NOS: 77, 78, 79, 80, and 81, respectively, which are exemplary amino acid sequences of a standard or wild-type (WT) CLλ domain.

Human Ig CLλ domain reference sequences:
(SEQ ID NO: 77)
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTECS
(SEQ ID NO: 78)
GQPKAAPSVTLFPPSSEELQANKATLVCLISDFYPGAVTVAWKADSSPVK
AGVETTTPSKQSNNKYAASSYLSLTPEQWKSHRSYSCQVTHEGSTVEKTV
APTECS
(SEQ ID NO: 79)
GQPKAAPSVTLFPPSSEELQANKATLVCLISDFYPGAVTVAWKADSSPVK
AGVETTTPSKQSNNKYAASSYLSLTPEQWKSHKSYSCQVTHEGSTVEKTV
APTECS
(SEQ ID NO: 80)
GQPKAAPSVTLFPPSSEELQANKATLVCLISDFYPGAVKVAWKADGSPVN
TGVETTTPSKQSNNKYAASSYLSLTPEQWKSHRSYSCQVTHEGSTVEKTV
APAECS
(SEQ ID NO: 81)
GQPKAAPSVTLFPPSSEELQANKATLVCLVSDFNPGAVTVAWKADGSPVK
VGVETTKPSKQSNNKYAASSYLSLTPEQWKSHRSYSCRVTHEGSTVEKTV
APAECS

Various standard sequences (corresponding to different allotypes) of the constant domains of human IgG1, IgG2, IgG3, and IgG4 are known in the field and may be found for example in Vidarsson et al., Front Immunol. 2014 Oct. 20; 5:520 and U.S. Pat. No. 9,150,663, the disclosures of which are hereby incorporated by reference herein in their entirety herein. Again these reference sequences are exemplary as human IgG1, IgG2, IgG3, and IgG4 sequences include any naturally occurring human IgG1, IgG2, IgG3, and IgG4 allotype.

A “Fc region” is a C-terminal region of an immunoglobulin heavy chain that contains at least a portion of the constant region, including native sequence Fc regions and variant Fc regions. A human IgG heavy chain Fc region can extend from Asp221, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, as described in Kabat et al., Sequences of Proteins of Immunological Interest, 5th Ed. Public Health Service, National Institutes of Health, Bethesda, Md., 1991.

The phrase “effector function” of an antibody refers to biological activities attributable to the Fc region of an antibody, which varies by antibody class or isotype. Exemplary effector functions include: complement (e.g., C1q) binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

A variant polypeptide, such as an antigen binding protein, may contain one or more substitutions (e.g., conservative substitutions), insertions, or deletions relative to the reference sequence. In certain embodiments, the variant polypeptide comprises one, two or three substitutions, insertions and/or deletions relative to the reference sequence. Substitutions, insertions, or deletions may result in the change of one or more biophysical parameters but particularly preferred are tolerated changes so that the polypeptide retains the desired activity. In some embodiments, the variant polypeptide comprises one or more tolerated substitutions, such as conservative substitutions. In certain embodiments, such variant polypeptide maintains physically, biologically, chemically and/or functionally the properties of the corresponding reference sequence. Thus, in certain embodiments, a variant antibody retains specific binding to its target (i.e., BCMA, CD3 or CD19, respectively) and/or competes with the antibody disclosed herein for binding to its target. In some embodiments, the variant antibody comprises an amino acid sequence being at least about 80%, 85%, 90%, 95%, 96%, 97%, 98%, 99% or 100% identical to an amino acid sequence disclosed herein.

As used herein, “sequence identity” between two polypeptides is determined by comparing the amino acid sequence of one polypeptide to the sequence of a second polypeptide. Similarly, “sequence identity” between two polynucleotides is determined by comparing the nucleotide sequence of one polynucleotide to the sequence of a second polynucleotide. The terms “% identical”, “% identity” or similar terms are intended to refer, in particular, to the percentage of nucleotides or amino acids (as applicable) which are identical after the sequences to be compared have been aligned to yield maximum identity, potentially introducing gaps. Said percentage may be purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. For example, the optimal alignment for a comparison may be carried out manually or with the aid of the local homology algorithm by Smith and Waterman, 1981, Ads App. Math. 2, 482, with the aid of the local homology algorithm by Needleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using the algorithms (GAP, BESTFIT, FASTA, BLAST P, BLAST N and TFASTA in Wisconsin Genetics Software Package, Genetics Computer Group, 575 Science Drive, Madison, Wis., or Clustal Omega). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LIN K_LOC-align2seq). Typically, the % identity is determined over the entire length of the sequence according to the present disclosure on which the analysis is performed.

In certain embodiments, the antigen binding proteins disclosed herein are humanized. As used herein, the term “humanized” or “humanization” refers to an antigen binding protein that has been altered to make it more like a human antibody. Non-human antigen binding proteins would elicit a negative immune reaction if administered to a human for therapy. It is therefore advantageous to humanize the antigen binding proteins of other than human origin for later therapeutic use. In certain embodiments, the parental antibody is murine. In other embodiments, the parental antibody is a rabbit antibody. In one embodiment, “humanized” as used herein refers to the number of fully human CDRs comprised in the antigen binding protein. For example, the antigen binding protein according to the present invention has 1, 2, 3, 4, 5, 6, 7, 8, 9, 10, 11 or 12 fully human CDRs. For example, the antigen binding protein sequence is more human than a competitor, e.g., comprises a higher number of fully human CDRs than a competitor. In certain embodiments, the antigen binding proteins are humanized through resurfacing (i.e., remodel the solvent-accessible residues of the non-human framework such that they become more human-like). Resurfacing strategies are described in more detail in WO2004/016740, WO2008/144757, and WO2005/016950, each of which is incorporated herein by reference. In certain embodiments, the antigen binding proteins are humanized through CDR grafting (i.e., inserting the rabbit antigen binding protein CDRs into a human antibody acceptor framework). Grafting strategies and human acceptor frameworks are described in more detail in WO2009/155726, incorporated herein by reference.

In certain embodiments, the disclosure provides isolated antigen binding proteins that bind to BCMA, CD19 and CD3, i.e., the isolated antigen binding protein is not associated or bound to the surface of a cell, such as a T cell. Isolated antigen binding proteins are separated from a component of its natural environment. In certain embodiments, the isolated antigen binding protein is purified to greater than 95% or 99% purity as determined by, for example, electrophoretic methods (e.g., SDS-PAGE, isoelectric focusing (IEF), capillary electrophoresis) or chromatography (e.g., ion exchange or reverse phase HPLC, affinity chromatography, size exclusion chromatography). In certain embodiments, the antigen binding protein is not a soluble TCR (e.g., a TCR lacking one or more of a transmembrane domain, an intracellular signaling domain, and constant domains). In certain embodiments, the antigen binding protein is a monoclonal antibody, in particular an antibody fragment such as a scFv.

As used herein, the term “monoclonal antibody” refers to an antibody obtained from a population of substantially homogeneous antibodies, i.e., antibodies that bind to the same epitope and/or are identical in sequence. A population of polyclonal antibodies, in contrast, will bind to multiple epitopes and comprises antibodies of different sequences. A monoclonal antibody preparation may or may not comprise to a minor extent variant antibodies, e.g., due to naturally occurring mutations. Such variants may e.g., be generated through posttranslational modifications, such as clipping at the N-terminal or C-terminal end of the light and/or heavy chain, or pyroglutamate formation on the N terminus of the polypeptide chain (see e.g., Liu Y D, et al. J Biol Chem. 2011 Apr. 1; 286(13):11211-7). Depending on the methods and antibody used, the percentage of variants in the mixture varies, and may involve substantially all antibodies produced, or a very low percentage.

“Specifically recognizes”, “specifically targets” or “specifically binds” refers to the ability of the antigen binding proteins to bind selectively to the antigen in contrast to non-specific interactions with unrelated proteins which do not comprise the binding epitope. Suitable assays for determining the specific binding are described below. In certain embodiments, the equilibrium dissociation constant (K_{D}) of an antigen binding protein to an unrelated protein is less than about 50-fold lower than the one of the antigen binding protein to its antigen, as e.g., determined by SPR.

As used herein, the term “affinity” (or “binding affinity” as used interchangeably herein) refers to the strength of the interaction between an antibody's antigen binding site and the epitope to which it binds. As readily understood by those skilled in the art, an antibody or antigen binding protein affinity may be reported as an equilibrium dissociation constant (K_{D}) in molarity (M). The equilibrium dissociation constant K_{D} is calculated from the association rate constant kₐ (having the unit M⁻¹s⁻¹) and the dissociation rate constant k_{d} (having the unit s⁻¹) by k_{d}/kₐ. The antibodies according to the present disclosure may have K_{D} values in the range of 10⁻⁸ to 10⁻¹⁴ M to the target.

The ability of an antibody to bind to a specific antigenic determinant can be measured either through an enzyme-linked immunosorbent assay (ELISA) or other techniques familiar to one of skill in the art, e.g., surface plasmon resonance (SPR) technique (conducted e.g., using a BIAcore instrument) (Liljeblad et al., Glyco J 17, 323-329 (2000)), and traditional binding assays (Heeley, Endocr Res 28, 217-229 (2002)). Generally, kinetic rate constants can be determined at temperatures in the range of 15 °C to 37 °C. The present specification makes reference to kinetic rate constants determined by SPR throughout. Typically, in embodiments pertaining to each reference to SPR throughout the present specification, association rate constant values, dissociation rate constant values and equilibrium dissociation constant values recited herein are determined by SPR at 25 °C based on the monovalent antigen binding protein. Preferably, the SPR-based system used is a Biacore SPR system. The skilled person will appreciate that the binding parameters can be measured in the context of the monovalent or bivalent bi-, tri- or multispecific constructs. For example, in certain embodiments, the affinity values recited herein of the bispecific compounds are determined in a Fab-scFv format, wherein the Fab targets CD3 and the scFv either CD19 or BCMA. In specific embodiments, the recited affinity values of trispecific compounds are determined in a Fab-(scFv)₂ format, wherein the Fab targets CD3, the first scFvs targets CD19 and the second scFv targets BCMA.

As used herein, the term “PBS” refers to phosphate buffered saline. PBS is a pH-adjusted blend of phosphate buffers and saline solutions. In certain embodiments, the PBS comprises about 100-150 mM NaCl, about 1-5 mM KCl, about 1-10 mM Na₂HPO₄, and 1-5 mM KH₂PO₄. In certain embodiments, the PBS comprises 130 mM NaCl, 10 mM Na₂HPO₄, and pH of 6.0.

The terms “control molecule”, “comparator” and/or “competitor” as interchangeably used herein refers to any reference molecule which may be used to test or compare the properties and/or efficacy of the antigen binding protein according to the present invention. The terms “control molecule”, “comparator” or “competitor” as used herein refers for example to another antigen binding protein, such as a mono- and/or multispecific antigen binding protein. For example, “control molecule” refers to an antigen binding protein (e.g., an antibody) against the same target protein (i.e., BCMA, CD19 and/or CD3). In one embodiment, a control molecule according to the present invention is a bispecific or trispecific antigen binding protein targeting BCMA, such as a bispecific BCMAxCD3 targeting protein. In one embodiment, a control molecule is combination of a bispecific BCMA-targeting antigen binding protein and a monospecific antigen binding protein, such as a bispecific BCMAxCD3 targeting protein in combination with a CD19 inhibitor.

### Target Antigen Binding Molecules

For avoidance of doubt, and unless otherwise indicated, “BCMA” as used herein refers to the B-cell maturation antigen, particularly human BCMA (Uniprot Acc. No. Q02223). BCMA is also known as tumor necrosis factor receptor superfamily member 17 (TNFRSF17) and is a cell surface receptor of the TNF receptor superfamily which recognizes B-cell activating factor (BAFF) and a proliferation-inducing ligand (APRIL), promoting B-cell survival and plays a role in the regulation of humoral immunity. Overexpression or activation of BCMA by its ligand APRIL was described to promote human multiple myeloma (MM) progression in vivo (see Yu-Tzu Tai et al., Blood, 2016 Jun. 23; 127(25):3225-36).

The term “CD3”, refers to the cluster of differentiation 3 co-receptor (or co-receptor complex) of the T cell receptor, and is a complex composed of four distinct chains. In mammals, the complex contains a CD3γ (gamma) chain/subunit, a CD3δ (delta) chain/subunit, and two CD3ε (epsilon) chains/subunits. Reference to CD3 as the cell surface protein of an immune cell is made herein throughout. The term “CD3” refers to any native CD3 from any vertebrate source, including primates. In certain embodiments, the antigen binding proteins according to the present invention specifically bind to human CD3, in particular to the CD3ε (epsilon) chain/subunit of CD3 (see e.g., UniProt (www.uniprot.org) accession no. P07766 (version 189), or NCBI (www.ncbi.nlm.nih.gov/) RefSeq NP_000724.1.) The CD3 molecule may be the full-length, unprocessed CD3 molecule or a fragment or variant thereof that e.g., results from processing in the cell. For example, such variants may be naturally occurring variants like splice variants or allelic variants. In certain embodiments, the antigen binding proteins disclosed herein bind to an epitope of CD3 that is conserved among the CD3 antigens from different species, such as non-human primates (e.g., cynomolgus monkeys) or rodents (e.g., mice, rats). In certain embodiments, the antigen binding proteins are not cross-reactive with a CD3 antigen from rodents (e.g., mouse or rat) or minipigs. Suitable CD3 binding domains of the multispecific antigen binding proteins disclosed herein are known in the art, particularly T-cell activating CD-epsilon binding domains. In certain embodiments of the present disclosure, the anti-CD3 binding domain contains the 6 CDRs of any appropriate anti-CD3 antibody or any known anti-CD3 antibodies such as but not limited to SP34, OKT3 or UCHT1, or is or comprises the VH and VL of any one of antibodies SP34, OKT3 or UCHT1, or a variant sequence thereof having at least about 80%, 85%, 90%, 95%, 96%, 97%, 98% or 99% identity thereto, while retaining the same specificity as its parent (e.g., by retaining the same 6 CDRs contained in the VH and VL of such a parent antibody). SP34, OKT3 or UCHT1 are murine antibodies; for therapeutic applications, humanized versions of SP34, OKT3 or UCHT1, i.e., huSP34, huOKT3 or huUCHT1, are preferred. In certain embodiments, the humanized variant sequence of SP34, OKT3 or UCHT1 is optimized for use in Fab format. For example, the humanized huSP34, huOKT3 or huUCHT1 may comprise 1, 2, 3, 4, 5, 6, 7, 8, 9 or more substitutions while retaining specific binding to CD3. Exemplary CD3 binding domains are disclosed in U.S. Pat. No. 6,750,325, WO2008079713, U.S. Pat. No. 7,635,475, WO2005040220, U.S. Pat. No. 7,728,114, WO9404679, U.S. Pat. No. 7,381,803, WO2008119567, WO2014110601, WO2014145806, WO2015095392, WO2016086189 and/or WO2019195535A1, each of which is incorporated herein by reference.

As used herein, the term “CD19” (cluster of differentiation 19), refers to the human protein designated under Uniprot Acc. No. P15391). The human CD19 protein is a B-lymphocyte surface protein that functions as a coreceptor for the B-cell antigen receptor complex (BCR).

In one aspect, an antigen binding protein that specifically binds to human BCMA is provided. A BCMA antigen binding protein may comprise: a variable light chain (VL) comprising a CDR-L1, a CDR-L2, and a CDR-L3; and a variable heavy chain (VH) comprising a CDR-H1, a CDR-H2, and a CDR-H3.

In some embodiments, in the VL of the BCMA binding domain, the CDR-L1, the CDR-L2, and the CDR-L3 may comprise the CDR-L1, CDR-L2, and CDR-L3 sequences, respectively, contained in SEQ ID NO: 7 or 82, and in the VH of the BCMA binding domain, the CDR-H1, the CDR-H2, and the CDR-H3 may comprise the CDR-H1, CDR-H2, and CDR-H3 sequences, respectively, contained in SEQ ID NO: 8. Such CDR-L1, CDR-L2, and CDR-L3 and CDR-H1, CDR-H2, and CDR-H3 sequences in may be the sequences defined by any appropriate CDR-defining scheme including but not limited to Kabat, Chothia, AHo, Contact, or IMGT.

In certain embodiments, a BCMA binding protein may comprise: (a) a VL comprising a CDR-L1 sequence of RASKSVSFLGINLIY (SEQ ID NO: 1), a CDR-L2 sequence of QASNRDT (SEQ ID NO: 2) and a CDR-L3 sequence of LQTPFFPRT (SEQ ID NO: 3), and/or (b) a VH comprising a CDR-H1 sequence of DYSIN (SEQ ID NO: 4), a CDR-H2 sequence of WINTETGNPTYAQGFTG (SEQ ID NO: 5), and a CDR-H3 sequence of DKHLAMDY (SEQ ID NO: 6).

In certain embodiments, such BCMA binding protein comprises: (a) a variable light chain (VL) comprising or consisting of an amino acid sequence that is at least about 90%, 95%, 96%, 97%, 98%, 99% or 100% identical to the amino acid sequence of SEQ ID NO: 7 or SEQ ID NO: 82 (which is SEQ ID NO: 7 with a C-terminal G added); and/or (b) a variable heavy chain (VH) comprising or consisting of an amino acid sequence that is at least about 90%, 95%, 96%, 97%, 98%, 99% or 100% identical to the amino acid sequence of SEQ ID NO: 8. For example, such VL and/or VH sequence identity may be met while comprising the CDR-L1, CDR-L2, and CDR-L3 sequences and/or CDR-H1, CDR-H2, and CDR-H3 sequences as described above.

In certain embodiments, the BCMA antigen binding protein has an affinity (K_{D}) between about 0.5 μM to about 0.5 nM to human BCMA, as determined by SPR. In certain embodiments, the BCMA antigen binding protein has an affinity (K_{D}) between about 10 μM to about 0.1 nM to human BCMA, as determined by SPR. In certain embodiments, the BCMA antigen binding protein has an affinity (K_{D}) of about 500 μM or lower, such as about 400 μM, 300 μM, 250 μM, 200 μM, 150 μM, 100 μM. 50 μM, 20 μM, 15 μM or about 10 μM or lower.

In another aspect, an antigen binding protein that specifically binds to human CD19 is provided. A CD19 antigen binding protein may comprise: a variable light chain (VL) comprising a CDR-L1, a CDR-L2, and a CDR-L3; and a variable heavy chain (VH) comprising a CDR-H1, a CDR-H2, and a CDR-H3.

In some embodiments, in the VL of the CD19 binding domain, the CDR-L1, the CDR-L2, and the CDR-L3 may comprise the CDR-L1, CDR-L2, and CDR-L3 sequences, respectively, contained in SEQ ID NO: 16 or 88, and in the VH of the CD19 binding domain, the CDR-H1, the CDR-H2, and the CDR-H3 may comprise the CDR-H1, CDR-H2, and CDR-H3 sequences, respectively, contained in SEQ ID NO: 17. Such CDR-L1, CDR-L2, and CDR-L3 sequences and CDR-H1, CDR-H2, and CDR-H3 sequences in may be the sequences defined by any appropriate CDR-defining scheme including but not limited to Kabat, Chothia, AHo, Contact, or IMGT.

In certain embodiments, a CD19 binding protein may comprise: (a) a VL comprising a CDR-L1 sequence of RASQSVDYDGDSYLN (SEQ ID NO: 10), a CDR-L2 sequence of DASNLVS (SEQ ID NO: 11) and a CDR-L3 sequence of QQSTEDPWT (SEQ ID NO: 12), and/or (b) a VH comprising a CDR-H1 sequence of SYWMN (SEQ ID NO: 13), a CDR-H2 sequence of QIWPGDGDTNYNGKFKG (SEQ ID NO: 14), and a CDR-H3 sequence of RETTTVGRYYYAMDY (SEQ ID NO: 15).

In certain embodiments, such CD19 binding protein comprises (a) a variable light chain (VL) comprising or consisting of an amino acid sequence that is at least about 90%, 95%, 96%, 97%, 98%, 99% or 100% identical to the amino acid sequence of SEQ ID NO: 16 or SEQ ID NO: 88 (which is SEQ ID NO: 16 without the C-terminal G); and/or (b) a variable heavy chain (VH) comprising or consisting of an amino acid sequence that is at least about 90%, 95%, 96%, 97%, 98%, 99% or 100% identical to the amino acid sequence of SEQ ID NO: 17. For example, such VL and/or VH sequence identity may be met while comprising the CDR-L1, CDR-L2, and CDR-L3 sequences and/or CDR-H1, CDR-H2, and CDR-H3 sequences as described above.

In certain embodiments, the CD19 binding protein has an affinity (K_{D}) between about 0.01 nM (i.e., 10 pM) to about 5 nM to human CD19, as determined by SPR.

In another aspect, an antigen binding protein that specifically binds to human CD3 is provided. A CD3 antigen binding protein may comprise: a variable light chain (VL) comprising a CDR-L1, a CDR-L2, and a CDR-L3; and a variable heavy chain (VH) comprising a CDR-H1, a CDR-H2, and a CDR-H3.

In some embodiments, in the VL of the CD3 binding domain, the CDR-L1, the CDR-L2, and the CDR-L3 may comprise the CDR-L1, CDR-L2, and CDR-L3 sequences, respectively, contained in SEQ ID NO: 25 or 94, and in the VH of the CD3 binding domain, the CDR-H1, the CDR-H2, and the CDR-H3 may comprise the hCDR-H1, CDR-H2, and CDR-H3 sequences, respectively, contained in SEQ ID NO: 26. Such 1 CDR-L1, CDR-L2, and CDR-L3 sequences and CDR-H1, CDR-H2, and CDR-H3 sequences in may be the sequences defined by any appropriate CDR-defining scheme including but not limited to Kabat, Chothia, AHo, Contact, or IMGT.

In certain embodiments, a CD3 binding protein may comprise: (a) a VL comprising a CDR-L1 sequence of GSSTGAVTTSNYAN (SEQ ID NO: 19), a CDR-L2 sequence of GTNKRAP (SEQ ID NO: 20) and a CDR-L3 sequence of ALWYSNHWV (SEQ ID NO: 21), and/or (b) a VH comprising a CDR-H1 sequence of TYAMN (SEQ ID NO: 22), a CDR-H2 sequence of RIRSKYNNYATYYADSVKG (SEQ ID NO: 23), and a CDR-H3 sequence of HGNFGDSYVSWFAY (SEQ ID NO: 24). In certain embodiments, said CD3 binding domain serves as immune cell binding domain.

In certain embodiments, such CD3 binding protein comprises: (a) a variable light chain (VL) comprising or consisting of an amino acid sequence that is at least about 90%, 95%, 96%, 97%, 98%, 99% or 100% identical to the amino acid sequence of SEQ ID NO: 25 or SEQ ID NO: 94 (which is SEQ ID NO: 25 without the C-terminal G); and/or (b) a variable heavy chain (VH) comprising or consisting of an amino acid sequence that is at least about 90%, 95%, 96%, 97%, 98%, 99% or 100% identical to the amino acid sequence of SEQ ID NO: 26. For example, such VL and/or VH sequence identity may be met while comprising the CDR-L1, CDR-L2, and CDR-L3 sequences and/or CDR-H1, CDR-H2, and CDR-H3 sequences as described above.

The CD3 binding protein disclosed herein is a humanized solubility optimized variant of sp34 and are not cross-reactive with a CD3 antigen from rodents (e.g., mouse or rat) or minipigs. In certain embodiments, the CD3 binding protein has an affinity (K_{D}) of about 1 to about 40 nM, as determined by SPR, such as about 1 nM, about 1.5 nM, about 2 nM, about 3 nM, about 5 nM, about 6 nM, about 10 nM, about 15 nM, about 20 nM, about 25 nM, about 30 nM, or about 35 nM. In certain embodiments, the CD3 affinity (K_{D}) is measured on a CD3δε or a CD3εγ complex. In certain embodiments, the CD3 binding domain shows at least a 5-fold improvement in affinity compared to the parental precursor. In certain embodiments, the CD3 binding protein binds to an epitope of CD3 that is conserved among the CD3 antigens from human and non-human primates (e.g., cynomolgus monkeys).

In some embodiments, the association rate constant kₐ of the anti-CD3 binding domain is between about 1×10⁵ to about 1×10⁷ M⁻¹s⁻¹, such as at least 1×10⁶ M⁻¹s⁻¹ or at least 2×10⁶ M⁻¹s⁻¹.

In some embodiments, the dissociation rate constant k_{d} of the anti-CD3 binding domain is between about 1×10⁻¹ to about 1×10⁻⁶ s⁻¹, such as at least 2×10⁻³ s⁻¹, or at least 3×10⁻³ s⁻¹ or at least 4×10⁻³ s⁻¹. Without being bound to theory, a fast dissociation rate, e.g., a k_{d}-value of 2-3×10⁻³ s⁻¹, may lead to less T cell overactivation and in consequence, less cytokine release.

In one embodiment, the association rate constant kₐ and/or the dissociation rate constant k_{d} are equivalent or similar for both CD3-heterodimers CD3εγ (epsilon/gamma) and CD3εδ (epsilon/delta), i.e., there is no significant difference for either the kₐ or the k_{d} or both of the anti-CD3 binding domain to CD3εγ (epsilon/gamma) and CD3εδ (epsilon/delta) when measured under the same conditions, in particular when determined by SPR at 25 °C. In certain embodiments, the association rate constant kₐ and/or the dissociation rate constant k_{d} values that are within 1-fold of each other, 1.5-fold of each other, 2-fold of each other, 2.5-fold of each other or 3-fold of each other, i.e., association rate constant kₐ values of 1×10⁵ M⁻¹s⁻¹ and 3×10⁵ M⁻¹s⁻¹.

In certain embodiments, the disclosure provides multispecific antigen binding proteins, such as bispecifics or trispecifics.

In certain embodiments, a multispecific antigen binding protein of the invention comprises the BCMA binding protein described above. In certain embodiments, the multispecific antigen binding protein comprises the BCMA binding protein described above, and further at least one second antigen binding domain targeting a second antigen.

In certain embodiments, said second antigen binding domain is an immune cell engager, i.e., the second antigen binding domain comprises specificity for a cell surface protein of an immune cell. In certain embodiments, the immune cell is a human cell. In certain embodiments, the immune cell is selected from the group consisting of a T cell, a B cell, a natural killer (NK) cell, a natural killer T (NKT) cell, a neutrophil cell, a monocyte, and a macrophage. In certain embodiments, the immune cell is a T cell. In certain embodiments, the cell surface protein of an immune cell is selected from the group consisting of CD3, TCRα, TCRβ, the α/β T Cell Receptor, CD16 (e.g., CD16a), NKG2D, CD94/NKG2C, NKp30, NKp46, CD89, CD64, and CD32 (e.g., CD32a) on the surface of an immune cell. In certain embodiments, the cell surface protein of an immune cell, i.e., the second antigen, is CD3. In certain embodiments, the immune cell engager is an antibody, such as a CD3 targeting antibody. Exemplary CD3 targeting antibodies include huSP34, huOKT or huUCHT1 or variants derived thereof.

In certain embodiments, the multispecific antigen binding protein is bispecific. In certain embodiments, the bispecific is a BCMAxCD3 targeting binding protein comprising the BCMA binding protein described above and the CD3 binding protein described above. Thus, in certain embodiments, a bispecific BCMAxCD3 binding protein is provided, comprising
(a) a BCMA binding domain having
(a1) a variable light chain (VL) comprising or consisting of an amino acid sequence that is at least about 90%, 95%, 96%, 97%, 98%, 99% or 100% identical to the amino acid sequence of SEQ ID NO: 7 or 82; and
(a2) a variable heavy chain (VH) comprising or consisting of an amino acid sequence that is at least about 90%, 95%, 96%, 97%, 98%, 99% or 100% identical to the amino acid sequence of SEQ ID NO: 8, wherein
(a3) said variable light chain (VL) comprises the CDR-L1, CDR-L2, and CDR-L3 sequences contained in SEQ ID NO: 7 or 82, for example, when the CDRs are defined based on Kabat, a CDR-L1 sequence of RASKSVSFLGINLIY (SEQ ID NO: 1), a CDR-L2 sequence of QASNRDT (SEQ ID NO: 2) and a CDR-L3 sequence of LQTPFFPRT (SEQ ID NO: 3); and
(a4) said variable heavy chain (VH) comprises the CDR-H1, CDR-H2, and CDR-H3 sequences contained in SEQ ID NO: 8, for example, when the CDRs are defined based on Kabat, a CDR-H1 sequence of DYSIN (SEQ ID NO: 4), a CDR-H2 sequence of WINTETGNPTYAQGFTG (SEQ ID NO: 5), and a CDR-H3 sequence of DKHLAMDY (SEQ ID NO: 6);
and
(b) a CD3 binding domain having
(b1) a variable light chain (VL) comprising or consisting of an amino acid sequence that is at least about 90%, 95%, 96%, 97%, 98%, 99% or 100% identical to the amino acid sequence of SEQ ID NO: 25 or 94; and
(b2) a variable heavy chain (VH) comprising or consisting of an amino acid sequence that is at least about 90%, 95%, 96%, 97%, 98%, 99% or 100% identical to the amino acid sequence of SEQ ID NO: 26, wherein
(b3) said VL comprises the CDR-L1, CDR-L2, and CDR-L3 sequences contained in SEQ ID NO: 25 or 94, e.g., when the CDRs are defined based on Kabat, a CDR-L1 sequence of GSSTGAVTTSNYAN (SEQ ID NO: 19), a CDR-L2 sequence of GTNKRAP (SEQ ID NO: 20) and a CDR-L3 sequence of ALWYSNHWV (SEQ ID NO: 21), and
(b4) said VH comprises the CDR-H1, CDR-H2, and CDR-H3 sequences contained in SEQ ID NO: 26, e.g., when the CDRs are defined based on Kabat, a CDR-H1 sequence of TYAMN (SEQ ID NO: 22), a CDR-H2 sequence of RIRSKYNNYATYYADSVKG (SEQ ID NO: 23), and a CDR-H3 sequence of HGNFGDSYVSWFAY (SEQ ID NO: 24).

In certain embodiments, said second antigen binding domain specifically binds to CD19. In certain embodiments, the multispecific antigen binding protein comprises the BCMA binding protein described above and the CD19 binding protein described above. Hence, in certain embodiments, a bispecific BCMAxCD19 binding protein is provided, comprising:
(a) a BCMA binding domain having
(a1) a variable light chain (VL) comprising or consisting of an amino acid sequence that is at least about 90%, 95%, 96%, 97%, 98%, 99% or 100% identical to the amino acid sequence of SEQ ID NO: 7 or 82; and
(a2) a variable heavy chain (VH) comprising or consisting of an amino acid sequence that is at least about 90%, 95%, 96%, 97%, 98%, 99% or 100% identical to the amino acid sequence of SEQ ID NO: 8, wherein
(a3) said variable light chain (VL) comprises the CDR-L1, CDR-L2, and CDR-L3 sequences contained in SEQ ID NO: 7 or 82, e.g., when the CDRs are defined based on Kabat, a CDR-L1 sequence of RASKSVSFLGINLIY (SEQ ID NO: 1), a CDR-L2 sequence of QASNRDT (SEQ ID NO: 2) and a CDR-L3 sequence of LQTPFFPRT (SEQ ID NO: 3); and (a4) said variable heavy chain (VH) comprises the CDR-H1, CDR-H2, and CDR-H3 sequences contained in SEQ ID NO: 8, e.g., when the CDRs are defined based on Kabat, a CDR-H1 sequence of DYSIN (SEQ ID NO: 4), a CDR-H2 sequence of WINTETGNPTYAQGFTG (SEQ ID NO: 5), and a CDR-H3 sequence of DKHLAMDY (SEQ ID NO: 6);
and
(b) a CD19 binding domain having
(b1) a variable light chain (VL) comprising or consisting of an amino acid sequence that is at least about 90%, 95%, 96%, 97%, 98%, 99% or 100% identical to the amino acid sequence of SEQ ID NO: 16 or 88; and
(b2) a variable heavy chain (VH) comprising or consisting of an amino acid sequence that is at least about 90%, 95%, 96%, 97%, 98%, 99% or 100% identical to the amino acid sequence of SEQ ID NO: 17, wherein
(b3) said VL comprises the CDR-L1, CDR-L2, and CDR-L3 sequences contained in SEQ ID NO: 16 or 88, e.g., when the CDRs are defined based on Kabat, a CDR-L1 sequence of RASQSVDYDGDSYLN (SEQ ID NO: 10), a CDR-L2 sequence of DASNLVS (SEQ ID NO: 11) and a CDR-L3 sequence of QQSTEDPWT (SEQ ID NO: 12), and
(b4) said VH comprises the CDR-H1, CDR-H2, and CDR-H3 sequences contained in SEQ ID NO: 17, e.g., when the CDRs are defined based on Kabat, a CDR-H1 sequence of SYWMN (SEQ ID NO: 13), a CDR-H2 sequence of QIWPGDGDTNYNGKFKG (SEQ ID NO: 14), and a CDR-H3 sequence of RETTTVGRYYYAMDY (SEQ ID NO: 15).

In certain embodiments, a bispecific antigen binding protein of the invention comprises an immune cell engager and CD19. In certain embodiments, said bispecific is a CD19xCD3 targeting protein comprising the CD19 binding protein described above and a CD3 targeting antibody. Exemplary CD3 targeting antibodies include huSP34, huOKT or huUCHT1 or variants derived thereof. In certain embodiments, the CD3 binding protein is the one described above. Hence, in certain embodiments, a bispecific CD19xCD3 binding protein of the invention comprises
(a) a CD19 binding domain having
(a1) a variable light chain (VL) comprising or consisting of an amino acid sequence that is at least about 90%, 95%, 96%, 97%, 98%, 99% or 100% identical to the amino acid sequence of SEQ ID NO: 16 or 88; and
(a2) a variable heavy chain (VH) comprising or consisting of an amino acid sequence that is at least about 90%, 95%, 96%, 97%, 98%, 99% or 100% identical to the amino acid sequence of SEQ ID NO: 17, wherein
(a3) said VL comprises the CDR-L1, CDR-L2, and CDR-L3 sequences contained in SEQ ID NO: 16 or 88, e.g., when the CDRs are defined based on Kabat, a CDR-L1 sequence of RASQSVDYDGDSYLN (SEQ ID NO: 10), a CDR-L2 sequence of DASNLVS (SEQ ID NO: 11) and a CDR-L3 sequence of QQSTEDPWT (SEQ ID NO: 12), and
(a4) said VH comprises the CDR-H1, CDR-H2, and CDR-H3 sequences contained in SEQ ID NO: 17, e.g., when the CDRs are defined based on Kabat, a CDR-H1 sequence of SYWMN (SEQ ID NO: 13), a CDR-H2 sequence of QIWPGDGDTNYNGKFKG (SEQ ID NO: 14), and a CDR-H3 sequence of RETTTVGRYYYAMDY (SEQ ID NO: 15);
and
(c) a CD3 binding domain having
(b1) a variable light chain (VL) comprising or consisting of an amino acid sequence that is at least about 90%, 95%, 96%, 97%, 98%, 99% or 100% identical to the amino acid sequence of SEQ ID NO: 25 or 94; and
(b2) a variable heavy chain (VH) comprising or consisting of an amino acid sequence that is at least about 90%, 95%, 96%, 97%, 98%, 99% or 100% identical to the amino acid sequence of SEQ ID NO: 26, wherein
(b3) said VL comprises the CDR-L1, CDR-L2, and CDR-L3 sequences contained in SEQ ID NO: 25 or 94, e.g., when the CDRs are defined based on Kabat, a CDR-L1 sequence of GSSTGAVTTSNYAN (SEQ ID NO: 19), a CDR-L2 sequence of GTNKRAP (SEQ ID NO: 20) and a CDR-L3 sequence of ALWYSNHWV (SEQ ID NO: 21), and
(b4) said VH comprises the CDR-H1, CDR-H2, and CDR-H3 sequences contained in SEQ ID NO: 26, e.g., when the CDRs are defined based on Kabat, a CDR-H1 sequence of TYAMN (SEQ ID NO: 22), a CDR-H2 sequence of RIRSKYNNYATYYADSVKG (SEQ ID NO: 23), and a CDR-H3 sequence of HGNFGDSYVSWFAY (SEQ ID NO: 24).

A bispecific protein of the invention may have or comprise multiple formats, such as e.g., a Fab-sdAb, a CrossMab, a Fab-scFv, a bivalent M-gager (Fab-(scFv)₂), a bis-scFv (or tandem scFv or BiTE), a DART, a diabody, scDb, DVD-Ig, IgG-scFab, IgG-scFv, scFv-Fc, FynomAB or DuoBody.

Suitable linkers to connect the different binding domains, apart from those outlined above, include GGGGS (SEQ ID NO: 31), GGGGSGGGGS (SEQ ID NO: 32), GGGGSGGGGSGGGGS (SEQ ID NO: 33), GGGGSGGGGSGGGGSGGGGS (SEQ ID NO: 34), GGGGGSGGGGSGGGGSGGGGS (SEQ ID NO: 35), or GGGGSGGGGSGGGGSGGGGAS (SEQ ID NO: 36).

In certain embodiments, the multispecific antigen binding protein is at least trispecific and comprises at least three antigen binding domains; in certain embodiments, each of the binding domains targets a different antigen. The third antigen binding domain may be attached to one of the bispecifics detailed above. In certain embodiments, the trispecific antigen binding protein comprises the BCMA binding protein described above and the CD3 binding protein described above.

In certain embodiments, the trispecific antigen binding protein comprises the bispecific BCMAxCD3 binding protein described above. In specific embodiments, the trispecific antigen binding protein comprises the BCMA binding protein described above, the CD3 binding protein described above and the CD19 binding protein described above.

A multispecific antigen binding protein according to the invention may comprise a CD19 binding scFv comprising or consisting of an amino acid sequence that is at least about 80%, 85%, 90%, 95%, 96%, 97%, 98%, 99% or 100% identical to the amino acid sequence of SEQ ID NO: 18 or 89, 90, 91, 92, or 93. In certain embodiments, the amino acid sequence that is at least about 80%, 85%, 90%, 95%, 96%, 97%, 98%, or 99% identical to the amino acid sequence of SEQ ID NO: 18 or 89.

Such an scFv may comprise the CDR-L1, CDR-L2, and CDR-L3 sequences contained in SEQ ID NO: 16 or 88 and the CDR-H1, CDR-H2, and CDR-H3 sequences contained in SEQ ID NO: 17, e.g., when the CDRs are defined based on Kabat, the CDR-L1 sequence of RASQSVDYDGDSYLN (SEQ ID NO: 10), the CDR-L2 sequence of DASNLVS (SEQ ID NO: 11), the CDR-L3 sequence of QQSTEDPWT (SEQ ID NO: 12), the CDR-H1 sequence of SYWMN (SEQ ID NO: 13), the CDR-H2 sequence of QIWPGDGDTNYNGKFKG (SEQ ID NO: 14), and the CDR-H3 sequence of RETTTVGRYYYAMDY (SEQ ID NO: 15).

A multispecific antigen binding protein according to the invention may comprise a CD3 binding Fab with a HC comprising or consisting of an amino acid sequence that is at least about 80%, 85%, 90%, 95%, 96%, 97%, 98%, 99% or 100% identical to the amino acid sequence of SEQ ID NO: 28 and a LC comprising or consisting of an amino acid sequence that is at least about 80%, 85%, 90%, 95%, 96%, 97%, 98%, 99% or 100% identical to the amino acid sequence of SEQ ID NO: 27. In certain embodiments, the CD3 binding Fab with a HC comprising or consisting of an amino acid sequence that is at least about 80%, 85%, 90%, 95%, 96%, 97%, 98%, 99% or 100% identical to the amino acid sequence of SEQ ID NO: 28 and a LC comprising or consisting of an amino acid sequence that is at least about 80%, 85%, 90%, 95%, 96%, 97%, 98%, 99% or 100% identical to the amino acid sequence of SEQ ID NO: 27.

In some alternative cases, a multispecific antigen binding protein according to the invention may comprise a CD3 binding scFv, which may optionally comprise an amino acid sequence that is at least about 80%, 85%, 90%, 95%, 96%, 97%, 98%, 99% or 100% identical to the amino acid sequence of SEQ ID NO: 95 or 96 (VL-linker-VH), or SEQ ID NO: 97, 98, 99, or 100 (VH-linker-VL).

Any of such anti-CD3 Fab or scFv may comprise the CDR-L1, CDR-L2, and CDR-L3 sequences contained in SEQ ID NO: 25 or 94 or the CDR-H1, CDR-H2, and CDR-H3 sequences contained in SEQ ID NO: 26, e.g., when the CDRs are defined based on Kabat, the CDR-L1 sequence of GSSTGAVTTSNYAN (SEQ ID NO: 19), the CDR-L2 sequence of GTNKRAP (SEQ ID NO: 20), the CDR-L3 sequence of ALWYSNHWV (SEQ ID NO: 21), the CDR-H1 sequence of TYAMN (SEQ ID NO: 22), the CDR-H2 sequence of RIRSKYNNYATYYADSVKG (SEQ ID NO: 23), and the CDR-H3 sequence of HGNFGDSYVSWFAY (SEQ ID NO: 24).

A multispecific antigen binding protein according to the invention may comprise a BCMA binding scFv comprising or consisting of an amino acid sequence that is at least about 80%, 85%, 90%, 95%, 96%, 97%, 98%, 99% or 100% identical to the amino acid sequence of SEQ ID NO: 9 or 83, 84, 85, 86, or 87. In certain embodiments, said amino acid sequence that is at least about 80%, 85%, 90%, 95%, 96%, 97%, 98%, 99% identical to the amino acid sequence of SEQ ID NO: 9 or 83.

Such an scFv may comprise the CDR-L1, CDR-L2, and CDR-L3 sequences contained in SEQ ID NO: 7 or 82 and the CDR-H1, CDR-H2, and CDR-H3 sequences contained in SEQ ID NO: 8, e.g., when the CDRs are defined based on Kabat, the CDR-L1 sequence of RASKSVSFLGINLIY (SEQ ID NO: 1), the CDR-L2 sequence of QASNRDT (SEQ ID NO: 2), the CDR-L3 sequence of LQTPFFPRT (SEQ ID NO: 3), the CDR-H1 sequence of DYSIN (SEQ ID NO: 4), the CDR-H2 sequence of WINTETGNPTYAQGFTG (SEQ ID NO: 5), and the CDR-H3 sequence of DKHLAMDY (SEQ ID NO: 6).

Thus, in one aspect, a trispecific antigen binding protein is provided which comprises a first binding domain, a second binding domain, and a third binding domain, each binding domain comprising a variable light chain (VL) and variable heavy chain (VH), wherein:
(a) the first binding domain specifically binds to human BCMA and comprises:
(a1) a VL comprising the CDR-L1, CDR-L2, and CDR-L3 sequences contained in SEQ ID NO: 7 or 82, e.g., when the CDRs are defined based on Kabat, a CDR-L1 sequence of RASKSVSFLGINLIY (SEQ ID NO: 1), a CDR-L2 sequence of QASNRDT (SEQ ID NO: 2) and a CDR-L3 sequence of LQTPFFPRT (SEQ ID NO: 3), and
(a2) a VH comprising the CDR-H1, CDR-H2, and CDR-H3 sequences contained in SEQ ID NO: 8, e.g., when the CDRs are defined based on Kabat, a CDR-H1 sequence of DYSIN (SEQ ID NO: 4), a CDR-H2 sequence of WINTETGNPTYAQGFTG (SEQ ID NO: 5), and a CDR-H3 sequence of DKHLAMDY (SEQ ID NO: 6);
(b) the second binding domain specifically binds to human CD3 and comprises:
(b1) a VL comprising the CDR-L1, CDR-L2, and CDR-L3 sequences contained in SEQ ID NO: 25 or 94, e.g., when the CDRs are defined based on Kabat, a CDR-L1 sequence of GSSTGAVTTSNYAN (SEQ ID NO: 19), a CDR-L2 sequence of GTNKRAP (SEQ ID NO: 20) and a CDR-L3 sequence of ALWYSNHWV (SEQ ID NO: 21), and
(b2) a VH comprising the CDR-H1, CDR-H2, and CDR-H3 sequences contained in SEQ ID NO: 26, e.g., when the CDRs are defined based on Kabat, a CDR-H1 sequence of TYAMN (SEQ ID NO: 22), a CDR-H2 sequence of RIRSKYNNYATYYADSVKG (SEQ ID NO: 23), and a CDR-H3 sequence of HGNFGDSYVSWFAY (SEQ ID NO: 24); and
(c) the third binding domain specifically binds to human CD19 and comprises:
(c1) a VL comprising the CDR-L1, CDR-L2, and CDR-L3 sequences contained in SEQ ID NO: 16 or 88, e.g., when the CDRs are defined based on Kabat, a CDR-L1 sequence of RASQSVDYDGDSYLN (SEQ ID NO: 10), a CDR-L2 sequence of DASNLVS (SEQ ID NO: 11) and a CDR-L3 sequence of QQSTEDPWT (SEQ ID NO: 12), and
(c2) a VH comprising the CDR-H1, CDR-H2, and CDR-H3 sequences contained in SEQ ID NO: 17, e.g., when the CDRs are defined based on Kabat, a CDR-H1 sequence of SYWMN (SEQ ID NO: 13), a CDR-H2 sequence of QIWPGDGDTNYNGKFKG (SEQ ID NO: 14), and a CDR-H3 sequence of RETTTVGRYYYAMDY (SEQ ID NO: 15).

In certain embodiments, the first binding domain that specifically binds to human BCMA comprises:
(i) a VL comprising an amino acid sequence that is at least about 90%, 95%, 96%, 97%, 98%, 99% or 100% identical to the amino acid sequence of SEQ ID NO: 7 or SEQ ID NO: 82 (which is SEQ ID NO: 7 with a C-terminal “G” added); and/or
(ii) a VH comprising an amino acid sequence that is at least about 90%, 95%, 96%, 97%, 98%, 99% or 100% identical to the amino acid sequence of SEQ ID NO: 8.

In certain embodiments, the second binding domain that specifically binds to human CD3 comprises:
(i) a VL comprising an amino acid sequence that is at least about 90%, 95%, 96%, 97%, 98%, 99% or 100% identical to the amino acid sequence of SEQ ID NO: 25 or SEQ ID NO: 94 (which is SEQ ID NO: 25 without the C-terminal “G”); and/or
(ii) a VH comprising an amino acid sequence that is at least about 90%, 95%, 96%, 97%, 98%, 99% or 100% identical to the amino acid sequence of SEQ ID NO: 26.

In certain embodiments, the third binding domain that specifically binds to human CD19 comprises:
(i) a VL comprising an amino acid sequence that is at least about 90%, 95%, 96%, 97%, 98%, 99% or 100% identical to the amino acid sequence of SEQ ID NO: 16 or SEQ ID NO: 88 (which is SEQ ID NO: 16 without the C-terminal “G”); and/or
(ii) a VH comprising an amino acid sequence that is at least about 90%, 95%, 96%, 97%, 98%, 99% or 100% identical to the amino acid sequence of SEQ ID NO: 17.

For example, any of such VL and/or VH sequence identity may be met while comprising the CDR-L1, CDR-L2, and CDR-L3 sequences and/or hCDR-H1, CDR-H2, and CDR-H3 sequences as described above.

Such trispecific antigen binding protein may have or comprise multiple formats, including but not limited to, Fab-(sdAb)₂, Fab-(scFv)₂ (or M-gager®), F(ab′)₂ fragment, quadroma, CrossMab, a Tri-MAB, a CODV-Ig, an OrthoTsAb, a 2+1 common LC, a Triplebody, a Triflex, a MATCH3, or a triabody, DuoBody, and tetrabody; in certain embodiments, a Fab-(scFv)₂ is provided.

In certain embodiments, the trispecific antigen binding protein comprises three ABUs wherein two ABUs are scFvs and one ABU is a Fab. In certain embodiments, the trispecific antigen binding protein is a Fab-(scFv)₂, comprising two scFv molecules attached to a Fab.

In certain embodiments, the two scFvs are fused to the Fab via a peptide linker, wherein one scFv is fused at the C-terminus of the Fab heavy chain and the other scFv is fused to the Fab light chain.

In certain embodiments, the antigen binding protein is a Fab-(scFv)₂, comprising two scFv molecules attached to a Fab. In certain embodiments, the first binding domain and the third binding domain are fused to the second binding domain via a peptide linker, particularly at the C-terminus of the Fab heavy and light chain, respectively. The scFv can be attached to the C-terminus of the Fab heavy chain, the C-terminus of the Fab light chain, the N-terminus of the Fab heavy chain and/or the N-terminus of the Fab light chain. In certain embodiments, a first scFv is operably linked to the C-terminus of the Fab heavy chain and a second scFv is operably linked to the N-terminus of the Fab light chain. In certain embodiments, a first scFv is operably linked to the C-terminus of the Fab light chain and a second scFv is operably linked to the N-terminus of the Fab light chain.

Although the three binding domains are in some cases referred to herein as the “first antigen binding domain”, “second antigen binding domain”, and “third antigen binding domain”, or the like, these terms do not necessarily indicate a particular order or a particular orientation of the binding domains within a multispecific antigen binding protein.

An advantage of the Fab-(scFvs)₂ scaffolds of the disclosure is the intermediate molecular size of approximately 95-110 kDa. Blinatumomab, a bispecific T cell engager (BiTE), has shown excellent results in patients with relapsed or refractory acute lymphoblastic leukemia. Because of its small size (60 kDa), blinatumomab is characterized by a short serum half-life of a few hours, and therefore continuous infusion is needed (see, U.S. Pat. No. 7,112,324 B1). Fab-(scFvs)₂ of the disclosure are expected to have a significantly longer half-life in comparison to smaller bispecific antibodies such as BiTEs like blinatumomab, and therefore do not require continuous infusion. An intermediate sized molecule may avoid kidney clearance and provide a half-life sufficient for improved tissue accumulation. While having increased plasma half-life compared to other small bispecific formats, the Fab-(scFvs)₂ of the disclosure still retain the tissue penetration ability, enabling B cell depletion deep in lymphoid tissue. Without being bound to theory, their fast clearance compared to larger IgGs for example may allow for accelerated de novo reconstitution of the B cell compartment from hematopoietic stem cells (HSCs). On the other hand, molecules of the instant disclosure lacking an Fc domain are expected to have a shorter half-life than larger molecules including an Fc domain. A prolonged half-life may overstimulate T cells and lead to T cell exhaustion. Molecules lacking the Fc domain generally do not mediate antibody-dependent cell-mediated cytotoxicity (ADCC) or complement-dependent cytotoxicity (CDC), thus having access to a different immune cell environment, more so, when considering the tissue penetration capabilities.

In certain embodiments, the multispecific antigen binding proteins according to the present invention have a Fab domain heavy chain comprising a CH1 domain and a VH domain. In certain embodiments, the Fab domain further comprises at least 5 amino acids of an antibody hinge region, in particular at the C-terminus of the heavy chain of the Fab domain. In certain embodiments, the Fab domain comprises the sequence stretch up to the first cysteine of the antibody hinge region. In certain embodiments, said sequence stretch is or comprises the sequence EPKSC (SEQ ID NO.: 37). The presence of cysteine allows for an additional disulfide bridge which may further stabilize the antigen binding protein. The antibody hinge region may additionally comprise the sequence GGGGS (SEQ ID NO.: 31) which may serve as a linker sequence to a second or third binding domain(s). In certain embodiments, the Fab domain light chain comprises a CL domain and a VL domain. The CL domain may be followed by a linker, such as GGGGS (SEQ ID NO.: 31), to which optionally a second scFv is attached.

In certain embodiments, the antigen binding protein is a Fab-(scFv)₂, wherein the first CD3-binding domain is a Fab molecule, the (second) BCMA-binding domain is a scFv and the (third) CD19-binding domain is a scFv. In other embodiments, the antigen binding protein is a Fab-(scFv)₂, wherein the BCMA-binding domain is a Fab molecule, the CD3-binding domain is a scFv and the CD19-binding domain is a scFv. In other embodiments, the antigen binding protein is a Fab-(scFv)₂, wherein the CD19-binding domain is a Fab molecule, the CD3-binding domain is a scFv and the BCMA-binding domain is a scFv.

In certain embodiments, the scFv binding domains are fused to the Fab domain via a peptide linker, particularly at the C-terminus of the Fab heavy and light chain, respectively. Suitable linkers are outlined supra. In certain embodiments, the scFv is in VL-linker-VH orientation.

In some embodiments, a trispecific antigen binding protein targeting BCMA, CD3 and CD19 according to the present disclosure may comprise a Fab which specifically binds to CD3, a scFv which specifically binds to BCMA, and a scFv which specifically binds to CD19. In some embodiments, a trispecific antigen binding molecule targeting BCMA, CD3 and CD19 according to the present disclosure may have the Fab-(scFv)₂ format, which comprises three antigen binding units (ABU) comprising a first ABU (ABU-1) comprising a Fab which specifically binds to CD3, a second ABU (ABU-2) comprising a scFv which specifically binds to BCMA, and a third ABU (ABU-3) comprising a scFv which specifically binds to CD19.

In certain embodiments, the CD3-binding Fab may comprise: a heavy chain comprising a VH and a CH1; and a light chain comprising a VL and a CL. In certain embodiments, the CD19-binding scFv may comprise a VL, a linker, and a VH in the direction from the N-terminus to the C-terminus, or a VH, a linker, and a VL in the direction from the N-terminus to the C-terminus. In certain embodiments, the BCMA-binding scFv may comprise a VL, a linker, and a VH in the direction from the N-terminus to the C-terminus, or a VH, a linker, and a VL in the direction from the N-terminus to the C-terminus.

A trispecific antigen binding protein may comprise: a first polypeptide (which may be referred to as a light chain (LC) of the trispecific antigen binding protein) which comprises the light chain of the CD3-binding Fab; and a second polypeptide (which may be referred to as a heavy chain (HC) of the trispecific antigen binding protein) which comprises the heavy chain of the CD3-binding Fab.

In particular embodiments, the CD19-binding scFv may be fused to a light chain of the CD3-binding Fab, e.g., via a linker (any appropriate linkers such as those disclosed herein, e.g., SEQ ID NO: 31). In some cases, the N-terminus of the scFv is fused to the C-terminus of the light chain of the Fab. In cases, the first polypeptide (or LC) will comprise: a CD3-binding VL, a CL, an optional linker, and a CD19-binding scFv (e.g., VL-linker-VH, or VH-linker-VL), in the direction from the N-terminus to the C-terminus. For example, the first polypeptide (or LC) may comprise SEQ ID NO: 30, 109, 102, 103, or 110, or a sequence at least about 80%, 85%, 90%, 95%, 96%, 97%, 98%, 99% or 100% identical thereto. In some cases, the C-terminus of the scFv is fused to the N-terminus of the light chain of the Fab. In some cases, the first polypeptide (or LC) will comprise: a CD19-binding scFv (e.g., VL-linker-VH, or VH-linker-VL), an optional linker, a CD3-binding VL, and a CL, in the direction from the N-terminus to the C-terminus.

In certain embodiments, the CD19-binding scFv may be fused to a heavy chain of the CD3-binding Fab, e.g., via a linker (any appropriate linkers such as those disclosed herein, e.g., SEQ ID NO: 31). In some cases, the N-terminus of the scFv is fused to the C-terminus of the heavy chain of the Fab. In cases, the second polypeptide (or HC) will comprise: a CD3-binding VH, a CH1, an optional linker, and a CD19-binding scFv (e.g., VL-linker-VH, or VH-linker-VL), in the direction from the N-terminus to the C-terminus. For example, the second polypeptide (or HC) may comprise SEQ ID NO: 104, 111, 106, 107, or 112, or a sequence at least about 80%, 85%, 90%, 95%, 96%, 97%, 98%, 99% or 100% identical thereto. In some cases, the C-terminus of the scFv is fused to the N-terminus of the heavy chain of the Fab. In some cases, the second polypeptide (or HC) will comprise: a CD19-binding scFv (e.g., VL-linker-VH, or VH-linker-VL), an optional linker, a CD3-binding VH, and a CH1, in the direction from the N-terminus to the C-terminus.

In particular embodiments, the BCMA-binding scFv may be fused to a heavy chain of the CD3-binding Fab, e.g., via a linker (any appropriate linkers such as those disclosed herein, e.g., SEQ ID NO: 31). In some cases, the N-terminus of the scFv is fused to the C-terminus of the heavy chain of the Fab. In cases, the second polypeptide (or HC) will comprise: a CD3-binding VH, a CH1, an optional linker, and a BCMA-binding scFv (e.g., VL-linker-VH, or VH-linker-VL), in the direction from the N-terminus to the C-terminus. For example, the second polypeptide (or HC) may comprise SEQ ID NO: 29 or 101, or a sequence at least about 80%, 85%, 90%, 95%, 96%, 97%, 98%, 99% or 100% identical thereto. In some cases, the C-terminus of the scFv is fused to the N-terminus of the heavy chain of the Fab. In some cases, the second polypeptide (or HC) will comprise: a BCMA-binding scFv (e.g., VL-linker-VH, or VH-linker-VL), an optional linker, a CD3-binding VH, and a CH1, in the direction from the N-terminus to the C-terminus.

In certain embodiments, the BCMA-binding scFv may be fused to a light chain of the CD3-binding Fab, e.g., via a linker (any appropriate linkers such as those disclosed herein, e.g., SEQ ID NO: 31). In some cases, the N-terminus of the scFv is fused to the C-terminus of the light chain of the Fab. In cases, the first polypeptide (or LC) will comprise: a CD3-binding VL, a CL, an optional linker, and a BCMA-binding scFv (e.g., VL-linker-VH, or VH-linker-VL), in the direction from the N-terminus to the C-terminus. For example, the first polypeptide (or LC) may comprise SEQ ID NO: 105 or 108, or a sequence at least about 80%, 85%, 90%, 95%, 96%, 97%, 98%, 99% or 100% identical thereto. In some cases, the C-terminus of the scFv is fused to the N-terminus of the light chain of the Fab. In some cases, the first polypeptide (or LC) will comprise: a BCMA-binding scFv (e.g., VL-linker-VH, or VH-linker-VL), an optional linker, a CD3-binding VL, and a CL, in the direction from the N-terminus to the C-terminus.

Any combinations of such first and second polypeptides (or LC and HC) which provide specificity to CD3, CD19, and BCMA may be used and paired to provide a multispecific or trispecific antigen binding protein according to the present disclosure. Such a protein may comprise a structure, for example shown in the top panel of FIG. 24A.

In particular embodiments, the N-termini of the CD19-binding scFv and the BCMA-binding scFv may be fused to the C-termini of the heavy and light chains, respectively, or the light and heavy chains, respectively, of the CD3-binding Fab.

In some particular cases, a first polypeptide (or LC) comprising SEQ ID NO: 30, 109, 102, 103, or 110, or a sequence at least about 80%, 85%, 90%, 95%, 96%, 97%, 98%, 99% or 100% identical thereto may be paired with a second polypeptide (or HC) comprising SEQ ID NO: 29 or 101, or a sequence at least about 80%, 85%, 90%, 95%, 96%, 97%, 98%, 99% or 100% identical thereto.

In some other particular cases, a second polypeptide (or HC) comprising SEQ ID NO: 104, 111, 106, 107, or 112, or a sequence at least about 80%, 85%, 90%, 95%, 96%, 97%, 98%, 99% or 100% identical thereto may be paired with a first polypeptide (or LC) comprising SEQ ID NO: 105 or 108, or a sequence at least about 80%, 85%, 90%, 95%, 96%, 97%, 98%, 99% or 100% identical thereto.

In some preferred embodiments, a first polypeptide (or LC) comprising SEQ ID NO: 30, 109, 102, 103, or 110, or a sequence at least about 80%, 85%, 90%, 95%, 96%, 97%, 98%, 99% or 100% identical thereto may be paired with a second polypeptide (or HC) comprising SEQ ID NO: 29 or 101, or a sequence at least about 80%, 85%, 90%, 95%, 96%, 97%, 98%, 99% or 100% identical thereto.

In certain preferred embodiments, the trispecific antigen binding molecule targeting BCMA, CD3 and CD19 comprises:
(a) an amino acid sequence that is at least about 80%, 85%, 90%, 91%, 92%, 93%, 94%, 95%, 96%, 97%, 98%, 99% or 100% identical to the amino acid sequence of SEQ ID NO: 29; and/or
(b) an amino acid sequence that is at least about 80%, 85%, 90%, 91%, 92%, 93%, 94%, 95%, 96%, 97%, 98%, 99% or 100% identical to the amino acid sequence of SEQ ID NO: 30. Such trispecific antigen binding proteins are exemplified by the molecule denominated M4148, see the example section below. In certain embodiments, the variant sequences of SEQ ID NO: 29 and/or SEQ ID NO: 30 comprise the CDR sequences of SEQ ID NOs: 1-6, SEQ ID NOs: 19-24, and SEQ ID NOs: 10-15.

As can be derived from the examples, the trispecific antigen binding proteins according to the present invention, and in particular M4148 (a Fab-(scFv)₂ comprising SEQ ID NO: 29 and SEQ ID NO: 30), have an affinity of the CD19 binding domain that is significantly lower than the affinity of the BCMA binding domain. Without being bound to theory, said difference accounts for the much higher CD19 expression level on B cells versus lower BCMA expression level on plasma cells, thereby reducing a potential CD19 driven drug sink and enabling the trispecific antigen binding protein to migrate deep into the lymphoid tissue (e.g., including bone marrow, spleen, and/or lymph node). In certain embodiments, the affinity of the CD19 binding domain is at least about 20 times, about 50 times, or at least about 100 times lower than the affinity of the BCMA binding domain (i.e., the equilibrium dissociation constant of the CD19 binding domain is at least about 20 times, about 50 times, or at least about 100 times higher than the one of the BCMA binding domain). Thus, in certain embodiments, the antigen binding domain comprises three binding domains, wherein
(i) a BCMA binding domain having an affinity (K_{D}) between about 0.5 μM to about 0.5 nM to human BCMA;
(ii) a CD19 binding domain having an affinity (K_{D}) between about 0.01 nM to about 5 nM to human CD19; and
(iii) a CD3 binding domain having an affinity (K_{D}) between about 1 nM to about 20 nM to human CD3, such as 1 nM, 2 nM, 5 nM, 10 nM, 15 nM, or 20 nM.

In certain embodiments, the CD3 affinity (K_{D}) is measured on a CD3δε or a CD3εγ complex.

In particular embodiments, a trispecific antigen binding protein of the disclosure comprises:
(i) a BCMA binding domain having an affinity (K_{D}) of about 0.02 nM to human BCMA;
(ii) a CD19 binding domain having an affinity (K_{D}) of about 1.1 nM to human CD19; and
(iii) a CD3 binding domain having an affinity (K_{D}) of about 3 nM to human CD3δε.

In certain embodiments, the antigen binding proteins disclosed herein may show a decreased incidence of immune related adverse events (irAEs) and better efficacy, translating into high clinical efficacy.

As shown in the examples, the trispecific antigen binding proteins of the disclosure show potent and nearly complete B cell depletion on both SLE and RA PBMCs in a dose-dependent manner.

In certain embodiments, the trispecific antigen binding proteins of the disclosure shows efficacy for human IgG depletion in a humanized SLE mouse model, e.g., the NCG-M model described Example 8 (see also CN117946962A for reference, incorporated herein in its entirety).

As evidenced in the Examples, the trispecific antigen binding proteins of the disclosure are highly specific. In certain embodiments, the trispecific antigen binding proteins do not elicit Granzyme B release on BCMA-negative/CD19-negative cancer cell lines, e.g., NCI-H1703, PC3, HL-60, A375 and/or SKMEL-30. In certain embodiments, the trispecific antigen binding proteins of the disclosure show greater potency on BCMA-positive/CD19-positive (e.g., Daudi and Raji cells) than bispecific CD19xCD3 or BCMAxCD3 controls.

The trispecific antigen binding proteins of the disclosure show favorable drug-like biophysical properties, (as e.g., characterized by solubility, accelerated shelf-life stability and/or stability at physiological conditions) and have a favorable manufacturability profile. In certain embodiments, the trispecific antigen binding protein of the disclosure remains at least 95%, 96%, 97%, 98%, 99% or 100% monomeric when being concentrated to 50 mg/ml in PBS, as e.g., determined by SEC-HPLC. In particular embodiments, the trispecific antigen binding protein remains at least 95%, 96%, 97%, 98%, 99% or 100% monomeric when being concentrated to 120 mg/ml in 10 mM Histidine buffer pH 5.5, as e.g., determined by SEC-HPLC.

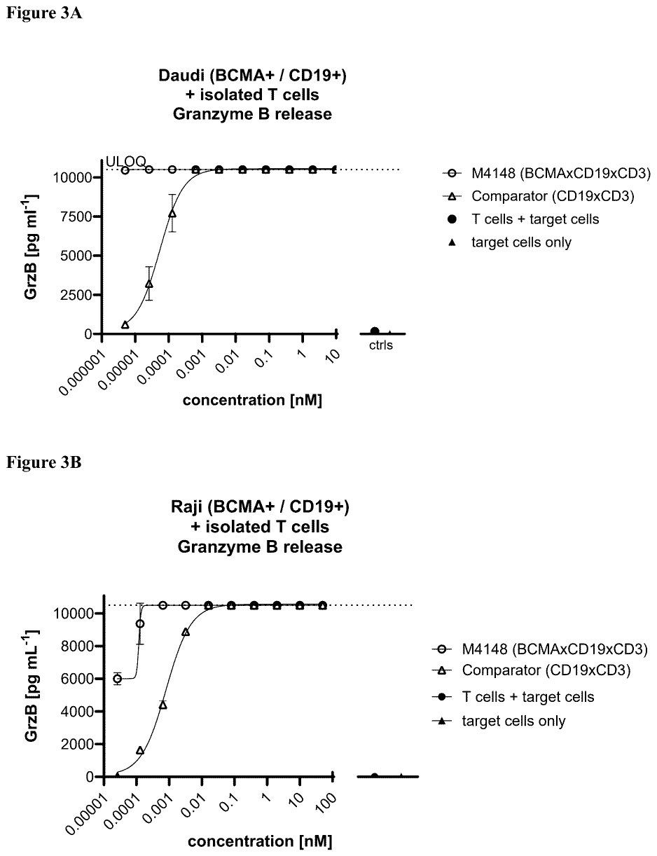

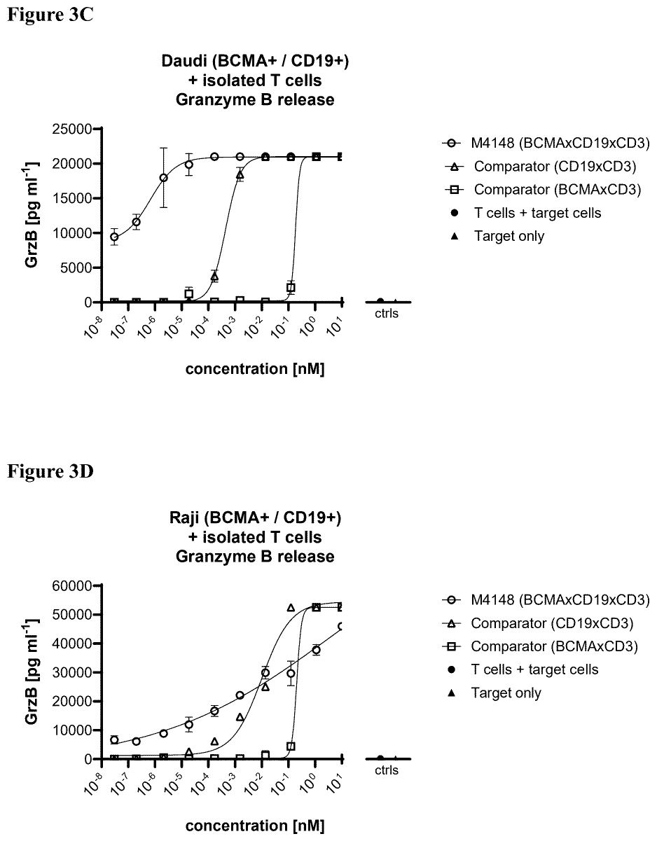

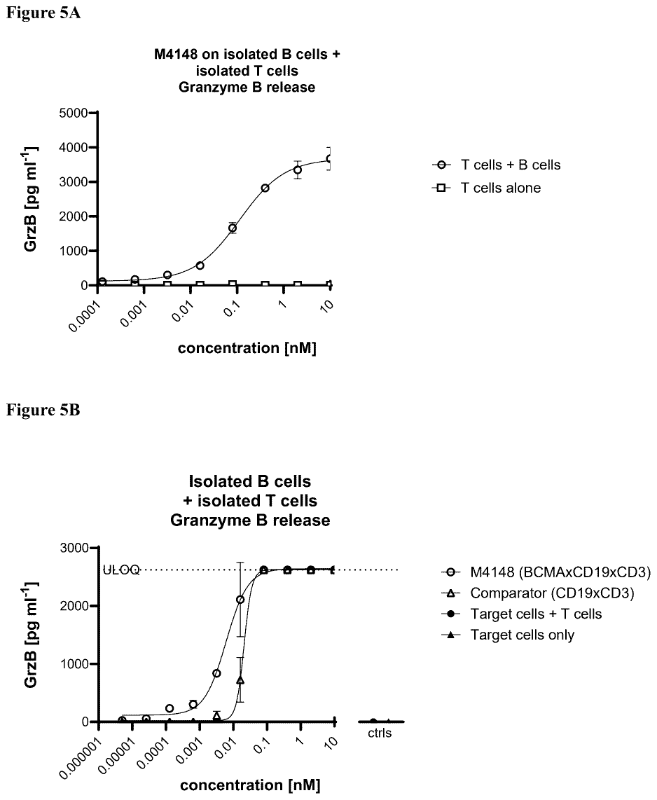

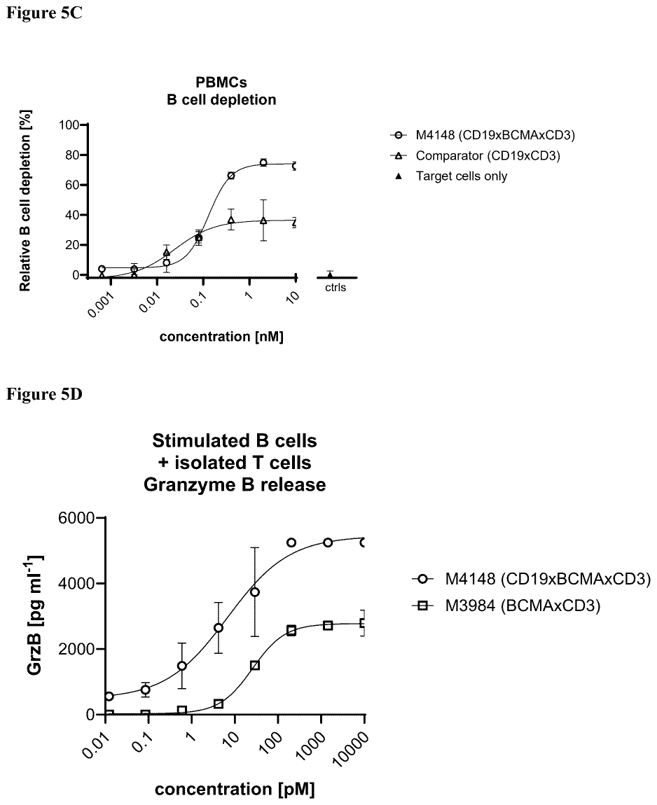

In certain embodiments, the trispecific antigen binding protein of the disclosure remains stable under accelerated conditions, for example, in certain embodiments, incubation of the trispecific antigen binding proteins of the disclosure at 50 mg/mL in 10 mM Histidine buffer at pH 5.5 at 40 °C for 7 days leads to oligomer formation of less than 1% and/or fragment formation of less than 1%. In certain embodiments, the trispecific antigen binding protein of the disclosure remains stable at simulated physiological conditions, for example, in certain embodiments, incubation of the trispecific antigen binding proteins of the disclosure at 1 mg/mL in artificial subcutaneous fluid buffer (see Schuster et al., Journal of Pharmaceutical Sciences 110 (2021):2386-2394) at 34 °C for 7 and 14 days leads to oligomer formation of less than 1% and/or fragment formation of less than 1%. In certain embodiments, the trispecific antigen binding proteins of the disclosure show increased potency over competitors. For example, as shown in FIG. 3, a trispecific BCMAxCD3XCD19 binding protein according to the present invention has increased potency when compared to a clinically validated bispecific CD19xCD3 binding protein. As shown in FIG. 5D, a trispecific BCMAxCD3XCD19 binding protein according to the present invention has increased potency when compared to a bispecific control molecule targeting BCMAxCD3.

In certain embodiments, the trispecific antigen binding proteins of the disclosure are capable of migrating into lymphoid tissue, such as bone marrow, spleen, and/or lymph node.

In certain embodiments, the antigen binding proteins of the disclosure, in particular the trispecific ones, show no reactivity to human primary cells, in particular to human lung fibroblasts (NHLF), human pulmonary artery smooth muscle cells (PASMC), human pulmonary fibroblasts (HPF), human cardiac fibroblasts (HCF) and/or human aortic smooth muscle cells (HAoSMC).

The sequences disclosed herein are given in Table 2:

TABLE 2
Amino Acid Sequences

| Description | Sequence ID NO | Sequence |
|---|---|---|
| M4148, M3984 | SEQ ID NO: 1 | RASKSVSFLGINLIY |
| BCMA_CDR-L1 | | |
| M4148, M3984 | SEQ ID NO: 2 | QASNRDT |
| BCMA_CDR-L2 | | |
| M4148, M3984 | SEQ ID NO: 3 | LQTPFFPRT |
| BCMA_CDR-L3 | | |
| M4148, M3984 | SEQ ID NO: 4 | DYSIN |
| BCMA_CDR-H1 | | |
| M4148, M3984 | SEQ ID NO: 5 | WINTETGNPTYAQGFTG |
| BCMA_CDR-H2 | | |
| M4148, M3984 | SEQ ID NO: 6 | DKHLAMDY |
| BCMA_CDR-H3 | | |
| M4148, M3984 | SEQ ID NO: 7 | DIVLTQSPASLAVSPGERATITCRASKSVSFL |
| BCMA VL | | GINLIYWYQQKPGQPPKLLIYQASNRDTGVP |
| | | ARFSGSGSGTDFTLTINPVEAEDTANYYCLQ |
| | | TPFFPRTFGCGTKLEIK |
| M4148, M3984 | SEQ ID NO: 8 | QVQLVQSGGGSVKPGASVKVSCKASGYTFT |
| BCMA_VH | | DYSINWVRQAPGKCLEWMGWINTETGNPTY |
| | | AQGFTGRFTISLDTSVSTAYLQISSLKAEDTA |
| | | SYYCALDKHLAMDYWGQGTSVTVSS |
| M4148, M3984 | SEQ ID NO: 9 | DIVLTQSPASLAVSPGERATITCRASKSVSFL |
| BCMA_scFv | | GINLIYWYQQKPGQPPKLLIYQASNRDTGVP |
| | | ARFSGSGSGTDFTLTINPVEAEDTANYYCLQ |
| | | TPFFPRTFGCGTKLEIKGGGGSGGGGSGGGG |
| | | SGGGGSQVQLVQSGGGSVKPGASVKVSCKA |
| | | SGYTFTDYSINWVRQAPGKCLEWMGWINTE |
| | | TGNPTYAQGFTGRFTISLDTSVSTAYLQISSL |
| | | KAEDTASYYCALDKHLAMDYWGQGTSVTV |
| | | SS |
| M4148, M4141 | SEQ ID NO: 10 | RASQSVDYDGDSYLN |
| CD19_CDR-L1 | | |
| M4148, M4141 | SEQ ID NO: 11 | DASNLVS |
| CD19_CDR-L2 | | |
| M4148, M4141 | SEQ ID NO: 12 | QQSTEDPWT |
| CD19_CDR-L3 | | |
| M4148, M4141 | SEQ ID NO: 13 | SYWMN |
| CD19_CDR-H1 | | |
| M4148, M4141 | SEQ ID NO: 14 | QIWPGDGDTNYNGKFKG |
| CD19_CDR-H2 | | |
| M4148, M4141 | SEQ ID NO: 15 | RETTTVGRYYYAMDY |
| CD19_CDR-H3 | | |
| M4148, M4141 | SEQ ID NO: 16 | EIVMTQSPSTLSASVGDRVTITCRASQSVDY |
| CD19_VL | | DGDSYLNWYQQKPGKAPKLLIYDASNLVSG |
| | | VPSRFSGSGSGTDFTLTISSLQPEDFATYYCQ |
| | | QSTEDPWTFGCGTKLTVLG |
| M4148, M4141 | SEQ ID NO: 17 | EVQLVESGGGSVQPGGSLRLSCKASGYAFSS |
| CD19_VH | | YWMNWVRQAPGKCLEWIGQIWPGDGDTNY |
| | | NGKFKGRFTISADESKNTAYLQMNSLRAEDT |
| | | ATYYCARRETTTVGRYYYAMDYWGQGTTV |
| | | TVSS |
| M4148, M4141 | SEQ ID NO: 18 | EIVMTQSPSTLSASVGDRVTITCRASQSVDY |
| CD19_scFv | | DGDSYLNWYQQKPGKAPKLLIYDASNLVSG |
| | | VPSRFSGSGSGTDFTLTISSLQPEDFATYYCQ |
| | | QSTEDPWTFGCGTKLTVLGGGGGSGGGGSG |
| | | GGGSGGGGSEVQLVESGGGSVQPGGSLRLS |
| | | CKASGYAFSSYWMNWVRQAPGKCLEWIGQ |
| | | IWPGDGDTNYNGKFKGRFTISADESKNTAYL |
| | | QMNSLRAEDTATYYCARRETTTVGRYYYA |
| | | MDYWGQGTTVTVSS |
| M4148, M4141, | SEQ ID NO: 19 | GSSTGAVTTSNYAN |
| M3984 | | |
| CD3_CDR-L1 | | |
| M4148, M4141, | SEQ ID NO: 20 | GTNKRAP |
| M3984 | | |
| CD3_CDR-L2 | | |
| M4148, M4141, | SEQ ID NO: 21 | ALWYSNHWV |
| M3984 | | |
| CD3_CDR-L3 | | |
| M4148, M4141, | SEQ ID NO: 22 | TYAMN |
| M3984 | | |
| CD3_CDR-H1 | | |
| M4148, M4141, | SEQ ID NO: 23 | RIRSKYNNYATYYADSVKG |
| M3984 | | |
| CD3_CDR-H2 | | |
| M4148, M4141, | SEQ ID NO: 24 | HGNFGDSYVSWFAY |
| M3984 | | |
| CD3_CDR-H3 | | |
| M4148, M4141, | SEQ ID NO: 25 | QAVVTQEPSLTVSPGGTVTLTCGSSTGAVTT |
| M3984 | | SNYANWVQQKPGKSPRGLIGGTNKRAPGVP |
| CD3_VL | | ARFSGSLLGGKAALTISGAQPEDEADYYCAL |
| | | WYSNHWVFGGGTKLTVLG |
| M4148, M4141, | SEQ ID NO: 26 | EVQLVESGGGLVQPGGSLRLSCAASGFTFST |
| M3984 | | YAMNWVRQAPGKGLEWVGRIRSKYNNYAT |
| CD3_VH | | YYADSVKGRFTISRDDSKNTLYLQMNSLRA |
| | | EDTAVYYCVRHGNFGDSYVSWFAYWGQGT |
| | | LVTVSS |
| M4148, M4141, | SEQ ID NO: 27 | QAVVTQEPSLTVSPGGTVTLTCGSSTGAVTT |
| M3984 | | SNYANWVQQKPGKSPRGLIGGTNKRAPGVP |
| CD3_LC | | ARFSGSLLGGKAALTISGAQPEDEADYYCAL |
| | | WYSNHWVFGGGTKLTVLGQPKAAPSVTLFP |
| | | PSSEELQANKATLVCLISDFYPGAVTVAWKA |
| | | DSSPVKAGVETTTPSKQSNNKYAASSYLSLT |
| | | PEQWKSHRSYSCQVTHEGSTVEKTVAPTECS |
| M4148, M4141, | SEQ ID NO: 28 | EVQLVESGGGLVQPGGSLRLSCAASGFTFST |
| M3984 | | YAMNWVRQAPGKGLEWVGRIRSKYNNYAT |
| CD3_HC | | YYADSVKGRFTISRDDSKNTLYLQMNSLRA |
| | | EDTAVYYCVRHGNFGDSYVSWFAYWGQGT |
| | | LVTVSSASTKGPSVFPLAPSSKSTSGGTAALG |
| | | CLVKDYFPEPVTVSWNSGALTSGVHTFPAVL |
| | | QSSGLYSLSSVVTVPSSSLGTQTYICNVNHKP |
| | | SNTKVDKKVEPKSC |
| M4148 | SEQ ID NO: 29 | EVQLVESGGGLVQPGGSLRLSCAASGFTFST |
| HC | | YAMNWVRQAPGKGLEWVGRIRSKYNNYAT |
| | | YYADSVKGRFTISRDDSKNTLYLQMNSLRA |
| | | EDTAVYYCVRHGNFGDSYVSWFAYWGQGT |
| | | LVTVSSASTKGPSVFPLAPSSKSTSGGTAALG |
| | | CLVKDYFPEPVTVSWNSGALTSGVHTFPAVL |
| | | QSSGLYSLSSVVTVPSSSLGTQTYICNVNHKP |
| | | SNTKVDKKVEPKSCGGGGSDIVLTQSPASLA |
| | | VSPGERATITCRASKSVSFLGINLIYWYQQKP |
| | | GQPPKLLIYQASNRDTGVPARFSGSGSGTDF |
| | | TLTINPVEAEDTANYYCLQTPFFPRTFGCGTK |
| | | LEIKGGGGSGGGGSGGGGSGGGGSQVQLVQ |
| | | SGGGSVKPGASVKVSCKASGYTFTDYSINW |
| | | VRQAPGKCLEWMGWINTETGNPTYAQGFT |
| | | GRFTISLDTSVSTAYLQISSLKAEDTASYYCA |
| | | LDKHLAMDYWGQGTSVTVSS |
| M4148 | SEQ ID NO: 30 | QAVVTQEPSLTVSPGGTVTLTCGSSTGAVTT |
| LC | | SNYANWVQQKPGKSPRGLIGGTNKRAPGVP |
| | | ARFSGSLLGGKAALTISGAQPEDEADYYCAL |
| | | WYSNHWVFGGGTKLTVLGQPKAAPSVTLFP |
| | | PSSEELQANKATLVCLISDFYPGAVTVAWKA |
| | | DSSPVKAGVETTTPSKQSNNKYAASSYLSLT |
| | | PEQWKSHRSYSCQVTHEGSTVEKTVAPTECS |
| | | GGGGSEIVMTQSPSTLSASVGDRVTITCRAS |
| | | QSVDYDGDSYLNWYQQKPGKAPKLLIYDAS |
| | | NLVSGVPSRFSGSGSGTDFTLTISSLQPEDFA |
| | | TYYCQQSTEDPWTFGCGTKLTVLGGGGGSG |
| | | GGGSGGGGSGGGGSEVQLVESGGGSVQPGG |
| | | SLRLSCKASGYAFSSYWMNWVRQAPGKCLE |
| | | WIGQIWPGDGDTNYNGKFKGRFTISADESKN |
| | | TAYLQMNSLRAEDTATYYCARRETTTVGRY |
| | | YYAMDYWGQGTTVTVSS |
| M4141 HC | SEQ ID NO: 48 | EVQLVESGGGLVQPGGSLRLSCAASGFTFST |
| | | YAMNWVRQAPGKGLEWVGRIRSKYNNYAT |
| | | YYADSVKGRFTISRDDSKNTLYLQMNSLRA |
| | | EDTAVYYCVRHGNFGDSYVSWFAYWGQGT |
| | | LVTVSSASTKGPSVFPLAPSSKSTSGGTAALG |
| | | CLVKDYFPEPVTVSWNSGALTSGVHTFPAVL |
| | | QSSGLYSLSSVVTVPSSSLGTQTYICNVNHKP |
| | | SNTKVDKKVEPKSCGGGGSEIVMTQSPSTLS |
| | | ASVGDRVTITCRASQSVDYDGDSYLNWYQQ |
| | | KPGKAPKLLIYDASNLVSGVPSRFSGSGSGT |
| | | DFTLTISSLQPEDFATYYCQQSTEDPWTFGC |
| | | GTKLTVLGGGGGSGGGGSGGGGSGGGGSEV |
| | | QLVESGGGSVQPGGSLRLSCKASGYAFSSY |
| | | WMNWVRQAPGKCLEWIGQIWPGDGDTNYN |
| | | GKFKGRFTISADESKNTAYLQMNSLRAEDTA |
| | | TYYCARRETTTVGRYYYAMDYWGQGTTVT |
| | | VSS |
| M4141, M3984 | SEQ ID NO: 49 | QAVVTQEPSLTVSPGGTVTLTCGSSTGAVTT |
| LC | | SNYANWVQQKPGKSPRGLIGGTNKRAPGVP |
| | | ARFSGSLLGGKAALTISGAQPEDEADYYCAL |
| | | WYSNHWVFGGGTKLTVLGQPKAAPSVTLFP |
| | | PSSEELQANKATLVCLISDFYPGAVTVAWKA |
| | | DSSPVKAGVETTTPSKQSNNKYAASSYLSLT |
| | | PEQWKSHRSYSCQVTHEGSTVEKTVAPTECS |
| M3984 HC | SEQ ID NO: 50 | EVQLVESGGGLVQPGGSLRLSCAASGFTFST |
| | | YAMNWVRQAPGKGLEWVGRIRSKYNNYAT |
| | | YYADSVKGRFTISRDDSKNTLYLQMNSLRA |
| | | EDTAVYYCVRHGNFGDSYVSWFAYWGQGT |
| | | LVTVSSASTKGPSVFPLAPSSKSTSGGTAALG |
| | | CLVKDYFPEPVTVSWNSGALTSGVHTFPAVL |
| | | QSSGLYSLSSVVTVPSSSLGTQTYICNVNHKP |
| | | SNTKVDKKVEPKSCGGGGSDIVLTQSPASLA |
| | | VSPGERATITCRASKSVSFLGINLIYWYQQKP |
| | | GQPPKLLIYQASNRDTGVPARFSGSGSGTDF |
| | | TLTINPVEAEDTANYYCLQTPFFPRTFGCGTK |
| | | LEIKGGGGSGGGGSGGGGSGGGGSQVQLVQ |
| | | SGGGSVKPGASVKVSCKASGYTFTDYSINW |
| | | VRQAPGKCLEWMGWINTETGNPTYAQGFT |
| | | GRFTISLDTSVSTAYLQISSLKAEDTASYYCA |
| | | LDKHLAMDYWGQGTSVTVSS |
| CD19 × CD3 | SEQ ID NO: 51 | DIQLTQSPASLAVSLGQRATISCKASQSVDY |
| bispecific | | DGDSYLNWYQQIPGQPPKLLIYDASNLVSGI |
| comparator | | PPRFSGSGSGTDFTLNIHPVEKVDAATYHCQ |
| | | QSTEDPWTFGGGTKLEIKGGGGSGGGGSGG |
| | | GGSQVQLQQSGAELVRPGSSVKISCKASGYA |
| | | FSSYWMNWVKQRPGQGLEWIGQIWPGDGD |
| | | TNYNGKFKGKATLTADESSSTAYMQLSSLA |
| | | SEDSAVYFCARRETTTVGRYYYAMDYWGQ |
| | | GTTVTVSSGGGGSDIKLQQSGAELARPGASV |
| | | KMSCKTSGYTFTRYTMHWVKQRPGQGLEW |
| | | IGYINPSRGYTNYNQKFKDKATLTTDKSSST |
| | | AYMQLSSLTSEDSAVYYCARYYDDHYCLDY |
| | | WGQGTTLTVSSVEGGSGGSGGSGGSGGVDD |
| | | IQLTQSPAIMSASPGEKVTMTCRASSSVSYM |
| | | NWYQQKSGTSPKRWIYDTSKVASGVPYRFS |
| | | GSGSGTSYSLTISSMEAEDAATYYCQQWSSN |
| | | PLTFGAGTKLELKHHHHHH |
| BCMA × CD3 | SEQ ID NO: 52 | QLQLQESGPGLVKPSETLSLTCTVSGGSISSG |
| bispecific | | SYFWGWIRQPPGKGLEWIGSIYYSGITYYNP |
| comparator anti- | | SLKSRVTISVDTSKNQFSLKLSSVTAADTAV |
| BCMA HC | | YYCARHDGAVAGLFDYWGQGTLVTVSSAS |
| | | TKGPSVFPLAPCSRSTSESTAALGCLVKDYFP |
| | | EPVTVSWNSGALTSGVHTFPAVLQSSGLYSL |
| | | SSVVTVPSSSLGTKTYTCNVDHKPSNTKVDK |
| | | RVESKYGPPCPPCPAPEAAGGPSVFLFPPKPK |
| | | DTLMISRTPEVTCVVVDVSQEDPEVQFNWY |
| | | VDGVEVHNAKTKPREEQFNSTYRVVSVLTV |
| | | LHQDWLNGKEYKCKVSNKGLPSSIEKTISKA |
| | | KGQPREPQVYTLPPSQEEMTKNQVSLTCLVK |
| | | GFYPSDIAVEWESNGQPENNYKTTPPVLDSD |
| | | GSFFLYSRLTVDKSRWQEGNVFSCSVMHEA |
| | | LHNHYTQKSLSLSLGK |
| BCMA × CD3 | SEQ ID NO: 53 | SYVLTQPPSVSVAPGQTARITCGGNNIGSKSV |
| bispecific | | HWYQQPPGQAPVVVVYDDSDRPSGIPERFS |
| comparator anti- | | GSNSGNTATLTISRVEAGDEAVYYCQVWDS |
| BCMA LC | | SSDHVVFGGGTKLTVLGQPKAAPSVTLFPPS |
| | | SEELQANKATLVCLISDFYPGAVTVAWKGD |
| | | SSPVKAGVETTTPSKQSNNKYAASSYLSLTP |
| | | EQWKSHRSYSCQVTHEGSTVEKTVAPTECS |
| BCMA × CD3 | SEQ ID NO: 54 | EVQLVESGGGLVQPGGSLRLSCAASGFTFNT |
| bispecific | | YAMNWVRQAPGKGLEWVARIRSKYNNYAT |
| comparator anti- | | YYAASVKGRFTISRDDSKNSLYLQMNSLKTE |
| CD3 HC | | DTAVYYCARHGNFGNSYVSWFAYWGQGTL |
| | | VTVSSASTKGPSVFPLAPCSRSTSESTAALGC |
| | | LVKDYFPEPVTVSWNSGALTSGVHTFPAVL |
| | | QSSGLYSLSSVVTVPSSSLGTKTYTCNVDHK |
| | | PSNTKVDKRVESKYGPPCPPCPAPEAAGGPS |
| | | VFLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQEEMTKNQ |
| | | VSLTCLVKGFYPSDIAVEWESNGQPENNYKT |
| | | TPPVLDSDGSFLLYSKLTVDKSRWQEGNVFS |
| | | CSVMHEALHNHYTQKSLSLSLGK |
| BCMA × CD3 | SEQ ID NO: 55 | QTVVTQEPSLTVSPGGTVTLTCRSSTGAVTT |
| bispecific | | SNYANWVQQKPGQAPRGLIGGTNKRAPGTP |
| comparator anti- | | ARFSGSLLGGKAALTLSGVQPEDEAEYYCAL |
| CD3 LC | | WYSNLWVFGGGTKLTVLGQPKAAPSVTLFP |
| | | PSSEELQANKATLVCLISDFYPGAVTVAWKA |
| | | DSSPVKAGVETTTPSKQSNNKYAASSYLSLT |
| | | PEQWKSHRSYSCQVTHEGSTVEKTVAPTECS |
| linker | SEQ ID NO: 31 | GGGGS |
| linker | SEQ ID NO: 32 | GGGGSGGGGS |
| linker | SEQ ID NO: 33 | GGGGSGGGGSGGGGS |
| linker | SEQ ID NO: 34 | GGGGSGGGGSGGGGSGGGGS |
| linker | SEQ ID NO: 35 | GGGGGSGGGGSGGGGSGGGGS |
| linker | SEQ ID NO: 36 | GGGGSGGGGSGGGGSGGGGAS |
| Hinge region | SEQ ID NO: 37 | EPKSC |
| extension | | |
| linker | SEQ ID NO: 38 | (GGGGS)n |
| linker | SEQ ID NO: 39 | DGGGS |
| linker | SEQ ID NO: 40 | TGEKP |
| linker | SEQ ID NO: 41 | GGRR |
| linker | SEQ ID NO: 42 | EGKSSGSGSESKVD |
| linker | SEQ ID NO: 43 | KESGSVSSEQLAQFRSLD |
| linker | SEQ ID NO: 44 | GGRRGGGS |
| linker | SEQ ID NO: 45 | LRQRDGERP |
| linker | SEQ ID NO: 46 | LRQKDGGGSERP |
| linker | SEQ ID NO: 47 | GSTSGSGKPGSGEGSTKG |

### Variants

Also encompassed are variants of the sequences disclosed herein. A variant amino acid or nucleic acid sequence differs from its parental sequence by virtue of insertion (including addition), deletion and/or substitution of one or more amino acid residues or nucleobases, respectively, while retaining at least one desired property of the parent sequence disclosed herein, e.g., specific antigen binding, efficacy on BCMA positive cells, stability (e.g., serum stability, thermal stability, and/or storage stability), producibility (e.g., expression levels), safety (e.g., reactivity in healthy tissues, Granzyme B release, proinflammatory cytokine IFN gamma antigen-positive or antigen-negative cell lines, and/or low to no induction of pro-inflammatory cytokines IL-2, IL-6 and TNF alpha cytokine release), efficacy (e.g., BCMA positive cell eradication and/or T cell activating properties as e.g. determined in vitro), or binds one, two or all three target proteins in a similar affinity range. In some embodiments, the variant antigen binding protein comprises an amino acid sequence being at least about 80%, 85%, 90%, 91%, 92%, 93%, 94%, 95%, 96%, 97%, 98%, or 99% identical to an amino acid sequence disclosed herein. In some embodiments, the variant antigen binding protein comprises an amino acid sequence having 1, 2, 3, 4, 5, 6, 7, 8, 9, 10, 11 or 12 substitutions, deletions or insertions when compared to a sequence disclosed herein. In some embodiments, the variant antigen binding protein comprises a sequence disclosed herein with up to 3, up to 5, up to 7 or up to 9 substitutions. Substitutions may be placed in one or more CDR regions, in one or more framework regions or in one or more linker. In some embodiments, the substitutions are not placed in the CDR regions. In some embodiments, substitutions, e.g., the 1, 2, 3, 4, 5, 6, 7, 8, 9, 10, 11 or 12 substitutions, are placed in one or more framework regions. In some embodiments, one or more CDR sequences comprise up to one, two or three substitutions, deletions or insertions, while the respective antigen binding protein retains specific binding to its target.

In certain embodiments, the variant antigen binding protein retains binding to the target protein of at least 50%, such as 60%, 70%, 80%, 90% or 95% of the equilibrium dissociation constant K_{D} of the reference antigen binding protein (i.e., the corresponding antigen binding protein without said substitutions, insertions, and/or deletions) when measured under identical conditions. Variants may be artificially engineered or naturally occurring, such as e.g., allelic or splice variants. In some embodiments, the variant antigen binding protein comprises an amino acid sequence being at least about 80%, 85%, 90%, 91%, 92%, 93%, 94%, 95%, 96%, 97%, 98% or 99% identical to an amino acid sequence disclosed herein, while retaining binding to the target. In certain embodiments, the variant sequence of the BCMA binding protein (or domain) disclosed herein comprises one or more, preferably all, CDR sequences of the group consisting of SEQ ID NOs: 1-6. In certain embodiments, the variant sequence of the CD19 binding protein (or domain) disclosed herein comprises one or more, preferably all, CDR sequences of the group consisting of SEQ ID NOs: 10-15. In certain embodiments, the variant sequence of the CD3 binding protein (or domain) disclosed herein comprises one or more, preferably all, CDR sequences of the group consisting of SEQ ID NOs: 19-24.

In certain embodiments, a variant antigen binding protein retains specific binding to one or more of the target proteins, i.e., BCMA, CD3 and/or CD19, and/or competes with the antigen binding protein disclosed herein for binding to its target.

In certain embodiments, the variant is a naturally occurring variant as outlined below.

In certain embodiments, the antigen binding proteins disclosed herein have at least one desired property over a competitor (e.g., an antibody) against the same target protein (i.e., BCMA, CD19 and/or CD3), e.g., specific antigen binding, efficacy on target positive cells, stability (e.g., serum stability, thermal stability, and/or storage stability), producibility (e.g., expression levels), safety (e.g., reactivity in healthy tissues, Granzyme B release, proinflammatory cytokine IFN gamma release in antigen-positive or antigen-negative cell lines, and/or low to no induction of pro-inflammatory cytokines IL-2, IL-6 and/or TNF alpha cytokine release), efficacy (e.g., BCMA positive cell eradication and/or T cell activating properties as e.g. determined in vitro), or binds one or more of the target proteins, i.e., BCMA, CD3 and/or CD19, with better affinity. In certain embodiments, the antigen binding protein according to the present invention is more specific than a competitor for one or more of the target proteins, i.e., BCMA, CD3 and/or CD19. In certain embodiments, the antigen binding protein according to the present invention shows a better off-target profile. In certain embodiments, the antigen binding protein according to the present invention shows a better on-target off-tumor profile. In certain embodiments, the antigen binding protein sequence is more human than a competitor; for example, has 1, 2, 3, 4, 5, 6, 7, 8, 9, 10, 11 or 12 fully human CDRs.

### Features and Functions of Antigen Binding Proteins

### Binding to and Depletion of CD19+ Cells, BCMA+ Cells, and CD19+BCMA+ Cells

A multispecific antigen binding protein according to the present disclosure may have specificity to all of CD3, CD19, and BCMA. Without wishing to be bound by theory, having such trispecificity within a single molecule may allow for stable and/or prolonged elimination of disease-causing cells, e.g., antibody-producing cells. The pathogenic B cell compartment in autoimmunity spans both CD19+ B cell subsets and BCMA expressing plasma cells. CD19 targeting enables broad targeting of precursor and mature B cells, while BCMA targeting provides selective access to plasma cells that maintain autoantibody production, which are believed to be key drivers of disease persistence (Wang et al., Ann Rheum Dis. 2024 Sep. 30; 83(10):1304-1314).

A multispecific antigen binding protein according to the present disclosure may uniquely allow binding to CD19+ cells, BCMA+ cells, and CD19+BCMA+ cells.

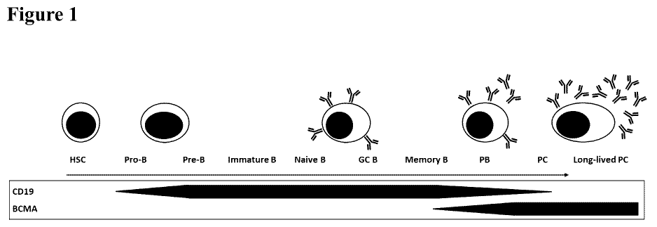

As shown in FIG. 1, BCMA is expressed by plasmablasts, plasma cells, and long-lived plasma cells, which are antibody-secreting cells. Memory B cells also express BCMA at relatively low levels. Therefore, a multispecific antigen binding protein with a specificity to BCMA binds to such antibody-secreting cells and memory B cells. The multispecific antigen binding protein, when it further has specificity to CD3, is capable of bridging T cells and such antibody-secreting cells and memory B cells and inducing cytotoxicity by the T cells against such cells.

As shown in FIG. 1, CD19 is expressed by cells of the B cell lineage at earlier stages, such as pro-B cells, pre-B cells, immature B cells, and naïve B cells, as well as antigen-activated B cells such as germinal center B cells and memory B cells. Plasmablasts and plasma cells also express CD19 at relatively low levels. Therefore, a multispecific antigen binding protein with specificity to CD19 binds to such early-stage cells and antigen-activated cells, and also to some extent antibody-secreting cells. The multispecific antigen binding protein, when it further has specificity to CD3, is capable of bridging T cells and such CD19+ cells and inducing cytotoxicity by the T cells against such cells.

Therefore, a multispecific antigen binding protein which has specificity to BCMA, CD19, and CD3 according to the present disclosure is capable of bridging T cells and such BCMA+ cells and bridging T cells and such CD19+ cells to induce cytotoxicity by the T cells against cells of the B cell lineage of a broad stage. Without wishing to be bound by theory, a multispecific antigen binding protein according to the present disclosure, may be particularly useful and effective because it induces cytotoxicity against not only antibody-secreting cells but also cells of the B cell lineage of earlier stages that are not yet secreting antibodies or not even antigen-experienced or activated by a cognate antigen. In the context of antibody-mediated or antibody-induced diseases (e.g., autoimmune diseases (especially those caused, mediated, or exacerbated by autoantibodies and/or autoreactive B cell receptors), allergies, inflammation), such a protein not only removes cells actually secreting the problematic antibodies but also eliminates at an early stage cells that would develop into such antibody-secreting cells.

Furthermore, some cells express both CD19 and BCMA (see e.g., FIG. 1). For example, plasmablasts and plasma cells express both CD19 and BCMA. These cells are antibody-secreting cells. Memory B cells also express CD19 and at a relatively lower level BCMA as well. Therefore, without wishing to be bound by theory, a multispecific antigen binding protein according to the present disclosure which has specificity to BCMA, CD19, and CD3 according to the present disclosure has a high chance of binding to such CD19+BCMA cells and thus is particularly effective in bridging T cells and such CD19+BCMA+ cells, thereby induing cytotoxicity by the T cells against CD19+BCMA+ cells, i.e., cells which secrete antibodies or which are ready to develop into antibody-secreting cells. Further without wishing to be bound by theory, such ability to target CD19+BCMA+ cells may provide an advantage over simultaneous use of two bispecific antibodies, i.e., an anti-CD3/CD19 bispecific antibody and an anti-CD3/BCMA bispecific antibody. Additionally, compared to such simultaneous use of two bispecific antibodies, use of an anti-CD3/CD19/BCMA trispecific antigen binding protein would have fewer anti-CD3 binding unit, which, without wishing to be bound by theory, may provide efficacy with less risk of unacceptable levels of cytokine release.

Therefore, a multispecific antigen binding protein according to the present disclosure may be particularly effective in preventing, ameliorating, treating, or reversing antibody-mediated or antibody-induced diseases such as but not limited to: systemic lupus erythematosus (SLE), rheumatoid arthritis, Sjögren's syndrome, scleroderma, Hashimoto thyroiditis, Grave's disease, Type 1 diabetes, Addison disease, autoimmune hemolytic anemia, immune thrombodytopenic purpura (ITP), cold agglutinin disease, myasthenia gravis, multiple sclerosis, anti-NMDA receptor encephalitis, Guillain-Barré syndrome, celiac disease, inflammatory bowel disease (Crohn's disease, ulcerative colitis), anti-glomerular basement membrane (anti-GBM) disease, anti-neutrophil cytoplasmic antibody (ANCA)-associated vasculitis, Goodpasture's syndrome, pemphigus vulgaris, psoriasis, autoimmune vitiligo (e.g., caused by antibodies specific to pigment-producing cells), pemphigoid, dermatitis herpetiformis, or epidermolysis bullosa acquisita; anaphylaxis, allergy, asthma, urticaria; or overt inflammation, e.g., associated with viral or bacterial infection, or complement activation and/or mast cell degranulation.

### Molecular Size

In some embodiments, a multispecific antigen binding protein according to the present disclosure may have a molecular size suited for infiltrating into secondary or peripheral tissues to deplete CD19+ cells, BCMA+ cells, and/or CD19+BCMA+ cells. In certain embodiments, the peripheral tissue may include a peripheral lymphoid tissue, e.g., lymph node or spleen. In certain embodiments, the peripheral tissue may include a mucosa-associated lymphoid tissue (MALT), e.g., gut-associated lymphoid tissue (GALT), bronchus-associated lymphoid tissue (BALT), nasal-associated lymphoid tissue (NALT), or tonsil.

In some embodiments, a multispecific antigen binding protein according to the present disclosure may have a molecular size suited for infiltrating into primary or central lymphoid tissues to deplete CD19+ cells, BCMA+ cells, and/or CD19+BCMA+ cells. In certain embodiments, the primary or central lymphoid tissue may include a bone marrow. In certain embodiments, the primary or central lymphoid tissue may include a thymus.

In some embodiments, a multispecific antigen binding protein according to the present disclosure may have a molecular size suited for infiltrating into a tertiary lymphoid tissue. In certain embodiments, the tertiary lymphoid tissue may be one which is formed during chronic inflammation.

In some embodiments, a multispecific antigen binding protein according to the present disclosure may be able to infiltrate into the brain.

In certain embodiments, a multispecific antigen binding protein according to the present disclosure may have a molecular size smaller than a human IgG. For example, the size may be smaller than about 150 kDa, smaller than about 140 kDa, smaller than about 130 kDa, smaller than about 120 kDa, or smaller than about 110 kDa. Without wishing to be bound by theory, an antigen binding protein with a molecule weight <150 kDa may efficiently diffuse into tissues of interest in the presence of inflammation.

In certain embodiments, the size of a multispecific antigen binding protein according to the present disclosure may be between about 70 and about 140 kDa, between about 80 kDa and about 130 kDa, between about 90 kDa and about 120 kDa, between about 100 kDa and about 110 kDa. In particular embodiments, the size may be about 109, about 108, about 107, about 106, about 105, about 104, about 103, about 102, about 101, or about 100 kDa.

Without wishing to be bound by theory, the size of a multispecific antigen binding protein according to the present disclosure may uniquely allow rapid and/or deep in vivo depletion of CD19+ and/or BCMA+ cells (e.g., B cells and plasma cells), e.g., as shown in Examples.

### Binding Valency

In some embodiments, a multispecific antigen binding protein according to the present disclosure with specificity to CD3, CD19, and BCMA may bind to CD3 monovalently.

In some embodiments, a multispecific antigen binding protein according to the present disclosure with specificity to CD3, CD19, and BCMA may bind to CD19 monovalently.

In some embodiments, a multispecific antigen binding protein according to the present disclosure with specificity to CD3, CD19, and BCMA may bind to BCMA monovalently.

In particular embodiments, a multispecific antigen binding protein according to the present disclosure with specificity to CD3, CD19, and BCMA may bind to CD3, CD19, and BCMA monovalently.

Without wishing to be bound by theory, binding monovalently by a multispecific antigen binding protein according to the present disclosure to each of CD3, CD19, and BCMA may provide appropriate bridging between a T cells and a CD19+ cell and/or between a T cell and a BCMA+ cell to induce cytotoxicity by T cells against and/or elimination of CD19+ cells, BCMA+ cells, and/or CD19+BCMA+ cells, and preferably does not elicit undesired or unacceptable level of cytokine release in a treated patient . . . .

In some embodiments, the level of cytokine release optionally may be determined by detecting whether treatment results in an unacceptable increase in inflammatory cytokines such as IL-6 and/or whether increased cytokine levels return to normal or acceptable levels in an acceptable period of time.

Generally, when a multispecific antigen binding protein according to the present disclosure is administered to a subject, even when the level of certain inflammatory cytokines (e.g., IFN-γ, IL-2, IL-6, IL-8, IL-10, MCP-1, MIP-1β, and/or TNF-α.) becomes elevated after treatment, these levels may return to baseline cytokine levels shortly after treatment, e.g., within a week, within about 5 days, within about 1-3 days, or within about 24 hours after treatment.

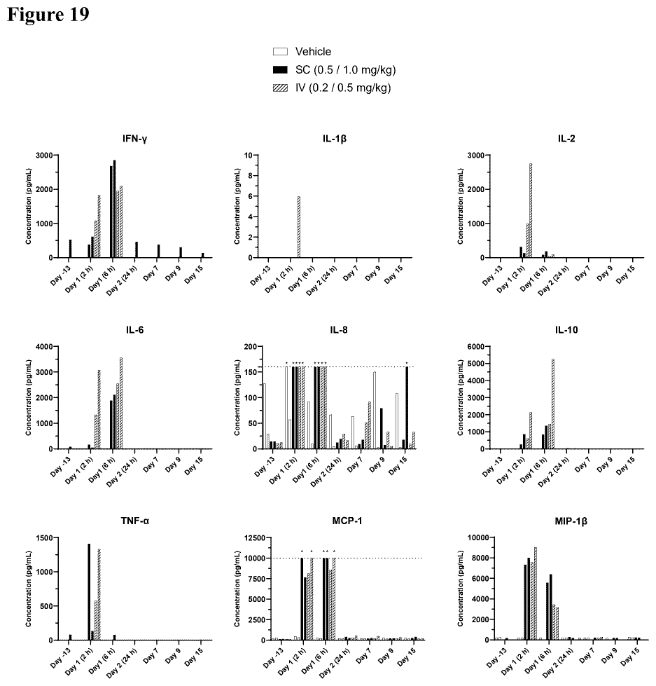

In certain embodiments, administration of a multispecific antigen binding protein according to the present disclosure may lead to acceptable levels of cytokine release, such as one characterized by any one or more of the following, unlimiting cytokine concentrations in the plasma (e.g., as measured in FIG. 19): (i) transient increase in IFN-γ) no significant increase or transient increase in IL-1β; iii) transient increase in IL-2; (iv) transient increase in IL-6 (v) transient increase in IL-8; (vi) transient increase in IL-10; (vii) transient increase in TNF-α; (viii) transient increase in MCP-1; and/or (ix) transient increase in MIP-1β.

Among the trispecific structures disclosed herein, the structures in FIG. 24A and the structures in the top and bottom panels in FIG. 24B provide monovalent binding to three antigens or epitopes.

### Format

In some embodiments, a multispecific antigen binding protein according to the present disclosure with specificity to CD3, CD19, and BCMA may have a molecular format (i.e., molecular structure) suited for inducing cytotoxicity by T cells against and/or elimination of CD19+ cells, BCMA+ cells, and/or CD19+BCMA+ cells, and preferably does not elicit undesired or unacceptable level of cytokine release in a treated patient.

In certain embodiments, administration of a multispecific antigen binding protein according to the present disclosure may lead to acceptable level of cytokine release, such as one characterized by any one or more of the plasma cytokine concentration features described in (i)-(ix) above.

In some embodiments, a multispecific antigen binding protein according to the present disclosure with specificity to CD3, CD19, and BCMA may have a molecular format (i.e., molecular structure) suited for infiltrating into secondary or peripheral tissues (e.g., any of those disclosed herein) to deplete CD19+ cells, BCMA+ cells, and/or CD19+BCMA+ cells.

In some embodiments, a multispecific antigen binding protein according to the present disclosure may have a molecular size suited for infiltrating into primary or central lymphoid tissues (e.g., any of those disclosed herein) to deplete CD19+ cells, BCMA+ cells, and/or CD19+BCMA+ cells.

In some embodiments, a multispecific antigen binding protein according to the present disclosure may have a molecular format suited for infiltrating into a tertiary lymphoid tissue (e.g., any of those disclosed herein.

In some embodiments, a multispecific antigen binding protein according to the present disclosure may have a molecular format suited for infiltrating into the brain.

In some embodiments, a multispecific antigen binding protein according to the present disclosure may have a structure provided in FIG. 24A or 24B or its description.

In some embodiments, a multispecific antigen binding protein according to the present disclosure may have a molecular format suited for infiltrating into lymphoid tissues.

Preferably treatment with such multispecific antigen binding protein will not elicit an undesired or unacceptable level of cytokine release in a treated patient. The level of cytokine release optionally may be determined by detecting whether treatment results in an unacceptable increase in inflammatory cytokines such as IL-6. Generally, even when the level of certain inflammatory cytokines (e.g., IFN-γ, IL-2, IL-6, IL-8, IL-10, MCP-1, MIP-1β, and/or TNF-α.) may be elevated after treatment, the levels may return to a baseline cytokine levels shortly after treatment. In certain cases, cytokine levels may return to normal or baseline within a week, within about 5 days, within about 1-3 days, or within about 24 hours after treatment.

In certain embodiments, acceptable level of cytokine releasemay be characterized by any one or more of the plasma cytokine concentration features described in (i)-(ix) above.

In certain embodiments, a multispecific antigen binding protein according to the present disclosure may not contain an Fc region, a CH2 domain, and/or a CH3 domain, or may contain a variant Fc region, a variant CH2 domain, and/or a variant CH3 domain which is/are modified to eliminate or reduce an effector function (e.g., complement-dependent cytotoxicity (CDC), antibody-dependent cellular cytotoxicity (ADCC), or antibody-dependent cellular phagocytosis (ADCP)) mediated by an unmodified Fc region, CH2 domain, and/or CH3 domain.

In certain embodiments, a multispecific antigen binding protein according to the present disclosure may have a structure provided in FIG. 24A or the description thereof and may efficiently diffuse into tissues of interest. In certain embodiments, a multispecific antigen binding protein according to the present disclosure may have the boxed (dashed) structure of FIG. 24A or its description or its variant corresponding to one of the three right structures provided in the second row of the top panel of FIG. 24. In particular embodiments, a multispecific antigen binding protein according to the present disclosure may have the boxed (solid) structure of FIG. 24A or its description or its variant corresponding to one of the three right structures provided in the top row of the top panel of FIG. 24. In particular embodiments, a multispecific antigen binding protein according to the present disclosure may have the boxed (solid) structure of FIG. 24A or its description of FIG. 24. In some cases, such diffusion may occur particularly efficiently in the presence of inflammation.

A multispecific antigen binding protein structure as provided the boxed (dashed) structure of FIG. 24A or its description or its variant corresponding to one of the three right structures provided in the second row of the top panel of FIG. 24. In particular embodiments, a multispecific antigen binding protein according to the present disclosure may have the boxed (solid) structure of FIG. 24A or its description or its variant corresponding to one of the three right structures provided in the top row of the top panel of FIG. 24.

Without wishing to be bound by theory, the boxed (dashed) structure of FIG. 24A or its description or its variant corresponding to one of the three right structures provided in the second row of the top panel of FIG. 24 (e.g., when ABU-A is anti-CD3 ABU, ABU-B is anti-CD19 ABU, and ABU-C is anti-BCMA ABU, or when ABU-A is anti-CD3 ABU, ABU-B is anti-BCMA ABU, and ABU-C is anti-CD19 ABU) may provide an appropriate distance between the CD3-binding site and the CD19-binding site and/or an appropriate distance between the CD3-binding site and the BCMA-binding site to provide bridging between a T cell and a CD19+ cell and/or between a T cell and a BCMA+ cell, respectively, particularly suited for inducing cytotoxicity by the T cell against the CD19+ cell and against the BCMA+ cell, respectively.

Without wishing to be bound by theory, the format of a multispecific antigen binding protein according to the present disclosure may uniquely allow rapid and/or deep in vivo depletion of CD19+ and/or BCMA+ cells (e.g., B cells and plasma cells), e.g., as shown in Examples.

Without wishing to be bound by theory, the format of a multispecific antigen binding protein according to the present disclosure may uniquely allow rapid and/or deep in vivo depletion of CD19+ and/or BCMA+ cells (e.g., B cells and plasma cells) without inducing unacceptable levels of cytokine release, e.g., as shown in Examples.

Without wishing to be bound by theory, the format of a multispecific antigen binding protein according to the present disclosure may uniquely allow rapid and/or deep in vivo depletion of CD19+ and/or BCMA+ cells (e.g., B cells and plasma cells) without significantly reducing CD34+ hematopoietic stem cells (HSCs) for example in the bone marrow.

Without wishing to be bound by theory, the format of a multispecific antigen binding protein according to the present disclosure may uniquely allow rapid and/or deep in vivo depletion of CD19+ and/or BCMA+ cells (e.g., B cells and plasma cells) without significantly reducing circulating T cells, or even when some reduction of circulating T cells is observed, the T cell count may rapidly recover to a baseline level, e.g., within about 2 weeks, within about 10 days, within about 1 week, within about 5 days, or within about 3 days.

Without wishing to be bound by theory, the format of a multispecific antigen binding protein according to the present disclosure may uniquely allow rapid and/or deep in vivo depletion of CD19+ and/or BCMA+ cells (e.g., B cells and plasma cells) without causing changes in food consumption, blood pressure, and/or body temperature.

### Degradation Propensity

In some embodiments, the antibodies and/or antigen binding fragments described herein, ABUs, and/or multi-specific antibodies may have reduced tendency/propensity to degrade.

Certain proteins, depending on the amino acid sequences, may be prone to degradation. For example, when a protein is placed in a stress condition (e.g., during prolonged storage in and/or exposure to high pH such as pH 8.0-8.5 or low pH such as pH 5.5-6.0 or high temperature such as 40 °C), asparagine (Asn (N)) and aspartic acid (Asp (D)) residues, particularly when the residues are present in a degradation motif (typically defined based on two consecutive amino acids containing Asn or Asp followed by its adjacent amino acid in a protein sequence), often go through chemical modification via Asn deamidation and Asp isomerization, respectively. Therefore, the presence of Asn and Asp degradation motifs impacts protein stability.

In case of antibodies and antibody-derived antigen binding proteins, if a degradation motif is present in a CDR, modification of the degradation motif can also impact binding to the target by the antibody, and binding may not be achieved at a desired level. Among the 6 CDRs, the CDR-H3 frequently plays the most important role in providing antigen specific binding. Therefore, antibodies with a CDR-H3 not containing or containing a reduced number of degradation motifs are generally expected to display more stable antigen binding and binding specificity. Compared to VL domains, VH domains (especially VH CDRs) typically have higher contribution to antigen binding. Therefore, antibodies with a VH or VH CDRs not containing or containing a reduced number of degradation motifs are generally expected to display more stable antigen binding and binding specificity.

Asn-based and Asp-based degradation motifs known to be by far most prone to degradation (have highest modification rates) are Asn-Gly (“NG”) and Asp-Gly (“DG”) (Sydow et al., PLoS One. 2014 Jun. 24; 9(6):e100736).

In some embodiments, an antibody, antibody fragment, ABU, or multispecific antigen binding protein according to the disclosure may not contain “NG” in the CDR-H3. In some embodiments, an antibody, antibody fragment, ABU, or multispecific antigen binding protein according to the disclosure may not contain “DG” in the CDR-H3. In some embodiments, an antibody, antibody fragment, ABU, or multispecific antigen binding protein according to the disclosure may lack both “NG” and “DG” in the CDR-H3.

In some embodiments, an antibody, antibody fragment, ABU, or multispecific antigen binding protein according to the disclosure may not contain “NG” in the VH or VH CDRs. In some embodiments, an antibody, antibody fragment, ABU, or multispecific antigen binding protein according to the disclosure may not contain “DG” in the VH or VH CDRs. In some embodiments, an antibody, antibody fragment, ABU, or multispecific antigen binding protein according to the disclosure may lack both “NG” and “DG” in the VH or VH CDRs.

In certain embodiments, an anti-BCMA antibody or antibody fragment or the anti-BCMA ABU in a multispecific antigen binding protein according to the present disclosure may not contain “NG” or “DG” in the CDR-H3. In certain embodiments, an anti-BCMA antibody or antibody fragment or the anti-BCMA ABU in a multispecific antigen binding protein according to the present disclosure may not contain “NG” or “DG” in the VH or VH CDRs. In certain embodiments, an anti-BCMA antibody or antibody fragment or the anti-BCMA ABU in a multispecific antigen binding protein according to the present disclosure may not contain “NG” or “DG” in any of the 6 CDRs.

In certain embodiments, an anti-CD19 antibody or antibody fragment or the anti-CD19 ABU in a multispecific antigen binding protein according to the present disclosure may not contain “NG” or “DG” in the CDR-H3.

In certain embodiments, an anti-CD3 antibody or antibody fragment or the anti-CD3 ABU in a multispecific antigen binding protein according to the present disclosure may not contain “NG” or “DG” in the CDR-H3. In certain embodiments, an anti-CD3 antibody or antibody fragment or the anti-CD3 ABU in a multispecific antigen binding protein according to the present disclosure may not contain “NG” or “DG” in the heavy chain CDRs. In certain embodiments, an anti-CD3 antibody or antibody fragment or the anti-CD3 ABU in a multispecific antigen binding protein according to the present disclosure may not contain “NG” or “DG” in any of the 6 CDRs.

In particular embodiments, a multispecific antigen binding protein according to the present disclosure may comprise an anti-BCMA ABU which lack both “NG” and “DG” in the CDR-H3, an anti-CD19 ABU which lack both “NG” and “DG” in the CDR-H3, and an anti-CD3 ABU which lack both “NG” and “DG” in the CDR-H3.

Additional Asn-based degradation motifs may include NH, NS, NN, NT, NQ, NF, NW, and NY, and additional Asp-based degradation motifs may include DS, DD, DN, DR, and DY. It has been reported that, in case of antibodies, different degradation motifs tend to be modified at different rates depending on which CDR the motif is in. For example, according to Lu X et al (see e.g., Lu X. et al., Mabs. 2019 January; 11 (1): 45-57.), Asn degradation motifs with relatively higher modification rates may be: NN (Asn-Asn) in CDR-H1; NG (Asn-Gly), NH (Asn-His), and NN (Asn-Asn) in CDR-H2; NT (Asn-Thr), NF (Asn-Phe), and NY (Asn-Tyr) in CDR-H3; NG (Asn-Gly), NH (Asn-His), NS (Asn-Ser), NN (Asn-Asn), and NT (Asn-Thr) in CDR-L1; NS (Asn-Ser) and NQ (Asn-Gln) in CDR-L2; and NS (Asn-Ser) and NW (Asn-Trp) in CDR-L3; and Asp degradation motifs with relatively higher modification rates may be: DD (Asp-Asp) in CDR-H1; DG (Asp-Gly) and DD (Asp-Asp) in CDR-H2; DG (Asp-Gly), DS (Asp-Ser), DD (Asp-Asp), and DR (Asp-Arg) in CDR-H3; DG (Asp-Gly), DS (Asp-Ser), DD (Asp-Asp), DN (Asp-Asn) in CDR-L1; DG (Asp-Gly) in CDR-L2; and DG (Asp-Gly) and DS (Asp-Ser) in CDR-L3.

In some embodiments, an antibody, antibody fragment, ABU, or multispecific antigen binding protein according to the disclosure may contain no or a reduced number of one or more of the above-specified motifs in the CDR-H3.

In some embodiments, an anti-BCMA antibody or antibody fragment or the anti-BCMA ABU in a multispecific antigen binding protein according to the present disclosure may contain no or a reduced number of one or more of the above-specified motifs in the CDR-H3. In some embodiments, an anti-BCMA antibody or antibody fragment or the anti-BCMA ABU in a multispecific antigen binding protein according to the present disclosure may contain no or a reduced number of one or more of the above-specified motifs in the VH CDRs. In some embodiments, an anti-BCMA antibody or antibody fragment or the anti-BCMA ABU in a multispecific antigen binding protein according to the present disclosure may contain no or a reduced number of one or more of the above-specified motifs in the 6 CDRs.

In some embodiments, an anti-CD19 antibody or antibody fragment or the anti-CD19 ABU in a multispecific antigen binding protein according to the present disclosure may contain no or a reduced number of one or more of the above-specified motifs in the CDR-H3. In some embodiments, an anti-CD3 antibody or antibody fragment or the anti-CD19 ABU in a multispecific antigen binding protein according to the present disclosure may contain a reduced number of one or more of the above-specified motifs in the CDR-H3.

In particular embodiments, a multispecific antigen binding protein according to the present disclosure may comprise an anti-BCMA ABU which lack the above-specified motifs in the CDR-H3, an anti-CD19 ABU which lack the above-specified motifs in the CDR-H3, and an anti-CD3 ABU which lack or have a reduced number of the above-specified motifs in the CDR-H3.

In certain embodiments, a multispecific antigen binding protein according to the present disclosure may comprise an anti-BCMA ABU which lacks the above-specified motifs in the CDRs, an anti-CD19 ABU which lacks or has a reduced number of the above-specified motifs in the CDRs, and an anti-CD3 ABU which lacks or have a reduced number of the above-specified motifs in the CDRs.

### Affinity

In some embodiments, a multispecific antigen binding protein according to the present disclosure with specificity to CD3, CD19, and BCMA may have affinity for CD3 suited for inducing cytotoxicity by T cells against and/or elimination of CD19+ cells, BCMA+ cells, and/or CD19+BCMA+ cells, without causing unacceptable levels of inflammation.

In certain embodiments, the affinity for CD3 may be about 0.5 to about 50 nM, about 1 to about 30 nM, about 1 to about 15 nM, about 1 to about 10 nM, about 1 to about 5 nM, or about 2 to about 4 nM. In particular embodiments, the affinity for CD3 may be about 3 nM.

Cytokine release syndrome (CRS) is a problematic side effect often associated with antibody-based therapy. Without wishing to be bound by theory, having anti-CD3 affinity that is sufficient for inducing T cell-mediated cytotoxicity against target cells but not as high as an affinity range that would cause CRS may uniquely allow a multispecific antigen binding protein according to the present disclosure to provide efficacy without causing significant side effects.

In some embodiments, a BCMA binding protein (e.g., an anti-BCMA antibody or a multispecific antigen binding protein having specificity to BCMA) according to the present disclosure may have very high affinity for BCMA. In some embodiments, a multispecific antigen binding protein according to the present disclosure with specificity to CD3, CD19, and BCMA may have affinity for BCMA suited for efficiently inducing cytotoxicity by T cells against and/or elimination of BCMA+ cells and/or CD19+BCMA+ cells.

In certain embodiments, the affinity for BCMA may be about 0.001 nM to about 1 nM, about 0.005 to about 0.5 nM, about 0.005 to about 0.3 nM, about 0.005 to about 0.1 nM, about 0.01 to about 0.05 nM, or about 0.01 to about 0.03 nM. In particular embodiments, the affinity for BCMA may be about 0.02 nM.

In some embodiments, a CD19 binding protein (e.g., an anti-CD19 antibody or a multispecific antigen binding protein having specificity to CD19) according to the present disclosure may have high affinity for CD19. In some embodiments, a multispecific antigen binding protein according to the present disclosure with specificity to CD3, CD19, and BCMA may have affinity for CD19 suited for efficiently inducing cytotoxicity by T cells against and/or elimination of CD19+ cells and/or CD19+BCMA+ cells.

In certain embodiments, the affinity for CD19 may be about 0.1 to about 50 nM, about 0.1 to about 10 nM, about 0.2 to about 5 nM, about 0.5 to about 2 nM. In particular embodiments, the affinity for CD19 may be about 1 or 1.1 nM.

In some embodiments, a multispecific antigen binding protein according to the present disclosure with specificity to CD3, CD19, and BCMA may have affinity for CD3, CD19, and BCMA suited for inducing efficient cytotoxicity by T cells against and/or elimination of CD19+ cells, BCMA+ cells, and/or CD19+BCMA+ cells, without causing unacceptable levels of cytokine release.

In certain embodiments, the affinity for CD3, CD19, and BCMA may be about 1 to about 5 nM, about 0.2 to about 5 nM, and about 0.01 to about 0.05 nM, respectively. In certain embodiments, the affinity for CD3, CD19, and BCMA may be about 2 to about 4 nM, about 0.5 to about 2 nM, and about 0.01 to about 0.03 nM, respectively. In particular embodiments, the affinity for CD3, CD19, and BCMA may be about 3 or 3.1 nM, about 1 or 1.1 nM, and about 0.02 nM, respectively.

### Expression of Antigen Binding Proteins

Polynucleotides or nucleic acids encoding the antigen binding proteins disclosed herein are provided, in particular isolated polynucleotides or nucleic acids. Methods of making an antigen binding protein encoded by these polynucleotides are also provided. Polynucleotides encoding the antigen binding proteins disclosed herein are typically inserted in a vector (e.g., an expression vector or a cloning vector) for introduction into host cells that may be used to produce the desired quantity of the antigen binding proteins. Accordingly, in certain aspects, the invention provides expression vectors comprising polynucleotides disclosed herein and host cells comprising these vectors and polynucleotides.

The term “vector” or “expression vector” is used herein to mean vectors used in accordance with the present invention as a vehicle for introducing into and expressing a desired gene in a cell. As known to those skilled in the art, such vectors may readily be selected from the group consisting of plasmids, phages, viruses and retroviruses. In general, vectors compatible with the instant invention will comprise a selection marker, appropriate restriction sites to facilitate cloning of the desired gene and the ability to enter and/or replicate in eukaryotic or prokaryotic cells.

Numerous expression vector systems may be employed for the purposes of this invention. For example, one class of vector utilizes DNA elements which are derived from animal viruses such as bovine papilloma virus, polyoma virus, adenovirus, vaccinia virus, baculovirus, retroviruses (e.g., RSV, MMTV, MOMLV or the like), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain constant region genes (e.g., human constant region genes) synthesized as discussed above.

In other embodiments, the antigen binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding an antigen binding protein has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, CHO cell lines including DG44 and DUXB11 (Chinese hamster ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney) and the like. In one embodiment, the cell line provides for altered glycosylation, e.g., afucosylation, of the antibody expressed therefrom (e.g., PER.C6® (Crucell) or FUT8-knock-out CHO cell lines (Potelligent® cells) (Biowa, Princeton, N.J.)). Host cell lines are typically available from commercial services, e.g., the American Tissue Culture Collection, or from published literature.

In certain embodiments, signal peptides may be encompassed in the reproduced sequences. In such case, the sequences shall be deemed disclosed with and without signal peptides. A readily available tool to identify signal peptides in a given protein sequence is SignalP-6.0 provided by Dansk Technical University under https://Inkd.in/e8mWfWv3.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

In Certain Aspects, a Method of Producing the Antigen Binding Protein Described Herein is Provided, Comprising the Steps of
(a) culturing the host cell disclosed herein under conditions suitable for the expression of the antigen binding protein and optionally
(b) recovering the antigen binding protein.

Genes encoding the antigen binding proteins featured in the invention can also be expressed in non-mammalian cells such as bacteria or yeast or plant cells. In this regard it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella, Bacillaceae, such as Bacillus subtilis; Pneumococcus; Streptococcus, and Haemophilus influenzae. It will further be appreciated that, when expressed in bacteria, the proteins can become part of inclusion bodies. The proteins must be isolated, purified and then assembled into functional molecules.

In addition to prokaryotes, eukaryotic microbes may also be used. Saccharomyces cerevisiae, or common baker's yeast, is the most commonly used among eukaryotic microorganisms, although a number of other strains are commonly available. For expression in Saccharomyces, the plasmid YRp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

### Engineering and Optimization of Antigen Binding Proteins

The antigen binding proteins according to the present invention may be engineered or optimized. As used herein, “optimized” or “optimization” refers to the alteration of an antigen binding protein to improve one or more functional properties. Alteration includes, but is not limited to, deletions, substitutions, additions, and/or modifications of one or more amino acids within an antigen binding protein.

As used herein, the term “functional property” is a property of an antigen binding protein for which an improvement (e.g., relative to a conventional antigen binding protein, such as an antibody) is desirable and/or advantageous to one of skill in the art, e.g., in order to improve the manufacturing properties or therapeutic efficacy of an antigen binding protein. In one embodiment, the functional property is stability (e.g., thermal stability). In another embodiment, the functional property is solubility (e.g., under cellular conditions). In yet another embodiment, the functional property is aggregation behavior. In still another embodiment, the functional property is protein expression (e.g., in a prokaryotic cell). In yet another embodiment the functional property is refolding behavior following inclusion body solubilization in a manufacturing process. In certain embodiments, the functional property is not an improvement in antigen binding affinity. In another embodiment, the improvement of one or more functional properties has no substantial effect on the binding affinity of the antigen binding protein.

In certain embodiments, a disulfide bridge is introduced into the antigen binding proteins described herein. Introducing disulfide bridges into proteins provides several advantages, primarily in terms of structural stability and functional performance, but also on pharmacokinetics and bioavailability. In certain embodiments, the introduction of one or more disulfide bridges into the antigen binding protein increases its stability and may allow for generating formulations with increased concentration. Generally, raising concentration in a biologic formulation can elevate the risk of aggregation, denaturation, and proteolysis. As protein concentration rises, molecules are more likely to interact and form aggregates. This can cause proteins to unfold, losing their native structure and leading to denatured proteins, which are typically inactive and potentially harmful. Compared to the non-modified antigen binding protein, an antigen binding protein comprising one or more disulfide bridges with increased stability may show fewer or no aggregates.

Multispecific antigen binding proteins as described herein may comprise one or more scFvs as building blocks. It is well established that the physical stability of scFv fragments can vary and may contribute to protein aggregation. Several strategies have been described for stabilizing scFv fragments, with regards to disulfide bonds there is great variability to tolerate these; therefore, disulfide-bond stabilization is not a universally applicable solution for scFvs (see, e.g., Miller B. et al, Prot Eng, Design & Selection vol. 23 no. 7 pp. 549-557, 2010).

In certain embodiments, antigen binding proteins disclosed herein comprise one or more disulfide bridges, in particular in a binding domain in scFv format. In certain embodiments, such disulfide bridge is formed between a cysteine in FW2 at Kabat position 44 of the VH and a cysteine in FW4 at Kabat position 100 of the VL; and/or between a cysteine in FW4 at Kabat position 105 of the VH and a cysteine in FW2 at Kabat position 43 of the VL.

In certain embodiments, the BCMA binding domain of the antigen binding proteins disclosed herein comprises a disulfide bridge between its VL and its VH. In certain embodiments, said BCMA-binding domain is a scFv having the SEQ ID NO: 9 or 83.

In certain embodiments, the CD3 binding domain of the antigen binding proteins disclosed herein comprises a disulfide bridge between its VL (SEQ ID NO.: 25 or 94) and its VH (SEQ ID NO.: 26). In certain embodiments, the CD19 binding domain of the antigen binding proteins disclosed herein comprises a disulfide bridge between its VL and its VH. In certain embodiments, said CD19-binding domain is a scFv having the SEQ ID NO: 18 or 89.

In certain embodiments, a trispecific antigen binding protein according to the present invention is a Fab-(scFv)₂ comprising:
(a) the amino acid sequence of SEQ ID NO: 29; and/or
(b) the amino acid sequence of SEQ ID NO: 30; or a variant of said Fab-(scFv)₂ having at least about 80%, 85%, 90%, 95%, 96%, 97%, 98% or 99% identity thereto, wherein said variant comprises at least one disulfide bridge.

In certain embodiments, the antigen binding protein according to the present invention comprises a scFv and is optimized by identifying preferred amino acid residues to be substituted, deleted, and/or added at amino acid positions of interest (e.g., amino acid positions identified by comparing a database of scFv sequences having at least one desirable property, e.g., as selected with Quality Control (QC) assay, versus a database of mature antibody sequences, e.g., the Kabat database) in an antigen binding protein. Thus, the disclosure further provides “enrichment/exclusion” methods for selecting a particular amino acid residue. Still further, the disclosure provides methods of engineering antigen binding proteins (e.g., scFvs) by mutating particular framework amino acid positions identified using the “functional consensus” approach described herein. In certain embodiments, the framework amino acid positions are mutated by substituting the existing amino acid residue by a residue which is found to be an “enriched” residue using the “enrichment/exclusion” analysis methods described herein. In one aspect, the disclosure provides a method of identifying an amino acid position for mutation in a single chain antibody (scFv), the scFv having VH and VL amino acid sequences, the method comprising: a) entering the scFv VH, VL or VH and VL amino acid sequences into a database that comprises a multiplicity of antibody VH, VL or VH and VL amino acid sequences such that the scFv VH, VL or VH and VL amino acid sequences are aligned with the antibody VH, VL or VH and VL amino acid sequences of the database; b) comparing an amino acid position within the scFv VH or VL amino acid sequence with a corresponding position within the antibody VH or VL amino acid sequences of the database; c) determining whether the amino acid position within the scFv VH or VL amino acid sequence is occupied by an amino acid residue that is conserved at the corresponding position within the antibody VH or VL amino acid sequences of the database; and d) identifying the amino acid position within the scFv VH or VL amino acid sequence as an amino acid position for mutation when the amino acid position is occupied by an amino acid residue that is not conserved at the corresponding position within the antibody VH or VL amino acid sequences of the database. ScFv optimization is described in further detail in WO2008110348, WO2009000099, WO2009000098, and WO2009155725, all of which are incorporated herein by reference.

In certain embodiments, the antigen binding protein comprises an Fc domain which is modified such that it does not induce cytotoxic immune responses and/or does not activate complement. For example, one or more substitutions may be introduced into the Fc domain so that its ADCC/ADCP or CDC effector function is inactivated. Such antigen binding protein has the advantage of increased half-life when compared to antibody fragments with a molecular weight below 60 kDa, without mediating cytotoxic immune responses.

Chemical and/or Biological Modifications

In one aspect, the antigen binding protein is chemically and/or biologically modified. For example, the antigen binding protein may be glycosylated, phosphorylated, hydroxylated, PEGylated, HESylated, PASylated, XTENylated, sulfated, labeled with dyes and/or radioisotopes, conjugated with enzymes and/or toxins, and/or Albumin fusion technology. Likewise, any nucleic acid sequence, plasmid or vector and/or host cell described herein may be modified accordingly.

Such modification may for example be done to optimize pharmacodynamics, its water solubility or to lower its side effects. For example, PEGylation, PASylation, HESylation and/or the fusion to serum albumin may be applied to slow down renal clearance, thereby increasing plasma half-life time of the antigen binding protein. In one embodiment, a modification adds a different functionality to the antigen binding protein, for example, a detection label for diagnostics or a toxin to combat cancer cells even more efficiently. In other embodiments, the modification occurs due to the expression system used.

Alternatively or additionally, in some embodiments, the trispecific antigen binding proteins and other polypeptides provided herein undergo co- and post-translational modifications as known in the art. Examples of post-translational modifications include, but are not limited to, disulfide bond formation, glycosylation, cyclization (such as e.g., N-terminal pyroglutamate formation), have a N-terminal or C-terminal residue removed or “clipped” (for example, C-terminal lysine residues are often removed during the manufacturing process), deamidation, isomerization, oxidation, glycation, acylation, fucosylation, peptide bond cleavage, non-reducible cross-linking, truncation, and/or have part or all of a signal sequence incompletely processed.

In one embodiment, the antigen binding protein is glycosylated. Glycosylation refers to a process that attaches carbohydrates to proteins. In biological systems, this process is performed enzymatically within the cell as a form of co-translational and/or post-translational modification. A protein can also be chemically glycosylated. The carbohydrates may be N-linked to a nitrogen of asparagine or arginine side-chains; O-linked to the hydroxy oxygen of serine, threonine, tyrosine, hydroxylysine, or hydroxyproline side-chains; employ xylose, fucose, mannose, and N-acetylglucosamine attached to a phospho-serine; and/or adding mannose sugar to a tryptophan residue found in a specific recognition sequence. Glycosylation patterns may, e.g., be controlled by choosing appropriate cell lines, culturing media, protein engineering manufacturing modes and process strategies (see., HOSSLER, P. Optimal and consistent protein glycosylation in mammalian cell culture. Glycobiology 2009, vol. 19, no. 9, p. 936-949.). In some embodiments, the glycosylation patterns of the antigen binding proteins described herein are modified to enhance ADCC and CDC effector function.

The antigen binding protein may be engineered to control or alter the glycosylation pattern, e.g., by deleting and/or adding of one or more glycosylation sites. The creation of glycosylation sites can e.g., be accomplished by introducing the corresponding enzymatic recognition sequence into the amino acid sequence of the antigen binding protein.

In some embodiments, the antigen binding protein is PEGylated. PEGylation may alter the pharmacodynamic and pharmacokinetic properties of a protein. Additionally, PEGylation may reduce the immunogenicity by shielding the PEGylated antigen binding protein from the immune system and/or alter its pharmacokinetics by, e.g., increasing the in vivo stability of the antigen binding protein, protecting it from proteolytic degradation, extending its half-life time and by altering its biodistribution. Typically, polyethylene-glycol (PEG) of an appropriate molecular weight is covalently attached to the protein. Similar effects may be achieved using PEG mimetics, e.g., HESylating, XTENylating or PASylating the antigen binding protein. HESylation utilizes hydroxyethyl starch (“HES”) derivatives. During PASylation, the antigen binding protein is linked to conformationally disordered polypeptide sequences composed of the amino acids proline (P), alanine (A) and serine(S), and XTENylation employs a similar, intrinsically disordered XTEN-polypeptide.

In certain embodiments, the antigen binding protein of the disclosure comprises a pyroglutamate (pE, pyrGlu, pyre or pGlu) instead of the N-terminal glutamine or the N-terminal glutamate. In certain embodiments, the light chain of the antigen binding protein comprises pyroglutamate (pE) instead of the N-terminal glutamine. In certain embodiments, the light chain of the antigen binding protein comprises pyroglutamate (pE) instead of the N-terminal glutamate. In certain embodiments, such pyroglutamate (pE) modification has no impact on the safety and/or efficacy of the antigen binding protein. In certain embodiments, the N-terminal glutamine (Q) of a light chain disclosed herein is clipped, e.g., to prevent pyroglutamate formation.

In certain embodiments, the CD3 binding domain comprises an N-terminal truncation of 1 or more amino acids (e.g., a N-terminal truncation of 1, 2, 3, 4, or 5 amino acids). In certain embodiments, the CD3 antigen binding domain comprises a C-terminal truncation of 1 or more amino acids (e.g., a C-terminal truncation of 1, 2, 3, 4, or 5 amino acids). In certain embodiments, the N-terminal and/or C-terminal truncation is a truncation of the Fab-(scFv)₂ of a heavy chain amino acid sequence of SEQ ID NO: 29 and a light chain amino acid sequence of SEQ ID NO: 30. In certain embodiments, the Q at position 1 of the antigen binding proteins is removed which may prevent pyroglutamate formation and protects the protein to be clipped by proteases; in certain embodiments, this may lower the possibility of having different charge variants which may pose challenges in the CMC process. An N-terminal R removal may in certain embodiments result in a lower pre-existing antibody response.

In certain embodiments, the variant sequence of SEQ ID NO: 25 or 94, SEQ ID NO: 26, SEQ ID NO: 27, SEQ ID NO: 28, SEQ ID NO: 29 and/or SEQ ID NO: 30 comprises a pyroglutamate at amino acid position 1. In certain embodiments, the light chain of the antigen binding protein comprises pyroglutamate (pE) instead of the N-terminal glutamate.

In certain embodiments, an antigen binding protein according to the present disclosure may be modified to reduce anti-drug antibodies. In some cases, an antigen binding protein contains a C-terminal modification, such as the addition or deletion of at least one amino acid residue (see e.g., WO2014043509A3 and WO2013024059A2). In some cases, an antigen binding protein contains a C-terminal extension of 1-5 amino acids. In some cases, an antigen binding protein may further contain a C-terminal glycine (G). In some cases, an antigen binding protein may further contain a C-terminal valine (V). In some cases, an antigen binding protein may further contain a C-terminal alanine (A). In some cases, an antigen binding protein may be modified to minimize aggregation-prone motifs, deamidation sites, oxidation sites, isomerization sites and/or unstable CDR residues. In some cases, an antigen binding protein may be modified to include human-like glycosylation pattern.

In certain embodiments, the antigen binding protein is labelled with or conjugated to a second moiety which attributes one or more ancillary functions to the antigen binding protein. For example, the second moiety may have an additional immunological effector function, be effective in drug targeting or useful for detection. The second moiety can, e.g., be chemically linked or fused genetically to the antigen binding protein using known methods in the art. As used herein, the term “label” refers to any substance or ion which is indicative of the presence of the antigen binding protein when detected or measured by physical or chemical means, either directly or indirectly. For example, the label may be directly detectable by, without being limited to, light absorbance, fluorescence, reflectivity, light scatter, phosphorescence, or luminescence properties, molecules or ions detectable by their radioactive properties or molecules or ions detectable by their nuclear magnetic resonance or paramagnetic properties. Examples of indirect detection include light absorbance or fluorescence; for example, various enzymes which cause appropriate substrates to convert, e.g., from non-light absorbing to light absorbing molecules, or from non-fluorescent to fluorescent molecules. A labelled antigen binding protein is particularly useful for in vitro and in vivo detection or diagnostic purposes. For example, an antigen binding protein labelled with a suitable radioisotope, enzyme, fluorophore or chromophore can be detected by radioimmunoassay (RIA), enzyme-linked immunosorbent assay (ELISA), or flow cytometry-based single cell analysis (e.g., FACS analysis), respectively. Similarly, the nucleic acids and/or vectors disclosed herein can be labeled for detection or diagnostic purposes, e.g., using labelled fragments thereof as probes in hybridization assays.

Non-limiting examples of second moieties include radioisotopes (35S, 32P, 14C, 18F, and/or 125I), apoenzymes, enzymes (e.g., alkaline phosphatase, horseradish peroxidase, beta-galactosidase and/or angiogenin), co-factors, peptide moieties (e.g., a HIS-tag), proteins (e.g. lectin, serum albumin), carbohydrates (e.g., mannose-6-phosphate tags), fluorophores (e.g., fluorescein isothiocyanate (FITC)), phycoerythrin, green/blue/red or other fluorescent proteins, allophycocyanin (APC), chromophores, vitamins (e.g., biotin), chelators, antimetabolites (e.g., methotrexate), toxins (e.g. a cytotoxic drug, or a radiotoxin).

In one aspect, the invention relates to drug conjugates (in particular antibody-drug conjugates ADCs) comprising an antigen binding proteins as described herein conjugated to a toxin which further enhances efficient killing of specific cells, such as e.g., BCMA positive cells. The toxin moiety is typically a small molecular weight moiety, such as MMAE/MMAF, DM1, chaliceamicin, anthracycline toxins, Taxol, gramicidin D and/or colchicine which may be linked via a peptide linker to the antigen binding protein. In certain embodiments, the ADC comprises a scFv comprising or consisting of the amino acid sequence disclosed herein, or a variant as described herein.

The toxin may be conjugated non-site-specifically or site-specifically to the antigen binding protein. Non-site-specific conjugation typically involves the use of chemical linkers, e.g., with maleimide functionality, that mediate conjugation to lysine or cysteine amino acid side chains of the antibody. Site-specific conjugation may be achieved using chemical, chemo-enzymatic, or enzymatic conjugations known in the art, e.g., employing bifunctional linkers, bacterial transglutaminase or sortase enzymes, linkers allowing Pictet-Spengler chemistry on formyl-glycine forming enzyme modified antigen binding proteins, or glycan-remodeled antigen binding proteins.

### Methods of Administering Antigen Binding Proteins

Methods of preparing and administering antigen binding proteins according to the present invention as well as the nucleic acids described herein (incl. isolated polynucleotides and vectors as described herein), the host cells described herein, the CARs described herein, the ADCs described herein, or the compositions described herein to a subject are well known to or are readily determined by those skilled in the art. The route of administration of the antigen binding proteins of the current disclosure may e.g., be oral, parenteral, by inhalation, or topical. The term parenteral as used herein includes intravenous, intraarterial, intraperitoneal, intramuscular, subcutaneous, rectal or vaginal administration. The term topical as used herein includes, but is not limited to, administration with liquid, emulsions (e.g., oil-in-water emulsions), suspensions, and ointments.

While all these forms of administration are clearly contemplated as being within the scope of the current disclosure, a form for administration would be a solution for injection. Usually, a suitable pharmaceutical composition for injection may comprise a buffer (e.g., acetate, phosphate or citrate buffer), a surfactant (e.g., polysorbate), optionally a stabilizer agent (e.g., mono-, poly-saccharides or human albumin), etc. However, in other methods compatible with the teachings herein, the modified antigen binding proteins can be delivered directly to the site of the adverse cellular population thereby increasing the exposure of the diseased tissue to the therapeutic agent. In specific embodiments, the antigen binding proteins are formulated for subcutaneous administration.

Effective doses of the compositions of the present disclosure, for the treatment of the related conditions vary depending upon many different factors, including means of administration, target site, physiological state of the patient, whether the patient is human or an animal, other medications administered, and whether treatment is prophylactic or therapeutic. Usually, the patient is a human, but non-human mammals, including transgenic mammals, can also be treated. Treatment dosages may be titrated using routine methods known to those of skill in the art to optimize safety and efficacy.

As previously discussed, the antigen binding proteins of the present disclosure, conjugates or recombinants thereof may be administered in a pharmaceutically effective amount for the in vivo treatment of mammalian disorders. In this regard, it will be appreciated that the disclosed antigen binding proteins will be formulated to facilitate administration and promote stability of the active agent.

Pharmaceutical compositions in accordance with the present disclosure typically include a pharmaceutically acceptable, non-toxic, sterile carrier such as physiological saline, nontoxic buffers, preservatives and the like. For the purposes of the instant application, a pharmaceutically effective amount of the antigen binding proteins shall be held to mean an amount sufficient to achieve effective binding to an antigen and to achieve a benefit, e.g., to ameliorate symptoms of a disease or disorder or to detect a substance or a cell. In the case of tumor cells, the antigen binding proteins will typically be capable of interacting with selected immunoreactive antigens on neoplastic or immunoreactive cells and provide for an increase in the death of those cells. Of course, the pharmaceutical compositions of the present disclosure may be administered in single or multiple doses to provide for a pharmaceutically effective amount of the modified binding polypeptide.

In one aspect, the disclosure provides for a pharmaceutical composition comprising an antigen binding protein disclosed herein, a polynucleotide or vector disclosed herein, a host cell disclosed herein, or an ADC disclosed herein, and a pharmaceutically acceptable carrier.

In keeping with the scope of the present disclosure, the antigen binding proteins according to the present invention may be administered to a human or other animal in accordance with the aforementioned methods of treatment in an amount sufficient to produce a therapeutic or prophylactic effect. The antigen binding proteins according to the present invention can be administered to such human or other animal in a conventional dosage form prepared by combining the antigen binding proteins according to the present invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of antigen binding proteins described in the current disclosure may prove to be particularly effective. Similarly, the nucleic acids described herein, the vectors described herein, the host cell cells described herein (in particular the immune cells bearing a CAR) or the compositions described herein may be administered to a human or other animal in accordance with the methods of treatment described above in an amount sufficient to produce a therapeutic or prophylactic effect.

“Efficacy” or “in vivo efficacy” as used herein refers to the response to a therapy by the pharmaceutical composition according to the present invention, using e.g., standardized response criteria The success or in vivo efficacy of the therapy using a pharmaceutical composition according to the present invention refers to the effectiveness of the composition for its intended purpose, i.e., the ability of the composition to cause its desired effect. The in vivo efficacy may be monitored by established standard methods for the specific diseases. In addition, various disease specific clinical chemistry parameters and other established standard methods may be used.

In some embodiments, the compounds and cells described herein are administered in combination with one or more different pharmaceutical compounds. Generally, therapeutic use of the compounds and cells described herein may be in combination with one or more therapies selected from the group of antibody therapy, chemotherapy, cytokine therapy, dendritic cell therapy, gene therapy, hormone therapy, laser light therapy, radiation therapy or vaccine therapy.

### Chimeric Antigen Receptors

In one aspect, the disclosure provides chimeric antigen receptors (CARs) and immune cells engineered to express such CARs, comprising the antigen binding proteins described herein, in particular a BCMA binding domain. As used herein, the term “chimeric antigen receptor” or “CAR” refers to a receptor that is capable of activating an immune cell in response to antigen binding. CARs are recombinant membrane spanning molecules and are advantageously expressed on immune cells. Their structure typically comprises (i) an extracellular domain (ectodomain or antibody domain), (ii) a transmembrane domain and (iii) a cytoplasmic domain (endodomain or intracellular signaling domain).

The ectodomain (i.e., antibody domain) typically comprises a scFv but other antigen binding proteins may also be used. A spacer connects the ectodomain and the transmembrane domain, which in turn is connected to an endodomain. Upon binding of the ectodomain to the antigen, the receptors cluster and an activation signal is transmitted to the cell which results in initiation of an immune response. First generation CARs have a simply structured endodomain comprising CD3-zeta. To increase the activation signal, a co-stimulatory domain was added in the second-generation CARs; and third generation CARs include two or more co-stimulatory domains (Maus M V et al (2014) Blood, 123:2625-2635). Said co-stimulatory domains may be selected from the group consisting of CD28, OX40 and/or 4-1BB. Apart from CD3-zeta, other ITAM-containing domains have been explored including the Fc receptor for IgE-γ domain.

Suitable immune cells include, without being limited to, T cells, Natural Killer T (NKT) cells, natural killer (NK) cells, human embryonic stem cells, hematopoietic stem cells (HSC) or induced pluripotent stem cells (iPS). Such T cell may be a cytotoxic T lymphocyte (CTL), a regulatory T lymphocyte, an inflammatory T-lymphocytes, or a helper T-lymphocyte or a gamma-delta T cell. The T cell may be a CD4+ or CD8+ or a mixed population of CD4+ and CD8+ cells.

In one aspect, the disclosure provides a chimeric antigen receptor (CAR) that specifically recognizes BCMA, comprising: i) an antigen binding protein described herein (e.g., comprising a VL of SEQ ID NO: 7 or 82 and a VH of SEQ ID NO: 8; ii) a transmembrane domain; and iii) an intracellular signaling domain.

In certain embodiments, the transmembrane domain is selected from the group consisting of an artificial hydrophobic sequence and transmembrane domains of a type I transmembrane protein, an alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, and CD154.

In certain embodiments, the intracellular signaling domain is selected from the group consisting of cytoplasmic signaling domains of a human CD3 zeta chain, FcyRIII, a cytoplasmic tail of an Fc receptor, an immunoreceptor tyrosine-based activation motif (ITAM) bearing cytoplasmic receptors, TCR zeta, FcR gamma, FcR beta, CD3 gamma, CD3 delta, CD3 epsilon, CD5, CD22, CD79a, CD79b, and CD66d.

The antibody domain may be any of the antigen binding proteins outlined above. Thus, in certain embodiments, the antibody domain comprises an antibody variable light domain (VL) comprising an amino acid sequence represented by the formula LFR1-CDR-L1-LFR2-CDR-L2-LFR3-CDR-L3-LFR4. In certain embodiments, the antibody domain comprises an antibody variable heavy domain (VH) comprising an amino acid sequence represented by the formula HFR1-CDR-H1-HFR2-CDR-H2-HFR3-CDR-H3-HFR4. In certain embodiments, the antibody domain comprises a scFv as described herein.

In certain embodiments, the CAR comprises a bispecific ectodomain. Bispecific CARs show promise for improving the effectiveness and safety of CAR-T cell therapies, particularly in cancers that present a high risk of antigen escape or express multiple tumor-associated antigens. In certain embodiments, the CAR comprises a BCMAxCD19 targeting protein according to the present invention.

### Methods of Treatment

Methods of treating a disease using the antigen binding proteins described above, the chimeric antigen receptor described above, the antibody drug conjugate described above, the isolated nucleic acid molecule described above, the vector described above, or host cell described above or the pharmaceutical composition described above, in a subject suffering thereof are provided. As outlined below, in some embodiments, the disease is an autoimmune disease. Exemplary diseases which may be treated or prevented by an antigen binding protein, such as a trispecific antigen binding protein, according to the present disclosure may include diseases caused, mediated, or exacerbated by an antibody such as an autoantibody. Such exemplary autoimmune diseases and antibody-mediated diseases include but are not limited to: systemic lupus erythematosus (SLE), rheumatoid arthritis, Sjögren's syndrome, scleroderma, Hashimoto thyroiditis, Grave's disease, Type 1 diabetes, Addison disease, autoimmune hemolytic anemia, immune thrombodytopenic purpura (ITP), cold agglutinin disease, myasthenia gravis, multiple sclerosis, anti-NMDA receptor encephalitis, Guillain-Barré syndrome, celiac disease, inflammatory bowel disease (Crohn's disease, ulcerative colitis), anti-glomerular basement membrane (anti-GBM) disease, anti-neutrophil cytoplasmic antibody (ANCA)-associated vasculitis, Goodpasture's syndrome, pemphigus vulgaris, psoriasis, autoimmune vitiligo (e.g., caused by antibodies specific to pigment-producing cells), pemphigoid, dermatitis herpetiformis, antiphospholipid syndrome, autoimmune hepatitis, stiff person syndrome, thyroid eye disease, or epidermolysis bullosa acquisita; anaphylaxis, allergy, asthma, urticaria; or overt inflammation.

In other embodiments, the disease is cancer, in particular a B cell malignancy. Examples of B cell malignancies include, without being limited to, B-cell lymphomas (such as diffuse large B-cell lymphoma, follicular lymphoma), B-cell leukemias (like chronic lymphocytic leukemia), and plasma cell disorders (such as multiple myeloma). Some specific examples of B cell malignancies include but are not limited to diffuse large B-cell Lymphoma (DLBCL), chronic lymphocytic leukemia (CLL), mantle cell lymphoma (MCL), follicular lymphoma, and Burkitt Lymphoma, small lymphocytic lymphoma (SLL), follicular lymphoma, marginal zone lymphomas, lymphoplasmacytic lymphoma (Waldenstrom macroglobulinemia), hairy cell leukemia, precursor B-cell acute lymphoblastic leukemia (B-ALL), lymphoblastic lymphoma (LBL), plasma cell neoplasms, primary central nervous system (CNS) lymphoma, primary cutaneous DLBCL, and/or nodular lymphocyte-predominant Hodgkin's lymphoma. Methods of targeting and killing tumor cells using the antigen binding proteins, in particular the trispecific antigen binding proteins described herein are also provided.

In certain aspects, the disclosure provides for the antigen binding proteins described above, the chimeric antigen receptor described above, the antibody drug conjugate described above, the isolated nucleic acid molecule described above, the vector described above, host cell described above or the pharmaceutical composition described above for use as a medicament.

In certain aspects, the disclosure provides for the antigen binding proteins described above, the chimeric antigen receptor described above, the antibody drug conjugate described above, the isolated nucleic acid molecule described above, the vector described above, the host cell described above or the pharmaceutical composition described above for use in the treatment of a disease. In certain aspects, the disclosure provides for the use of the antigen binding proteins described above, the chimeric antigen receptor described above, the antibody drug conjugate described above, the isolated nucleic acid molecule described above, the vector described above, the host cell described above or the pharmaceutical composition described above in the manufacture of a medicament.

In certain aspects, the disclosure provides a method of treating a disease in a subject, e.g., cancer or autoimmunity, comprising administering to said subject an effective amount of the antigen binding proteins described above, the chimeric antigen receptor described above, the antibody drug conjugate described above, the isolated nucleic acid molecule described above, the vector described above, the host cell described above or the pharmaceutical composition described above.

In certain embodiments, the disclosure provides for the use of the antigen binding proteins described above, the chimeric antigen receptor described above, the antibody drug conjugate described above, the isolated nucleic acid molecule described above, the vector described above, the host cell described above or the pharmaceutical composition described above, in the manufacture of a medicament for the prevention and/or treatment of a disease in a subject (e.g., a human).

In certain embodiments, the disease referred to above is a B cell-related disease. In certain embodiments, the disease is an autoimmune disease. In other embodiments, the disease is cancer, particularly a B cell malignancy.

BCMA expression was found across multiple hematologic malignancies of plasma cells or late-stage B cells (see, Dogan A. et al, Blood Cancer J. 2020 Jun. 30; 10(6):73). In certain embodiments, the disease is cancer. In certain embodiments, the cancer is multiple myeloma (MM; also known as Kahler's disease), non-Hodgkin's lymphoma (NHL), leukemia (such as acute B-lymphoblastic leukemia (B-ALL), T-cell acute lymphocytic leukemia (T-ALL) or acute myeloid leukemia (AML)). Hence, methods of treating a disease using the antigen binding proteins described herein, particularly the trispecific antigen binding proteins as described above, in a subject suffering from cancer are provided. Furthermore, CD19 plays a critical role in both the biology and treatment of B cell malignancies, serving as a key diagnostic marker and a therapeutic target. The CD19 receptor is ubiquitously expressed on B-lymphocytes and maintained across the majority of a wide variety of B-cell malignancies (see e.g., Sermer, D. et al, Blood Reviews, Vol. 57, 2023, 101002). Moreover, increased expression of CD19 was found to correlate with higher levels of autoantibody production. Many autoimmune diseases, including systemic lupus erythematosus (SLE), rheumatoid arthritis (RA), and multiple sclerosis (MS), are characterized by aberrant activation and survival of B cells, leading to the production of autoantibodies that attack the body's tissues. Accordingly, the ability of multispecific antigen binding proteins to deplete pathogenic plasma cells that produce autoantibodies, while engaging the body's own T cells to facilitate this process, may be applied to other autoimmune diseases. Central to the pathogenesis of systemic lupus erythematosus (SLE) are autoantibodies secreted by plasma cells, which arise as a terminal differentiation step from B cells (Malkiel, S, et al, Front Immunol. 2018; 9:427). However, plasma cells are non-reactive to conventional immunosuppressive therapy, i.e., using either steroids or cytostatic drugs. BCMA is exclusively expressed on mature B cells, hence by targeting BCMA with bi- or multispecific T cell engagers (TCEs), the production of harmful autoantibodies can be reduced as the TCE specifically engages and eliminates pathogenic B cells and plasma cells, without affecting other immune cells.

Thus, in certain embodiments, the B cell-related disease is an autoimmune disease, for example Lupus Erythematosus (such as systemic discoid, drug-induced or neonatal lupus, in particular systemic Lupus Erythematosus), lupus nephritis, a relapsed or refractory AQP4-IgG seropositive neuromyelitis optica spectrum disorder, rheumatoid arthritis, systemic sclerosis, Myasthenia gravis, idiopathic thrombocytopeniarpura, autoimmune hemolytic anemia, allergy (e.g., food allergy, pollen allergy, or allergy caused by a non-food allergen).

Moreover, a B cell-targeting strategy using the molecules disclosed herein could serve as a desensitization approach before transplantation to prevent transplant rejection. For example, the CD3xBCMA targeting bispecific linvoseltamab is being clinically evaluated as a desensitization strategy for autologous stem cell transplantation (ASCT; see, e.g., Muralidharan K, et al, J Immunol. 2022 Nov. 15; 209 (10): 1811-1816; Ferreri C., et al, Hemasphere. 2023 August; 7 (Suppl: e521235).

### Kits

Also contemplated are kits comprising at least one antigen binding protein as described herein, typically together with a packaged combination of reagents with instructions. In one embodiment, the kit includes a composition containing an effective amount of said antigen binding protein in unit dosage form. Such kit may comprise a sterile container comprising the composition; non-limiting examples of such containers include, without being limited to, vials, ampoules, bottles, tubes, syringes, blister-packs. In some embodiments, the composition is a pharmaceutical composition and the containers is made of a material suitable for holding medicaments. In one embodiment, the kit may comprise in a first container the antigen binding protein in lyophilized form and a second container with a diluent (e.g., sterile water) for reconstitution or dilution of the antigen binding protein. In some embodiments, said diluent is a pharmaceutically acceptable diluent. In one embodiment, the kit is for diagnostic purposes and the antigen binding protein is formulated for diagnostic applications. In one embodiment, the kit is for therapeutic purposes and the antigen binding protein is formulated for therapeutic applications.

Typically, the kit will further comprise a separate sheet, pamphlet or card supplied in or with the container with instructions for use. If the kit is intended for pharmaceutical use, it may further comprise one or more of the following: information for administering the composition to a subject having a related disease or disorder and a dosage schedule, description of the therapeutic agent, precautions, warnings, indications, counter-indications, overdosage information and/or adverse reactions.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting.

### EXAMPLES

### Example 1—Generation of a CD19xBCMAxCD3 T Cell Engager Molecule

Autoimmune diseases are driven by autoreactive B cells and T cells. The concept of targeting B cells in individuals suffering from autoimmune diseases is practiced in the clinic with e.g., depletion of CD19 or CD20 positive B cells. While CD19 or CD20 are broadly expressed during early and later stages of B cell differentiation, their expression is downregulated on autoreactive antibody-producing plasma cells (PC) and plasma blasts (PB). B-Cell Maturation Antigen (BCMA) is a cell surface antigen that is predominantly expressed in differentiated PC and PB with an increasing expression towards final differentiated long-lived plasma cells.

A trispecific antibody T cell engager that targets CD19xBCMAxCD3 can activate T cells for targeted cell killing of CD19 and BCMA positive B cells. Without being bound to theory, this enables complete depletion of the B cell compartment including PC, PB and all precursors B cells. FIG. 1 depicts CD19 and BCMA expressions at all stages of B cell development.

Previously generated antibodies against CD19 and BCMA (U.S. Pat. No. 7,112,324B1, U.S. Pat. No. 9,034,324B2, incorporated herein by reference) were optimized and combined with a humanized SP34-derived anti-CD3 antibody (Pessano et al. EMBO J., 4 (2); 337-344 1985) to generate a humanized and stable trispecific T cell engager for broad depletion of the B cell compartment.

CDRs and canonical residues of the murine anti-CD19 antibody (U.S. Pat. No. 7,112,324B1) were grafted onto human germline variable and joining genes IGKV1-5 and IGLJ2 for the light chain, and IGHV3-66 and IGHJ4 for the heavy chain, resulting in a human sequence identity of 82.2% in the scFv format.

Antibody phage display libraries were designed based on the CDR L3 and H3 anti-BCMA antibody (PCT/US2010/026825) grafted on human germline variable and joining genes IGKV7-3 and IGKJ4 for the light chain, and IGHV7-4-1 and IGHJ4 for the heavy chain. Randomizations covering the full length of CDR L3 and H3 were introduced by triple consecutive amino acid mutagenesis. Resulting VL and VH libraries were shuffled and phage display libraries were screened against BCMA (recombinant protein was purchased from ACROBiosystems AG, Catalogue number: BCA-H82E4) for the selection of a BCMA-specific hit. Additional sequence randomizations were introduced via error-prone PCR. Three rounds of phage display biopanning were performed to further enhance affinity of the binder towards BCMA. Reformatting of the anti-BCMA Fab to a scFv and further yield and stability increasing point mutations led to the final anti-BCMA in scFv format with human sequence identity of 89.6%.

Both scFvs were stabilized with the incorporation of a VH44-VL100 disulfide bond. The trispecific T cell engager was produced in a Fab-(scFv)₂ format, where the Fab comprises the CD3 binding moiety. Heavy chain and light chain of the Fab are C-terminally linked through a GGGGS (SEQ ID NO: 31)-linker to the scFvs binding to BCMA and CD19, respectively.

Bispecific control molecules were produced as monovalent CD19xCD3 (M4141) or BCMAxCD3 (M3984) as Fab-scFv by fusing the anti-CD19 or anti-BCMA scFv to the heavy chain of the anti-CD3 Fab. Bispecific CD19xCD3 (SEQ ID NO: 51) (purchased from Lucerna-Chem AG, Switzerland, Catalog number: HY-P9963) or BCMAxCD3 (SEQ ID NO: 52, 53, 54, 55) (purchased from Lucerna-Chem AG, Switzerland, Catalog number: HY-P99392) were used as comparator molecules.

### Recombinant Production and Purification of T Cell Engager Molecules

Bispecific CD19xCD3 or BCMAxCD3 T cell engager in Fab-scFv format were expressed by transient co-transfection in HEK293-6E cells. Cells were cultured in suspension using polyethylenimine (PEI 40 kD linear) as transfectant. HEK293-6E cells were seeded at 1.7×10⁶ cells/mL in Freestyle F17 medium supplemented with 2 mM L-Glutamine and 25 ug/mL G418. DNA encoding the light and heavy chains of the molecules and PEI were added separately to 50 μL/mL medium without supplement. Both fractions were mixed at 1:2.5 DNA:PEI ratio, vortexed and rested for 15 minutes. Cells and DNA/PEI mixture were combined (1 μg DNA/mL cells) and incubated at 37 °C, 5% CO2, 80% RH. After 24 hours, cells were supplemented with Tryptone N1 at 25 μL/mL production volume. After 7 days, cells were harvested by centrifugation and the supernatant was sterile filtered. The antigen binding proteins were purified by an affinity chromatography from the supernatant. Supernatant was loaded on a CaptureSelect CH1-XL column (Thermo Fisher Scientific) equilibrated with 6 column volumes (CVs) of PBS (pH 7.4). After a washing step with the same buffer, protein was eluted from the column by step elution with 100 mM citric acid (pH 3.0). Fractions with the desired antigen binding protein were immediately neutralized by 1 M Tris Buffer (pH 9.0) at 1:17 ratio. Size exclusion chromatography was performed as an additional purification step. Samples were run on the Superdex 200 10/300 GL column with PBS (pH7.4) as a running buffer. Collected fractions were analyzed by SE-HPLC for monomer content and pooled accordingly. Final protein purity was assessed by SDS-PAGE and SE-HPLC.

Trispecific CD19xBCMAxCD3 T cell engagers in Fab-(scFv)₂ format were produced by transient co-transfection in ExpiCHO expression system (Thermo Fisher Scientific). The genes for heavy and light chains were expressed using a HC:LC2:1 DNA ratio in shake flask cultures for 7 days. The target proteins were captured from the clarified, sterile-filtered culture supernatants by affinity chromatography (CaptureSelect CH1-XL column, Thermo Fisher Scientific). Size exclusion chromatography was performed as an additional purification step. Samples were run on the Superdex 200 10/300 GL column with PBS (pH7.4) as a running buffer. Collected fractions were analyzed by SE-HPLC for monomer content and pooled accordingly. Final protein purity was assessed by SDS-PAGE and SE-HPLC.

### Affinity Characterization of Antibody T Cell Engager

Antigen binding characterization of recombinantly produced bispecific or trispecific T cell engager molecules as well as the comparator molecule was performed at 25 °C by surface plasmon resonance (SPR) using a Biacore™ 8 k (Cytiva) device. Briefly, appox. 3000 RU of Biotin CAPture reagent (Cytiva) was immobilized on a Series S Sensor Chip CAP (Cytiva) and the biotinylated ligand CD19, BCMA and CD3 was diluted in HPS-EP+ running buffer (TEKNOVA) and captured for 300 s at a flow rate of 2 μL/min at flow cell 2 (recombinant protein was purchased from ACROBiosystems AG, Catalogue numbers: CD19: CD9-H82E9, BCMA: BCA-H82E4, CD3εδ: CDD-H82W6, CD3εγ: CDG-H82W3). Subsequently, at least three consecutive analyte injections were performed at suitable concentrations applying the single-cycle-kinetics mode at a flow rate of 30 μL/min with an association time of 90 s and the dissociation time set to ≥400 s. The chip surface was regenerated according to the manufacturer's instructions. After reference flow cell signal and blank injections subtraction, the data was fit to a 1:1 Langmuir binding model to determine the association rate constant kₐ and dissociation rate constant k_{d} and calculate the equilibrium dissociation constant K_{D} (kd/ka), also denoted as affinity, using the Biacore™ Insight Evaluation software. The resulting K_{D} values of trispecific T cell engager molecule M4148, bispecific controls M4141 and M3984 as well as bispecific comparator molecules are shown in Table 3. M4148 displayed a very high affinity, i.e., low K_{D} value of 20 μM, for the binding to BCMA (SPR reaches detection limit in lower picomolar range) and retained the anti-CD19 affinity after humanization.

TABLE 3
K_{D} values of optimized trispecific T cell engager M4148, bispecific
control molecules M4141, M3984 and the CD19 × CD3 and BCMA ×
CD3 bispecific comparators to CD19, BCMA and CD3εδ/CD3εγ
determined by SPR. (tba = to be analyzed)

| | K_{D} (nM) | | | |
|---|---|---|---|---|
| Molecule | CD19 | BCMA | CD3εδ | CD3εγ |
| M4148 | 1.1 | 0.02 | 3.0 | 3.1 |
| M4141 | 0.51 | — | tba | tba |
| M3984 | — | 0.005 | 1.7 | 2.5 |
| Comparator | 0.43 | — | 70 | 36 |
| CD19 × CD3 | | | | |
| Comparator | — | 0.28 | 6.4 | 9.9 |
| BCMA × CD3 | | | | |

### Example 2—In Vitro Potency Characterization of M4148 on Cancer Cell Lines

To assess the cytotoxicity of CD19xBCMAxCD3 trispecific molecule M4148 and compare it to the bispecific controls and comparator molecule, their potency was tested in a T cell co-culture assay with CD19 and BCMA single-positive, double-positive and negative cells. In healthy individuals, the percentage of BCMA-positive B cells within the whole B cell population is less than 5%. Of those only 10-20% are long-lived PC that are CD19-negative and BCMA-positive (Abeles et al., Annu Rev Immunol. 2024 June; 42 (1): 103-126).

As proof of concept, initial potency assessment and comparison were therefore performed on CD19 and/or BCMA-positive and negative cancer cell lines summarized in Table 4. RPKM values were extracted from RNA-Seq data using TRON CELL LINE PORTAL (Scholtalbers et al., Genome Med. 2015). Copy numbers per cell were quantified with Quantibrite PE beads (BD Biosciences) via flow cytometry according to the manufacturer's protocol. Overall, copy numbers of CD19 are higher compared to BCMA copy numbers on antigen-positive cell lines.

TABLE 4
CD19 and BCMA RNA expression levels (RPKM) and cell copy
number levels of selected cancer cell lines. (copy number levels
compared to isotype control: “−” = <500, “+” = 500-5000, “++” =
5001-50000, “+++” = >50000, “b.l.” = below lower
limit of quantification, “n.m.” = not measured)

| | | | | | CD19 | BCMA |
|---|---|---|---|---|---|---|
| | | | | | copy | copy |
| | CD19 | BCMA | CD19 | BCMA | number | number |
| Cell line | (+/−) | (+/−) | RPKM | RPKM | levels | levels |
| RPMI-8226 | − | + | 0.1 | 53.3 | − | + |
| NALM-6 | + | − | 109.0 | 1.2 | +++ | − |
| Reh | + | − | 15.7 | 0.1 | ++ | − |
| Raji | + | + | 144.5 | 7.0 | +++ | b.l. |
| Daudi | + | + | 133.9 | 18.4 | ++ | + |
| NCI-H1703 | − | − | 0.0 | 0.1 | n.m. | n.m. |
| HL-60 | − | − | 0.1 | 0.25 | b.l. | b.l. |
| PC-3 | − | − | 0.1 | 0.1 | n.m. | n.m. |
| A375 | − | − | 0.0 | 0.0 | n.m. | n.m. |
| SKMEL-30 | − | − | 0.01 | 0.03 | n.m. | n.m. |

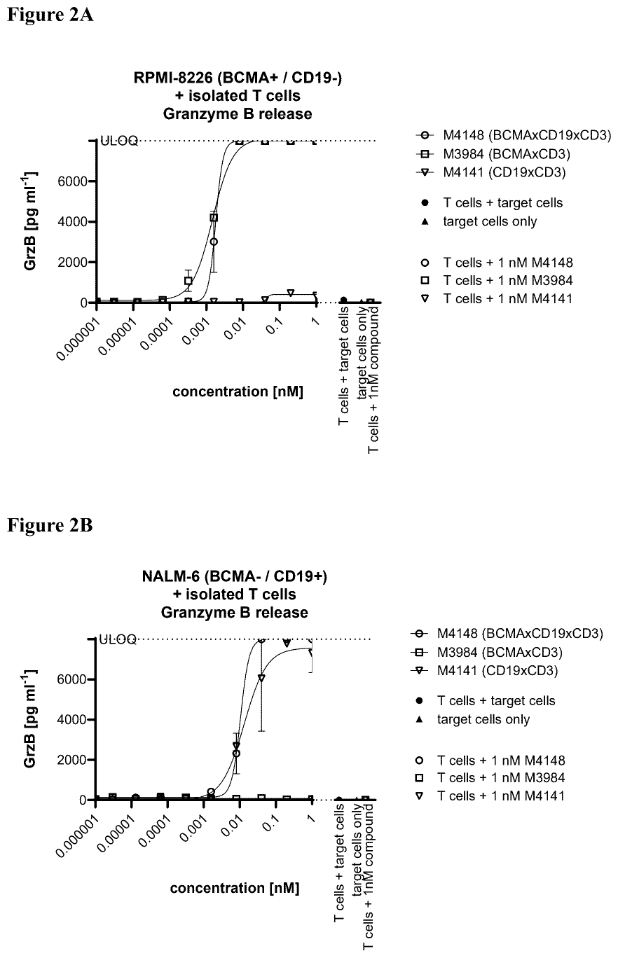

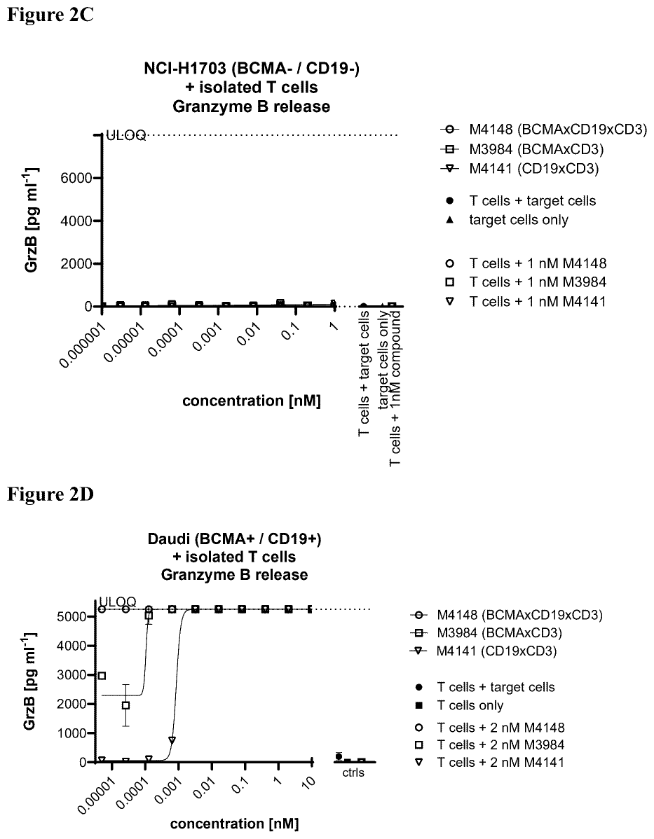

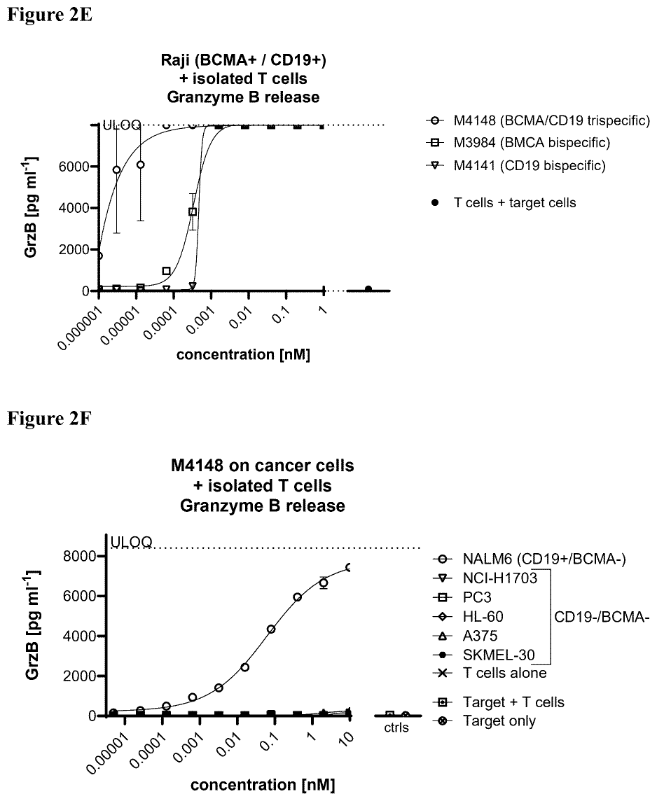

Cancer cells (target cells) were plated at 10,000 cells per well together with isolated T cells (effector cells) at 50,000 cells/well in a volume of 50 μl and 40 μl assay medium (RPMI 1640 containing 10% FBS and 1% penicillin-streptomycin), respectively. Varying concentrations of compounds M4148, M3984 and M4141 were added to the plated wells in 10 μl assay medium, starting at 10 nM final concentration followed by a 1:5 serial dilution. All reactions were performed in duplicates. The plates were incubated for 48 h at 37 °C/5% CO₂. Supernatants were collected and analyzed by human Granzyme B ELISA kit (MabTech) according to the manufacturer's instructions. The upper limit of quantification (ULOQ) was determined in each assay with an internal standard and the Granzyme B concentration in every sample was calculated according to the dilution of the cell supernatant. Target cells and effector cells alone or effector cells with compound served as a negative control. Results are depicted in FIG. 2. The trispecific molecule M4148 was as potent as the respective bispecific control molecules in BCMA-positive/CD19-negative RPMI-8226 or BCMA-negative/CD19-positive NALM-6 cell lines (FIG. 2A, B). There was no Granzyme B release in BCMA-negative/CD19-negative NCI-H1703 cells (FIG. 2C). In BCMA-positive/CD19-positive Daudi and Raji cells, M4148 showed greater potency than bispecific controls M3984 and M4141, indicating the additive effect of the CD19 and BCMA targeting T cell engager (FIG. 2D, E). To ensure high specificity of M4148, the compound was tested on additional BCMA-negative/CD19-negative cancer cell lines PC3, HL-60, A375 and SKMEL-30 and was compared to the positive control cell line NALM-6 (FIG. 2F). There was no Granzyme B release on the antigen-negative cell lines, demonstrating high specificity of the compound.

The potency of M4148 was compared to the CD19xCD3 bispecific comparator in BCMA-positive/CD19-positive cell lines Daudi and Raji (FIGS. 3A and 3B). Granzyme B ELISA assays revealed a stronger potency of M4148 compared to the CD19xCD3 bispecific comparator for both cell lines, showing its beneficial effect of additional BCMA binding for T cell activation. In those assays, the upper limit of quantification was already reached at the lowest concentrations of M4148. An additional assay with increased dilutions of the supernatants for Granzyme B quantification and the inclusion of BCMAxCD3 bispecific comparator was performed on BCMA-positive/CD19-positive cell lines Daudi and Raji (FIGS. 3C and 3D).

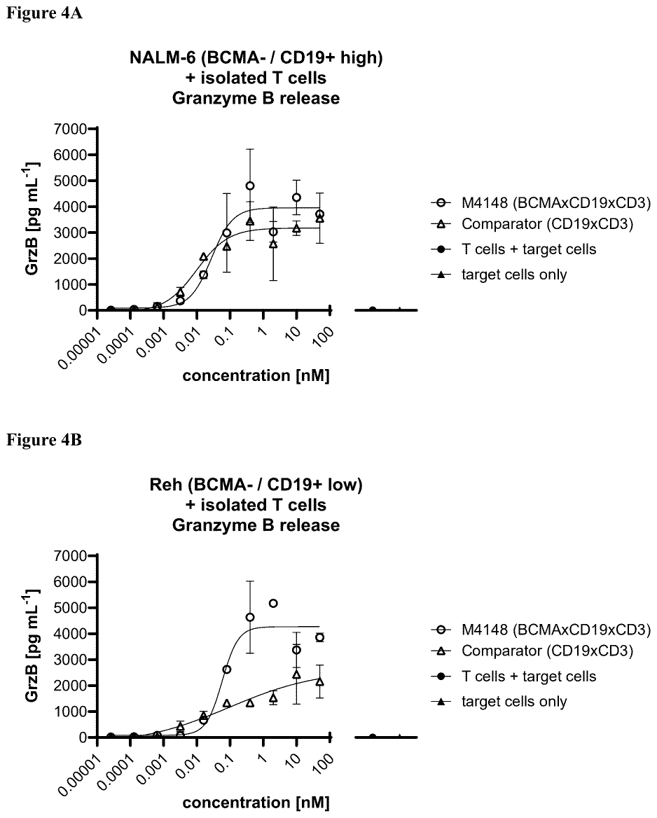

Furthermore, M4148 and the CD19xCD3 bispecific comparator were tested and compared for their ability to induce Granzyme B release in CD19 high versus CD19 low expressing cells NALM-6 and Reh, respectively (FIGS. 4A and 4B). M4148 was highly potent on both cell lines, with strong Granzyme B release at picomolar compound concentrations and independent of CD19 expression levels. CD19xCD3 bispecific comparator compound showed decreased potency in CD19 low expressing cells Reh compared to NALM-6.

### Example 3—In Vitro Potency Characterization of M4148 on B Cells from Healthy Donors

To confirm the potency of M4148 on B cells, the biologically relevant target cells, and compare it to bispecific T cell engager, B cells were isolated from healthy donors and tested in a co-culture assay with isolated effector T cells. Granzyme B release assay was performed as essentially described in Example 2. FIG. 5A shows Granzyme B release of T cells that were co-cultured with isolated B cells under varying concentrations of M4148, confirming the potency of the compound on B cells. As a control, T cells alone were not activated by the compound M4148.

Next, the potency of M4148 on isolated B cells was compared to CD19xCD3 bispecific comparator. Trispecific molecule M4148 showed higher potency compared to the comparator. Results of the Granzyme B release assay are shown in FIG. 5B.

To evaluate B cell depletion of M4148 and the CD19xCD3 bispecific comparator from whole PBMCs, flow cytometry analysis was performed. Cultivation of PBMCs from healthy donors with varying compound concentrations was performed as essentially described in example 2. In each well, 100,000 PBMCs were plated. After 48 h, the cells were analyzed by flow cytometry with the MACSQuant Analyzer 10 (Miltenyi Biotec) to quantify living CD20-positive B cells and CD3-positive T cells. The relative B cell depletion was calculated based on the B cell to T cell ratio relative to an untreated control sample with the following formula: 100−([100/B-/T-cell ratio ctrl]×[B-/T-cell ratio in sample]). FIG. 5C depicts the relative B cell depletion in whole PBMCs, showing a higher depletion of B cells with M4148 compared to the CD19xCD3 bispecific comparator.

As described in Example 2, in PBMCs there is only a minor population of BCMA-positive PC. Therefore, healthy isolated B cells were expanded and stimulated for seven days with the ImmunoCult Human B cell expansion kit (Stemcell Technologies) according to manufacturer's protocol to generate a PC-enriched B cell population. The cells were co-cultured with isolated T cells and varying concentrations of M4148 and the BCMAxCD3 bispecific control M3984. Granzyme B release assay was performed as described above for FIG. 5A. FIG. 5D shows higher potency of the trispecific molecule M4148 compared to the bispecific control M3984.

### Example 4—In Vitro Potency Characterization of M4148 on Bone Marrow Aspirate from Donors without Autoimmune Diseases

The composition of B cell sub-populations differs between peripheral blood and lymphoid organs. The potency of M4148 was therefore further tested on a fresh bone marrow aspirate from a donor without known autoimmune diseases. Granzyme B and Interferon Y release were assessed as surrogate markers for cell killing and T cell activation and compared to BCMAxCD3 bispecific comparator. Briefly, bone marrow cells were treated with red blood cell lysis buffer to remove red blood cells, then centrifuged to remove platelets. Cells were then resuspended in assay medium (RPMI 1640 containing 10% FBS and 1% penicillin-streptomycin) and plated at 100,000 cells per well together with 50,000 isolated T cells. Varying concentrations of compound M4148 and BCMAxCD3 bispecific comparator were added to the plated wells, starting at 10 nM final concentration followed by a 1:8 serial dilution. Bone marrow cells alone without compounds, T cells alone without compounds, or both bone marrow cells and T cells without compounds were added as negative control. All reactions were performed in duplicates. The plates were incubated for 48 h at 37 °C/5% CO₂. Supernatants were collected and Granzyme B and Interferon γ were analyzed with MSD U-plex plates (Meso Scale Discovery) according to the manufacturer's instructions. The upper limit of quantification (ULOQ) was determined in each assay with an internal standard and Granzyme B and Interferon γ concentration in every sample was calculated according to the dilution of the cell supernatant.

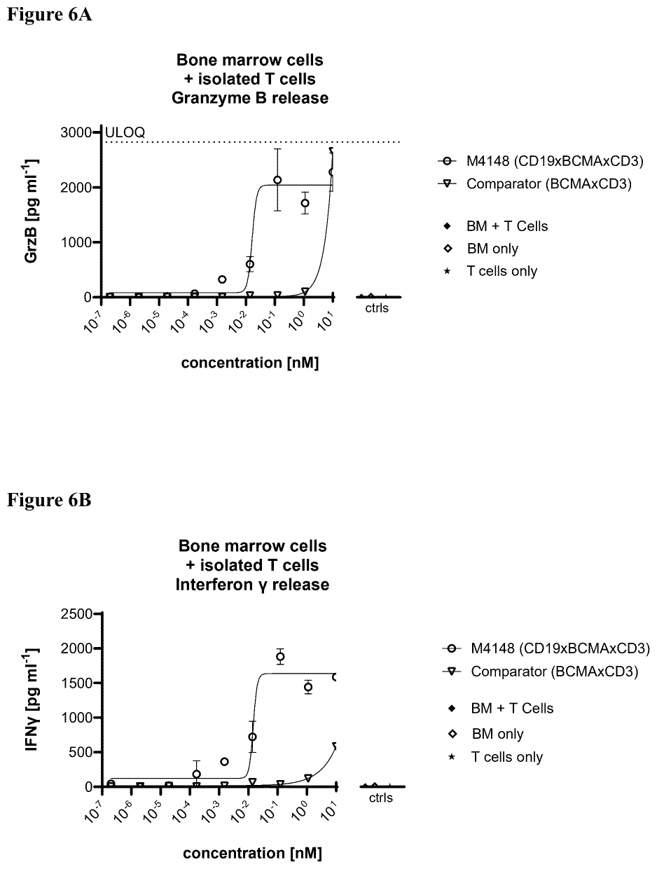

FIG. 6A depicts the potency of M4148 on bone marrow cells and the superior Granzyme B release compared to BCMAxCD3 bispecific comparator. FIG. 6B confirms the increased Interferon γ release of M4148 compared to BCMAxCD3 bispecific comparator, indicating increased T cell activation.

### Example 5—In Vitro Safety Characterization of M4148 in Human Primary Cells

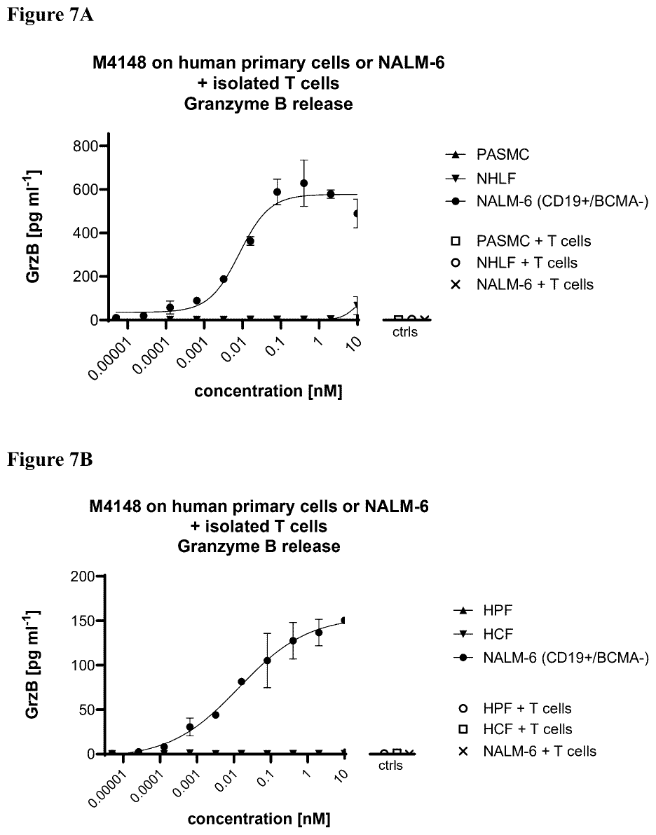

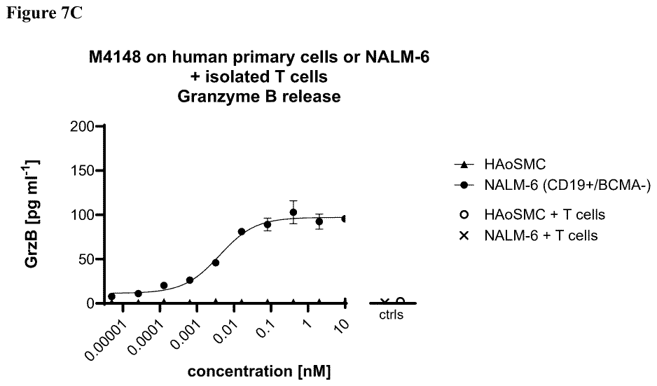

M4148 showed no reactivity on antigen-negative cancer cells (FIG. 2C, F). To further assess the safety of the compound in normal human primary cells co-cultured with isolated T cells, Granzyme B release assay was performed with supernatants of cells of different origin after 24 h, as essentially described in Example 2. The following primary cell types were tested: human lung fibroblasts (NHLF), human pulmonary artery smooth muscle cells (PASMC), human pulmonary fibroblasts (HPF), human cardiac fibroblasts (HCF) and human aortic smooth muscle cells (HAoSMC) (see FIGS. 7A, 7B, 7C, respectively). NALM-6 cells were used as positive control. M4148 showed no reactivity and an excellent safety profile on all tested primary cells.

### Example 6—In Vitro Potency Characterization of M4148 on B Cells from Systemic Lupus Erythematosus (SLE) Donor

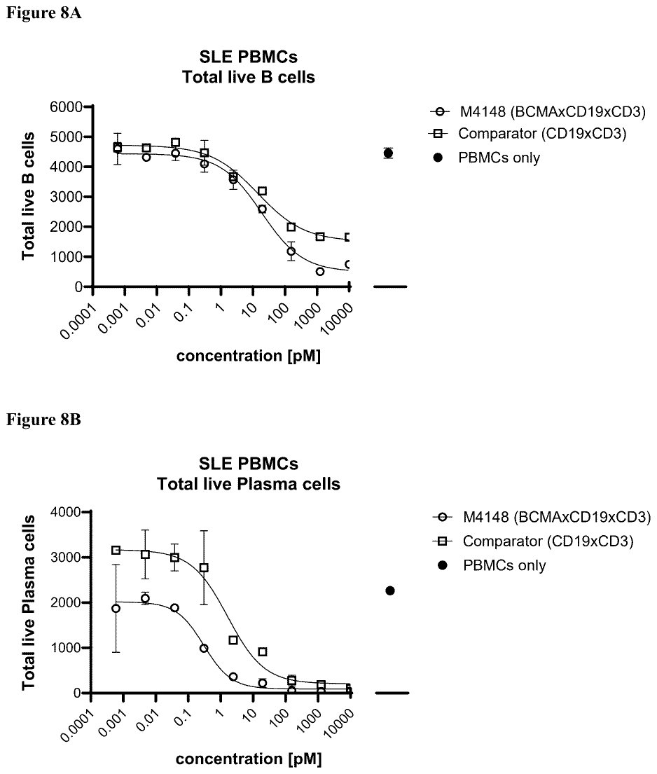

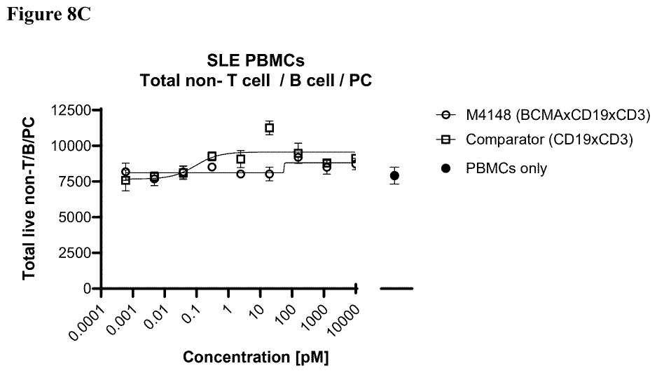

After proof of concept of M4148 potency on cancer cell lines and B cells from healthy donors, B cell depletion was further assessed and compared to CD19xCD3 bispecific comparator in PBMCs from a donor with Systemic Lupus Erythematosus (SLE). B cells depletion assays were performed as essentially described in Example 2 with 100,000 PBMCs under varying concentrations of M4148 and CD19xCD3 bispecific comparator. After 48 h, the cells were analyzed by flow cytometry with the MACSQuant Analyzer 10 (Miltenyi Biotec). Quantification of total B cells occurred via CD3-negative/CD20-positive/CD22-positive gating and quantification of plasma cells (PC) occurred via CD3-negative/CD20-negative/CD22-negative/CD38-positive/CD138-positive gating. To assess specificity of B cell depletion inside the total PBMCs, a population which was negative for T cell, B cell and PC marker was gated and taken as negative control. While there was a slightly higher depletion of total B cells for M4148 compared to the CD19xCD3 bispecific comparator (FIG. 8A), the effect was superior on the PC population showing efficient depletion of PC at lower compound concentrations for M4148 than for the CD19xCD3 bispecific comparator (FIG. 8B). The antigen-negative control population was not affected by both compounds (FIG. 8C).

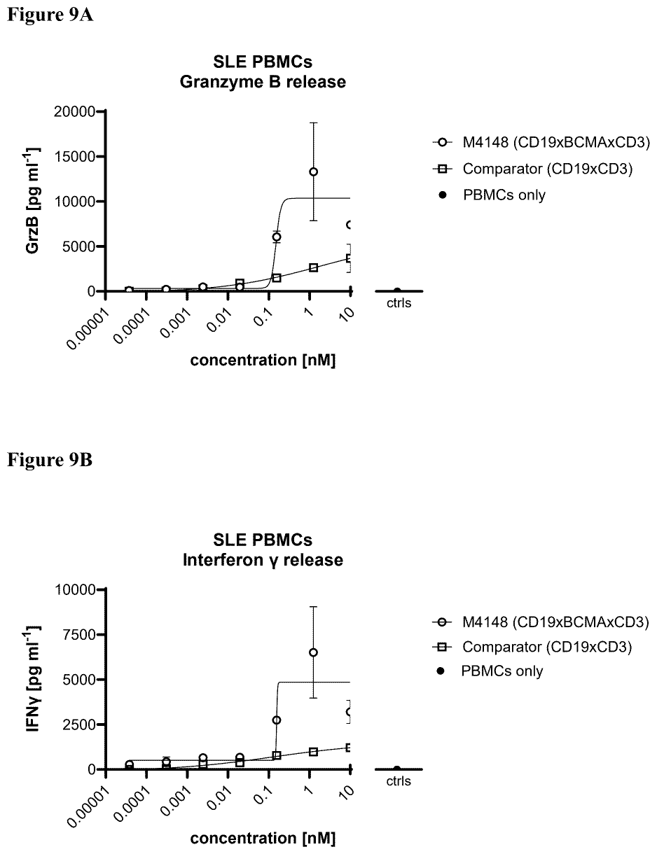

To further quantify M4148-mediated Granzyme B and Interferon γ release compared to CD19xCD3 bispecific comparator in PBMCs from the same donor with SLE, MSD U-plex immune assay was performed as essentially described in Example 4. Briefly, PBMCs were prepared in assay medium (RPMI 1640 containing 10% FBS and 1% penicillin-streptomycin) and plated at 100,000 cells per well in a volume of 90 μL assay medium. Varying concentrations of compound M4148 and CD19xCD3 bispecific comparator were added to the plated wells in 10 μL assay volume, starting at 10 nM final concentration followed by a 1:8 serial dilution. PBMCs alone were added as negative control. All reactions were performed in duplicates. M4148 showed superior potency on SLE PBMCs compared to CD19xCD3 bispecific comparator in the Granzyme B release assay (FIG. 9A) as well as a higher Interferon γ release (FIG. 9B).

### Example 7—In Vitro Potency Characterization of M4148 on CD19-Positive B Cell Depletion with PBMCs from Systemic Lupus Erythematosus (SLE) and Rheumatoid Arthritis (RA) Donors

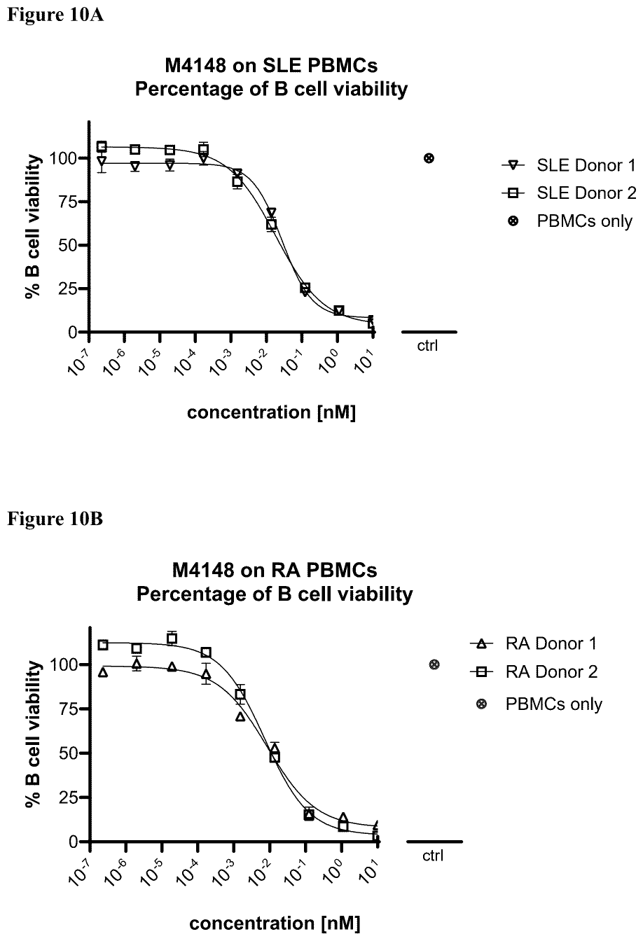

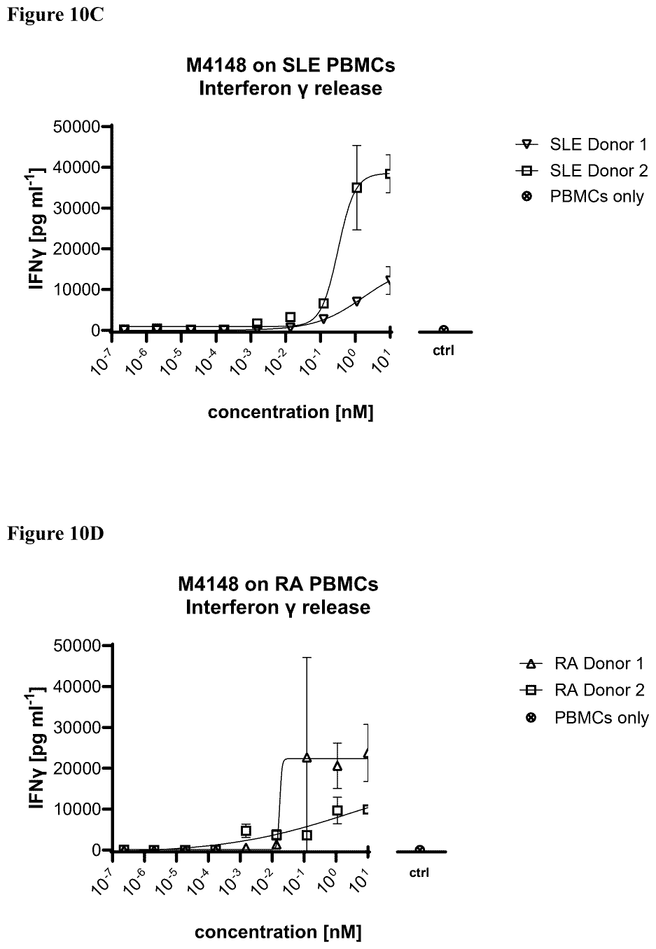

Further validation of M4148 potency was performed on additional representative PBMC samples from donors with SLE and Rheumatoid Arthritis (RA). B cells depletion assays were performed as essentially described in Example 2 and 6 but with the application of an anti-CD19 antibody reagent for the quantification of B cells via flow cytometry. The anti-CD19 antibody is not competing with M4148 on CD19. Quantification of B cells occurred via CD3-negative/CD19-positive gating and the percentage of CD19-positive B cells were calculated based on the viability of PBMCs without compound, referring to 100% viability. M4148 showed potent and nearly complete B cell depletion on both SLE and RA PBMCs in a dose-dependent manner (FIG. 10A, 10B).

Interferon γ release was quantified in the SLE and RA PBMCs as essentially described in Example 4 via MSD U-plex immune assay. Briefly, PBMCs were prepared in assay medium (RPMI 1640 containing 10% FBS and 1% penicillin-streptomycin) and plated at 100,000 cells per well in a volume of 90 μL assay medium. Varying concentrations of compound M4148 were added to the plated wells in 10 μL assay volume, starting at 10 nM final concentration followed by a 1:9 serial dilution. PBMCs alone were added as negative control. All reactions were performed in duplicates. M4148 induced Interferon γ release in all four PBMC samples with a measurable onset at picomolar concentrations (FIG. 10C, 10D).

### Example 8—In Vivo Efficacy Evaluation of M4148 in a SLE-Induced Humanized NCG-M Mouse Model

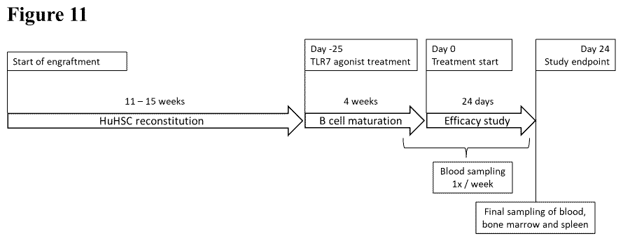

The in vivo efficacy of M4148 in achieving deep B cell depletion in a systemic lupus erythematosus (SLE)-induced mouse model was determined at GemPharmatech, Co. Ltd, Najing, China. The in vivo efficacy experiments were conducted in immune-deficient NCG-M mice that express the human genes for the cytokines Interleukin 3 (IL3), Granulocyte-Macrophage Colony Stimulating Factor (GM-CSF) and Stem Cell Factor (SCF) to support the haematopoiesis and the stable engraftment of multiple immune cell lineages including lymphocytes and myeloid cells after adoptive transfer of CD34⁺ human hematopoietic stem cells (huHSC). Female NCG-M mice were stably engrafted with CD34⁺ HuHSC from three female donors and the hematopoietic reconstitution occurred over a period of 11-15 weeks. Mice with a successful engraftment, defined as ≥25% viable hCD45⁺ cells in peripheral blood, were treated with the Toll-like receptor 7 (TLR7) agonist Resiquimod (R148) for four weeks to induce systemic lupus erythematosus (SLE) symptoms such as increased immune cell activation and elevated antibody production. Peripheral blood was collected on day-4 (pre-treatment) to quantify B cell subsets and human IgG baseline levels. Eleven mice were then entering the efficacy study and were randomized into M4148 treatment group (n=5) or vehicle control group (n=6) based on the number of human CD19⁺ B cells, body weight and Graft-versus-Host disease (GvHD) score (assessment and scoring of mouse health status after HuHSC engraftment based on weight loss, posture, activity and the condition of hair and skin). Mice were treated biweekly for three weeks with buffer control (PBS) or 0.1 mg/kg M4148 (by IV administration) on days 0, 4, 7, 11, 14, 18 and 21. Peripheral blood samples were collected on days 3, 11, 17 and at study endpoint on day 24. Additionally, final sampling included bone marrow and spleen of the animals. A schematic overview of the study design is depicted in FIG. 11.

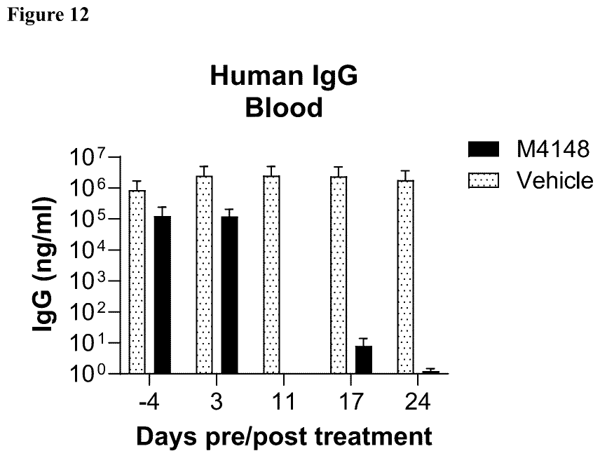

To assess the efficacy of M4148 for human IgG depletion in the humanized SLE mouse model, human IgG levels were quantified in peripheral blood via ELISA of plasma samples. Human IgG levels were strongly reduced by day 11 and sustained low until study endpoint on day 24 in the M4148-treated group, whereas no change of human IgG levels was measured in the vehicle control-treated group (FIG. 12).

The effect of M4148 treatment on the depletion of different B cell populations (B cells, plasma cells and long-lived plasma cells) was determined via flow cytometry with the conceptual gating strategy shown in Table 5.

TABLE 5
Conceptual flow cytometry gating strategy for the analysis
of B cell populations in blood, spleen and bone marrow.

| Cell type | Tissue | Human surface markers |
|---|---|---|
| B cell | Blood, spleen, | CD45⁺/CD3⁻/CD19⁺ |
| | bone marrow | |
| Plasma cell | Blood, spleen, | CD45⁺/CD3⁻/CD19⁺/BCMA⁺ |
| | bone marrow | |
| Long-lived plasma cell | Bone marrow | CD45⁺/CD3⁻/CD19⁻/BCMA⁺ |

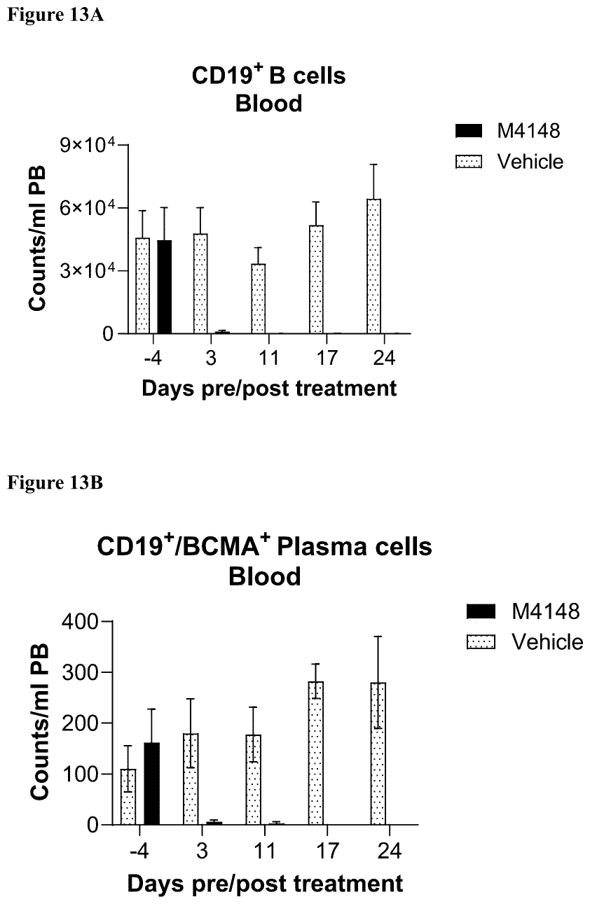

To monitor the efficacy of B cell depletion in the M4148-treated group compared to the vehicle control-treated group, the amount of CD19⁺ B cells and CD19⁺/BCMA⁺ plasma cells was analyzed in the peripheral blood samples taken before and during the treatment. CD19⁺ B cells were markedly reduced on day 3 in the M4148-treated group, thus showing a strong depletion of peripheral B cells already after the first compound injection and sustained further depletion through day 24 (FIG. 13A). There was no depletion of CD19⁺ B cells in the vehicle control-treated group. Similar results were obtained for CD19⁺/BCMA⁺ plasma cells with a strong depletion of those cells on day 3 in the M4148-treated group and sustained low levels through day 24 when compared to the vehicle control-treated group (FIG. 13B).

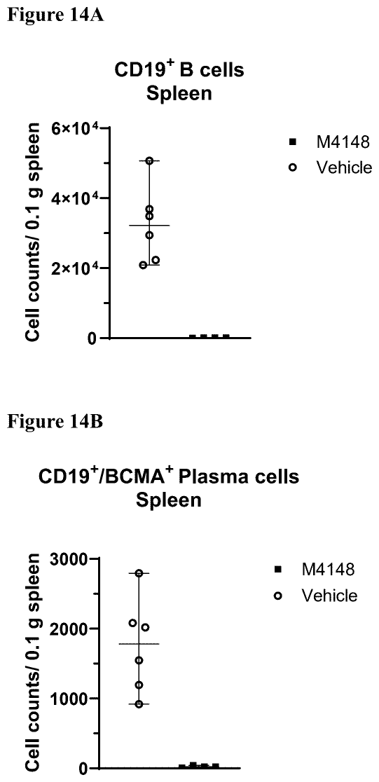

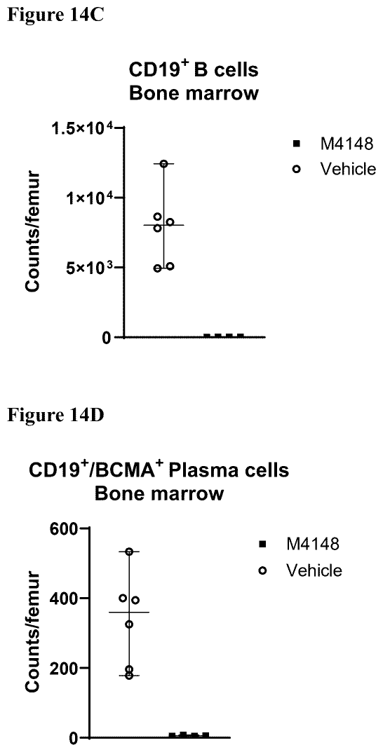

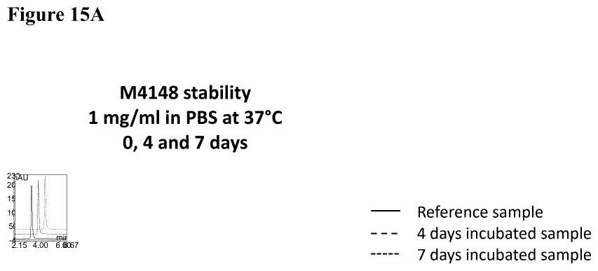

B cell depletion efficacy of M4148 in lymphoid tissues was analyzed by flow cytometry of excised and processed spleen and bone marrow samples from the femur at study endpoint. Both CD19⁺ B cells and CD19⁺/BCMA⁺ plasma cells were efficiently depleted in spleen and bone marrow of the M4148-treated group compared to the vehicle control-treated group (FIG. 14A, 14B, 14C, 14D), showing the ability of M4148 to induce deep depletion in spleen and bone marrow.

### Example 9—Stability and Solubility of M4148

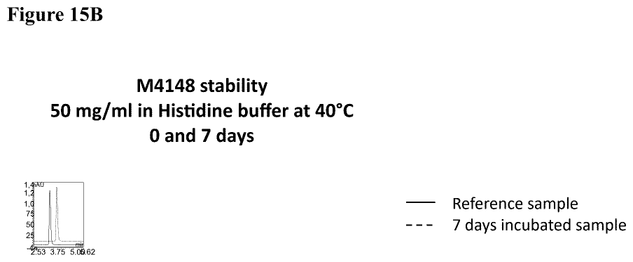

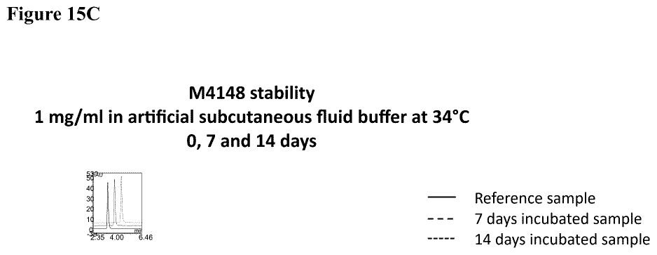

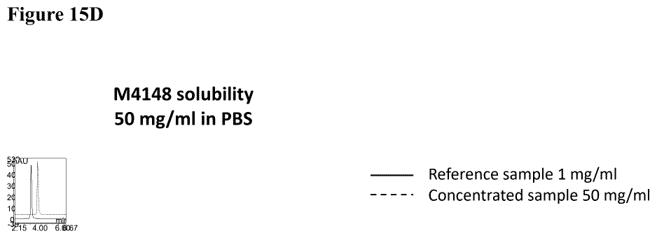

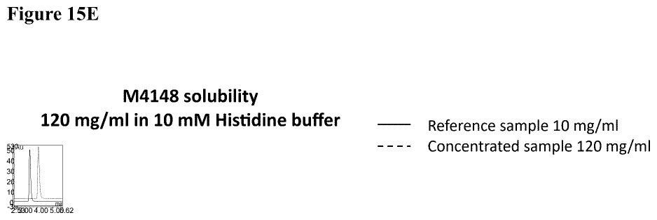

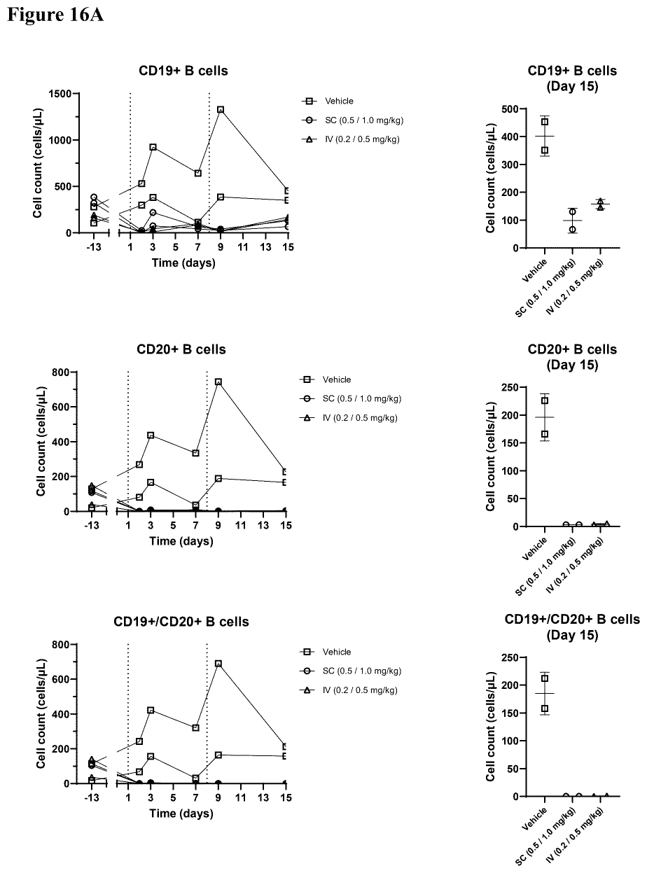

To ensure a good developability profile of the compound, high stability and solubility are desired key biophysical properties. For initial stability testing, M4148 was incubated in PBS for up to seven days at 37 °C and a concentration of 1 mg/ml. The monomer content was measured at day zero, four and seven by SEC analysis depicted in FIG. 15A. M4148 showed excellent stability. There was no detectable oligomerization or fragmentation of the protein over the course of the experiment. Further measurements of M4148 comprised the shelf-life stability under accelerated conditions (50 mg/ml, 10 mM Histidine buffer pH 5.5, 40 °C, 7 days) and stability at simulated physiological conditions (1 mg/ml, artificial subcutaneous fluid buffer (Schuster et al., Journal of Pharmaceutical Sciences 110 (2021):2386-2394), 34 °C, 7 and 14 days) depicted in FIGS. 15B and 15C, respectively. SEC analysis revealed a good, accelerated shelf-life stability and high stability of M4148 at physiological conditions.

Subcutaneous injection of the trispecific is favorable for the treatment of patients with autoimmune diseases. Since injection volumes are limited for subcutaneous administrations, high solubility of the compound is necessary. To determine the solubility of M4148, the molecule was concentrated through centrifugal filters to 50 mg/ml in PBS and 120 mg/ml in 10 mM Histidine buffer pH 5.5. The solubility of the molecule was assessed by measuring the oligomer content through SEC analysis of a reference sample and the concentrated sample. M4148 did not show any oligomerization at high concentrations even in a standard buffer like PBS and at very high concentrations of 120 mg/ml in 10 mM Histidine buffer pH 5.5 (FIGS. 15D and 15E).

While there are shown and described particular embodiments of the invention, it is to be understood that the invention is not limited thereto but may be otherwise variously embodied and practiced within the scope of the following claims. Since numerous modifications and alternative embodiments of the present invention will be readily apparent to those skilled in the art, this description is to be construed as illustrative only and is for the purpose of teaching those skilled in the art the best mode for carrying out the present invention. Accordingly, all suitable modifications and equivalents may be considered to fall within the scope of the following claims.

### Example 10—Evaluation of an M4148 Surrogate in Cynomolgus Monkeys

M4437, a cynomolgus cross-reactive surrogate of M4148, was evaluated in six, adult female cynomolgus monkeys (Macaca fascicularis). M4437 has an identical molecular format to that of M4148. The CD3-binding arm is the same as the CD3-binding arm of M4148 and is cross-reactive to human and cynomolgus CD3. The CD19-binding arm and the BCMA-binding arm are also cross-reactive to human and cynomolgus targets with affinities highly comparable to those of M4148. The potency and biophysical properties including stability are also highly comparable to those of M4148.

### 10-1: Treatment and Sample Collection

Six cynomolgus monkeys were split into the following treatment groups: Group 1 (Subject IDs 1601 and 1602), which received vehicle control via intravenous (IV) slow bolus injection for 20 minutes; Group 2 (Subject IDs 2501 and 2502), which received M4437 by subcutaneous (SC) administration at 0.5 mg/kg on Day 1 and 1 mg/kg on Day 8; and Group 3 (Subject IDs 3601 and 3602), which received M4437 by intravenous (IV) slow bolus injection for 20 minutes at 0.2 mg/kg on Day 1 and 0.5 mg/kg on Day 8.

Peripheral blood samples were collected at multiple timepoints throughout the study: pre-dose collection on Day-13; and post-dose collection on Day 1 (at 2 hours and 6 hours post 1st dose), Day 2 (24 hours post 1st dose), Day 3 (48 hours post 1st dose), Day 7 (24 hours before 2nd dose), Day 9 (24 hours post 2nd dose), and Day 15. Following euthanasia on Day 15, lymph nodes, spleen, and bone marrow samples, as well as brain, adrenal gland, heart, kidney, liver, lung, injection site tissue (SC or IV), small intestine (ileum), stomach, and thymus samples, were collected for subsequent analysis (e.g., on B, plasma, and T cell populations, T cell activation, cytokine levels, cynomolgus IgGs (surrogate marker for plasma cell depletion), pharmacokinetic (PK) analysis, and clinical pathology parameters).

The administration routes, doses, and dosing regimens, as well as sample collection timepoints, were selected based on various factors such as affinity, in vitro potency, efficacy data from humanized mice treated with M4148, and baseline B cell levels in the blood and lymphoid tissues and baseline cytokine levels of cynomolgus monkeys.

Animal treatments and sample collection were completed as planned. Overall, weekly SC or IV administration of M4437 was well tolerated in all animals without causing changes in food consumption, blood pressure, and body temperature throughout the study duration.

### 10-2: Evaluation of B Cells, Plasma Cells, and T Cells by Flow Cytometry

### Methods

Effects on B cells, plasma cells, and T cells in the peripheral blood, bone marrow, spleen and inguinal lymph nodes were assessed by flow cytometry using MACSQuant 16 analyzer (Miltenyi Biotec). All populations were pre-gated on lymphocytes (FSC/SSC), viable cells, and CD14/CD16 negativity to exclude monocytes and natural killer (NK) cells. The following lineage-defining surface marker combinations were used to identify cynomolgus B cell, plasma cell, and T cell subpopulations: CD19+ B cells (CD3−/CD19+); CD20+ B cells (CD3−/CD20+); CD20+ CD19+ B cells (CD3−/CD20+/CD19+); BCMA+ cells (CD3−/BCMA+); BCMA+ Long-lived plasma cells (CD3−/CD19−/BCMA+); T cells (CD3+); Helper T cells (CD3+/CD4+); Cytotoxic T cells (CD3+/CD4−); CD25+ T cells (CD3+/CD25+); and CD69+ T cells (CD3+/CD69+). For CD19 staining, an anti-CD19 antibody which targets a distinct epitope on CD19 from that engaged by M4437 was used. Absolute counts, relative counts, and mean fluorescence intensity (MFI) were measured.

### Results and Interpretation

### B Cells (CD19+, CD20+, and CD19+/CD20+ Cells):

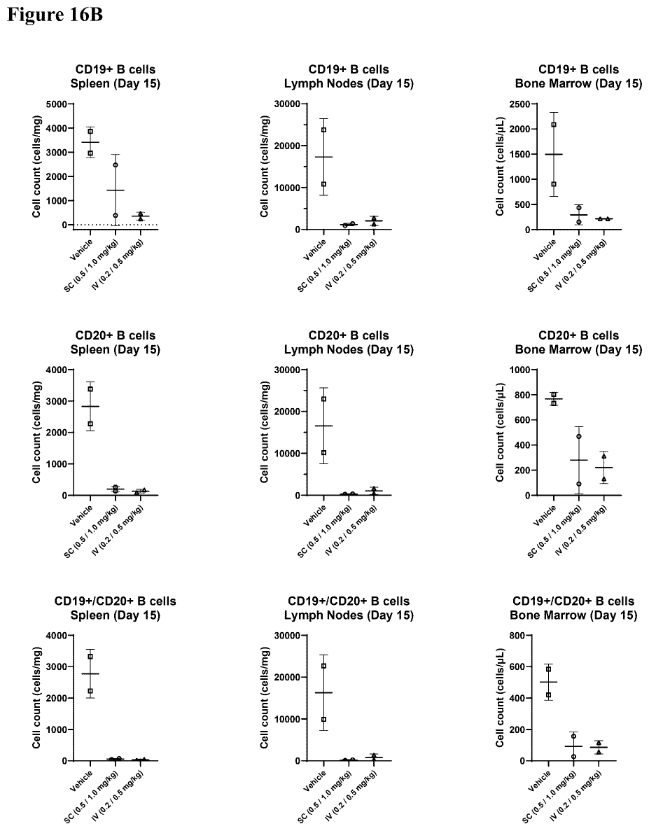

Exemplary results are shown in FIG. 16. The assessment based on CD19+, CD20+, and CD19+/CD20+ cells in the peripheral blood confirmed efficient depletion in all treated animals of Groups 2 and 3, 24 hours after the first dose, based on absolute counts, compared to vehicle control and pre-dose values (FIG. 16A), with a similar trend using relative counts (not shown). In the spleen, lymph nodes, and bone marrow, both IV and SC dosing achieved deep depletion of CD19⁺, CD20⁺, and CD19⁺/CD20⁺ cells, compared to the vehicle control group (FIG. 16B). Overall, B cells were successfully depleted.

### Plasma Cells (BCMA+ Cells):

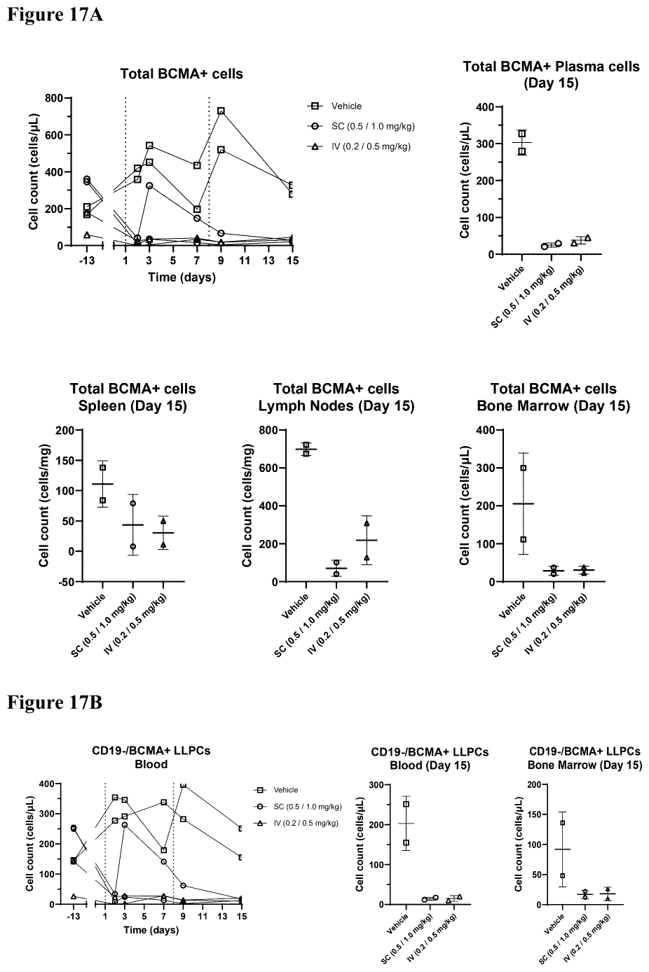

Exemplary results are shown in FIG. 17. A rapid and efficient depletion of the total BCMA⁺ cell population was observed in the peripheral blood in both Groups 2 and 3 from Day 2 through Day 15 relative to pre-dose values (FIG. 17A). Total BCMA⁺ cells were efficiently depleted in the lymph nodes and bone marrow and also reduced in the spleen. Depletion of total BCMA⁺ cells was consistent with very efficient reduction in CD19⁻/BCMA⁺ long-lived plasma cells (LLPCs) in the peripheral blood and bone marrow (FIG. 17B). Similar trends were observed when analyzed using relative cell counts (not shown). A reduction trend was also observed in Groups 2 and 3 when alternative markers for plasma cells (CD138+/CD38+) were used (not shown).

### T Cells (CD3+, CD3+/CD4+, and CD3+/CD4− Cells) and Activated (CD25+, CD69+) T Cells

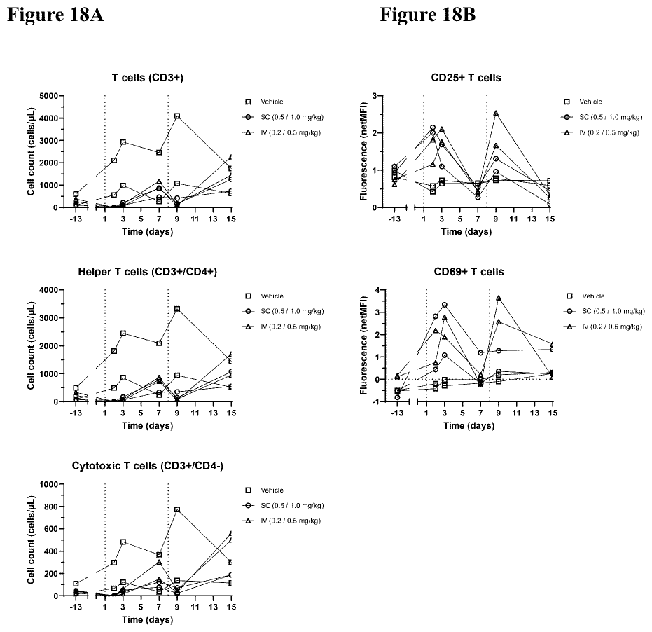

Exemplary results are shown in FIG. 18.

A transient reduction in circulating T cells (CD3+, CD3+/CD4+, and CD3+/CD4− populations) was observed following each administration of M4437 in Groups 2 and 3, on Days 2 and 3 (24 and 48 hours post-first dose) and Day 9 (24 hours post-second dose), relative to pre-dose levels (FIG. 18A). Circulating T cell counts returned to pre-dose levels by Days 7 and 15 in both groups, with a more pronounced recovery observed in Group 3. This transient reduction and return of CD3⁺ T cells in circulation aligns with the T cell margination pattern observed with T cell engagers (TCEs), supporting and further validating the mechanism of action of the tested molecule. Despite the limited residual T cells in circulation in Groups 2 and 3 on Days 2, 3 and 9, both groups showed increased expression of T cell activation markers CD69 and CD25 on these days (FIG. 18B), which declined by Day 7 and 15, respectively. These findings are consistent with the expected mechanism of action of TCEs. No changes in CD25 or CD69 expression were observed in Group 1.

Additionally, effects on hematopoietic stem cells (HSCs), e.g., in the peripheral blood and bone marrow, were analyzed (results not shown). Overall, no clinically meaningful concerns were observed.

### 10-3: Evaluation of B Cells, Plasma Cells, and T Cells by Immunohistochemistry

### Methods

Effects on B cells, plasma cells, and T cells in various tissues were assessed by immunohistochemistry (IHC). The spleen, mesenteric lymph nodes, and sternum (containing bone marrow) samples from Day 15 were fixed with 10% buffered formalin for no longer than 72 hours (bone marrow samples were further decalcified using Osteosoft), and paraffin-embedded (FFPE) blocks were prepared. Sections of approximately 4 μm were prepared and stained for CD19, CD20, BCMA, and CD3 using cynomolgus-specific antibodies. All tissues were also stained with hematoxylin-eosin (H&E). All examinations were performed by a trained veterinary pathologist at the CRO.

### Results and Interpretation

### IHC of CD19 and CD20:

Exemplary IHC scoring results from CD19 and CD20 staining are shown in Table 6.

In all Group 1 tissues, CD19 and CD20 showed comparable staining distribution patterns, characterized by predominantly membranous staining with occasional cytoplasmic localization. In contrast, Groups 2 and 3 exhibited marked to severe reduction in staining intensity for both CD19 and CD20, consistent with the flow cytometric assessment.

In the spleens from Group 1 animals, both markers showed abundant staining within B cell areas (follicles with germinal centers and the marginal zone), and less frequently in the red pulp. In Group 2 animals, staining was almost absent. In Group 3 animals, staining was markedly decreased to nearly absent, with residual positivity primarily confined to follicles.

In the mesenteric lymph nodes from Group 1 animals, abundant staining was present in cortical follicles (including germinal centers), with less frequent staining in medullary cords and sinuses and occasional staining in paracortical regions. In Group 2 animals, staining was severely reduced across all compartments, with minimal residual positivity mainly in follicles. In Group 3 animals, staining was moderately to markedly reduced within follicles or throughout all compartments.

In the sternal bone marrow from Group 1 animals, approximately 10% of cells stained positive for both markers. In Group 2 animals, staining was absent, while in Group 3 animals the staining was nearly to completely absent.

TABLE 6
IHC scoring of CD19 and CD20

| | | | CD19 | | | CD20 | | |
|---|---|---|---|---|---|---|---|---|
| | Dose (mg/kg) | Animal | | Mesenteric | Bone | | Mesenteric | Bone |
| Group | and Route | ID | Spleen | Lymph Node | Marrow | Spleen | Lymph Node | Marrow |
| 1 | Vehicle | 1601 | 0^{a} | 0 | 0 | 0^{a} | 0 | 0 |
| | | 1602 | 0 | 0 | 0 | 0 | 0 | 0 |
| 2 | SC | 2501 | 5 | 5 | 5 | 5 | 5 | 5 |
| | (0.5/1.0 mg/kg) | 2502 | 5 | 5 | 5 | 5 | 5 | 5 |
| 3 | IV | 3601 | 5 | 3 | 5 | 5 | 4 | 5 |
| | (0.2/0.5 mg/kg) | 3602 | 4 | 4 | 5 | 4 | 4 | 5 |

^{a}Values in treated animals represent the decreased staining density compared with concurrent controls, where 0 = Within normal range; 1 = Minimal decrease (<20%); 2 = Mild decrease (20-40%); 3 = Moderate decrease (41-60%); 4 = Marked decrease (61-80%); 5 = Severe decrease (>80%).

Exemplary IHC scoring results from BCMA staining are shown in Table 7.

In tissues from Group 1 animals, BCMA staining showed membranous and cytoplasmic positivity. Both Groups 2 and 3 showed decreased BCMA staining density, consistent with the flow cytometry assessment, indicating effective BCMA⁺ cell depletion.

In Group 1 spleens, BCMA staining was low, with scattered positive round cells mainly in the periarteriolar sheaths and red pulp and fewer cells in follicles, including germinal centers. In Group 2 animals, staining was markedly to severely reduced and, when present, was primarily confined to the red pulp. In Group 3 animals, staining was markedly reduced but remained detectable in the periarteriolar sheaths and red pulp.

In Group 1 mesenteric lymph nodes, staining was low to moderate, with positive cells distributed throughout medullary cords and sinuses, cortical follicles (including germinal centers), and the paracortex. In Group 2 animals, staining was almost completely absent, while in Group 3 animals the staining was minimal to mildly reduced.

In Group 1 sternal bone marrow, approximately 10% of cells were BCMA-positive. In Group 2 animals, staining was almost completely absent, while in Group 3 animals the staining was moderately to markedly reduced.

TABLE 7
IHC scoring of BCMA

| | | | BCMA | | |
|---|---|---|---|---|---|
| | Dose | | | Mesenteric | |
| | (mg/kg) | Animal | | Lymph | Bone |
| Group | and Route | ID | Spleen | Node | Marrow |
| 1 | Vehicle | 1601 | 0^{a} | 0 | 0 |
| | | 1602 | 0 | 0 | 0 |
| 2 | SC (0.5/1.0 | 2501 | 5 | 5 | 5 |
| | mg/kg) | 2502 | 4 | 5 | 5 |
| 3 | IV (0.2/0.5 | 3601 | 4 | 1 | 3 |
| | mg/kg) | 3602 | 4 | 2 | 4 |

^{a}Values in treated animals represent the decreased staining density compared with concurrent controls, where 0 = Within normal range; 1 = Minimal decrease (<20%); 2 = Mild decrease (20-40%); 3 = Moderate decrease (41-60%); 4 = Marked decrease (61-80%); 5 = Severe decrease (>80%).

### 10-4: Cytokine Analysis

### Methods

Cytokine concentrations in plasma samples collected from all animals at pre-dose, Day 1 (2 h and 6 h after the first dose), Day 7, and Day 9 (24 hours after the second dose) were quantified using a Luminex MAP® technology assay kit (MILLIPLEX® Non-Human Primate Cytokine/Chemokine/Growth Factor Panel A) measuring IFN-γ, IL-1β, IL-2, IL-6, IL-8, IL-10, MCP-1, MIP-1β, and TNF-α.

### Results and Interpretation

Exemplary results are shown in FIG. 19.

In Groups 2 and 3, transient increases in IFN-γ, IL-2, IL-6, IL-8, IL-10, MCP-1, MIP-1β, and TNF-α were observed at 2 and 6 hours post-dose. Cytokine concentrations returned to baseline within 24 hours, with no evidence of accumulation following the second dosing.

IL-1β levels remained below the limit of quantification or showed no treatment-related changes under the analytical conditions tested. For IL-8, high variability was observed across animals (including Group 1 animals). The time-dependent profile exhibited fluctuations that appeared unrelated to treatment and did not follow the pattern seen for other cytokines.

### 10-5: Cynomolgus IgG Quantification

### Methods

Cynomolgus IgG (cyIgG) concentrations were quantified in serum samples collected at pre-dose, Day 7, Day 9 (24 hours after the second dose), and Day 15 as a surrogate marker of plasma cell depletion. Analyses were performed using a commercially available ELISA kit.

To assess the magnitude of cyIgG reduction, absolute concentrations for each animal were normalized to pre-dose values. A threshold of 70% of the pre-dose values was applied to determine the incidence of cyIgG reduction and was established based on Group 1 values on Day 15.

### Results and Interpretation

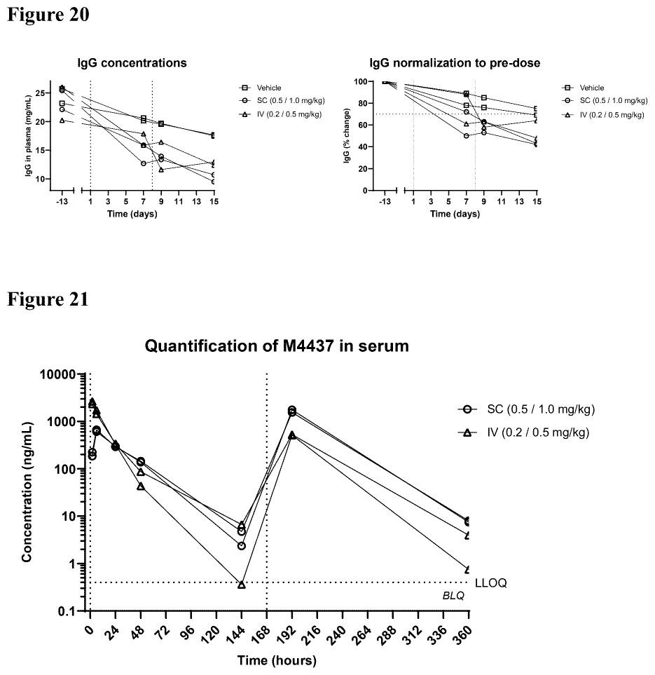

Exemplary results are shown in FIG. 20. Treatment-related decreases of cyIgG were observed in all animals in Group 2 and 3 animals, with all animals falling below the threshold by Day 9, consistent with the expected pharmacological activity of M4437 involving depletion of antibody-generating plasma cells.

### 10-6: Pharmacokinetics

### Methods

Serum concentrations of M4437 were measured using a fit-for-purpose (FFP) validated immunoassay on the Meso Scale Discovery (MSD) platform. The assay had an analytical range of 0.4 to 2000 ng/mL and employed biotinylated human BCMA protein and a SULFO-tagged idiotypic antibody (M1964) directed against the CD3-binding region of M4437 as reagents.

### Results

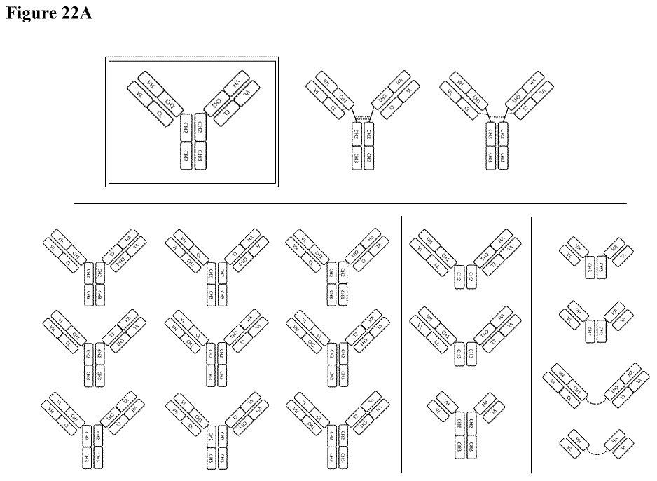

Exemplary results are shown in FIG. 21 and Table 8.

TABLE 8
Quantification of M4437 in serum samples

| | | Mean measured concentration [ng/mL] | | | |
|---|---|---|---|---|---|
| Timepoints | | SC (0.5/1.0 mg/kg) | | IV (0.2/0.5 mg/kg) | |
| Days | Hours | 2501 | 2502 | 3601 | 3602 |
| Pre-dose | −312 | BLQ | BLQ | BLQ | BLQ |
| Day 1 | 2 | 184.59 | 224.56 | 2334.34 | 2644.21 |
| Day 1 | 6 | 608.95 | 664.5 | 1439.35 | 1724.44 |
| Day 2 | 24 | 298.66 | 290.55 | 338.94 | 326.38 |
| Day 3 | 48 | 138.02 | 144.94 | 86.81 | 43.47 |
| Day 7 | 144 | 2.36 | 4.73 | 6.65 | 0.36 |
| Day 9 | 192 | 1753.66 | 1545.31 | 528.98 | 511.51 |
| Day 15 | 360 | 7.47 | 8.03 | 3.95 | 0.75 |

Italics: Sample below the limit of quantification (BLQ).

### Additional Exemplary Embodiments

### Additional Embodiments A

Embodiment A-1. A BCMA binding protein (e.g., anti-BCMA antibody or antigen binding fragment thereof), comprising:
(A) a VL comprising a CDR-L1, a CDR-L2, and a CDR-L3; and
(B) a VH comprising a CDR-H1, a CDR-H2, and a CDR-H3,
   wherein:
(A) the CDR-L1, the CDR-L2, and the CDR-L3 comprise the light chain CDR1, 2, and 3 sequences, respectively, contained in SEQ ID NO: 7 or 82; and
(B) the CDR-H1, a CDR-H2, and a CDR-H3 comprise the heavy chain CDR1, 2, and 3 sequences, respectively, contained in SEQ ID NO: 8,
   optionally wherein the CDRs are defined according to Kabat, Chothia, Contact, IMGT, or AHo numbering scheme.

Embodiment A-2. The BCMA binding protein of Embodiment A-1, wherein:
(A) the CDR-L1, the CDR-L2, and the CDR-L3 comprise the amino acid sequences of SEQ ID NOS: 1, 2, and 3, respectively; and
(B) the CDR-H1, the CDR-H2, and the CDR-H3 comprise the amino acid sequences of SEQ ID NOS: 4, 5, and 6, respectively.

Embodiment A-3. The BCMA binding protein of Embodiment A-1 or A-2, wherein:
(A) the VL comprises an amino acid sequence having at least about 90%, 95%, 96%, 97%, 98%, 99% or 100% identity to SEQ ID NO: 7 or 82; and/or
(B) the VL comprises an amino acid sequence having at least about 90%, 95%, 96%, 97%, 98%, 99% or 100% identity to SEQ ID NO: 8.

Embodiment A-4. The BCMA binding protein of any one of Embodiments A-1 through A-3, which:
(i) comprises a scFv, a Fab, Fab′, F(ab′)₂, Fv fragment, or scFab comprising the VL and the VH;
(ii) comprises:
   (a) a light chain which comprises the VL and a light chain constant region comprising a light chain constant domain (CL); and
   (b) a heavy chain which comprises the VH and a heavy chain constant region comprising at least a heavy chain constant domain (CH) and/or a hinge (e.g., CH1, hinge, CH2, and/or CH3),
   optionally comprising an immunoglobulin molecule comprising two light chains and two heavy chains, optionally an IgG (e.g., IgG1, IgG2, IgG3, IgG4), IgE, or IgD, or a multimer of the immunoglobulin molecule, optionally an IgA (e.g., IgA (e.g., IgA1, IgA2) or IgM;
(iii) is or comprises a structure depicted in any of FIGS. 22A-22B or description thereof;
(iv) is conjugated to a drug or a moiety;
(v) is glycosylated, PEGylated, PASylated, XTENylated or HESylated; and/or
(vi) is comprised in a CAR which comprises an anti-BCMA domain, a transmembrane domain, and a cytoplasmic signaling domain, and optionally a intracellular signaling domain.

Embodiment A-5. The BCMA binding protein thereof of any one of Embodiments A-1 through A-4, which comprises a scFv comprising the VL and the VH optionally connected via a linker, optionally wherein:
(i) the scFv comprises the VL, a linker, and the VH in the direction from the N-terminus to the C-terminus, optionally comprising SEQ ID NO: 9 or 83 or an amino acid sequence having at least about 90%, 95%, 96%, 97%, 98%, 99% or 100% identity thereto; or
(ii) the scFv comprises the VH, a linker, and the VL in the direction from the N-terminus to the C-terminus, the scFv comprises SEQ ID NO: 84, 85, 86, or 87 or an amino acid sequence having at least about 90%, 95%, 96%, 97%, 98%, 99% or 100% identity thereto,
   optionally wherein the linker:
(a) comprises about 1 to about 30 amino acids, about 3 to about 30 amino acids, about 5 to about 25 amino acids, about 10 to about 25 amino acids, about 12 to about 25 amino acids, about 12 to about 23 amino acids, about 12 to about 22 amino acids, about 12 to about 20 amino acids, preferably about 21, about 20, or about 19 amino acids;
(b) comprises or consists of glycine and/or serine;
(c) comprises SEQ ID NO: 31 or two or more repeats thereof or SEQ ID NO: 38 (e.g., n=1, 2, 3, 4, 5, 6, 7, or 8); and/or
(d) comprises SEQ ID NO: 34, 35, 36, 33, or 32, or any of SEQ ID NOS: 39-47.

Embodiment A-6. The BCMA binding protein of any one of Embodiments A-1 through A-5, which is comprised in a multispecific antigen binding protein (e.g., multispecific antibody or antibody fragment), optionally wherein the multispecific antibody or antibody fragment:
(I) comprises:
   (i) a structure depicted in any of FIGS. 23A-23C and 24A-24B or description thereof;
   (ii) a bispecific antigen binding protein (e.g., bispecific antibody or antibody fragment) comprising a structure depicted in any of FIGS. 23A-23C or description thereof, optionally wherein the VL and the VH correspond to VL-B and VH-B of FIGS. 23A-23B, preferably one of the boxed structures of FIG. 23A or description thereof, optionally wherein the VL and the VH correspond to the VL-B and the VH-B of FIG. 24A;
   (iii) a trispecific antigen binding protein (e.g., trispecific antibody or antibody fragment) comprising a structure depicted in any of FIGS. 24A-24B or description thereof, optionally wherein the VL and the VH correspond to the VL-C and the VH-C of FIGS. 24A-24B; and/or
   (iv) a trispecific antigen binding protein (e.g., trispecific antibody or antibody fragment) comprising a structure depicted in the top section (left or right panel) of FIG. 24A or description thereof, preferably the boxed structure, optionally wherein the VL and the VH correspond to the VL-C and the VH-C of FIG. 24A; and/or
(II) is capable of binding to BCMA and one or more of CD3, CD19, CD20, CD28, TCRα, TCRβ, NKp30, NKp44, NKp46, NKG2D, CD93/NKG2C, CD94/NKG2R, a Killer Immunoglobulin-like Receptor (KIR) (e.g., KIR2DS1, KIR2DS4, KIR3DS1), an Fc receptor (e.g., CD16. CD32, CD64, CD89), DNAM-1 (CD226), 2B4 (CD244), NKR-P1 (CD161), CD2, CD44, NKp80, a Toll-like receptor (TLR) (e.g., TLR4, TLR 2, TLR3), mannose receptor, a complement receptor (e.g., CR1, CR3), an IFNγ receptor, a TNF receptor, CD206, dectin-1, CD14, CD11b, CD11c, siglec receptor (e.g., siglect-1, 3, 14, 15, or 16).

Embodiment A-7. The BCMA binding protein thereof of any one of Embodiments A-1 through A-6, which comprises one or more of a CH1 domain, a hinge or a partial hinge, a CH2 domain, a CH3 domain, a CL domain, and/or an Fc region, optionally wherein:
(i) the CH1 domain, the hinge, the CH2 domain, and/or the CH3 domain is/are individually of or derived from any isotype, e.g., IgG (e.g., IgG1, IgG4, IgG2, or IgG3), IgA, IgE, IgD, or IgM, optionally wherein:
   (a) the CH1 comprises SEQ ID NO: 56, 57, 58, 59, or 60 or an amino acid sequence having at least about 90%, 95%, 96%, 97%, 98%, 99% or 100% identity thereto;
   (b) the hinge comprises SEQ ID NO: 61, 62, 63, or 64 or an amino acid sequence having at least about 90%, 95%, 96%, 97%, 98%, 99% or 100% identity thereto, and/or the partial hinge comprises SEQ ID NO: 37 or partial sequence of SEQ ID NO: 61, 62, 63, or 64 or an amino acid sequence having at least about 90%, 95%, 96%, 97%, 98%, 99% or 100% identity thereto;
   (c) the CH2 comprises SEQ ID NO: 65, 66, 67, or 68 or an amino acid sequence having at least about 90%, 95%, 96%, 97%, 98%, 99% or 100% identity thereto;
   (d) the CH3 comprises SEQ ID NO: 69, 70, 71, 72, 73, 74, or 75 or an amino acid sequence having at least about 90%, 95%, 96%, 97%, 98%, 99% or 100% identity thereto; and/or
(ii) the CL domain is of or derived from any isotype, e.g., kappa CL or lambda CL, optionally wherein:
   (a) the kappa CL comprises SEQ ID NO: 76 or an amino acid sequence having at least about 90%, 95%, 96%, 97%, 98%, 99% or 100% identity thereto; and/or
   (b) the lambda CL comprises SEQ ID NO: 77, 78, 79, 80, or 81 or an amino acid sequence having at least about 90%, 95%, 96%, 97%, 98%, 99% or 100% identity thereto;
(iii) the Fc region is of or derived from any isotype, e.g., IgG (e.g., IgG1, IgG4, IgG2, or IgG3), IgA, IgE, IgD, or IgM; and/or
(iv) the Fc region or the CH2 and/or CH3 domain(s) comprises one or more amino acid substitutions:
   (iv-1) which reduce an Fc effector function;
   (iv-2) at position(s) selected from the group consisting of positions 234, 235, 236, 237, 265, 297 and 329, according to EU numbering;
   (iv-3) comprising at least the following amino acid substitutions according to EU numbering: (a) L234A and L235A; (b) L234A, L235A, and P329G; (c) I253A, H310A, and H435Q; and/or (d) I253A, H310A, and H435A, optionally relative to: (a) a human IgG (optionally IgG1) Fc region sequence; (b) a human IgG (optionally IgG1) CH2 sequence, optionally any one of SEQ ID NOS: 65-68; and/or (c) a human IgG (optionally IgG1) CH3 sequence, optionally any one of SEQ ID NOS: 69-75;
(v) the Fc region is of:
   (v-1) an IgG1 or a variant thereof, optionally comprising one or more of the following amino acid substitutions: L234A, L235A, N297A, N297Q, D265A, C226S, C229S, P238S, E233P, L234V, G236-deleted, P238A, A327Q, A327G, P329A, P329G, K322A, L234F, L235E, P331S, T394D, A330L, P331S, F243L, R292P, Y300L, V305I, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, H310A, L328F, M252Y, I253A, S254T, T256E, H435Q, H435A, and/or any combination thereof, according to EU numbering;
   (v-2) an IgG2 or a variant thereof, optionally comprising one or more of the following amino acid substitutions: P238S, V234A, G237A, H268A, H268Q, H268E, V309L, N297A, N297Q, A330S, P331S, C232S, C233S, M252Y, S254T, T256E, and/or any combination thereof, according to EU numbering;
   (v-3) an IgG3 or a variant thereof, optionally comprising the amino acid substitution E235Y, according to EU numbering; or
   (v-4) an IgG4 or a variant thereof, optionally comprising one or more of the following amino acid substitutions: E233P, F234V, L235A, G237A, E318A, S228P, L236E, S241P, L248E, T394D, M252Y, S254T, T256E, N297A, N297Q, and/or any combination thereof, according to EU numbering.

Embodiment A-8. The BCMA binding protein thereof of any one of Embodiments A-1 through A-7, which comprises one or more of the following features:
(i) binds to human BCMA monovalently;
(ii) binds to human BCMA with a K_{D} of about 0.5 pM to about 0.5 nM, about 1 pM to about 300 pM, about 1 pM to about 100 pM, about 5 pM to about 50 pM, about 10 pM to about 30 pM, about 15 pM to about 25 pM, or about 20 pM;
(ii) binds to human BCMA with a K_{D} of about 1 pM to about 50 pM, about 2 pM to about 30 pM, about 5 pM to about 20 pM, about 20 pM or about 5 pM as an scFv, optionally wherein the K_{D} is determined using SPR.

Embodiment A-9. An isolated or recombinant nucleic acid encoding a BCMA binding protein of any one of Embodiments A-1 through A-8, or a vector comprising the isolated or recombinant nucleic acid.

Embodiment A-10. A host cell comprising the isolated or recombinant nucleic acid or a vector of Embodiments A-9, or a cell population comprising such host cells.

Embodiment A-11. A pharmaceutical composition comprising:
(A) a BCMA binding protein of any one of Embodiments A-1 through A-8, an isolated or recombinant nucleic or a vector of Embodiment A-9, and/or a host cell or a cell population of Embodiment A-10; and
(B) a pharmaceutically acceptable carrier.

Embodiment A-12. A method of producing a host cell of Embodiments A-10, comprising introducing an isolated or recombinant nucleic or a vector of Embodiments A-9 into a cell.

Embodiment A-13. A method of producing a BCMA binding protein of any one of Embodiments A-1 through A-8, comprising culturing a host cell or a cell population of Embodiments A-10 in a condition which allows for expression of the BCMA binding protein and optionally purifying the BCMA binding protein.

### Additional Embodiments B

Embodiment B-1. A CD19 binding protein (e.g., an anti-CD19 antibody or antigen binding fragment thereof), comprising:
(A) a VL comprising a CDR-L1, a CDR-L2, and a CDR-L3; and
(B) a VH comprising a CDR-H1, a CDR-H2, and a CDR-H3,
   wherein:
(A) the CDR-L1, the CDR-L2, and the CDR-L3 comprise the light chain CDR1, 2, and 3 sequences, respectively, contained in SEQ ID NO: 16 or 88; and
(B) the CDR-H1, a CDR-H2, and a CDR-H3 comprise the heavy chain CDR1, 2, and 3 sequences, respectively, contained in SEQ ID NO: 17, optionally wherein the CDRs are defined according to Kabat, Chothia, Contact, IMGT, or AHo numbering scheme.

Embodiment B-2. The CD19 binding protein of Embodiment B-1, wherein:
(A) the CDR-L1, the CDR-L2, and the CDR-L3 comprise the amino acid sequences of SEQ ID NOS: 10, 11, and 12, respectively; and
(B) the CDR-H1, the CDR-H2, and the CDR-H3 comprise the amino acid sequences of SEQ ID NOS: 13, 14, and 15, respectively.

Embodiment B-3. The CD19 binding protein of Embodiment B-1 or B-2, wherein:
(A) the VL comprises an amino acid sequence having at least about 90%, 95%, 96%, 97%, 98%, 99% or 100% identity to SEQ ID NO: 16 or 88; and/or
(B) the VL comprises an amino acid sequence having at least about 90%, 95%, 96%, 97%, 98%, 99% or 100% identity to SEQ ID NO: 17.

Embodiment B-4. The CD19 binding protein of any one of Embodiments B-1 through B-3, which:
(i) comprises a scFv, a Fab, Fab′, F(ab′)₂, Fv fragment, or scFab comprising the VL and the VH;
(ii) comprises:
   (a) a light chain which comprises the VL and a light chain constant region comprising a light chain constant domain (CL); and
   (b) a heavy chain which comprises the VH and a heavy chain constant region comprising at least a heavy chain constant domain (CH) and/or a hinge (e.g., CH1, hinge, CH2, and/or CH3),
   optionally comprising an immunoglobulin molecule comprising two light chains and two heavy chains, optionally an IgG (e.g., IgG1, IgG2, IgG3, IgG4), IgE, or IgD, or a multimer of the immunoglobulin molecule, optionally an IgA (e.g., IgA (e.g., IgA1, IgA2) or IgM;
(iii) is comprises a structure depicted in any of FIGS. 22A-22B or description thereof;
(iv) is conjugated to a drug or a moiety;
(v) is glycosylated, PEGylated, PASylated, XTENylated or HESylated; and/or
(vi) is comprised in a CAR which comprises an anti-CD19 domain, a transmembrane domain, and a cytoplasmic signaling domain, and optionally an intracellular signaling domain.

Embodiment B-5. The CD19 binding protein of any one of Embodiments B-1 through B-4, which comprises a scFv comprising the VL and the VH optionally connected via a linker, optionally wherein:
(i) the scFv comprises the VL, a linker, and the VH in the direction from the N-terminus to the C-terminus, optionally comprising SEQ ID NO: 18 or 89 or an amino acid sequence having at least about 90%, 95%, 96%, 97%, 98%, 99% or 100% identity thereto; or
(ii) the scFv comprises the VH, a linker, and the VL in the direction from the N-terminus to the C-terminus, the scFv comprises SEQ ID NO: 90, 91, 92, or 93 or an amino acid sequence having at least about 90%, 95%, 96%, 97%, 98%, 99% or 100% identity thereto, optionally wherein the linker:
(a) comprises about 1 to about 30 amino acids, about 3 to about 30 amino acids, about 5 to about 25 amino acids, about 10 to about 25 amino acids, about 11 to about 25 amino acids, about 12 to about 23 amino acids, about 12 to about 22 amino acids, about 12 to about 20 amino acids, preferably about 21, about 20, or about 19 amino acids;
(b) comprises or consists of glycine and/or serine;
(c) comprises SEQ ID NO: 31 or two or more repeats thereof or SEQ ID NO: 38 (e.g., n=1, 2, 3, 4, 5, 6, 7, or 8); and/or
(d) comprises SEQ ID NO: 34, 35, 36, 33, or 32, or any of SEQ ID NOS: 39-47.

Embodiment B-6. The CD19 binding protein of any one of Embodiments B-1 through B-5, which is comprised in a multispecific antigen binding protein (e.g., a multispecific antibody or antibody fragment), optionally wherein the multispecific antigen binding protein:
(I) comprises:
   (i) a structure depicted in any of FIGS. 23A-23C and 24A-24B or description thereof;
   (ii) a bispecific antigen binding protein (e.g., bispecific antibody or antibody fragment) comprising a structure depicted in any of FIGS. 23A-23C or description thereof, optionally wherein the VL and the VH correspond to VL-B and VH-B of FIGS. 23A-23B, preferably one of the boxed structures of FIG. 23A or description thereof, optionally wherein the VL and the VH correspond to the VL-B and the VH-B of FIG. 24A;
   (iii) a trispecific antigen binding protein (e.g., trispecific antibody or antibody fragment) comprising a structure depicted in any of FIGS. 24A-24B or description thereof, optionally wherein the VL and the VH correspond to the VL-B and the VH-B of FIGS. 24A-24B; and/or
   (iv) a trispecific antigen binding protein (e.g., trispecific antibody or antibody fragment) comprising a structure depicted in the top section (left or right panel) of FIG. 24A or description thereof, preferably the boxed structure, optionally wherein the VL and the VH correspond to the VL-B and the VH-B of FIG. 24A; and/or
(II) is capable of binding to CD19 and one or more of CD3, BCMA, CD20, CD28, TCRα, TCRβ, NKp30, NKp44, NKp46, NKG2D, CD93/NKG2C, CD94/NKG2R, a Killer Immunoglobulin-like Receptor (KIR) (e.g., KIR2DS1, KIR2DS4, KIR3DS1), an Fc receptor (e.g., CD16. CD32, CD64, CD89), DNAM-1 (CD226), 2B4 (CD244), NKR-P1 (CD161), CD2, CD44, NKp80, a Toll-like receptor (TLR) (e.g., TLR4, TLR 2, TLR3), mannose receptor, a complement receptor (e.g., CR1, CR3), an IFNγ receptor, a TNF receptor, CD206, dectin-1, CD14, CD11b, CD11c, siglec receptor (e.g., siglect-1, 3, 14, 15, or 16).

Embodiment B-7. The CD19 antigen binding protein of any one of Embodiments B-1 through B-6, which comprises one or more of a CH1 domain, a hinge or a partial hinge, a CH2 domain, a CH3 domain, a CL domain, and/or an Fc region, optionally wherein:
(i) the CH1 domain, the hinge, the CH2 domain, and/or the CH3 domain is/are individually of or derived from any isotype, e.g., IgG (e.g., IgG1, IgG4, IgG2, or IgG3), IgA, IgE, IgD, or IgM, optionally wherein:
   (a) the CH1 comprises SEQ ID NO: 56, 57, 58, 59, or 60 or an amino acid sequence having at least about 90%, 95%, 96%, 97%, 98%, 99% or 100% identity thereto;
   (b) the hinge comprises SEQ ID NO: 61, 62, 63, or 64 or an amino acid sequence having at least about 90%, 95%, 96%, 97%, 98%, 99% or 100% identity thereto, and/or the partial hinge comprises SEQ ID NO: 37 or partial sequence of SEQ ID NO: 61, 62, 63, or 64 or an amino acid sequence having at least about 90%, 95%, 96%, 97%, 98%, 99% or 100% identity thereto;
   (c) the CH2 comprises SEQ ID NO: 65, 66, 67, or 68 or an amino acid sequence having at least about 90%, 95%, 96%, 97%, 98%, 99% or 100% identity thereto;
   (d) the CH3 comprises SEQ ID NO: 69, 70, 71, 72, 73, 74, or 75 or an amino acid sequence having at least about 90%, 95%, 96%, 97%, 98%, 99% or 100% identity thereto; and/or
(ii) the CL domain is of or derived from any isotype, e.g., kappa CL or lambda CL, optionally wherein:
   (a) the kappa CL comprises SEQ ID NO: 76 or an amino acid sequence having at least about 90%, 95%, 96%, 97%, 98%, 99% or 100% identity thereto; and/or
   (b) the lambda CL comprises SEQ ID NO: 77, 78, 79, 80, or 81 or an amino acid sequence having at least about 90%, 95%, 96%, 97%, 98%, 99% or 100% identity thereto;
(iii) the Fc region is of or derived from any isotype, e.g., IgG (e.g., IgG1, IgG4, IgG2, or IgG3), IgA, IgE, IgD, or IgM; and/or
(iv) the Fc region or the CH2 and/or CH3 domain(s) comprises one or more amino acid substitutions:
   (iv-1) which reduce an Fc effector function;
   (iv-2) at position(s) selected from the group consisting of positions 234, 235, 236, 237, 265, 297 and 329, according to EU numbering;
   (iv-3) comprising at least the following amino acid substitutions according to EU numbering: (a) L234A and L235A; (b) L234A, L235A, and P329G; (c) I253A, H310A, and H435Q; and/or (d) I253A, H310A, and H435A, optionally relative to: (a) a human IgG (optionally IgG1) Fc region sequence; (b) a human IgG (optionally IgG1) CH2 sequence, optionally any one of SEQ ID NOS: 65-68; and/or (c) a human IgG (optionally IgG1) CH3 sequence, optionally any one of SEQ ID NOS: 69-75;
(v) the Fc region is of:
   (v-1) an IgG1 or a variant thereof, optionally comprising one or more of the following amino acid substitutions: L234A, L235A, N297A, N297Q, D265A, C226S, C229S, P238S, E233P, L234V, G236-deleted, P238A, A327Q, A327G, P329A, P329G, K322A, L234F, L235E, P331S, T394D, A330L, P331S, F243L, R292P, Y300L, V305I, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y M428L, N434S, H310A, L328F, M252Y, I253A, S254T, T256E, H435Q, H435A, and/or any combination thereof, according to EU numbering;
   (v-2) an IgG2 or a variant thereof, optionally comprising one or more of the following amino acid substitutions: P238S, V234A, G237A, H268A, H268Q, H268E, V309L, N297A, N297Q, A330S, P331S, C232S, C233S, M252Y, S254T, T256E, and/or any combination thereof, according to EU numbering;
   (v-3) an IgG3 or a variant thereof, optionally comprising the amino acid substitution E235Y, according to EU numbering; or
   (v-4) an IgG4 or a variant thereof, optionally comprising one or more of the following amino acid substitutions: E233P, F234V, L235A, G237A, E318A, S228P, L236E, S241P, L248E, T394D, M252Y, S254T, T256E, N297A, N297Q, and/or any combination thereof, according to EU numbering.

Embodiment B-8. The CD19 binding protein of any one of Embodiments B-1 through B-7, which comprises one or more of the following features:
(i) binds to human CD19 monovalently;
(ii) binds to human CD19 with a K_{D} of about 0.5 pM to about 0.5 nM, about 1 pM to about 300 pM, about 1 pM to about 100 pM, about 5 pM to about 50 pM, about 10 pM to about 30 pM, about 15 pM to about 25 pM, or about 20 pM;
(ii) binds to human CD19 with a K_{D} of about 1 pM to about 50 pM, about 2 pM to about 30 pM, about 5 pM to about 20 pM, about 20 pM or about 5 pM as an scFv, optionally wherein the K_{D} is determined using SPR.

Embodiment B-9. An isolated or recombinant nucleic acid encoding a CD19 binding protein of any one of Embodiments B-1 through B-8, or a vector comprising the isolated or recombinant nucleic acid.

Embodiment B-10. A host cell comprising the isolated or recombinant nucleic acid or a vector of Embodiments B-9.

Embodiment B-11. A pharmaceutical composition comprising:
(A) a CD19 binding protein of any one of Embodiments B-1 through B-8, an isolated or recombinant nucleic or a vector of Embodiment B-9, and/or a host cell or a cell population of Embodiment B-10; and
(B) a pharmaceutically acceptable carrier.

Embodiment B-12. A method of producing a host cell of Embodiments B-10, comprising introducing an isolated or recombinant nucleic or a vector of Embodiments B-9 into a cell.

Embodiment B-13. A method of producing a CD19 binding protein of any one of Embodiments B-1 through B-8, comprising culturing a host cell or a cell population of Embodiments B-10 in a condition which allows for expression of the CD19 binding protein and optionally purifying the CD19 binding protein.

### Additional Embodiments C

Embodiment C-1. A trispecific antigen binding protein (e.g., a trispecific antibody or antibody fragment), which comprises a first antigen binding unit (ABU-1) which specifically binds to CD3, a second antigen binding unit (ABU-2) which specifically binds to CD19, and a third antigen binding unit (ABU-3) which specifically binds to BCMA.

Embodiment C-2. The trispecific antigen binding protein of Embodiment C-1, wherein:
(i) the ABU-1 binds to CD3 monovalently;
(ii) the ABU-2 binds to CD19 monovalently; and/or
(iii) the ABU-3 binds to BCMA monovalently.

Embodiment C-3. The trispecific antigen binding protein of Embodiment C-1 or C-2, wherein:
(I) (i) the ABU-1 binds to CD3 monovalently;
   (ii) the ABU-2 binds to CD19 monovalently; and
   (iii) the ABU-3 binds to BCMA monovalently;
(II) the trispecific antigen binding protein is trivalent; and/or
(II) the trispecific antigen binding protein comprises one ABU-1, one ABU-2, and one ABU-3.

Embodiment C-4. The trispecific antigen binding protein of any one of Embodiments C-1 to C-3, wherein:
(I) (i) the trispecific antigen binding protein binds to CD19 more efficiently (with a lower K_{D}, e.g., about 2-fold to about 10-fold, about 2-fold to about 5-fold, about 2-fold to about 4-fold, or about 3-fold lower KD) compared to CD3;
   (ii) the trispecific antigen binding protein binds to BCMA more efficiently (with a lower KD, e.g., about 1000-fold to about 10-fold, about 500-fold to about 50-fold, about 300-fold to about 100-fold, or about 150-fold lower KD) compared to CD3; and/or
   (iii) the trispecific antigen binding protein binds to CD19 and BCMA more efficiently (with a lower KD) compared to CD3; and/or
(II) (i) the ABU-1 binds to CD3 with a KD of about 0.5 to about 20 nM, about 1 to about 10 nM, about 2 to about 5 nM, about 2, about 3, about 4, or about 5 nM;
   (ii) the ABU-2 binds to CD19 with a KD of about 0.01 to about 50 nM, about 0.05 to about 10 nM, about 0.1 to about 5 nM, about 0.5 nM to about 2 nM, or about 1 or about 1.1 nM; and/or
   (iii) the ABU-3 binds to BCMA with a KD of about 0.5 pM to about 0.5 nM, about 1 pM to about 300 pM, about 1 pM to about 100 pM, about 5 pM to about 50 pM, about 10 pM to about 30 pM, about 15 pM to about 25 pM, or about 20 pM.

Embodiment C-5. The trispecific antigen binding protein of any one of Embodiments C-1 to C4, which:
(i) comprises a molecular weight of about 75 to about 125 kDa, about 80 to about 120 kDa, about 85 to about 115 kDa, about 90 to about 110 kDa, about 95 to about 105 kDa, or about 100 kDa;
(ii) comprises a molecular weight smaller than about 150 kDa, smaller than about 140 kDa, smaller than about 130 kDa, smaller than about 120 kDa, smaller than about 115 kDa, or smaller than about 110 kDa;
(iii) has a theoretical molecular weight of 100610.8 Da;
(iv) has a molecular weight of about 100 to about 110 kDa, about 103 to about 109 kDa, about 105 to about 107 kDa, or about 106 kDa, e.g., 100593.8 Da;
(v) is not glycosylated;
(vi) does not comprise an Fc region;
(vii) does not comprise a CH2 domain; and/or
(viii) does not comprise a CH3 domain.

Embodiment C-6. The trispecific antigen binding protein of any one of Embodiments C-1 to C5, wherein:
(i) the ABU-1 comprises:
   (a) a first VH (VH-1) comprising a CDR-H1-1, a CDR-H2-1, and a CDR-H3-1; and
   (b) optionally a first VL (VL-1) comprising a CDR-L1-1, a CDR-L2-1, and a CDR-L3-1;
(ii) the ABU-2 comprises:
   (a) a second VH (VH-2) comprising a CDR-H1-2, a CDR-H2-2, and a CDR-H3-2; and
   (b) optionally a second VL (VL-2) comprising a CDR-L1-2, a CDR-L2-2, and a CDR-L3-2; and/or
(iii) the ABU-3 comprises:
   (a) a third VH (VH-3) comprising a CDR-H1-3, a CDR-H2-3, and a CDR-H3-3; and
   (b) optionally a third VL (VL-3) comprising a CDR-L1-3, a CDR-L2-3, and a CDR-L3-3.

Embodiment C-7. The trispecific antigen binding protein of any one of Embodiments C1 to C-6, which comprises a structure depicted in any of FIGS. 24A-24B or description thereof, optionally wherein the trispecific antigen binding protein is according to Embodiment C-6 and:
(i) the VH-1 and the optional VL-1 correspond to the VH-A and the optional VL-A of FIGS. 24A-24B, the VH-2 and the optional VL-2 correspond to the VH-B and the optional VL-B of FIGS. 24A-24B, and the VH-3 and the optional VL-3 correspond to the VH-C and the optional VL-C of FIGS. 24A-24B (i.e., ABU-1, ABU-2, and ABU-3 correspond to ABU-A, ABU-B, and ABU-C, respectively);
(ii) the VH-1 and the optional VL-1 correspond to the VH-A and the optional VL-A of FIGS. 24A-24B, the VH-2 and the optional VL-2 correspond to the VH-C and the optional VL-C of FIGS. 24A-24B, and the VH-3 and the optional VL-3 correspond to the VH-B and the optional VL-B of FIGS. 24A-24B (i.e., ABU-1, ABU-2, and ABU-3 correspond to ABU-A, ABU-C, and ABU-B, respectively);
(iii) the VH-1 and the optional VL-1 correspond to the VH-B and the optional VL-B of FIGS. 24A-24B, the VH-2 and the optional VL-2 correspond to the VH-A and the optional VL-A of FIGS. 24A-24B, and the VH-3 and the optional VL-3 correspond to the VH-C and the optional VL-C of FIGS. 24A-24B (i.e., ABU-1, ABU-2, and ABU-3 correspond to ABU-B, ABU-A, and ABU-C, respectively);
(iv) the VH-1 and the optional VL-1 correspond to the VH-B and the optional VL-B of FIGS. 24A-24B, the VH-2 and the optional VL-2 correspond to the VH-C and the optional VL-C of FIGS. 24A-24B, and the VH-3 and the optional VL-3 correspond to the VH-A and the optional VL-A of FIGS. 24A-24B (i.e., ABU-1, ABU-2, and ABU-3 correspond to ABU-B, ABU-C, and ABU-A, respectively);
(v) the VH-1 and the optional VL-1 correspond to the VH-C and the optional VL-C of FIGS. 24A-24B, the VH-2 and the optional VL-2 correspond to the VH-A and the optional VL-A of FIGS. 24A-24B, and the VH-3 and the optional VL-3 correspond to the VH-B and the optional VL-B of FIGS. 24A-24B (i.e., ABU-1, ABU-2, and ABU-3 correspond to ABU-C, ABU-A, and ABU-B, respectively); or
(v) the VH-1 and the optional VL-1 correspond to the VH-C and the optional VL-C of FIGS. 24A-24B, the VH-2 and the optional VL-2 correspond to the VH-B and the optional VL-B of FIGS. 24A-24B, and the VH-3 and the optional VL-3 correspond to the VH-A and the optional VL-A of FIGS. 24A-24B (i.e., ABU-1, ABU-2, and ABU-3 correspond to ABU-C, ABU-B, and ABU-A, respectively).

Embodiment C-8. The trispecific antigen binding protein of any one of Embodiments C-1 to C7, which comprises a structure depicted in FIG. 24A or description thereof.

Embodiment C-9. The trispecific antigen binding protein of any one of Embodiments C-1 to C8, which comprises a structure depicted in the top section (left or right panel) of FIG. 24A or description thereof.

Embodiment C-10. The trispecific antigen binding protein of any one of Embodiments C-1 to C9, which comprises the boxed (solid or dashed) structure of FIG. 24A or description thereof or a structure depicted in the top section right panel of FIG. 24A or description thereof, optionally wherein the trispecific antigen binding protein is according to Embodiment C-6 and:
(i) the VH-1 and the optional VL-1 correspond to the VH-A and the optional VL-A of FIGS. 24A-24B, the VH-2 and the optional VL-2 correspond to the VH-B and the optional VL-B of FIGS. 24A-24B, and the VH-3 and the optional VL-3 correspond to the VH-C and the optional VL-C of FIGS. 24A-24B (i.e., ABU-1, ABU-2, and ABU-3 correspond to ABU-A, ABU-B, and ABU-C, respectively);
(ii) the VH-1 and the optional VL-1 correspond to the VH-A and the optional VL-A of FIGS. 24A-24B, the VH-2 and the optional VL-2 correspond to the VH-C and the optional VL-C of FIGS. 24A-24B, and the VH-3 and the optional VL-3 correspond to the VH-B and the optional VL-B of FIGS. 24A-24B (i.e., ABU-1, ABU-2, and ABU-3 correspond to ABU-A, ABU-C, and ABU-B, respectively);
(iii) the VH-1 and the optional VL-1 correspond to the VH-B and the optional VL-B of FIGS. 24A-24B, the VH-2 and the optional VL-2 correspond to the VH-A and the optional VL-A of FIGS. 24A-24B, and the VH-3 and the optional VL-3 correspond to the VH-C and the optional VL-C of FIGS. 24A-24B (i.e., ABU-1, ABU-2, and ABU-3 correspond to ABU-B, ABU-A, and ABU-C, respectively);
(iv) the VH-1 and the optional VL-1 correspond to the VH-B and the optional VL-B of FIGS. 24A-24B, the VH-2 and the optional VL-2 correspond to the VH-C and the optional VL-C of FIGS. 24A-24B, and the VH-3 and the optional VL-3 correspond to the VH-A and the optional VL-A of FIGS. 24A-24B (i.e., ABU-1, ABU-2, and ABU-3 correspond to ABU-B, ABU-C, and ABU-A, respectively);
(v) the VH-1 and the optional VL-1 correspond to the VH-C and the optional VL-C of FIGS. 24A-24B, the VH-2 and the optional VL-2 correspond to the VH-A and the optional VL-A of FIGS. 24A-24B, and the VH-3 and the optional VL-3 correspond to the VH-B and the optional VL-B of FIGS. 24A-24B (i.e., ABU-1, ABU-2, and ABU-3 correspond to ABU-C, ABU-A, and ABU-B, respectively); or
(v) the VH-1 and the optional VL-1 correspond to the VH-C and the optional VL-C of FIGS. 24A-24B, the VH-2 and the optional VL-2 correspond to the VH-B and the optional VL-B of FIGS. 24A-24B, and the VH-3 and the optional VL-3 correspond to the VH-A and the optional VL-A of FIGS. 24A-24B (i.e., ABU-1, ABU-2, and ABU-3 correspond to ABU-C, ABU-B, and ABU-A, respectively).

Embodiment C-11. The trispecific antigen binding protein of any one of Embodiments C-1 to C10, which comprises the boxed (solid) structure of FIG. 24A or description thereof or one of the top three structures in the top section right panel of FIG. 24A or description thereof and is according to Embodiment C-6, wherein:
(i) the VH-1 and the optional VL-1 correspond to the VH-A and the optional VL-A of FIGS. 24A-24B, the VH-2 and the optional VL-2 correspond to the VH-B and the optional VL-B of FIGS. 24A-24B, and the VH-3 and the optional VL-3 correspond to the VH-C and the optional VL-C of FIGS. 24A-24B (i.e., ABU-1, ABU-2, and ABU-3 correspond to ABU-A, ABU-B, and ABU-C, respectively); or
(ii) the VH-1 and the optional VL-1 correspond to the VH-A and the optional VL-A of FIGS. 24A-24B, the VH-2 and the optional VL-2 correspond to the VH-C and the optional VL-C of FIGS. 24A-24B, and the VH-3 and the optional VL-3 correspond to the VH-B and the optional VL-B of FIGS. 24A-24B (i.e., ABU-1, ABU-2, and ABU-3 correspond to ABU-A, ABU-C, and ABU-B, respectively).

Embodiment C-12. The trispecific antigen binding protein of any one of Embodiments C-1 to C11, wherein:
(i) the ABU-1 is connected to the ABU-2 with a linker and/or to the ABU-3 with a linker;
(ii) the ABU-2 is connected to the ABU-1 with a linker and/or to the ABU-1 with a linker; and/or
(iii) the ABU-3 is connected to the ABU-1 with a linker and/or to the ABU-2 with a linker, further optionally wherein the linkers are individually selected from the following:
(a) a linker comprising about 1 to about 50 amino acids, about 1 to about 30 amino acids, about 1 to about 20 amino acids, about 1 to about 15 amino acids, about 1 to about 10 amino acids, about 1, 2, 3, 4, 5, 6, 7, 8, 9, or 10 amino acids, or about 5 or 10 amino acids;
(b) a linker comprising or consisting of glycine and/or serine;
(c) a linker comprising SEQ ID NO: 31 or two or more repeats thereof or SEQ ID NO: 38 (e.g., n=1, 2, 3, 4, 5, 6, 7, or 8); and/or
(d) a linker comprising a partial hinge, optionally a partial sequence of any of SEQ ID NOS: 61-64, optionally 37;
(e) a linker comprising any of SEQ ID NOS: 32-36 and 39-47;
(f) a linker comprising SEQ ID NO: 37 followed by SEQ ID NO: 31.

Embodiment C-13. The trispecific antigen binding protein of any one of Embodiments C-1 to C12, wherein:
(i) the ABU-1 comprises a Fab, a scFv, a Fab′, a F(ab′)₂, a Fv fragment, or a scFab;
(ii) the ABU-2 comprises a scFv, a Fab, a Fab′, a F(ab′)₂, a Fv fragment, or a scFab; and/or
(iii) the ABU-3 comprises a Fab, a scFv, a Fab′, a F(ab′)₂, a Fv fragment, or a scFab.

Embodiment C-14. The trispecific antigen binding protein of any one of Embodiments C-1 to C13, wherein:
(i) the ABU-1 comprises a Fab (Fab-1);
(ii) the ABU-2 comprises a scFv (scFv-2); and/or
(iii) the ABU-3 comprises a scFv (scFv-3),
   optionally wherein:
(i) the ABU-1 is connected to the ABU-2 with a linker and/or to the ABU-3 with a linker;
(ii) the ABU-2 is connected to the ABU-1 with a linker and/or to the ABU-1 with a linker; and/or
(iii) the ABU-3 is connected to the ABU-1 with a linker and/or to the ABU-2 with a linker.

Embodiment C-15. The trispecific antigen binding protein of Embodiment C14, which is according to Embodiment C-6, wherein:
(i) the Fab-1 comprises a first light chain comprising the VL-1 and a CL and a first heavy chain comprising the VH-1 and a CH1 (and optionally a partial hinge);
(ii) the scFv-2 comprises the VL-2, a linker, and the VH-2 in the direction from the N-terminus or the VH-2, a linker, and the VL-2 in the direction from the N-terminus; and/or
(iii) the scFv-3 comprises the VL-3, a linker, and the VH-3 in the direction from the N-terminus or the VH-3, a linker, and the VL-3 in the direction from the N-terminus,
   optionally wherein the linker in (ii) and the linker in (iii) are individually selected from the following:
(a) comprises about 1 to about 50 amino acids, about 3 to about 40 amino acids, about 5 to about 40 amino acids, about 10 to about 30 amino acids, about 15 to about 25 amino acids, about 17 to about 23 amino acids, about 18 to about 22 amino acids, about 19, preferably about 21, about 20, or 19 amino acids;
(b) comprises or consists of glycine and/or serine;
(c) comprises SEQ ID NO: 31 or two or more repeats thereof or SEQ ID NO: 38 (e.g., n=1, 2, 3, 4, 5, 6, 7, or 8); and/or
(d) comprises SEQ ID NO: 34, 35, 36, 33, or 32, or any of SEQ ID NOS: 39-47.

Embodiment C-16. The trispecific antigen binding protein of Embodiment C15, wherein:
(i) the N-terminus of the scFv-2 is connected to the C-terminus of the first light chain of the Fab-1 via a linker and the N-terminus of the scFv-3 is connected to the C-terminus of the first heavy chain of the Fab-1 via a linker; or
(ii) the C-terminus of the scFv-2 is connected to the N-terminus of the first light chain of the Fab-1 via a linker and the C-terminus of the scFv-3 is connected to the N-terminus of the first heavy chain of the Fab-1 via a linker,
   optionally wherein in (i) or (ii) the linkers are individually selected from the following:
(a) a linker comprising about 1 to about 10 amino acids, or about 5 or 10 amino acids;
(b) a linker comprising or consisting of glycine and/or serine;
(c) a linker comprising SEQ ID NO: 31 or two or more repeats thereof or SEQ ID NO: 38 (e.g., n=1, 2, 3, 4, 5, 6, 7, or 8); and/or
(d) a linker comprising a partial hinge, optionally 37;
(e) a linker comprising SEQ ID NO: 37 followed by SEQ ID NO: 31.

Embodiment C-17. The trispecific antigen binding protein of Embodiments C-15 or C-16, wherein:
(i) in the Fab-1, the first light chain comprising the VL-1 and a CL and the first heavy chain comprises the VH-1, a CH1, and a partial hinge (e.g., SEQ ID NO: 37);
(ii) the scFv-2 comprises the VL-2, a linker (e.g., SEQ ID NO: 34 or 35), and the VH-2 in the direction from the N-terminus; and/or
(iii) the scFv-3 comprises the VL-3, a linker (e.g., SEQ ID NO: 34 or 35), and the VH-3 in the direction from the N-terminus,
   wherein the N-terminus of the scFv-2 is connected to the C-terminus of the first light chain of the Fab-1 via a linker (e.g., SEQ ID NO: 31), and the N-terminus of the scFv-3 is connected to the C-terminus of the first heavy chain of the Fab-1 via a linker (e.g., SEQ ID NO: 31).

Embodiment C-18. The trispecific antigen binding protein of any one of Embodiments C-1 to C17, wherein:
the ABU-2 comprises a CD19 binding protein of any one of Embodiments B-1 to B-8; and/or
the ABU-3 comprises a BCMA binding protein of any one of Embodiments A-1 to A-8.

Embodiment C-19. The trispecific antigen binding protein of any one of Embodiments C-1 to C18, wherein the ABU-1 comprises:
(a) a first VL (VL-1) comprising a CDR-L1-1, a CDR-L2-1, and a CDR-L3-1; and
(b) a first VH (VH-1) comprising a CDR-H1-1, a CDR-H2-1, and a CDR-H3-1.
   and wherein:
(a) the CDR-L1, the CDR-L2, and the CDR-L3 comprise the light chain CDR1, 2, and 3 sequences, respectively, contained in SEQ ID NO: 25 or 94; and
(b) the CDR-H1, a CDR-H2, and a CDR-H3 comprise the heavy chain CDR1, 2, and 3 sequences, respectively, contained in SEQ ID NO: 26.

Embodiment C-20. The trispecific antigen binding protein of Embodiment C-19, wherein:
(a) the CDR-L1-1, the CDR-L2-1, and the CDR-L3-1 comprise the amino acid sequences of SEQ ID NOS: 19, 20, and 21, respectively; and
(b) the CDR-H1-1, the CDR-H2-1, and the CDR-H3-1 comprise the amino acid sequences of SEQ ID NOS: 23, 24, and 25, respectively.

Embodiment C-21. The trispecific antigen binding protein of Embodiment C-19 or C-20, wherein:
(A) the VL comprises an amino acid sequence having at least about 90%, 95%, 96%, 97%, 98%, 99% or 100% identity to SEQ ID NO: 25 or 94; and/or
(B) the VL comprises an amino acid sequence having at least about 90%, 95%, 96%, 97%, 98%, 99% or 100% identity to SEQ ID NO: 26.

Embodiment C-21. The trispecific antigen binding protein of any one of Embodiments C-1 to C20, which comprises a first polypeptide and a second polypeptide, wherein:
(I) (A) the first polypeptide comprises in the direction from the N-terminus to the C-terminus:
   (i) the VL-1, a CL, a linker, the VL-2, a linker, and the VH-2; or
   (ii) the VL-1, a CL, a linker, the VH-2, a linker, and the VL-2; and
   (B) the second polypeptide comprises in the direction from the N-terminus to the C-terminus:
   (i) the VH-1, a CH1, an optional partial hinge, a linker, the VL-3, a linker, and the VH-3; or
   (ii) the VH-1, a CH1, an optional partial hinge, a linker, the VH-3, a linker, and the VL-3; or
(II) (A) the first polypeptide comprises in the direction from the N-terminus to the C-terminus:
   (i) the VH-1, a CH1, an optional partial hinge, a linker, the VL-2, a linker, and the VH-2; or
   (ii) the VH-1, a CH1, an optional partial hinge, a linker, the VH-2, a linker, and the VL-2; and
   (B) the second polypeptide comprises in the direction from the N-terminus to the C-terminus:
   (i) the VL-1, a CL, a linker, the VL-3, a linker, and the VH-3; or
   (ii) the VL-1, a CL, a linker, the VH-3, a linker, and the VL-3.

Embodiment C-22. The trispecific antigen binding protein of any one of Embodiments C-1 to C20, which comprises a first polypeptide and a second polypeptide, wherein:
(A) the first polypeptide comprises in the direction from the N-terminus to the C-terminus:
   (i) the VL-1, a CL, a linker (e.g., SEQ ID NO: 31), the VL-2, a linker (e.g., SEQ ID NO: 34 or 35), and the VH-2; or
   (ii) the VL-1, a CL, a linker (e.g., SEQ ID NO: 31), the VH-2, a linker (e.g., SEQ ID NO: 34 or 35), and the VL-2; and
(B) the second polypeptide comprises in the direction from the N-terminus to the C-terminus:
   (i) the VH-1, a CH1, an optional partial hinge (e.g., SEQ ID NO: 37), a linker (e.g., SEQ ID NO: 31), the VL-3, a linker, and the VH-3; or
   (ii) the VH-1, a CH1, an optional partial hinge (e.g., SEQ ID NO: 37), a linker (e.g., SEQ ID NO: 31), the VH-3, a linker, and the VL-3.

Embodiment C-23. The trispecific antigen binding protein of Embodiment C22, wherein:
(i) the VL-1 and the VH-1 are according to Embodiments C-19 to C-21;
(ii) the VL-2 and the VH-2 are according to the VL and the VH of an CD19 binding protein thereof of any one of Embodiments B-1 to B-8; and/or
(iii) the VL-3 and the VH-3 are according to the VL and the VH of a BCMA binding protein of any one of Embodiments B-1 to B-8.

Embodiment C-24. The trispecific antigen binding protein of any one of Embodiments C-1 to C-23, comprising:
(I) (A) a first polypeptide comprises SEQ ID NO: 30, 109, 102, 103, 110 or an amino acid sequence having at least about 80%, 85%, 90%, 95%, 96%, 97%, 98%, 99% or 100% identity thereto; and
   (B) a second polypeptide comprises SEQ ID NO: 29 or 101 or an amino acid sequence having at least about 80%, 85%, 90%, 95%, 96%, 97%, 98%, 99% or 100% identity thereto; or
(II) (A) a first polypeptide comprises SEQ ID NO: 105 or 108 or an amino acid sequence having at least about 80%, 85%, 90%, 95%, 96%, 97%, 98%, 99% or 100% identity thereto; and
   (B) a second polypeptide comprises SEQ ID NO: 104, 111, 106, 107, 112 or an amino acid sequence having at least about 80%, 85%, 90%, 95%, 96%, 97%, 98%, 99% or 100% identity thereto;

Embodiment C-25. An isolated or recombinant nucleic acid encoding a trispecific antigen binding protein of any one of Embodiments C-1 through C-24, or a vector comprising the isolated or recombinant nucleic acid.

Embodiment C-26. A host cell comprising the isolated or recombinant nucleic acid or a vector of Embodiments C-25, or a cell population comprising such host cells.

Embodiment C-27. A pharmaceutical composition comprising:
(A) a trispecific antigen binding protein of any one of Embodiments C-1 through C-24, an isolated or recombinant nucleic or a vector of Embodiment C-25, and/or a host cell or a cell population of Embodiment C-26; and
(B) a pharmaceutically acceptable carrier.

Embodiment C-28. A method of producing a host cell of Embodiments C-26, comprising introducing an isolated or recombinant nucleic or a vector of Embodiments C-25 into a cell.

Embodiment C-29. A method of producing a trispecific antigen binding protein of any one of Embodiments C-1 through C-24, comprising culturing a host cell or a cell population of Embodiments C-26 in a condition which allows for expression of the trispecific antigen binding protein and optionally purifying trispecific antigen binding protein.

### Additional Embodiments D

Embodiment D-1. A method of preventing or treating a disease or depleting a target cell in a subject in need thereof, comprising administering to the subject an effective amount of:
(I) a trispecific antigen binding protein of any one of Embodiments C-1 through C-24, an isolated or recombinant nucleic or a vector of Embodiment C-25, a host cell or a cell population of Embodiment C-26 and/or a pharmaceutical composition of Embodiment C-27;
(II) a CD19 binding protein of any one of Embodiments B-1 through B-8, an isolated or recombinant nucleic or a vector of Embodiment B-9, a host cell or a cell population of Embodiment B-10 and/or a pharmaceutical composition of Embodiment B-11; and/or
(III) a BCMA binding protein of any one of Embodiments A-1 through A-8, an isolated or recombinant nucleic or a vector of Embodiments A-9, a host cell or a cell population of Embodiment A-10.

Embodiment D-2. The method of Embodiment D-1, wherein the disease comprise:
(a) a disease caused by, mediated by, or exacerbated by B cells or antibodies;
(b) an autoimmune disease;
(c) systemic lupus erythematosus (SLE), rheumatoid arthritis, Sjögren's syndrome, scleroderma, Hashimoto thyroiditis, Grave's disease, Type 1 diabetes, Addison disease, autoimmune hemolytic anemia, immune thrombodytopenic purpura (ITP), cold agglutinin disease, myasthenia gravis, multiple sclerosis, anti-NMDA receptor encephalitis, Guillain Barré syndrome, celiac disease, inflammatory bowel disease (Crohn's disease, ulcerative colitis), anti glomerular basement membrane (anti GBM) disease, anti-neutrophil cytoplasmic antibody (ANCA)-associated vasculitis, Goodpasture's syndrome, pemphigus vulgaris, psoriasis, autoimmune vitiligo (e.g., caused by antibodies specific to pigment-producing cells), pemphigoid, dermatitis herpetiformis, antiphospholipid syndrome, autoimmune hepatitis, stiff person syndrome, thyroid eye disease, or epidermolysis bullosa acquisita; anaphylaxis, allergy, asthma, urticaria; or overt inflammation; and/or
(d) cancer of B cell lineage;
(e) diffuse large B-cell Lymphoma (DLBCL), chronic lymphocytic leukemia (CLL), mantle cell lymphoma (MCL), follicular lymphoma, and Burkitt Lymphoma, small lymphocytic lymphoma (SLL), follicular lymphoma, marginal zone lymphomas, lymphoplasmacytic lymphoma (Waldenstrom macroglobulinemia), hairy cell leukemia, precursor B-cell acute lymphoblastic leukemia (B-ALL), lymphoblastic lymphoma (LBL), plasma cell neoplasms, primary central nervous system (CNS) lymphoma, primary cutaneous DLBCL, and/or nodular lymphocyte-predominant Hodgkin's lymphoma; and/or
(f) the target cell comprises a CD19+ cell, a BCMA+ cell, a CD19+BCMA+ cell, or a cell which causes, mediates, or exacerbate a disease, e.g., a disease listed in (b)-(e).

Embodiment D-3. The method of Embodiment D-1 or D-2, wherein:
(i) the subject is:
   (a) a mammal, optionally a human, a non-human primate, a monkey, a horse, a cow, sheep, a goat, a pig, a dog, a cat, a rabbit, a rodent, a hamster, a rat, or a mouse; or
   (b) a non-mammalian vertebrate, optionally a bird, fish, an amphibian, or a reptile;
(ii) the administering comprises:
   (a) parenterally administering, optionally by injection (optionally intravenous, subcutaneous, intramuscular, intradermal, intrathecal, intra-arterial, intraarticular, intraosseous, intraparenchymal, or intraperitoneal administration) or by inhalation;
   (b) locally administering, optionally by inhalation or administering to the liver, brain, bone marrow, lymphoid tissue, gut, ovary, testis, eye, ear, nose (optionally intranasally), skin (optionally transdermally or epicutaneously), mucosa, skin, or vagina; or
   (c) enterally administering, optionally orally, sublingually, buccally, or rectally;
(iii) the administering occurs about every 7 days, about weekly, about four times per month, about every 6-8 days, about three times per month, about every 5-10 days, about every 3-15 days, or about twice per month;
(iv) the effective amount comprises about 0.05 mg/kg to about 10 mg/kg, about 0.1 mg/kg to about 5 mg/kg, about 0.2 mg/kg to about 1 mg/kg, about 1 mg/kg, about 0.5 mg/kg to about 1 mg/kg, about 0.2 mg/kg to about 0.5 mg/kg, about 0.5 mg/kg, or about 0.2 mg/kg;
(v) the administering comprises:
   (v-1) subcutaneously administering, optionally about weekly, the trispecific antigen binding protein at about 0.1 mg/kg to about 5 mg/kg, optionally about 0.2 mg/kg to about 1 mg/kg, further optionally about 0.5 mg/kg to about 1 mg/kg (e.g., about 0.5 mg/kg as the first dose and about 1 mg/kg as the second dose); or
   (v-2) intravenously administering, optionally about weekly, the trispecific antigen binding protein at about 0.1 mg/kg to about 5 mg/kg, optionally about 0.2 mg/kg to about 1 mg/kg, further optionally about 0.2 mg/kg to about 0.5 mg/kg (e.g., about 0.2 mg/kg as the first dose and about 0.5 mg/kg as the second dose);
(vi) the administering comprises:
   (vi-1) subcutaneously administering the trispecific antigen binding protein at a dose which achieves (as a first dose) a highest serum concentration of the trispecific antigen binding protein (Cmax) of about 100 to about 5000 ng/ml, about 200 to about 2000 ng/mL, about 300 to about 1000 ng/mL, about 400 to about 800 ng/mL, or about 500 to about 700 ng/ml in the subject; or
   (vi-2) intravenously administering the trispecific antigen binding protein at a dose which achieves (as a first dose) a Cmax of about 100 to about 20000 ng/ml, about 200 to about 10000 ng/mL, about 500 to about 5000 ng/ml, about 1000 to about 4000 ng/mL, or about 2000 to about 3000 ng/ml in the subject; and/or
(vii) the method further comprises administering to the subject an additional agent.

Embodiment D-4. The method of any one of Embodiments D-1 through D-3, which:
(i) reduces the number and/or frequency of B cells, CD19+ cells, CD20+ cells, and/or CD19+CD20+ cells in the peripheral blood, spleen, lymph node(s), and/or bone marrow of the subject;
(ii) reduces the number and/or frequency of plasma cells, BCMA+ cells, long-lived plasma cells, and/or CD19-BCMA+ cells in the peripheral blood, spleen, lymph node(s), and/or bone marrow of the subject; and/or
(iii) does not significantly alter or only transiently alter (e.g., return to the baseline in about 1, 2, 3, 4, 5, 6, or 7 days) the number and/or frequency of T cells, CD3+ cells, CD3+CD4+ cells, and/or CD3+CD4− cells in the peripheral blood of the subject;
(iv) does not significantly activate or only transiently activate (e.g., return to the baseline in about 1, 2, 3, 4, 5, 6, or 7 days) T cells (e.g., activation defined based on CD25+ and/or CD69+) in the peripheral blood of the subject;
(v) does not significantly or only transiently reduce (e.g., return to the baseline in about 1, 2, 3, 4, 5, 6, or 7 days) the amount of an inflammatory cytokine(s) (e.g., IFN-γ, IL-2, IL-6, IL-8, IL-10, MCP-1, MIP-1β, TNF-α, or IL-1β) in the peripheral blood; and/or
(vi) reduces circulating antibody of the subject (e.g., IgGs, such autoantibodies, of the subject), optionally by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%.

Embodiment D-5. A trispecific antigen binding protein of any one of Embodiments C-1 through C-24, an isolated or recombinant nucleic or a vector of Embodiment C-25, a host cell or a cell population of Embodiment C-26 and/or a pharmaceutical composition of Embodiment C-27; a CD19 binding protein of any one of Embodiments B-1 through B-8, an isolated or recombinant nucleic or a vector of Embodiment B-9, a host cell or a cell population of Embodiment B-10 and/or a pharmaceutical composition of Embodiment B-11; and/or a BCMA binding protein of any one of Embodiments A-1 through A-8, an isolated or recombinant nucleic or a vector of Embodiments A-9, a host cell or a cell population of Embodiment A-10, which is for use in medicine, optionally for use in preventing or treating any of the diseases recited in Embodiment D-2.

Embodiment D-6. Use of a trispecific antigen binding protein of any one of Embodiments C-1 through C-24, an isolated or recombinant nucleic or a vector of Embodiment C-25, a host cell or a cell population of Embodiment C-26 and/or a pharmaceutical composition of Embodiment C-27; a CD19 binding protein of any one of Embodiments B-1 through B-8, an isolated or recombinant nucleic or a vector of Embodiment B-9, a host cell or a cell population of Embodiment B-10 and/or a pharmaceutical composition of Embodiment B-11; and/or a BCMA binding protein of any one of Embodiments A-1 through A-8, an isolated or recombinant nucleic or a vector of Embodiments A-9, a host cell or a cell population of Embodiment A-10, for therapy, optionally in preventing or treating a disease, optionally any of the diseases recited in Embodiment D-2.

Embodiment D-7. Use of a trispecific antigen binding protein of any one of Embodiments C-1 through C-24, an isolated or recombinant nucleic or a vector of Embodiment C-25, a host cell or a cell population of Embodiment C-26 and/or a pharmaceutical composition of Embodiment C-27; a CD19 binding protein of any one of Embodiments B-1 through B-8, an isolated or recombinant nucleic or a vector of Embodiment B-9, a host cell or a cell population of Embodiment B-10 and/or a pharmaceutical composition of Embodiment B-11; and/or a BCMA binding protein of any one of Embodiments A-1 through A-8, an isolated or recombinant nucleic or a vector of Embodiments A-9, a host cell or a cell population of Embodiment A-10, in the manufacture of a medicament, optionally wherein the medicament is for preventing or treating a disease, optionally any of the diseases recited in Embodiment D-2.

What is claimed is:

1. A multispecific antigen binding protein, comprising a first binding domain and further comprising at least one second antigen binding domain against a second antigen, wherein the first binding domain specifically binds to human BCMA and comprises:
(a) a variable light chain (VL) comprising a CDR-L1, a CDR-L2, and a CDR-L3, which comprise:
(i) the CDR-L1, CDR-L2, and CDR-L3 sequences contained in SEQ ID NO: 7; or
(ii) RASKSVSFLGINLIY (SEQ ID NO: 1), QASNRDT (SEQ ID NO: 2), and LQTPFFPRT (SEQ ID NO: 3), respectively; and
(b) a variable heavy chain (VH) comprising a CDR-H1, a CDR-H2, and a CDR-H3, which comprise:
(i) the CDR-H1, CDR-H2, and CDR-H3 sequences contained in SEQ ID NO: 8; or
(ii) DYSIN (SEQ ID NO: 4), WINTETGNPTYAQGFTG (SEQ ID NO: 5), and DKHLAMDY (SEQ ID NO: 6), respectively,
optionally wherein the CDR-L1, CDR-L2, and CDR-L3 sequences in (a)(i) and the CDR-H1, CDR-H2, and CDR-H3 sequences in (b)(i) are as defined by Kabat, Chothia, Contact, or IMGT;
optionally wherein:
(a) the VL comprises an amino acid sequence that is at least about 90%, 95%, 96%, 97%, 98%, 99% or 100% identical to the amino acid sequence of SEQ ID NO: 7; and
(b) the VH comprises an amino acid sequence that is at least about 90%, 95%, 96%, 97%, 98%, 99% or 100% identical to the amino acid sequence of SEQ ID NO: 8; and/or
optionally the first binding domain:
(I) is or comprises:
(i) a full-length immunoglobulin or an antibody fragment, optionally a scFv, a Fab, a Fab′, a F(ab′)₂, a Fv fragment, or a scFab; or
(ii) a scFv, comprising an amino acid sequence that is at least about 80%, 85%, 90%, 95%, 96%, 97%, 98%, 99% or 100% identical to the amino acid sequence of SEQ ID NO: 9; and/or
(II) has an affinity (K_{D}) between about 0.5 pM to about 0.5 nM to human BCMA; and/or
the multispecific antigen binding protein optionally is bispecific or trispecific; and
further optionally the second antigen binding domain comprises specificity for a cell surface protein expressed by an immune cell, optionally CD3, TCRα, TCRβ, CD16, NKG2D, CD89, CD64, or CD32, or is CD3; or a polynucleotide encoding any of the foregoing.

2-35. (canceled)

36. The multispecific antigen binding protein of claim 1, wherein the second antigen binding domain:
(I) is a CD3 binding domain, which is derived from huSP34, huOKT, or huUCHT1;
(II) comprises:
(a) a VL comprising a CDR-L1, a CDR-L2, and a CDR-L3, which comprise:
(i) the CDR-L1, CDR-L2, and CDR-L3 sequences contained in SEQ ID NO: 25; or
(ii) GSSTGAVTTSNYAN (SEQ ID NO: 19), GTNKRAP (SEQ ID NO: 20), and ALWYSNHWV (SEQ ID NO: 21), respectively; and
(b) a VH comprising a CDR-H1, a CDR-H2, and a CDR-H3, which comprise:
(i) the CDR-H1, CDR-H2, and CDR-H3 sequences contained in SEQ ID NO: 26; or
(ii) TYAMN (SEQ ID NO: 22), RIRSKYNNYATYYADSVKG (SEQ ID NO: 23), and HGNFGDSYVSWFAY (SEQ ID NO: 24), respectively,
optionally wherein the CDR-L1, CDR-L2, and CDR-L3 sequences in (a)(i) and the CDR-H1, CDR-H2, and CDR-H3 sequences in (b)(i) of the CD3 binding domain are as defined by Kabat, Chothia, Contact, or IMGT; and/or
(III) comprises:
(i) a VL comprising an amino acid sequence that is at least about 90%, 95%, 96%, 97%, 98%, 99% or 100% identical to the amino acid sequence of SEQ ID NO: 25; and
(ii) a VH comprising an amino acid sequence that is at least about 90%, 95%, 96%, 97%, 98%, 99% or 100% identical to the amino acid sequence of SEQ ID NO: 26; optionally wherein said CD3 binding domain is or comprises a Fab, comprising: a Fab heavy chain that is at least about 80%, 85%, 90%, 95%, 96%, 97%, 98%, 99% or 100% identical to the amino acid sequence of SEQ ID NO: 28; and a Fab light chain that is at least about 80%, 85%, 90%, 95%, 96%, 97%, 98%, 99% or 100% identical to the amino acid sequence of SEQ ID NO: 27, further optionally wherein said CD3 binding domain has an affinity (K_{D}) to human CD3 of about 1 nM to about 10 nM.

37. The multispecific antigen binding protein of claim 36, which comprises a third binding domain targeting CD19;
optionally wherein the third binding domain comprises a VL and VH wherein:
(I):
(a) the VL comprises a CDR-L1, a CDR-L2, and a CDR-L3, which comprise:
(i) the CDR-L1, CDR-L2, and CDR-L3 sequences contained in SEQ ID NO: 16; or
(ii) RASQSVDYDGDSYLN (SEQ ID NO: 10), DASNLVS (SEQ ID NO: 11), and QQSTEDPWT (SEQ ID NO: 12), respectively; and
(b) the VH comprises a CDR-H1, a CDR-H2, and a CDR-H3, which comprise:
(i) a CDR-H1, CDR-H2, and CDR-H3 sequences contained in SEQ ID NO17; or
(ii) SYWMN (SEQ ID NO: 13), QIWPGDGDTNYNGKFKG (SEQ ID NO: 14), and RETTTVGRYYYAMDY (SEQ ID NO: 15), respectively,
optionally wherein the CDR-L1, CDR-L2, and CDR-L3 sequences in (a)(i) and the CDR-H1, CDR-H2, and CDR-H3 sequences in (b)(i) of the CD19 binding domain are as defined by Kabat, Chothia, Contact, or IMGT; or
(II)
(a) the VL comprises an amino acid sequence that is at least about 90%, 95%, 96%, 97%, 98%, 99% or 100% identical to the amino acid sequence of SEQ ID NO: 16; and
(b) the VH comprises an amino acid sequence that is at least about 90%, 95%, 96%, 97%, 98%, 99% or 100% identical to the amino acid sequence of SEQ ID NO: 17; and/or
(III) the CD19 binding domain comprises a scFv which is at least about 80%, 85%, 90%, 95%, 96%, 97%, 98%, 99% or 100% identical to the amino acid sequence of SEQ ID NO: 18, further optionally wherein, in (III), said third binding domain has an affinity (K_{D}) to human CD19 of about 0.01 to about 5 nM.

38. The multispecific antigen binding protein of claim 37 which is a trispecific antigen binding protein, wherein:
(i) the first binding domain specifically binds to human BCMA;
(ii) the second antigen binding domain specifically binds to human CD3; and
(iii) the third binding domain specifically binds to human CD19;
optionally wherein said trispecific antigen binding protein is a Fab-(scFv)₂, a Tri-MAB, a CODV-Ig, an OrthoTsAb, a 2+1 common LC, a Triplebody, a Triflex, a MATCH3, or a triabody, in particular a Fab-(scFv)2; or optionally is a Fab-(scFv)₂, optionally wherein:
(i) the first binding domain is a scFv;
(ii) the second antigen binding domain is a Fab; and
(iii) the third binding domain is a scFv.

39. The multispecific antigen binding protein of claim 38, which is a Fab-(scFv)₂, wherein:
(i) the first binding domain is a scFv;
(ii) the second antigen binding domain is a Fab; and
(iii) the third binding domain is a scFv;
wherein:
in (i), the scFv which specifically binds to human BCMA optionally comprises:
(a) a variable light chain (VL) comprising a CDR-L1, a CDR-L2, and a CDR-L3, which comprise:
(i) the CDR-L1, CDR-L2, and CDR-L3 sequences contained in SEQ ID NO: 7; or
(ii) RASKSVSFLGINLIY (SEQ ID NO: 1), QASNRDT (SEQ ID NO: 2), and LQTPFFPRT (SEQ ID NO: 3), respectively; and
(b) a variable heavy chain (VH) comprising a CDR-H1, a CDR-H2, and a CDR-H3, which comprise:
(i) the CDR-H1, CDR-H2, and CDR-H3 sequences contained in SEQ ID NO: 8; or
(ii) DYSIN (SEQ ID NO: 4), WINTETGNPTYAQGFTG (SEQ ID NO: 5), and DKHLAMDY (SEQ ID NO: 6), respectively, or
(a) the VL optionally comprises an amino acid sequence that is at least about 90%, 95%, 96%, 97%, 98%, 99% or 100% identical to the amino acid sequence of SEQ ID NO: 7; and/or
(b) the VH optionally comprises an amino acid sequence that is at least about 90%, 95%, 96%, 97%, 98%, 99% or 100% identical to the amino acid sequence of SEQ ID NO: 8, or
optionally the scFv is at least about 80%, 85%, 90%, 95%, 96%, 97%, 98%, 99% or 100% identical to the amino acid sequence of SEQ ID NO: 9, 83, 84, 85, 86, or 87;
in (ii) the Fab molecule optionally comprises a CD3 binding domain, which is derived from huSP34, huOKT, or huUCHT1; which optionally comprises:
(a) a VL comprising a CDR-L1, a CDR-L2, and a CDR-L3, which comprise:
(i) the CDR-L1, CDR-L2, and CDR-L3 sequences contained in SEQ ID NO: 25; or
(ii) GSSTGAVTTSNYAN (SEQ ID NO: 19), GTNKRAP (SEQ ID NO: 20), and ALWYSNHWV (SEQ ID NO: 21), respectively; and
further comprises a CL domain;
(b) a VH comprising a CDR-H1, a CDR-H2, and a CDR-H3, which optionally comprise:
(i) the CDR-H1, CDR-H2, and CDR-H3 sequences contained in SEQ ID NO: 26; or
(ii) TYAMN (SEQ ID NO: 22), RIRSKYNNYATYYADSVKG (SEQ ID NO: 23), and HGNFGDSYVSWFAY (SEQ ID NO: 24), respectively, and
Further comprises a CH1 domain, optionally comprising EPKSC (SEQ ID NO: 37);
further optionally wherein the CDR-L1, CDR-L2, and CDR-L3 sequences in (a)(i) and the CDR-H1, CDR-H2, and CDR-H3 sequences in (b)(i) of the CD3 binding domain are as defined by Kabat, Chothia, Contact, or IMGT; and/or
optionally comprises:
(i) a VL comprising an amino acid sequence that is at least about 90%, 95%, 96%, 97%, 98%, 99% or 100% identical to the amino acid sequence of SEQ ID NO: 25; and a CL domain;
(ii) a VH comprising an amino acid sequence that is at least about 90%, 95%, 96%, 97%, 98%, 99% or 100% identical to the amino acid sequence of SEQ ID NO: 26 and a CH1 domain, optionally further comprising EPKSC (SEQ ID NO: 37) and a CL domain;
optionally wherein the heavy chain is at least about 80%, 85%, 90%, 95%, 96%, 97%, 98%, 99% or 100% identical to the amino acid sequence of SEQ ID NO: 28, and the light chain is at least about 80%, 85%, 90%, 95%, 96%, 97%, 98%, 99% or 100% identical to the amino acid sequence of SEQ ID NO: 27; and
in (iii) the scFv which binds to CD19
(I) optionally comprises:
(a) a VL comprising a CDR-L1, a CDR-L2, and a CDR-L3, which comprise:
(i) the CDR-L1, CDR-L2, and CDR-L3 sequences contained in SEQ ID NO: 16; or
(ii) RASQSVDYDGDSYLN (SEQ ID NO: 10), DASNLVS (SEQ ID NO: 11), and QQSTEDPWT (SEQ ID NO: 12), respectively; and
(b) a VH comprising a CDR-H1, a CDR-H2, and a CDR-H3, which comprise:
(i) a CDR-H1, CDR-H2, and CDR-H3 sequences contained in SEQ ID NO17; or
(ii) SYWMN (SEQ ID NO: 13), QIWPGDGDTNYNGKFKG (SEQ ID NO: 14), and RETTTVGRYYYAMDY (SEQ ID NO: 15), respectively,
optionally wherein the CDR-L1, CDR-L2, and CDR-L3 sequences in (a)(i) and the CDR-H1, CDR-H2, and CDR-H3 sequences in (b)(i) of the CD19 binding domain are as defined by Kabat, Chothia, Contact, or IMGT; or
(II) optionally comprises:
(a) the VL comprises an amino acid sequence that is at least about 90%, 95%, 96%, 97%, 98%, 99% or 100% identical to the amino acid sequence of SEQ ID NO: 16; and
(b) the VH comprises an amino acid sequence that is at least about 90%, 95%, 96%, 97%, 98%, 99% or 100% identical to the amino acid sequence of SEQ ID NO: 17; and/or
(III) optionally is or comprises a scFv which is at least about 80%, 85%, 90%, 95%, 96%, 97%, 98%, 99% or 100% identical to the amino acid sequence of SEQ ID NO: 18, further optionally wherein said third binding domain has an affinity (K_{D}) to human CD19 of about 0.01 to about 5 nM; and/or
optionally the scFv is at least about 80%, 85%, 90%, 95%, 96%, 97%, 98%, 99% or 100% identical to the amino acid sequence of SEQ ID NO: 18, 89, 90, 91, 92, or 93; and
further optionally the first binding domain and the third binding domain are fused to the second antigen binding domain via a peptide linker, respectively at the C-terminus of the heavy chain and the light chain, optionally GGGGS (SEQ ID NO: 31).

40. The multispecific antigen binding protein of claim 38, which comprises one or more of the following features:
(I) comprises:
(a) an amino acid sequence that is at least about 80%, 85%, 90%, 95%, 96%, 97%, 98%, 99% or 100% identical to the amino acid sequence of SEQ ID NO: 29; and
(b) an amino acid sequence that is at least about 80%, 85%, 90%, 95%, 96%, 97%, 98%, 99% or 100% identical to the amino acid sequence of SEQ ID NO: 30;
(II) glutamine (Q) or glutamate (E) at position 1 of the light chain and/or heavy chain is replaced by pyroglutamate (pE), optionally wherein the light chain comprises a pyroglutamate (pE) at position 1 of the light chain instead of glutamine (Q);
(III) consists of:
(a) the amino acid sequence of SEQ ID NO: 29; and/or
(b) the amino acid sequence of SEQ ID NO: 30;
(IV) (a) the affinity of the third binding domain for CD19 is at least about 20 times, about 50 times, or at least about 100 times lower than the affinity of the first binding domain for BCMA; and/or
(b) the affinity of the second antigen binding domain for CD3 is at least about 20 times, 50 times, 100 times, or at least about 150 times lower than the affinity of the first binding domain for BCMA; and/or
(V) comprises:
(i) an affinity (K_{D}) between about 0.5 pM to about 0.5 nM to human BCMA;
(ii) an affinity (K_{D}) between about 0.01 nM to about 5 nM to human CD19; and/or
(iii) an affinity (K_{D}) between about 1 nM to about 10 nM to human CD3;
(VI) is chemically or biologically modified, optionally glycosylated, PEGylated, PASylated, XTENylated or HESylated;
(VII) comprises a light chain and/or a heavy chain comprising an N-terminal and/or C-terminal truncation of 1, 2, 3, 4 or 5 amino acids;
(VIII) is linked to or combined with a functional entity such as a detectable label, a therapeutic agent or a PK modifying moiety; and/or
(IX) further comprises one or more additional binding domains.

41. An isolated polynucleotide encoding the multispecific antigen binding protein of claim 1, or a vector comprising the polynucleotide, optionally an expression vector or a viral vector, or a host cell comprising the isolated polynucleotide or the vector.

42. An isolated polynucleotide encoding the trispecific antigen binding protein of claim 37, or a vector comprising the polynucleotide, optionally an expression vector or a viral vector, or a host cell comprising the isolated polynucleotide or the vector.

43. An isolated polynucleotide encoding the trispecific antigen binding protein of claim 38, or a vector comprising the polynucleotide, optionally an expression vector or a viral vector, or a host cell comprising the isolated polynucleotide or the vector.

44. An isolated polynucleotide encoding the trispecific antigen binding protein of claim 39, or a vector comprising the polynucleotide, optionally an expression vector or a viral vector, or a host cell comprising the isolated polynucleotide or the vector.

45. A pharmaceutical composition comprising one or more of:
(i) the trispecific antigen binding protein of claim 37, or
(ii) a polynucleotide encoding the trispecific antigen binding protein of claim 37; or
(iii) a vector comprising the polynucleotide of (ii), or
(iv) a host cell comprising the polynucleotide of (ii) or the vector of (iii); and a pharmaceutically acceptable carrier.

46. A pharmaceutical composition comprising one or more of:
(i) the trispecific antigen binding protein of claim 38, or
(ii) a polynucleotide encoding the trispecific antigen binding protein of claim 38; or
(iii) a vector comprising the polynucleotide of (ii), or
(iv) a host cell comprising the polynucleotide of (ii) or the vector of (iii); and a pharmaceutically acceptable carrier.

47. A pharmaceutical composition comprising one or more of:
(i) the trispecific antigen binding protein of claim 39, or
(ii) a polynucleotide encoding the trispecific antigen binding protein of claim 39; or
(iii) a vector comprising the polynucleotide of (ii), or
(iv) a host cell comprising the polynucleotide of (ii) or the vector of (iii); and a pharmaceutically acceptable carrier.

48. An antigen binding protein that specifically binds to human BCMA, comprising:
(a) a VL comprising a CDR-L1, a CDR-L2, and a CDR-L3, which comprise:
(i) the CDR-L1, CDR-L2, and CDR-L3 sequences contained in SEQ ID NO: 7; or
(ii) RASKSVSFLGINLIY (SEQ ID NO: 1), QASNRDT (SEQ ID NO: 2), and LQTPFFPRT (SEQ ID NO: 3), respectively; and
(b) a VH comprising a CDR-H1, a CDR-H2, and a CDR-H3, which comprise:
(i) the CDR-H1, CDR-H2, and CDR-H3 sequences contained in SEQ ID NO: 8; or
(ii) DYSIN (SEQ ID NO: 4), WINTETGNPTYAQGFTG (SEQ ID NO: 5), and DKHLAMDY (SEQ ID NO: 6), respectively,
optionally wherein the CDR-L1, CDR-L2, and CDR-L3 sequences in (a) (i) and the CDR-H1, CDR-H2, and CDR-H3 sequences in (b) (i) are as defined by Kabat, Chothia, Contact, or IMGT;
further optionally comprising one or more of the following features:
(I)
(a) the VL comprises an amino acid sequence that is at least about 90%, 95%, 96%, 97%, 98%, 99% or 100% identical to the amino acid sequence of SEQ ID NO: 7; and/or
(b) the VH comprises an amino acid sequence that is at least about 90%, 95%, 96%, 97%, 98%, 99% or 100% identical to the amino acid sequence of SEQ ID NO: 8;
(II) has an affinity (K_{D}) between about 0.5 pM to about 0.5 nM to human BCMA;
(III) is an antibody, such as a full-length immunoglobulin or an antibody fragment, optionally a scFv, a Fab, a Fab′, a F(ab′)₂, a Fv fragment, or a scFab;
(IV) is or comprises a scFv with an amino acid sequence that is at least about 80%, 85%, 90%, 95%, 96%, 97%, 98% or 100% identical to the amino acid sequence of SEQ ID NO: 9;
(V) is chemically or biologically modified, optionally glycosylated, PEGylated, PASylated, XTENylated or HESylated;
(VI) comprises a light chain and/or a heavy chain comprising an N-terminal and/or C-terminal truncation of 1, 2, 3, 4 or 5 amino acids;
(VII) is linked to or combined with a functional entity such as a detectable label, a therapeutic agent or a PK modifying moiety.

49. A chimeric antigen receptor (CAR) comprising the antigen binding protein of claim 48, or an immune cell expressing the CAR, optionally a T cell; or an antibody drug conjugate (ADC) comprising the antigen binding protein of claim 48.

50. An antigen binding protein that specifically binds to human CD19, comprising:
(a) a VL comprising a CDR-L1, a CDR-L2, and a CDR-L3, which comprise:
(i) the CDR-L1, CDR-L2, and CDR-L3 sequences contained in SEQ ID NO: 16; or
(ii) RASQSVDYDGDSYLN (SEQ ID NO: 10), DASNLVS (SEQ ID NO: 11), and QQSTEDPWT (SEQ ID NO: 12), respectively; and
(b) a VH comprising a CDR-H1, a CDR-H2, and a CDR-H3, which comprise:
(i) the CDR-H1, CDR-H2, and CDR-H3 sequences contained in SEQ ID NO17; or
(ii) SYWMN (SEQ ID NO: 13), QIWPGDGDTNYNGKFKG (SEQ ID NO: 14), and RETTTVGRYYYAMDY (SEQ ID NO: 15), respectively,
optionally wherein the CDR-L1, CDR-L2, and CDR-L3 sequences in (a) (i) and the CDR-H1, CDR-H2, and CDR-H3 sequences in (b) (i) of the CD19 binding domain are as defined by Kabat, Chothia, Contact, or IMGT;
further optionally comprising one or more of the following features:
(I) (a) the VL comprises an amino acid sequence that is at least about 90%, 95%, 96%, 97%, 98%, 99% or 100% identical to the amino acid sequence of SEQ ID NO: 16; and/or
(b) the VH comprises an amino acid sequence that is at least about 90%, 95%, 96%, 97%, 98%, 99% or 100% identical to the amino acid sequence of SEQ ID NO: 17,
optionally wherein the antigen binding protein is or comprises a scFv having at least about 80%, 85%, 90%, 95%, 96%, 97%, 98%, 99% or 100% identical to the amino acid sequence of SEQ ID NO: 18;
(II) has an affinity (K_{D}) to human CD19 of about 0.01 to about 5 nM;
(III) the antigen binding protein is an antibody, such as a full-length immunoglobulin or an antibody fragment, optionally a scFv, a Fab, a Fab′, a F(ab′)₂, a Fv fragment or a scFab;
(IV) is chemically or biologically modified, optionally glycosylated, PEGylated, PASylated, XTENylated or HESylated;
(V) comprises a light chain and/or a heavy chain comprising an N-terminal and/or C-terminal truncation of 1, 2, 3, 4 or 5 amino acids;
(VI) is linked to or combined with a functional entity such as a detectable label, a therapeutic agent or a PK modifying moiety.

51. A chimeric antigen receptor (CAR) comprising the antigen binding protein of claim 50, or an immune cell expressing the CAR, optionally a T cell; or an antibody drug conjugate (ADC) comprising the antigen binding protein of claim 50.

52. A multispecific binding protein comprising:
(i) a first binding domain, being the CD19 antigen binding protein of claim 50; and
(ii) further comprising at least one second antigen binding domain against a second antigen;
optionally comprising one or more of the following features:
(I) is bispecific or trispecific;
(II) comprises an immune cell engager, such as a CD3, TCRα, TCRβ, CD16, NKG2D, CD89, CD64, or a CD32 binding domain, preferably a CD3 binding domain, optionally wherein:
(1) said immune cell engager is a CD3 binding domain derived from huSP34, huOKT, or huUCHT1;
(2) said CD3 binding domain is or comprises:
(a) a VL comprising a CDR-L1, a CDR-L2, and a CDR-L3, which comprise:
(i) the CDR-L1, CDR-L2, and CDR-L3 sequences contained in SEQ ID NO: 25; or
(ii) GSSTGAVTTSNYAN (SEQ ID NO: 19), GTNKRAP (SEQ ID NO: 20), and ALWYSNHWV (SEQ ID NO: 21), respectively; and
(b) a VH comprising a CDR-H1, a CDR-H2, and a CDR-H3, which comprise:
(i) the CDR-H1, CDR-H2, and CDR-H3 sequences contained in SEQ ID NO: 26; or
(ii) TYAMN (SEQ ID NO: 22), RIRSKYNNYATYYADSVKG (SEQ ID NO: 23), and HGNFGDSYVSWFAY (SEQ ID NO: 24), respectively,
optionally wherein the CDR-L1, CDR-L2, and CDR-L3 sequences in (a) (i) and the CDR-H1, CDR-H2, and CDR-H3 sequences in (b) (i) of the CD3 binding domain are as defined by Kabat, Chothia, Contact, or IMGT;
(3) is or comprises:
(a) a VL comprising an amino acid sequence that is at least about 90%, 95%, 96%, 97%, 98%, 99% or 100% identical to the amino acid sequence of SEQ ID NO: 25; and/or
(b) a VH comprising an amino acid sequence that is at least about 90%, 95%, 96%, 97%, 98%, 99% or 100% identical to the amino acid sequence of SEQ ID NO: 26; and/or
(4) is or comprises a Fab comprising a Fab heavy chain that is at least about 80%, 85%, 90%, 95%, 96%, 97%, 98%, 99% or 100% identical to the amino acid sequence of SEQ ID NO: 28, and/or a Fab light chain that is at least about 80%, 85%, 90%, 95%, 96%, 97%, 98%, 99% or 100% identical to the amino acid sequence of SEQ ID NO: 27;
(5) in (3) or (4), has an affinity (K_{D}) to human CD3 of about 1 nM to about 10 nM;
(III) optionally further comprises a BCMA binding domain.

53. An isolated polynucleotide encoding (i) the antigen binding protein of claim 48 or a multispecific binding protein comprising the antigen binding protein of claim 48, (ii) a CAR comprising the antigen binding protein of claim 48, or (iii) an ADC comprising the antigen binding protein of claim 48; or a vector comprising the polynucleotide, optionally an expression vector or a viral vector; or a host cell comprising the isolated polynucleotide or the vector.

54. An isolated polynucleotide encoding (i) the antigen binding protein of claim 50 or a multispecific binding protein comprising the antigen binding protein of claim 50, (ii) a CAR comprising the antigen binding protein of claim 50, or (iii) an ADC comprising the antigen binding protein of claim 50; or a vector comprising the polynucleotide, optionally an expression vector or a viral vector; or a host cell comprising the isolated polynucleotide or the vector.

55. A method of producing the multispecific antigen binding protein of claim 1, comprising the steps of:
(a) culturing a host cell comprising an isolated polynucleotide encoding the multispecific binding protein or a vector comprising the polynucleotide in particular an expression vector or a viral vector, under conditions suitable for the expression of the protein; and optionally
(b) recovering the protein.

56. A pharmaceutical composition comprising (i) the multispecific binding protein of claim 1 or (ii) a polynucleotide or vector encoding for said multispecific binding protein or (iii) a cell comprising said polynucleotide or vector and a pharmaceutically acceptable carrier.

57. A method the treatment of a disease, optionally wherein the disease is a B cell-related disease, optionally an autoimmune disease or cancer, further optionally a B cell malignancy, comprising the administration of (i) the antigen binding protein of claim 48 or a multispecific binding protein comprising the antigen binding protein of claim 48, or (ii) a CAR comprising the antigen binding protein of claim 48, or (iii) an ADC comprising the antigen binding protein of claim 48, or (iv) a polynucleotide encoding (i), (ii) or (iii), or (v) a vector comprising the polynucleotide of (iv), or (vi) a host cell comprising the polynucleotide of (iv) or the vector of (v) to a subject in need thereof.

58. A method the treatment of a disease, optionally wherein the disease is a B cell-related disease, further optionally an autoimmune disease or cancer, further optionally a B cell malignancy, comprising the administration of the trispecific antigen binding protein of claim 37 to a subject in need thereof.

59. A method the treatment of a disease, optionally wherein the disease is a B cell-related disease, further optionally an autoimmune disease or cancer, further optionally a B cell malignancy, comprising the administration of the trispecific antigen binding protein of claim 38 to a subject in need thereof.

60. A method the treatment of a disease, optionally wherein the disease is a B cell-related disease, further optionally an autoimmune disease or cancer, further optionally a B cell malignancy, comprising the administration of the trispecific antigen binding protein of claim 39 to a subject in need thereof.
